(19) United States
(12) Patent Application Publication
(10) Pub. No.: US 2023/0174977 A1
(43) Pub. Date: Jun. 8, 2023

(54) ENGINEERED CIRCULAR POLYNUCLEOTIDES

(71) Applicant: Shape Therapeutics Inc., Seattle, WA (US)

(72) Inventors: Thomas PACKARD, Seattle, WA (US); Yiannis SAVVA, Seattle, WA (US); Roberto PERALES, Seattle, WA (US); Richard SULLIVAN, Seattle, WA (US)

(21) Appl. No.: 17/998,322

(22) PCT Filed: May 26, 2021
(86) PCT No.: PCT/US2021/034301

§ 371 Date: Nov. 9, 2022

Related U.S. Application Data
(60) Provisional application No. 63/178,056, filed on Apr. 22, 2021.
(60) Provisional application No. 63/119,902, filed on Dec. 1, 2020.
(60) Provisional application No. 63/112,488, filed on Nov. 11, 2020.
(60) Provisional application No. 63/029,996, filed on May 26, 2020.

(51) Int. Cl. C12N 15/11; C12N 15/86

(52) U.S. Cl. CPC C12N 15/11; C12N 15/86; C12N 2310/532; C12N 2740/15043; C12N 2310/11

(57) ABSTRACT

Disclosed herein are engineered guide RNAs, engineered polynucleotides, precursor engineered polynucleotide, vectors omprising engineered polynucleotide, nucleic acids of engineered polynucleotide, pharmaceutical compositions thereof, methods of making the engineered polynucleotides and methods of treating or preventing a disease or condition by administering above described thereof.

### CROSS REFERENCE TO RELATED APPLICATIONS

This application claims priority under 35 U.S.C. § 119 from Provisional Application Ser. No. 63/029,996, filed May 26, 2020, Provisional Application Ser. No. 63/112,488, filed Nov. 11, 2020, Provisional Application Ser. No. 63/119,902, filed Dec. 1, 2020, and Provisional Application Ser. No. 63/178,056, filed Apr. 22, 2021, the disclosures of which are incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Disclosed herein are precursor engineered linear polynucleotides which comprise: a first spacer domain; a targeting domain that is substantially complementary to a target RNA implicated in a disease or condition; and a second spacer domain, wherein the first or second spacer domain is not substantially complementary to the target RNA, wherein a transcript of the precursor engineered linear polynucleotide circularizes upon insertion of the precursor engineered linear polynucleotide in a mammalian cell, thereby forming a circularized engineered polynucleotide; and wherein hybridization of the targeting domain with the target RNA facilitates an edit of a base of a nucleotide of the target RNA by an RNA editing enzyme. In some embodiments, the precursor engineered linear polynucleotide can comprise, in order of 5′ to 3′: a first spacer domain, a targeting domain, and a second spacer domain. In some embodiments, the precursor engineered linear polynucleotide can comprise a ribozyme domain 5′ to the first spacer domain or 3′ to the second spacer domain. In some embodiments, the precursor engineered linear polynucleotide can comprise a ligation domain between the ribozyme domain and the first spacer domain or between the ribozyme domain and the second spacer domain. In some embodiments, after self-circularization, the first spacer domain and the second spacer domain be a filler sequence that can be from about 40% to about 70% of a total length of the circularized engineered polynucleotide. In some embodiments, after self-circularization, the first spacer domain and the second spacer domain can be a filler sequence that can be from about 50% to about 67% of the total sequence of the circularized engineered polynucleotide. In some embodiments, the filler sequence can increase hybridization of the targeting domain to the target RNA, relative to an otherwise comparable circularized polynucleotide that lacks the filler sequence. In some embodiments, after self-circularization the total length of the circularized engineered polynucleotide can comprise about 150 nucleotides to about 400 nucleotides. In some embodiments, after self-circularization the total length of the circularized engineered polynucleotide can comprise about 200 nucleotides to about 300 nucleotides. In some embodiments, the targeting domain can comprise at least about 80%, at least about 85%, at least about 90%, at least about 92%, at least about 95%, at least about 97%, at least about 99%, or 100% complementarity to the target RNA. In some embodiments, the target RNA can be an RNA selected from the group consisting of a pre-messenger RNA, a messenger RNA, a ribosomal RNA, a transfer RNA, a long non-coding RNA, and a small RNA. In some embodiments, a targeting domain can be substantially complementary to a 3′ or 5′ untranslated region (UTR) of the target RNA. In some embodiments, a targeting domain can be substantially complementary to an intronic region of the target RNA. In some embodiments, a targeting domain can be substantially complementary to a translation initiation site (TIS). In some embodiments, a targeting domain can be substantially complementary to an upstream open reading frame (uORF) of the target RNA. In some embodiments, a targeting domain can comprise at least a single nucleotide that can be mismatched to the target RNA. In some embodiments, an edit of the base can increase a level of a protein or fragment thereof, can increase a length of a protein or fragment thereof, can increase a functionality of a protein or fragment thereof, can increase a stability of a protein or fragment thereof, or any combination thereof, after translation of the target RNA with the edit of the base, relative to a translated protein of an otherwise comparable target RNA lacking the edit. In some embodiments, an edit of a base can convert a sense codon into a stop codon. In some embodiments, a sense codon can be implicated in a disease pathogenic pathway. In some embodiments, converting the sense codon to the stop codon can reduce the disease pathogenic pathway. In some embodiments, an edit of a base can convert a stop codon into a sense codon. In some embodiments, a stop codon can be implicated in a disease pathogenic pathway. In some embodiments, converting the stop codon to the sense codon can reduce the disease pathogenic pathway. In some embodiments, an edit of a base converts a first sense codon into a second sense codon. In some embodiments, a first sense codon can be implicated in a disease pathogenic pathway. In some embodiments, converting the first sense codon to the second sense codon can reduce the disease pathogenic pathway. In some embodiments, a targeting domain can be from about 20 nucleotides to about 150 nucleotides; or from about 100 nucleotides to about 200 nucleotides. In some embodiments, an RNA editing enzyme can comprise an ADAR protein or an APOBEC protein. In some embodiments, an RNA editing enzyme can comprise ADAR and ADAR can be ADAR1. In some embodiments, an RNA editing enzyme can comprise ADAR and ADAR can be ADAR2. In some embodiments, the RNA editing enzyme can comprise ADAR and ADAR can be ADAR3. In some embodiments, a targeting domain may not comprise an aptamer. In some embodiments, the disease or condition can comprise Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, Hypomyelination with Atrophy of Basal Ganglia and Cerebellum (H-ABC) or cystic fibrosis. In some embodiments, the target RNA can comprise TUBB4A, and TUBB4A can comprise a D249N mutation. In some embodiments, a targeting domain can comprise at least about 80%, at least about 85%, at least about 90%, at least about 92%, at least about 95%, at least about 97%, at least about 99%, or 100% sequence homology to SEQ ID NO: 32, SEQ ID NO: 33, or SEQ ID NO: 34. In some embodiments, a target RNA can comprise LRRK2 and LRRK2 can comprise a G2019S mutation. In some embodiments, a targeting domain can comprise at least about 80%, at least about 85%, at least about 90%, at least about 92%, at least about 95%, at least about 97%, at least about 99%, or 100% sequence homology to SEQ ID NO: 39, SEQ ID NO: 53, or SEQ ID NO: 54. In some embodiments, a target RNA can comprise SERPINA1 and SERPINA1 can comprise a E342K mutation. In some embodiments, a targeting domain can comprise at least about 80%, at least about 85%, at least about 90%, at least about 92%, at least about 95%, at least about 97%, at least about 99%, or 100% sequence homology to SEQ ID NO: 64, or SEQ ID NO: 65. In some embodiments, a target RNA can comprise SNCA. In some embodiments, a targeting domain an comprise at least about 80%, at least about 85%, at least about 90%, at least about 92%, at least about 95%, at least about 97%, at least about 99%, or 100% sequence homology to SEQ ID NO: 40, SEQ ID NO: 41, SEQ ID NO: 42, SEQ ID NO: 43, SEQ ID NO: 44. In some embodiments, a targeting domain can be designed to target the translation initiation site (TIS). In some embodiments, a target RNA can comprise APP. In some embodiments, a targeting domain can comprise at least about 80%, at least about 85%, at least about 90%, at least about 92%, at least about 95%, at least about 97%, at least about 99%, or 100% sequence homology to SEQ ID NO: 61, SEQ ID NO: 62, or SEQ ID NO: 63. In some embodiments, a target RNA can comprise ABCA4. In some embodiments, a targeting domain can comprise at least about 80%, at least about 85%, at least about 90%, at least about 92%, at least about 95%, at least about 97%, at least about 99%, or 100% sequence homology to SEQ ID NO: 58, SEQ ID NO: 59, or SEQ ID NO: 60. In some embodiments, a the target RNA can comprise DMD. In some embodiments, a targeting domain can comprise at least about 80%, at least about 85%, at least about 90%, at least about 92%, at least about 95%, at least about 97%, at least about 99%, or 100% sequence homology to SEQ ID NO: 56, or SEQ ID NO: 57.

Also disclosed herein, are precursor engineered polynucleotides which comprise, in order of 5′ to 3′: a first ribozyme domain; a first ligation domain; a first spacer domain; a targeting domain that is substantially complementary to a target RNA implicated in a disease or condition, a second ligation domain, and a second ribozyme domain. In some embodiments, the first spacer domain may not be substantially complementary to the target RNA. In some embodiments, a transcript of the precursor engineered linear polynucleotide can circularize upon insertion of the precursor engineered linear polynucleotide in a mammalian cell, thereby forming a circularized engineered polynucleotide. In some embodiments, hybridization of the targeting domain with the target RNA can facilitate an edit of a base of a nucleotide of the target RNA by an RNA editing enzyme.

Also disclosed herein are engineered circular polynucleotides comprising: a targeting domain that is substantially complementary to a target RNA implicated in a disease or condition, and a spacer domain that is not substantially complementary to the target RNA, wherein the spacer domain enlarges the engineered circular polynucleotide by the addition of one or more nucleotides, wherein hybridization of the targeting domain with the target RNA facilitates an edit of a base of a nucleotide of the target RNA by an RNA editing enzyme. In some embodiments, an edit of the base of a nucleotide of the target RNA by the RNA editing entity can be determined in an in vitro assay comprising: (i) directly or indirectly introducing the target RNA into a primary cell line, (ii) directly or indirectly introducing the engineered polynucleotide into a primary cell line, and (iii) sequencing the target RNA. In some embodiments, an engineered circular polynucleotide may not comprise a 5′ reducing hydroxyl, a 3′ reducing hydroxyl, or both, capable of being exposed to a solvent. In some embodiments, the engineered circular polynucleotide can further comprise an RNA editing enzyme recruiting domain. In some embodiments, an RNA editing enzyme recruiting domain can recruit an RNA editing enzyme that, when associated with the engineered polynucleotide, can perform a chemical transformation on a base of a nucleotide in the target RNA. In some embodiments, a targeting domain can be about 20 nucleotides to about 150 nucleotides. In some embodiments, a target RNA can comprise a nonsense mutation. In some embodiments, a targeting domain can comprise at least a single nucleotide that can be mismatched to the target RNA. In some embodiments, the disease or condition can comprise Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, Hypomyelination with Atrophy of Basal Ganglia and Cerebellum (H-ABC), or cystic fibrosis.

Also disclosed herein are vectors comprising, the precursor engineered linear polynucleotides disclosed herein, or the engineered circular polynucleotide disclosed herein. In some embodiments, a vector can comprise an adeno-associated virus (AAV) vector. In some embodiments, an AAV vector can be an AAV1 vector, AAV2 vector, AAV3 vector, AAV4 vector, AAV5 vector, AAV6 vector, AAV7 vector, AAV8 vector, an AAV9 vector, a chimera of any of these, or a variant of any of these. In some embodiments, a viral vector can be a self-complementary adeno-associated viral (scAAV) vector. In some embodiments, a viral vector can be a single-stranded AAV vector.

Also disclosed herein are pharmaceutical compositions in unit dose form comprising the precursor engineered linear polynucleotides disclosed herein, the engineered circular polynucleotides disclosed herein, or the vectors disclosed herein; and a pharmaceutically acceptable: excipient, diluent, or carrier.

Also disclosed herein are methods of treating or preventing a disease or condition in a subject in need thereof comprising: administering a therapeutically effective amount of: the precursor engineered linear polynucleotides disclosed herein, the engineered circular polynucleotides disclosed herein, the vectors disclosed herein, or the pharmaceutical compositions disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a bar-graph of percent RNA editing, as measured by Sanger sequencing, 48 hours post-transfection of guide RNA design 1 (linear guide), design 2 (linear guide), and design 3 (circular guide) in 293T (human embryonic kidney), RD (human embryo rhabdomyosarcoma) undifferentiated, LHCN (human skeletal myoblast) undifferentiated, and LHCN differentiated. Controls are shown as “no transfection. FIG. 1B is a bar-graph of percent RNA editing, as measured by Sanger sequencing, 96 hours post-transfection of guide RNA design 1 (linear guide), design 2 (linear guide), and design 3 (circular guide) in 293T (human embryonic kidney), RD (human embryo rhabdomyosarcoma) undifferentiated, LHCN (human skeletal myoblast) undifferentiated, and LHCN differentiated. Controls are shown as “no transfection.

FIG. 2 is an exemplary schematic of an engineered guide RNA. The guide RNA comprises 2 ribozyme domains, 2 ligation sequence domains, 2 spacer domains, and a targeting domain “100.50”.

FIG. 3A is an exemplary RT-PCR-based strategy to confirm the circularization of an engineered guide RNA. FIG. 3B shows a gel electrophoresis image of reverse transcriptase (RT)-PCR products. The results show confirmation of the circularization of the engineered guide RNAs Design 5 and Design 3.

FIG. 4 shows a bar-graph of the percent RNA editing of different adenosine nucleotides in the RAB7A 3′UTR from Design 3 (circular guide) and a control GFP plasmid. RNA editing can occur over several adenosines on the target RNA.

FIG. 5 shows a bar-graph of the percent RNA editing of nucleotides in the RAB7A 3′UTR from linear guide RNAs Design 1 and Design 4, compared to the circular guide RNAs Design 5 and Design 3. Circular guide RNAs have increased RNA editing overtime than linear guide RNAs in cells.

FIG. 6A is a graphical schematic of percent editing of 3′ UTR alpha synuclein (SNCA) mRNA (right panel) after 40 and 72 hours utilizing linear guide RNAs (guides 1-7) and circular guide RNAs (guide 8, guide 9 and Rab7). FIG. 6B is a graphical schematic of percent knockdown, as compared to control, of SNCA mRNA as measured by qPCR after 40 and 72 hours post transfection of linear guide RNAs (guides 1-7) and circular guide RNAs (guide 8, guide 9 and Rab7). Both circular and linear guide RNAs can edit and knockdown alpha synuclein (SNCA) RNA by targeting the 3′ UTR.

FIG. 7 shows a bar graph of the percent of GFP positive cells as a read out for successful transduction of circular and linear guide RNA vectors.

FIG. 8 shows a bar graph of the percent RNA editing by circular and linear engineered guides. The circular engineered guide design had continuous editing for at least 72 hours.

FIG. 9 shows an agarose gel and PCR products from a PCR assay designed to detect circularization of guide RNAs. Circular guide RNAs were detected at 24 hours, 48 hours and 72 hours after transduction.

FIG. 10 shows a schematic of a method of making a circularized guide RNA with back-splicing.

FIG. 11A shows a graph of percent Rab7 editing as determined by ddPCR in neural cells after transduction with AAV2/2 virions encapsidating GFP (control) or Rab7 guides (Rab7U6 linear, Rab7 U7 smOPT linear, or Rab7 U6 circular). FIG. 11B shows a graph of percent Rab7 editing as determined by Sanger sequencing in neural cells after transduction with AAV2/2 virions encapsidating GFP (control) or Rab7 guides (Rab7U6 linear, Rab7 U7 smOPT linear, or Rab7 U6 circular). FIG. 11C shows a graph of percent Rab7 editing as determined by ddPCR in mature neural cells after transduction with AAV2/2 virions encapsidating GFP (control) or Rab7 guides (Rab7U6 linear, Rab7 U7 smOPT linear, or Rab7 U6 circular).

FIG. 12A shows a graph of percent Rab7 editing by a Rab7 U6 circular guide in neural cells and percent transduction based on the titer of virus used to transduce the cells. FIG. 12B shows a graph of percent Rab7 editing by a RAB7 U6 circular guide and percent transduction based on the viral titer and days of differentiation for the neural cells.

FIG. 13A shows a graph of percent Rab7 editing as determined by ddPCR in neural cells after transduction with AAV2/2 virions encapsidating GFP (control) or Rab7 guides (U6 linear, U7 smOPT linear) at different viral titers. FIG. 13B shows a graph of percent Rab7 editing as determined by Sanger sequencing in neural cells after transduction with AAV2/2 virions encapsidating GFP (control) or Rab7 guides (U6 linear, U7 smOPT linear) at different viral titers. FIG. 13C shows a graph of percent Rab7 editing as determined by ddPCR in neural cells after transduction with AAV2/2 virions encapsidating GFP (control) or Rab7 guides (U6 linear, U7 smOPT linear) at different viral titers.

FIG. 14A shows a graph of percent Rab7 editing by a U7 smOPT circular guide in neural cells and percent transduction based on the titer of virus used to transduce the cells. FIG. 14B shows a graph of percent Rab7 editing by a U7 smOPT circular guide and percent transduction based on the viral titer and days of differentiation for the neural cells.

FIG. 15 shows off-target editing profiles of GFP control, uninfected cells, U6 circular guides and U7 smOPT linear guides.

FIG. 16 shows a diagram of the piggyBAC TUBB4A D249N minigene and an overview of an experimental design.

FIG. 17 at left shows a graph of percent editing of TUBB4A D249N with a circular guide (0_100_50 Circle) and linear guides (0_20_6, 0_100_50) and at right shows an agarose gel with PCR products to confirm the presence of circular guides.

FIG. 18 shows percent RNA editing of RAB7 with a linear control guide (Design A) and circular guides with varying lengths of spacer sequences (Design B, Design C, Design D, and Design E).

FIG. 19 left shows graphs of on-target and off-target ADAR1 and ADAR1+ADAR2 editing of LRRK2 and depicts a circular LRRK2 guide (0.100.80) used in the experiment.

FIG. 20 shows plots of off target editing profiles of an Exb75 circular guide for the target SNCA and a depiction of the guide.

FIG. 21 shows plots of off target editing profiles of an Exb76 circular guide for the target SNCA and a depiction of the guide.

FIG. 22 shows plots of off target editing profiles of an Exb77 circular guide for the target SNCA and a depiction of the guide.

FIG. 23 shows plots of off target editing profiles of an Exb78 circular guide for the target SNCA and a depiction of the guide.

FIG. 24 shows plots of off target editing profiles of an Exb79 circular guide for the target SNCA and a depiction of the guide.

FIG. 25 shows that a guide RNA containing a 3′ SmOPT sequence and U7 hairpin can be circularized and expressed by U7 or U6 promoters to produce ADAR editing. FIG. 25A illustrates a 100 nt guide RNA (purple) with or without a 3′ SmOPT U7 hairpin (teal) flanked by RtcB circular ribozyme sites (orange). Sanger sequencing with a guide-specific primer (black) shows that the ribozyme sites have been successfully joined together, with the guide RNA and 3′ SmOPT U7 hairpin present inside the circular RNA. FIG. 25B compares different variations of the SmOPT U7 circular guide RNA using either the mU7 or hU6 promoter, different Sm binding domains and U7 hairpins, and various length linkers between the U7 hairpin and P1 circular ribozyme (upper panel). As above, a linear 100 nt guide RNA with a 3′ SmOPT sequence and U7 hairpin could cause ADAR RNA editing all six gene targets: human RAB7A exon 4, RAB7A 3′UTR, SNCA exon 4, SNCA 3′UTR, DMD exon 71 Splice Acceptor, or DMD exon 74 Splice Acceptor (middle panel). Circular variations of a 100 nt guide RNA with a 3′ SmOPT sequence and U7 hairpin could also generate substantial editing, whether expressed by the mU7 or hU6 promoters. Side effects of target transcript knockdown or inadvertent exon skipping were minimal (bottom panel).

FIG. 26 shows a graph of percent RNA editing of cells by Rab7 targeting circular guides delivered by an AAV2 vector and delivered absent from an AAV2 vector.

FIG. 27 shows graphs of percent RNA editing in different cell types by Rab7 targeting circular guides delivered by an AAV2 vector at different multiplicity of infections and delivered absent from an AAV2 vector.

FIG. 28 shows AAV2 delivered circular guide RNAs targeting RAB7A in HepG2 cells at 48 hours post-infection.

FIG. 29 shows, at left, a bar graph of percent editing of the RAB7A 3′UTR for various circular guide RNAs of different sizes and where different percentages of the entire circular chassis are the guide RNA. At right is a gel of the circular guide RNA PCR products.

FIG. 30 shows LRRK2 editing with linear and circular guide RNAs.

### DETAILED DESCRIPTION

### Overview

RNA-editing can offer a therapeutic opportunity to alter genetic information. A significant hurdle in the RNA-editing space can be inefficient editing. Ways to improve RNA-editing efficiency can comprise increasing the synthesis, stability, targeting efficiency, or localization of a guide RNA (gRNA) or an engineered polynucleotide. Modifications can be made to an engineered polynucleotide to increase the RNA-editing efficiency.

In some embodiments, engineered guide RNAs (gRNAs) and engineered polynucleotides can be disclosed herein and can comprise a targeting domain and a spacer domain. Such engineered polynucleotides can enhance an editing efficiency of a target RNA with respect to an RNA editing entity.

In some embodiments, precursor engineered guide polynucleotides can be disclosed herein. A precursor engineered polynucleotide or a precursor engineered linear polynucleotide can be used to produce an engineered guide RNA or engineered guide polynucleotide as described herein.

In some embodiments, vectors can be disclosed herein and can comprise an engineered polynucleotide, an engineered guide RNA or a precursor engineered guide RNA.

In some embodiments, nucleic acids can be disclosed herein and can comprise an engineered polynucleotide, an engineered guide RNA, or a precursor engineered guide RNA.

In some embodiments, pharmaceutical compositions can be disclosed herein and can comprise an engineered polynucleotide, an engineered guide RNA, a precursor engineered guide RNA, a vector, or a nucleic acid; and a pharmaceutically acceptable excipient, diluent, or carrier.

In some embodiments, kits can be disclosed herein and can comprise an engineered polynucleotide, an engineered guide RNA, a precursor engineered guide RNA, a vector, a nucleic acid, a pharmaceutical composition; and a container.

In some embodiments, methods of treating a disease or condition in a subject in need thereof or preventing a disease or condition in a subject in need thereof can be disclosed herein. Such methods can comprise administering to a subject an engineered polynucleotide, a precursor engineered polynucleotide, precursor engineered linear polynucleotide, a vector, a nucleic acid, or a pharmaceutical composition.

In some embodiments, methods of forming an engineered polynucleotide can be disclosed herein. Such methods can comprise directly or indirectly ligating a first nucleotide on a 3′ end of a precursor engineered linear polynucleotide to a second nucleotide on a 5′ end of the precursor engineered linear polynucleotide with a ligating entity, thereby forming the engineered polynucleotide.

The section headings used herein can be for organizational purposes and are not to be construed as limiting the subject matter described. In some cases, the sectional headings may not be constructed as limiting the subject matter described.

As used in the specification and claims, the singular form “a”, “an” and “the” include plural references unless the context clearly dictates otherwise. For example, the term “a polypeptide” includes a plurality of polypeptides, including mixtures thereof

The term “about” or “approximately” as used herein when referring to a measurable value such as an amount or concentration and the like, in some cases, can be meant to encompass variations of about 20%, 10%, 5%, 1%, 0.5%, or even 0.1% of the specified amount. In some instances, the term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values can be described in the application and claims, unless otherwise stated the term “about” meaning within an acceptable error range for the particular value should be assumed. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be within a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

As used herein, the term “comprising” can be intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

The term “subject,” “host,” “individual,” and “patient” can be used interchangeably herein to refer to animals, typically mammalian animals. In some cases, a subject or host can comprise a prokaryote. In some cases, an engineered polynucleotide, or a precursor engineered polynucleotide described herein can be administered to a prokaryote. For example, administration to a prokaryote can include administration of an engineered polynucleotide, or a precursor engineered polynucleotide by a phage or by electroporation of the polynucleotide. Any suitable mammal can be treated by a method, cell or composition described herein. A mammal can be administered a vector, an engineered guide RNA, a precursor guide RNA, an engineered polynucleotide, a nucleic acid, or a pharmaceutical composition, as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal can be a human. A mammal can be any age or at any stage of development (e.g., an adult, teen, child, infant, or a mammal in utero). A mammal can be male or female. A mammal can be a pregnant female. In some embodiments a subject can be a human. In some embodiments, a subject has or may be suspected of having a disease such as a neurodegenerative disease. In some embodiments, a subject has or can be suspected of having a cancer or neoplastic disorder. In other embodiments, a subject has or can be suspected of having a disease or disorder associated with aberrant protein expression. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

The terms “treating,” “treatment,” and the like can be used herein to mean obtaining a desired pharmacologic effect, physiologic effect, or any combination thereof. In some instances, a treatment can reverse an adverse effect attributable to the disease or disorder. In some cases, the treatment can stabilize the disease or disorder. In some cases, the treatment can delay progression of the disease or disorder. In some instances, the treatment can cause regression of the disease or disorder. In some instances, the treatment can prevent the occurrence of the disease or disorder. In some embodiments, a treatment's effect can be measured. In some cases, measurements can be compared before and after administration of the composition. For example, a subject can have medical images prior to treatment compared to images after treatment to show cancer regression. In some instances, a subject can have an improved blood test result after treatment compared to a blood test before treatment. In some instances, measurements can be compared to a standard.

The term “sample” as used herein, generally can refer to any sample of a subject (such as a blood sample or a tissue sample). A sample or portion thereof may comprise a cell, such as a stem cell. In some cases, a portion of a sample may be enriched for a cell, such as a cancer cell. A sample may comprise a tissue, a cell, serum, plasma, exosomes, a bodily fluid, or any combination thereof. A bodily fluid may comprise urine, blood, serum, plasma, saliva, mucus, spinal fluid, tears, semen, bile, amniotic fluid, or any combination thereof. A sample or portion thereof may comprise an extracellular fluid obtained from a subject. A sample or portion thereof may comprise cell-free nucleic acid, DNA or RNA. A sample or portion thereof may be analyzed for a presence or absence or one or more mutations. Genomic data may be obtained from the sample or portion thereof. A sample may be a sample suspected or confirmed of having a disease or condition. A sample may be a sample removed from a subject via a non-invasive technique, a minimally invasive technique, or an invasive technique. A sample or portion thereof may be obtained by a tissue brushing, a swabbing, a tissue biopsy, an excised tissue, a fine needle aspirate, a tissue washing, a cytology specimen, a surgical excision, or any combination thereof. A sample or portion thereof may comprise tissues or cells from a tissue type. For example, a sample may comprise a nasal tissue, a trachea tissue, a lung tissue, a pharynx tissue, a larynx tissue, a bronchus tissue, a pleura tissue, an alveoli tissue, breast tissue, bladder tissue, kidney tissue, liver tissue, colon tissue, thyroid tissue, cervical tissue, prostate tissue, heart tissue, muscle tissue, pancreas tissue, anal tissue, bile duct tissue, a bone tissue, brain tissue, spinal tissue, kidney tissue, uterine tissue, ovarian tissue, endometrial tissue, vaginal tissue, vulvar tissue, uterine tissue, stomach tissue, ocular tissue, sinus tissue, penile tissue, salivary gland tissue, gut tissue, gallbladder tissue, gastrointestinal tissue, bladder tissue, brain tissue, spinal tissue, a blood sample, or any combination thereof.

“Eukaryotic cells” comprise all of the life kingdoms except monera. They can be easily distinguished through a membrane-bound nucleus. Animals, plants, fungi, and protists can be eukaryotes or organisms whose cells can be organized into complex structures by internal membranes and a cytoskeleton. A characteristic membrane-bound structure can be the nucleus. The term “host” can include a eukaryotic host, including, for example, yeast, higher plant, insect and mammalian cells. Non-limiting examples of eukaryotic cells or hosts include simian, bovine, porcine, murine, rat, avian, reptilian and human.

The term “protein”, “peptide” and “polypeptide” can be used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide may contain at least two amino acids and no limitation can be placed on the maximum number of amino acids which may comprise a protein's or peptide's sequence. As used herein the term “amino acid” can refer to natural, unnatural, or synthetic amino acids. Natural, unnatural, or synthetic amino acids can include glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, linker can refer to a protein fragment that can be used to link these domains together—optionally to preserve the conformation of the fused protein domains and/or prevent unfavorable interactions between the fused protein domains which may compromise their respective functions.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed.

In some cases, the identity between a reference sequence (query sequence, e.g., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

The terms “polynucleotide” and “oligonucleotide” can be used interchangeably and refer to a polymeric form of nucleotides of any length, either deoxyribonucleotides or ribonucleotides or analogs thereof. Polynucleotides can have any three-dimensional structure and may perform any function, known or unknown. The following can be non-limiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, a primer, an snRNA, a long non-coding RNA, a snoRNA, a siRNA, a miRNA, a tRNA-derived small RNA (tsRNA), an antisense RNA, a small RNA, an shRNA, or a small rDNA-derived RNA (srRNA). A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also can refer to both double- and single-stranded molecules. Nucleic acids, including e.g., nucleic acids with a phosphothioate backbone, can include one or more reactive moieties. As used herein, the term reactive moiety can include any group capable of reacting with another molecule, e.g., a nucleic acid or polypeptide through covalent, non-covalent or other interactions. By way of example, the nucleic acid can include an amino acid reactive moiety that can react with an amino acid on a protein or polypeptide through a covalent, non-covalent, or other interaction. Unless otherwise specified or required, any embodiment of this disclosure that can be a polynucleotide encompasses both the double-stranded form and each of two complementary single-stranded forms known or predicted to make up the double-stranded form.

Polynucleotides useful in the methods of the disclosure can comprise natural nucleic acid sequences and variants thereof, artificial nucleic acid sequences, or a combination of such sequences.

A polynucleotide can be composed of a specific sequence of four nucleotide bases: adenine (A); cytosine (C); guanine (G); thymine (T); and uracil (U) for thymine when the polynucleotide may be RNA. Any DNA sequence provided herein also refers to the RNA sequence wherein the thymines are substituted for uracils. Any RNA sequence provided herein also refers to the DNA sequence wherein the uracils are substituted for thymines. In some embodiments, the polynucleotide may comprise one or more other nucleotide bases, such as inosine (I), a nucleoside formed when hypoxanthine can be attached to ribofuranose via a β-N9-glycosidic bond, resulting in the chemical structure:

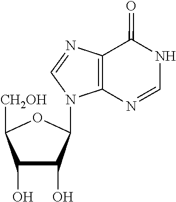

Inosine can be read by the translation machinery as guanine (G).

The term “polynucleotide sequence” can be the alphabetical representation of a polynucleotide molecule. This alphabetical representation can be input into databases in a computer having a central processing unit and used for bioinformatics applications such as functional genomics and homology searching.

As used herein, “expression” can refer to the process by which polynucleotides can be transcribed into mRNA and/or the process by which the transcribed mRNA can be subsequently translated into peptides, polypeptides, or proteins. If the polynucleotide can be derived from genomic DNA, expression may include splicing of the mRNA in an eukaryotic cell.

The term “sequencing” as used herein, may comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, or any combination thereof.

The terms “equivalent” or “biological equivalent” can be used interchangeably when referring to a particular molecule, biological, or cellular material and intend those having minimal homology while still maintaining desired structure or functionality.

The term “encode” as it may be applied to polynucleotides can refer to a polynucleotide which can be said to “encode” a polypeptide if, in its native state or when manipulated by methods well known to those skilled in the art, it can be transcribed, translated, or transcribed and translated to produce the mRNA for the polypeptide or a fragment thereof. The antisense strand can be the complement of such a nucleic acid, and the encoding sequence can be deduced therefrom.

As used herein, the term “functional” may be used to modify any molecule, biological, or cellular material to intend that it accomplishes a particular, specified effect.

The term “effective amount” can refer to a quantity sufficient to achieve a desired effect. In the context of therapeutic or prophylactic applications, the effective amount can depend on the type and severity of the condition at issue and the characteristics of the individual subject, such as general health, age, sex, body weight, and tolerance to pharmaceutical compositions. In the context of an immunogenic composition, in some embodiments the effective amount can be the amount sufficient to result in a protective response against a pathogen. In other embodiments, the effective amount of an immunogenic composition can be the amount sufficient to result in antibody generation against the antigen. In some embodiments, the effective amount can be the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. In some cases, the skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. In some cases, the skilled artisan will be able to determine the effective amount based on these and other considerations. The effective amount may comprise one or more administrations of a composition depending on the embodiment.

As used herein the term “restoring” in relation to expression of a protein can refer to the ability to establish expression of full-length protein where previously protein expression was truncated due to mutation.

The term “mutation” as used herein, can refer to an alteration to a nucleic acid sequence encoding a protein relative to the consensus sequence of said protein. “Missense” mutations can result in the substitution of one codon for another; “nonsense” mutations can change a codon from one encoding a particular amino acid to a stop codon. Nonsense mutations can often result in truncated translation of proteins. “Silent” mutations can be those which have no effect on the resulting protein. As used herein the term “point mutation” can refer to a mutation affecting only one nucleotide in a gene sequence. “Splice site mutations” can be those mutations present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. The reference DNA sequence can be obtained from a reference database. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (e.g., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation may indicate that a tissue or sample can be benign. As an alternative, absence of a mutation may not indicate that a tissue or sample can be benign. Methods as described herein can comprise identifying a presence of a mutation in a sample.

“Messenger RNA” or “mRNA” can be a nucleic acid molecule that can be transcribed from DNA and then processed to remove non-coding sections known as introns. The resulting mRNA can be exported from the nucleus (or another locus where the DNA can be present) and translated into a protein. The term “pre-mRNA” can refer to the strand prior to processing to remove non-coding sections.

“Canonical amino acids” refer to those 20 amino acids found naturally in the human body as shown in TABLE 1 below with each of their three letter abbreviations, one letter abbreviations, structures, and corresponding codons:

TABLE 1
Canonical Amino Acids

non-polar, aliphatic residues

| | | | | |
|---|---|---|---|---|
| Glycine | Gly | G | 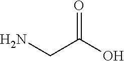 | GGU GGC GGA GGG |
| Alanine | Ala | A | 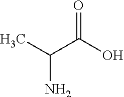 | GCU GCC GCA GCG |
| Valine | Val | V | 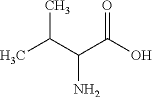 | GUU GUC GUA GUG |
| Leucine | Leu | L | 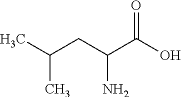 | UUA UUG CUU CUC CUA CUG |
| Isoleucine | Ile | I | 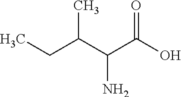 | AUU AUC AUA |
| Proline | Pro | P | 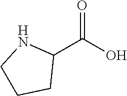 | CCU CCC CCA CCG |

aromatic residues

| | | | | |
|---|---|---|---|---|
| Phenylalanine | Phe | F | 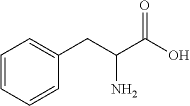 [SMILES: NC(Cc1ccccc1)C(=O)O] | UUU UUC |
| Tyrosine | Tyr | Y | 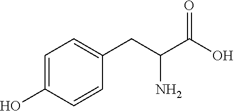 [SMILES: NC(Cc1ccc(O)cc1)C(=O)O] | UAU UAC |
| Tryptophan | Trp | W | 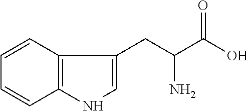 [SMILES: NC(Cc1c[nH]c2ccccc12)C(=O)O] | UGG |

polar, non-charged residues

| | | | | |
|---|---|---|---|---|
| Serine | Ser | S | 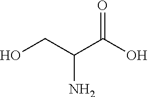 | UCU UCC UCA UCG AGU AGC |
| Threonine | Thr | T | 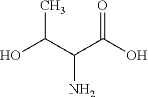 | ACU ACC ACA ACG |
| Cysteine | Cys | C | 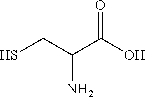 | UGU UGC |
| Methionine | Met | M | 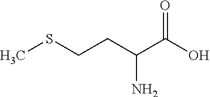 | AUG |
| Asparagine | Asn | N | 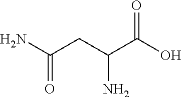 | AAU AAC |
| Glutamine | Gln | Q | 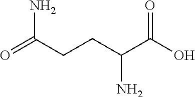 | CAA CAG |

positively charged residues

| | | | | |
|---|---|---|---|---|
| Lysine | Lys | K | 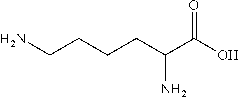 | AAA AAG |
| Arginine | Arg | R | 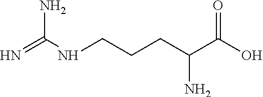 [SMILES: N=C(N)NCCCC(N)C(=O)O] | CGU CGC CGA CGG AGA AGG |
| Histidine | His | H | 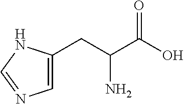 [SMILES: NC(Cc1cnc[nH]1)C(=O)O] | CAU CAC |

negatively charged residues

| | | | | |
|---|---|---|---|---|
| Aspartate | Asp | D | 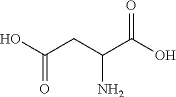 | GAU GAC |
| Glutamate | Glu | E | 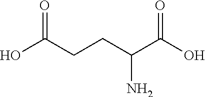 | GAA GAG |

The term “non-canonical amino acids” can refer to those synthetic or otherwise modified amino acids that fall outside this group, typically generated by chemical synthesis or modification of canonical amino acids (e.g., amino acid analogs). The present disclosure employs proteinogenic non-canonical amino acids in some of the methods and vectors disclosed herein. A non-limiting exemplary non-canonical amino acid can be pyrrolysine (Pyl or O), the chemical structure of which is provided below:

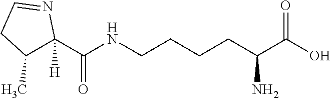 [SMILES: C[C@@H]1CC=N[C@H]1C(=O)NCCCC[C@H](N)C(=O)O]

Inosine (I) can be another exemplary non-canonical amino acid, which can be commonly found in tRNA and can be essential for proper translation according to “wobble base pairing.” The structure of inosine is provided above.

The term “ADAR” as used herein can refer to an adenosine deaminase that can convert adenosines (A) to inosines (I) in an RNA sequence. ADAR1 and ADAR2 can be two exemplary enzymes of ADAR that can be involved in mRNA editing in vivo. Non-limiting exemplary sequences for ADAR1 may be found under the following reference numbers: HGNC: 225; Entrez Gene: 103; Ensembl: ENSG 00000160710; OMIM: 146920; UniProtKB: P55265; and GeneCards: GC01M154554, as well as biological equivalents thereof. Non-limiting exemplary sequences for ADAR2 may be found under the following reference numbers: HGNC: 226; Entrez Gene: 104; Ensembl: ENSG00000197381; OMIM: 601218; UniProtKB: P78563; and GeneCards: GC21P045073, as well as biological equivalents thereof.

The term “deficiency” as used herein can refer to lower than normal (physiologically acceptable) levels of a particular agent. In context of a protein, a deficiency can refer to lower than normal levels of the full-length protein.

The term “complementary” or “complementarity” can refer to the ability of a nucleic acid to form hydrogen bond(s) with another nucleic acid sequence by either traditional Watson-Crick or other non-traditional types. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (e.g., stringent hybridization conditions). In some embodiments, hybridize can refer to a single stranded nucleic acid annealing to an at least partially complementary single stranded nucleic acid to form a double stranded nucleic acid. In some instances, hybridization can comprise binding of a nucleic acid to a complementary nucleic acid. For example, hybridization of two sequences of RNA can form a double stranded duplex (e.g. dsRNA). Nucleic acids can include nonspecific sequences. As used herein, the term “nonspecific sequence” or “not specific” can refer to a nucleic acid sequence that contains a series of residues that can be not designed to be complementary to or can be only partially complementary to any other nucleic acid sequence.

The term “ornithine transcarbamylase” or “OTC” as used herein can refer to the protein corresponding with that name and encoded by the gene Otc; a non-limiting example of which can be found under UniProt Reference Number P00480 (for humans) and P11725 (for mice). OTC deficiency can be an X-linked genetic condition resulting in high concentrations of ammonia in blood. In some cases, OTC deficiency can be caused by a G->A splice site mutation in the donor splice site of exon 4 that results in mis-splicing of the pre-mRNA. This mutation results in the formation of a protein that either can be elongated or bears a point mutation. There can be a 15-20-fold reduction in the OTC protein levels. See, e.g., Hodges, P. E. & Rosenberg, L. E. The spfash mouse: a missense mutation in the ornithine transcarbamylase gene also causes aberrant mRNA splicing. Proc. Natl. Acad. Sci. U.S.A. 86, 4142-4146 (1989)) (showing the alternative forms of OTC produced). The sequences thereof are provided below:

OTCpre-mRNA (wild type)
SEQ ID NO: 1
.....CTCACAGACACCGCTCGGTTTGTAAAACTTTTCTTC.....
OTCpre-mRNA (mutant)
SEQ ID NO: 2
.....CTCACAGACACCGCTC [symbol] GTTTGTAAAACTTTTCTTC.....
OTC mRNA (incorrectly spliced, mutant)
SEQ ID NO: 3
.....CTCACAGACACCGCTCAGTTTGTAAAACTTTTCTTC.....
OTC mRNA (correctly spliced, mutant)
SEQ ID NO: 4
.....CTCACAGACACCGCTCATGTCTTATCTAGCATGACA.....
OTC mRNA (correctly spliced, wild type)
SEQ ID NO: 5
.....CTCACAGACACCGCTCGTGTCTTATCTAGCATGACA.....

As shown above, a correct splice variant may be produced when the mutation may be present; however, such production results in a missense mutation, which also can contribute to OTC deficiency.

The terms “hairpin,” “hairpin loop,” “stem-loop,” and/or “loop” used alone or in combination with “motif” can be used in context of an oligonucleotide to refer to a structure formed in single stranded oligonucleotide when sequences within the single strand which can be complementary when read in opposite directions base pair to form a region whose conformation resembles a hairpin or loop.

As used herein, the term “domain” can refer to a particular region of a protein or polypeptide and can be associated with a particular function. For example, “a domain which associates with an RNA hairpin motif” can refer to the domain of a protein that binds one or more RNA hairpin. This binding may optionally be specific to a particular hairpin.

The term “APOBEC” as used herein can refer to any protein that falls within the family of evolutionarily conserved cytidine deaminases involved in mRNA editing—catalyzing a C to U conversion—and equivalents thereof. In some aspects, the term APOBEC can refer to any one of APOBEC1, APOBEC2, APOBEC3A, APOBEC3B, APOBEC3C, APOBEC3E, APOBEC3F, APOBEC3G, APOBEC3H, APOBEC4, or equivalents each thereof. Non-limiting exemplary sequences of fusion proteins comprising one or more APOBEC domains may be provided herein both fused to an ADAR domain or fused to alternative domains to render them suitable for use in an RNA editing system. To this end, APOBECs can be considered an equivalent of ADAR—catalyzing editing albeit by a different conversion.

The term “Glur2 mRNA” as used herein can refer to the mRNA encoding ionotropic AMPA glutamate receptor 2 (“Glur2”) which undergoes adenosine to inosine (A→I) editing. This mRNA can recruit ADARs in a site-specific manner.

As used herein, the term “interferon” can refer to a group of signaling proteins known to be associated with the immune response. In context of this application, the interferons of interest can be those that result in enhanced expression of an ADAR. The correlation between interferon a and ADAR1 may be known, and, thus, the present disclosure contemplates use of interferon a as a means of increasing endogenous ADAR1 expression. Commercial sources of isolated or recombinant interferon a include but may not be limited to Sigma-Aldrich, R&D Systems, Abcam, and Thermo Fisher Scientific. Alternatively, interferon a may be produced using a known vector and given protein sequence, e.g., Q6QNB6 (human IFNA).

It can be to be inferred without explicit recitation and unless otherwise intended, that when the present disclosure relates to a polypeptide, protein, polynucleotide or antibody, an equivalent or a biologically equivalent of such can be intended within the scope of this disclosure. As used herein, the term “biological equivalent thereof” can be intended to be synonymous with “equivalent thereof” when referring to a reference protein, antibody, polypeptide or nucleic acid, intends those having minimal homology while still maintaining desired structure or functionality. Unless specifically recited herein, it can be contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof can be a polynucleotide that hybridizes under stringent conditions to the reference polynucleotide or its complement.

### Engineered Guide Polynucleotides

Disclosed herein are engineered polynucleotides (may also be referred to as an “engineered guide” or an “engineered guide RNA” or a “guide RNA”) for treating a disease or condition in a subject via editing of a target polynucleotide (e.g., a pre-RNA or an mRNA) implicated in the disease or condition. Such engineered polynucleotides are configured to bind to the target polynucleotide via a portion that is complementary to target polynucleotide. Binding of the engineered polynucleotide to the target polynucleotide facilitates editing of a base of the target polynucleotide via an RNA editing entity, which thereby treats the disease or condition in the subject. In some cases, an engineered polynucleotide can comprise DNA, RNA or both.

An engineered guide RNA or an engineered polynucleotide as described herein can include various domains. A “domain” can refer to a region of an engineered polynucleotide. In some cases, a domain can be described in terms of a function of the domain. For instance, a “targeting domain” can refer to a region of the engineered polynucleotide that can be at least partially complementary to a target RNA; an “RNA editing entity recruiting domain” can refer to a domain that can be capable of associating with or recruiting an RNA editing entity as described herein; and a “spacer domain” can refer to a domain that provides space between other domains. In some instances, recitation of a domain name does not limit the domain to a particular function. For example, a “targeting domain” that can be at least partially complementary to a target RNA can in some instances recruit an RNA editing entity.

### Circular Guide RNAs

As described herein, an engineered guide can comprise a circular or looped structure. An engineered polynucleotide can be circularized from a precursor engineered polynucleotide. Such a precursor engineered polynucleotide can be a precursor engineered linear polynucleotide. In some cases, a precursor engineered linear polynucleotide can be a precursor for a circular engineered polynucleotide. For example, a precursor engineered linear polynucleotide can be a linear mRNA transcribed from a plasmid, which can circularize within a cell. In another example, a precursor engineered linear polynucleotide can be constructed to be a linear polynucleotide with domains such as a ribozyme domain and a ligation domain that allow for circularization when inserted into a cell. A ribozyme domain can include a domain that is capable of cleaving the linear precursor RNA at specific sites (e.g., adjacent to the ligation domain). Where a precursor domain comprises, from 5′ to 3′: a 5′ ribozyme domain, a 5′ ligation domain, a circularized region, a 3′ ligation domain, and a 3′ ribozyme domain, the precursor polynucleotide can be specifically processed at both sites by the 5′ and the 3′ ribozymes, respectively, to free exposed ends on the 5′ and 3′ ligation domains. The free exposed ends can be ligation competent, such that the ends can be circularized to form a mature circularized structure. For instance, the free ends can include a 5′-OH and a 2′, 3′-cyclic phosphate that are ligated via RNA ligation in the cell. The linear polynucleotide with the ligation and ribozyme domains can be transfected into a cell where it can circularize via endogenous cellular enzymes.

In some cases, a precursor engineered polynucleotide can be circular. In some cases, a precursor engineered polynucleotide can be linear as a precursor linear engineered polynucleotide. In some cases, a precursor engineered polynucleotide can comprise DNA, RNA or both. In some cases, a precursor engineered polynucleotide can comprise a precursor engineered guide RNA. In some cases, a precursor engineered guide RNA can be used to produce an engineered guide RNA.

In some embodiments, an engineered polynucleotide, a precursor engineered linear polynucleotide, or a precursor engineered polynucleotide can be more than about: 10, 20, 30, 40, 50, 60, 70, 80, 90, 100, 200, 300, 400, 500, 600, 700, 800, 900, 1000, 2500 or 5000 nucleotides in length. In some embodiments, an engineered polynucleotide (e.g. an engineered guide polynucleotide), a precursor engineered linear polynucleotide, or a precursor engineered polynucleotide can be less than about: 20, 30, 40, 50, 60, 70, 80, 90, 100, 200, 300, 400, 500, 600, 700, 800, 900, 1000, 2500 or 5000 nucleotides in length. In some cases, an engineered polynucleotide (e.g. an engineered guide polynucleotide), a precursor engineered linear polynucleotide, or a precursor engineered polynucleotide can comprise about: 20 nucleotides to about 5000 nucleotides, 20 nucleotides to about 50 nucleotides, 20 nucleotides to about 150 nucleotides, 40 nucleotides to about 80 nucleotides, 40 nucleotides to about 100 nucleotides, 40 nucleotides to about 200 nucleotides, 70 nucleotides to about 140 nucleotides, 80 nucleotides to about 160 nucleotides, 80 nucleotides to about 150 nucleotides, 80 nucleotides to about 300 nucleotides, 80 nucleotides to about 600 nucleotides, 90 nucleotides to about 200 nucleotides, 100 nucleotides to about 250 nucleotides, 140 nucleotides to about 200 nucleotides, 150 nucleotides to about 350 nucleotides, 200 nucleotides to about 400 nucleotides, 200 nucleotides to about 500 nucleotides, 400 nucleotides to about 600 nucleotides, 450 nucleotides to about 800 nucleotides, 750 nucleotides to about 1250 nucleotides, 1000 nucleotides to about 2000 nucleotides, or about 2000 nucleotides to about 5000 nucleotides.

In some embodiments, an engineered polynucleotide, a precursor engineered linear polynucleotide, or a precursor engineered polynucleotide may not comprise a sequence encoding a sequence configured for RNA interference (RNAi). In some embodiments, an engineered polynucleotide may not comprise a sequence configured for RNAi. In some cases, an engineered polynucleotide may not comprise a sequence encoding a short interfering RNA (siRNA), microRNA (miRNA), short hairpin RNA (shRNA), or Dicer substrate.

In some embodiments, an engineered polynucleotide, such as an engineered guide RNA can be circular. In some cases, an engineered polynucleotide can be a loop. In some instances, a circular shape can comprise a loop shape. In some instances, a loop shape can comprise a circular shape. Loop formation or circularization can prevent exposed ends of a polynucleotide from being degraded and can significantly increase the half-life of a polynucleotide, in vivo or in vitro. In some embodiments, a circular or looped engineered polynucleotide can prevent one or more exposed ends from hydrolytic degradation. In some embodiments an engineered polynucleotide, such as an engineered circular guide RNA may not comprise a 5′ reducing hydroxyl, a 3′ reducing hydroxyl, or both, capable of being exposed to a solvent. In some instances, an engineered polynucleotide can comprise a secondary structure that can be less susceptible to hydrolytic degradation than an mRNA naturally present in a human cell.

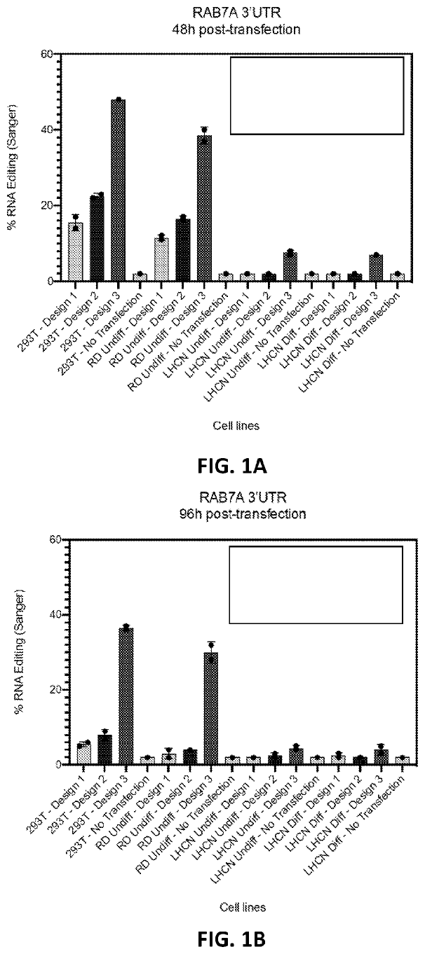

In some instances, a circular or looped engineered guide can comprise different qualities compared to a linear engineered polynucleotide. In some instances, a circular or looped engineered polynucleotide can significantly increase a half-life of the polynucleotide as compared to a comparable polynucleotide that may not be circular or may not be a loop. In some embodiments, forming a circular or looped engineered polynucleotide can significantly increase a half-life of an engineered polynucleotide (e.g., guide RNA) when delivered in vivo, such as to a subject, as compared to a comparable engineered polynucleotide (e.g. guide RNA) that can be not circular or not a loop. In some cases, forming a circular or looped engineered polynucleotide can significantly reduce an amount (such as a therapeutically effective amount) of the engineered polynucleotide dosed to a subject, as compared to a comparable engineered polynucleotide that can be not circular or not a loop. In some cases, a circular or looped engineered polynucleotide can have increased knockdown of a target RNA, as compared to a comparable engineered polynucleotide that can be not circular or not a loop. In some embodiments, forming a circular or looped engineered polynucleotide can significantly enhance efficiency of editing, can significantly reduce off-target editing, enhance efficiency of recruiting an RNA editing entity, or a combination thereof, as compared to a comparable engineered polynucleotide that may not be circular or may not be a loop. FIG. 1A and FIG. 1B show that circular guide RNAs can increase RNA editing in multiple cell lines compared to linear guide RNAs. In some cases, a circular or looped engineered polynucleotide can significantly increase the synthesis of the engineered polynucleotide as compared to a comparable engineered polynucleotide that can be not circular or not a loop. In some embodiments, a circular or looped engineered polynucleotide can significantly increase the transport of the engineered polynucleotide into a cell, as compared to a comparable engineered polynucleotide that can be not circular or not a loop. In some cases, a circular or looped engineered polynucleotide can significantly increase the intracellular retention of the engineered polynucleotide, as compared to a comparable engineered polynucleotide that can be not circular or not a loop.

A looped or circular engineered guide polynucleotide, such as an engineered guide RNA can provide various benefits as compared to a non-circular or non-looped guide polynucleotide. A looped or circular engineered polynucleotide can provide greater stability, improved recruitment of RNA-editing entities (such as endogenous RNA editing enzymes), longer half-lives, improved RNA-editing efficiency, or any combination thereof, as compared to a comparable engineered polynucleotide that can be not circular or not a loop. A looped or circular engineered guide polynucleotide can provide one or more of these improved qualities and can retain genetic encodability as compared guide polynucleotides comprising other types of modifications designed to improve guide stability—such as chemical modifications or sugar additions. A looped or circular engineered guide polynucleotide can be capable of being genetically encoded, capable of being delivered by a vector, and retain improved stability.

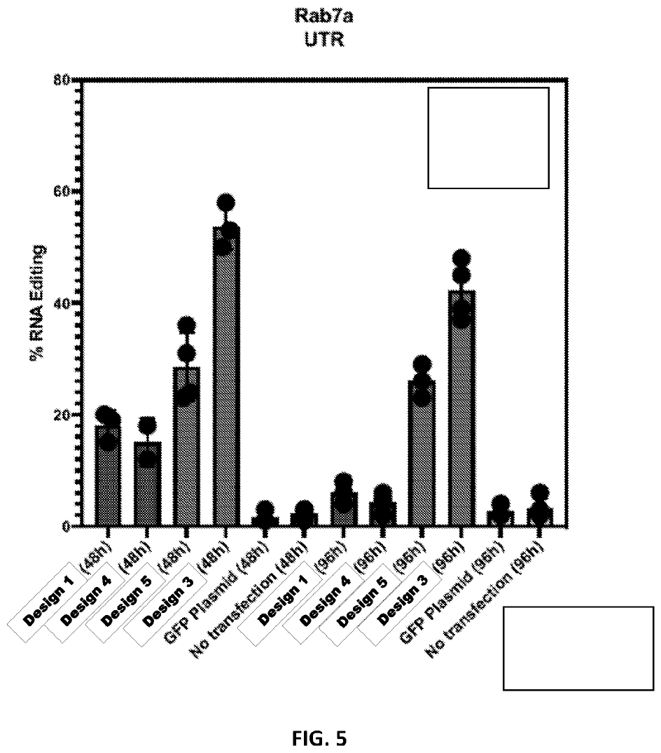

In some embodiments, a circular engineered guide RNA may have increased stability in a cell as compared to a linear guide RNA. In some embodiments, a circular engineered polynucleotide may have increased stability in a cell as compared to a linear polynucleotide. Increased stability of an RNA can be determined by measuring the percent of RNA editing over a period of time in cells transfected with either circular engineered guide RNA or linear guide RNA, or vectors encoding for circular guide RNA or linear guide RNA. For example, an assay can measure percent editing at 48 hours, 96 hours, and 144 hours. Increased stability can be indicated by increased percent RNA editing at later time points. Increased stability can also be measured by determining the copy number of circular guide RNA and linear guide RNA over a period of time (e.g. 48 hours, 96 hours, and 144 hours) in cells as determined by digital droplet PCR after reverse transcription of the guide RNA constructs. The increase in stability can be demonstrated in FIG. 5. FIG. 5 shows the percent RNA editing of nucleotides in the RAB7A 3′UTR from linear guide RNAs compared to the circular guide RNAs in cells. The circular guide RNAs were shown to have higher levels of RNA editing at 48 hour and 96 hours compared to the linear guide RNAs. The increase in editing may be from the increased stability of the circular guide RNAs compared to the linear guide RNAs.

An engineered guide RNA, an engineered polynucleotide, a precursor engineered linear polynucleotide, or a precursor engineered polynucleotide delivered to a cell or to a subject can comprise a half-life in the cell or the subject of at least about: 40 minutes, 50 minutes, 60 minutes, 1.5 hours (hrs), 2 hrs, 4 hrs, 6 hrs, 8 hrs, 10 hrs, 12 hrs, 16 hrs, 18 hrs, 20 hrs, 24 hrs, 1.25 days, 1.5 days, 2 days, 3 days, 4 days, 5 days, 6 days, 7 days, 10 days, 14 days, or more. A half-life of an engineered guide RNA, an engineered polynucleotide, or a precursor engineered polynucleotide delivered to a cell or to a subject can be from about 1 hour (hr) to about 6 hrs. A half-life of an engineered guide RNA, an engineered polynucleotide, or a precursor engineered polynucleotide delivered to a cell or to a subject can be from about 1 hr to about 24 hrs. A half-life of an engineered guide RNA, an engineered polynucleotide, or a precursor engineered polynucleotide delivered to a cell or to a subject can be from about 1 hr to about 2 days. A half-life of an engineered guide RNA, an engineered polynucleotide, or a precursor engineered polynucleotide delivered to a cell or to a subject can be from about 6 hrs to about 24 hrs. A half-life of an engineered guide RNA, an engineered polynucleotide, or a precursor engineered polynucleotide delivered to a cell or to a subject can be from about 6 hrs to about 5 days.

An engineered polynucleotide, such as an engineered guide RNA delivered to a cell or to a subject can recruit an RNA editing entity, such as an endogenous RNA editing entity. In some instances, an engineered guide polynucleotide can be co-delivered with an RNA editing entity. Circular polynucleotides, such as circular guide RNAs can recruit a greater amount of an RNA editing entity as compared to an engineered polynucleotide that may not be circular. In some embodiments, an engineered polynucleotide can be configured to recruit a sufficient amount of an endogenous RNA editing entity to perform the editing, such as delivery of the engineered polynucleotide to a tissue location that can be comprise a low amount of endogenous RNA editing enzymes.

### Circular Guide Structure

In some embodiments, a circular or looped engineered guide polynucleotide may not comprise a 5′ reducing hydroxyl, a 3′ reducing hydroxyl, or both, capable of being exposed to a solvent. In some instances, each 5′ hydroxyl, and each 3′ hydroxyl can be independently bonded to a phosphorous by a covalent oxygen phosphorous bond. In some cases, the phosphorous can be contained in a phosphodiester group. In some cases, a 5′ hydroxyl, a 3′ hydroxyl, or both, can be joined through a phosphorus-oxygen bond. In some cases, a 5′ hydroxyl, a 3′ hydroxyl, or both, can be modified into a phosphoester with a phosphorus-containing moiety.

In some cases, a 5′ end can be an end nucleotide of a polynucleotide with a terminal, exposed, unbound, or unlinked phosphate group on the 5′ carbon of the sugar of the end nucleotide. In some embodiments, a 3′ end can be an end nucleotide of a polynucleotide with a terminal, exposed, unbound, or unlinked hydroxyl group on the 3′ carbon of the sugar of the end nucleotide. In some instances, a 5′ end can be the first nucleotide of a polynucleotide to be transcribed, if or when the polynucleotide can be transcribed. In some embodiments, a 3′ end can be the last nucleotide of a polynucleotide to be transcribed, if or when the polynucleotide can be transcribed. In some cases, a 5′ end of a dinucleotide linked by a phosphodiester bond can be a nucleotide that contributes a hydroxyl group on the 3′ carbon of a sugar. In some cases, a 3′ end of a dinucleotide linked by a phosphodiester bond can be a nucleotide that contributes a hydroxyl group in a phosphoric acid on the 5′ carbon of a sugar.

In some embodiments, an engineered polynucleotide disclosed herein may comprise or encode for the structure of Formula (I).

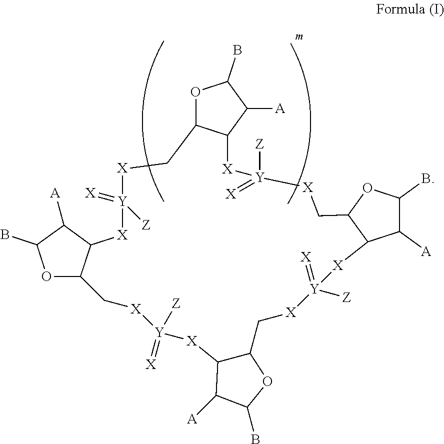

In some cases, the precursor engineered polynucleotide of Formula (I) may be encoded by a polynucleotide (e.g. a viral polynucleotide). In some cases, Formula (I) can comprise a targeting domain that can be at least partially complementary to a target RNA and Formula (I) can comprise a spacer domain as described herein. In some cases, Formula (I) can comprise a targeting domain that can be capable of hybridizing to a sequence of a target RNA. In some instances, at least partially complementary can comprise a targeting domain comprising a polynucleotide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%, sequence homology to the reverse complement of the target RNA . In some instances, each X can be independently O, S, or NR; each Y can be independently P or S; each Z can be independently OM, SM, or NRM; each A can be independently H, D, halogen, OM, SM, NRM, or NRR′; each B can be independently uracil, thymine, adenine, cytosine, guanine, a salt of any of these, or a derivative of any of these; m can be independently an integer from 0-1,000. In some instances, each X can be an O. In some instances, each Z can be O, S, or both. In some instances, each Y can be a P. In some instances, M can be at least 1. In some instances, each unit m can be independently in the (D)- or (L)-configuration. In some cases, m can be an independent integer from about: 5 to about 250, 30 to about 600, 100 to about 500, 400 to about 800, 600 to about 1200, 800 to about 2000, 1500 to about 4000 or about 300 to about 10000. In some cases, each M can be independently an inorganic or organic cation, H, or D and each R and R′ can be independently H, D, halogen, or C₁-C₆ alkyl. In some cases, each Z can be OM, each R can be H, and each Y can be P.

In some cases, association of a targeting domain with a sequence of a target RNA can facilitate an edit of a base of a nucleotide of the target RNA by an RNA editing entity. In some cases, the targeting domain can be at least partially complementary to the target RNA. In some cases, the targeting domain can be configured to at least partially associate with a coding region of a target RNA. In some cases, the association of a targeting domain with a coding region of a target RNA can facilitate an edit of a base of a nucleotide of the target RNA by an RNA editing entity. In some cases, the RNA editing entity can comprise an ADAR protein (e.g. ADAR1, ADAR2), APOBEC, a biologically active fragment thereof, or any combination thereof. In some cases, the RNA editing entity can be a human protein. In some cases, the RNA editing entity can be an endogenous protein. In some cases, the RNA editing entity can be an exogenous protein. In some cases, the targeting domain can be configured to at least partially associate with an untranslated region of a target RNA. In some instances, the targeting domain can be configured to at least partially associate with at least a portion of a 3′ or 5′ untranslated region (UTR) of a target RNA. In some instances, the association of a targeting domain with an untranslated region of a target RNA can facilitate a reduction in the expression level of a polypeptide encoded for by the target RNA, relative to a level of the polypeptide encoded for by the target RNA in the absence of the engineered polynucleotide, as determined by an in vitro assay comprising: (i) directly or indirectly introducing the target RNA into a first cell and a second cell of a primary cell line, (ii) directly or indirectly introducing the engineered polynucleotide into the first cell of the primary cell line, and (iii) comparing an amount of the polypeptide expressed from the target RNA in the first cell and the second cell.

### Spacer Domains

An engineered polynucleotide as described herein (or a precursor engineered linear polynucleotide, of circularization when inserted into a cell) can include spacer domains. As described herein, a spacer domain can refer to a domain that provides space between other domains. A spacer domain can be used to between a region to be circularized and flanking ligation sequences to increase the overall size of the mature circularized polynucleotide. Where the region to be circularized includes a targeting domain as described herein that is configured to associate to a target polynucleotide, the addition of spacers can provide improvements (e.g. increased specificity, enhanced editing efficiency, etc.) for the engineered polynucleotide to the target polynucleotide, relative to a comparable engineered polynucleotide that lacks a spacer domain. In some cases, a spacer domain can comprise a filler sequence. In some cases, a filler sequence can comprise a spacer domain. A filler sequence or filler domain can be a sequence in circular guide chassis that is at least partially non-complementary to the target sequence. In some instances, a filler sequence can comprise a portion of a circular guide chassis. In some instances, the spacer domain is configured to not hybridize with the target RNA in a contiguous fashion with the target polynucleotide. In such a configuration, the spacer domain is not simply increasing the amount of overlap between the target domain of the engineered polynucleotide and the target polynucleotide. Rather, the spacer domain can be used to elongate the circularized polynucleotide outside of the overlap region between the targeting domain and the target polynucleotide (e.g. increase the size of a circular chassis). By increasing the size of this circular polynucleotide outside of the overlap region, the present application demonstrates the surprising finding that overall binding efficiency between the targeting domain and target polynucleotide is improved. In some cases, this improvement may result from providing a more optimal geometry for the targeting domain of the circularized polynucleotide to bind to the target polynucleotide.

In some cases, the engineered polynucleotide comprising the spacer domain can have a lower Gibbs free energy (ΔG) of binding of the engineered polynucleotide to the target RNA, relative to a ΔG of binding of a corresponding polynucleotide that lacks the spacer domain, to the target RNA, as determined by KPFM. In some cases, the targeting domain can be configured to at least partially associate with an untranslated region of the target RNA, wherein the association of the targeting domain with the untranslated region of the target RNA facilitates a reduction in an expression level of a polypeptide encoded for by the target RNA and wherein association of the targeting domain with a sequence of the target RNA facilitates an edit of a base of a nucleotide of the target RNA by an RNA editing entity.

In some embodiments, an engineered polynucleotide, a precursor engineered linear polynucleotide, a precursor engineered polynucleotide or both can comprise a targeting domain that can be at least partially complementary to a target RNA, an RNA editing entity recruiting domain, and a spacer domain. In some instances, an engineered polynucleotide can be represented as a polynucleotide sequence in a circular 2-dimensional format with one nucleotide after the other. In some instances, an engineered polynucleotide can be represented as a polynucleotide sequence in a looped 2-dimensional format with one nucleotide after the other. In some cases, a spacer domain of an engineered polynucleotide can enlarge the engineered polynucleotide by the addition of one or more nucleotides. In some cases, the engineered polynucleotide does not comprise a 5′ reducing hydroxyl, a 3′ reducing hydroxyl, or both, capable of being exposed to a solvent. In some instances, the targeting domain can be configured to at least partially associate with a coding region of the target RNA. In some cases, the association of the targeting domain with the coding region of the target RNA can facilitate an edit of a base of a nucleotide of the target RNA by an RNA editing entity

In some cases, a spacer domain can have a sequence length of from about: 1 nucleotide to about 1,000 nucleotides, 2 nucleotides to about 20 nucleotides, 10 nucleotides to about 100 nucleotides, 50 nucleotides to about 500 nucleotides or about 400 nucleotides to about 1000 nucleotides in length. In some cases, a spacer domain can have a sequence length of at least 1, 2, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, or 2000 nucleotides.

In some embodiments, about 80% of the nucleotides of a spacer domain can be non-complementary to the target RNA. In some cases, a spacer domain can have a sequence length of about 5 nucleotides. In some cases, a spacer domain can have a sequence length of about 10 nucleotides. In some cases, a spacer domain can have a sequence length of about 15 nucleotides. In some cases, a spacer domain can have a sequence length of about 20 nucleotides. In some embodiments, a spacer domain can comprise a polynucleotide sequence of 5′ ATATA 3′ (SEQ ID 6), 5′ ATAAT 3′ (SEQ ID 7), or any combination thereof. In some cases, a spacer domain can comprise a sequence of 5′AUAAU 3′ (SEQ ID 8), 5′AUAUA 3′ (SEQ ID 9), 3′AUAUA 5′ (SEQ ID 32), or 3′AUAAU 5′ (SEQ ID 33). In some embodiments, a spacer domain can be at least a single nucleotide, such as A, T, G, C or U.

A spacer domain can be located proximal to a targeting domain, proximal to a ligation domain, proximal to a ribozyme domain, proximal to a RNA editing recruiting domain, or proximal to another spacer domain, where proximal can mean separated by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides.

In some embodiments, a spacer domain can be separated from a targeting domain by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides.

An engineered polynucleotide, a precursor engineered linear polynucleotide, or a precursor engineered polynucleotide can comprise a single spacer domain. In some cases, an engineered polynucleotide, a precursor engineered linear polynucleotide, or a precursor engineered polynucleotide can comprise a second spacer domain. In some cases, the first spacer domain, the second spacer domain or both can be configured to not bind to the target RNA when the targeting domain binds to the target RNA. In some embodiments an engineered polynucleotide, or a precursor engineered polynucleotide can comprise multiple spacer domains, for example 2, 3, 4, 5, 6, 7, or 8 spacer domains.

An engineered polynucleotide a precursor engineered linear polynucleotide, or precursor engineered polynucleotide can comprise a filler sequence. In some instances, a filler sequence can comprise a nucleic acid sequence that is not substantially complementary to the target RNA. In some instances, a filler sequence is configured to not hybridize with the target RNA in a contiguous fashion with the target polynucleotide. In some instances, a circular guide chassis can comprise any one of the engineered linear polynucleotides disclosed herein (e.g. guide RNA) and the remaining sequence can comprise a filler sequence. In some cases, a filler sequence can comprise about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% the total sequence of a circular guide chassis. In some cases, a guide RNA can comprise about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% the total sequence of a circular guide chassis. In some instances, a filler sequence that comprises about: 34% to about 72%, 40% to about 75%, or 50% to about 67% of the sequence of a circular guide chassis can produce the highest levels of RNA editing. In some instances, a targeting domain that comprises about: 28% or about 66%, 25% to about 60%, or 33% to about 50% of the sequence of a circular guide chassis can produce the highest levels of editing. In some cases, a circular guide chassis can comprise a total sequence length of about: 50 nucleotides to about 2000 nucleotides, 150 nucleotides to about 400 nucleotides, 100 nucleotides to about 450 nucleotides, 500 nucleotides to about 900 nucleotides, 900 nucleotides to about 1500 nucleotides, 1400 nucleotides to about 2000 nucleotides or 200 nucleotides to about 300 nucleotides. In some cases, a circular guide chassis can comprise 1, 2, 3, 4, or more filler sequences.

A spacer domain can be configured to facilitate an engineered polynucleotide adopting a conformation that facilitates at least partial binding to a target RNA. In some cases, a spacer domain can change the geometry of a targeting domain of a polynucleotide so that the targeting domain of the polynucleotide can be substantially linear. In some embodiments, a spacer domain can facilitate the synthesis of an engineered polynucleotide. In some instances, a spacer domain can facilitate the linkage of the solvent-exposing ends of a precursor engineered linear polynucleotide. In some embodiments, a spacer domain can bring two ligating ends of a precursor engineered polynucleotide closer than those that lack the spacer domain. In some embodiments, a linkage in an engineered polynucleotide can be covalent or non-covalent. In some cases, a linkage can be formed by a ligation reaction. In some embodiments, a linkage can be formed by a homologous recombination reaction.

An engineered polynucleotide, comprising a spacer domain can have an increase in the binding specificity to a target RNA, among a plurality of other RNAs, relative to the binding specificity of a corresponding polynucleotide that lacks the spacer domain. In some embodiments, an increase in the binding specificity to a target RNA can be determined by sequencing of a target RNA and plurality of other RNAs after contacting with an engineered polynucleotide comprising a spacer domain or a corresponding polynucleotide that lacks the spacer domain. In some instances, a spacer domain can be configured to facilitate a lower entropy (ΔS) of binding of an engineered polynucleotide to a target RNA. In some embodiments, a spacer domain can be configured to at least maintain an editing efficiency of an engineered polynucleotide to a target RNA, relative to the editing efficiency of a corresponding polynucleotide that lacks the spacer domain. In some cases, an editing efficiency can be determined by sequencing of a target RNA after contacting with an engineered polynucleotide with a spacer domain or a corresponding polynucleotide that lacks the spacer domain. In some embodiments, an at least maintain can comprise an increase. In some instances, an editing efficiency can be determined by mass spectroscopy of a target RNA after contacting with an engineered polynucleotide with a spacer domain or a corresponding polynucleotide that lacks the spacer domain.

In some embodiments, the editing efficiency of an engineered guide RNA, an engineered polynucleotide, or a precursor engineered guide RNA with a spacer domain can be from about 2× to about 5× greater than a comparable guide RNA, or a comparable engineered polynucleotide, that lacks the spacer. In some instances, the editing efficiency of an engineered guide RNA, an engineered polynucleotide, a precursor engineered linear polynucleotide, or a precursor engineered polynucleotide with a spacer delivered to a cell or to a subject can be from about 3× to about 6× greater than a comparable guide RNA, or a comparable engineered polynucleotide, that lacks the spacer. In some cases, the percent editing of a target RNA by an engineered guide RNA, an engineered polynucleotide, a precursor engineered linear polynucleotide, or a precursor engineered polynucleotide with a spacer can be about: 5%, 10%, 15%, 20%, 25%, 30%, 35%, 40%, 45%, 50%, 55%, 60%, 65%, 70%, 75%, 80%, 85%, 90%, 95%, or about 100% greater than a comparable guide RNA, or a comparable engineered polynucleotide, that lacks the spacer.

In some cases, Kelvin Probe Force Microscopy (KPFM) can be used to determine the Gibbs free energy (ΔG) of binding of an engineered guide RNA, or engineered polynucleotide, with a spacer domain to a target RNA, relative to a ΔG of binding of a corresponding engineered guide RNA, or engineered polynucleotide, that lacks the spacer domain: A target RNA can be immobilized on a bare gold nanoparticle. An engineered guide RNA, or an engineered polynucleotide, with or without the spacer domain, can be added and allowed to bind to the target RNA immobilized on the gold particle. The topography and surface potential images can then be measured with KPFM and used to calculate the ΔG of binding between the engineered guide RNA or the engineered polynucleotide, with or without the spacer domain, and the target RNA.

An in vitro half-life of an engineered guide RNA, an engineered polynucleotide, or a precursor engineered polynucleotide with a spacer domain can be at least about: 1×, 1.5×, 2×, 2.5×, 3×, 3.5×, 4×, 5×, 10×, 20× longer or more as compared to a substantially comparable RNA or comparable polynucleotide that lacks the spacer domain. An in vivo half-life of an engineered guide RNA, an engineered polynucleotide, or a precursor engineered polynucleotide with a spacer domain can be at least about: 1×, 1.5×, 2×, 2.5×, 3×, 3.5×, 4×, 5×, 10×, 20× longer or more as compared to a substantially comparable RNA or comparable polynucleotide that lacks the spacer domain. A dosage of a composition comprising an engineered guide RNA, an engineered polynucleotide, or a precursor engineered polynucleotide with a spacer domain administered to a subject in need thereof can be at least about: 1×, 1.5×, 2×, 2.5×, 3×, 3.5×, 4×, 5×, 10×, or 20× less as compared to a composition comprising a substantially comparable RNA or comparable polynucleotide that lacks the spacer domain administered to a subject in need thereof. A composition comprising an engineered guide RNA, an engineered polynucleotide, or a precursor engineered polynucleotide with a spacer domain administered to a subject in need thereof can be given as a single time treatment as compared to a composition comprising a substantially comparable RNA or comparable polynucleotide that lacks the spacer domain given as a two-time treatment or more.

An engineered guide RNA, an engineered polynucleotide, or a precursor engineered guide RNA with a spacer domain can comprise a half-life at least about: 2×, 3×, 4×, 5×, 6×, 7×, 8×, 9×, or 10× greater than a comparable guide RNA, or a comparable engineered polynucleotide, that lacks the spacer. A half-life of an engineered guide RNA, an engineered polynucleotide, or a precursor engineered guide RNA with a spacer domain can be from about 2× to about 5× greater than a comparable guide RNA, or a comparable engineered polynucleotide, that lacks the spacer. A half-life of an engineered guide RNA, an engineered polynucleotide, a precursor engineered linear polynucleotide, or a precursor engineered polynucleotide with a spacer delivered to a cell or to a subject can be from about 3× to about 6× greater than a comparable guide RNA, or a comparable engineered polynucleotide, that lacks the spacer.

In some cases, Formula (I) can be according to Formula (II)

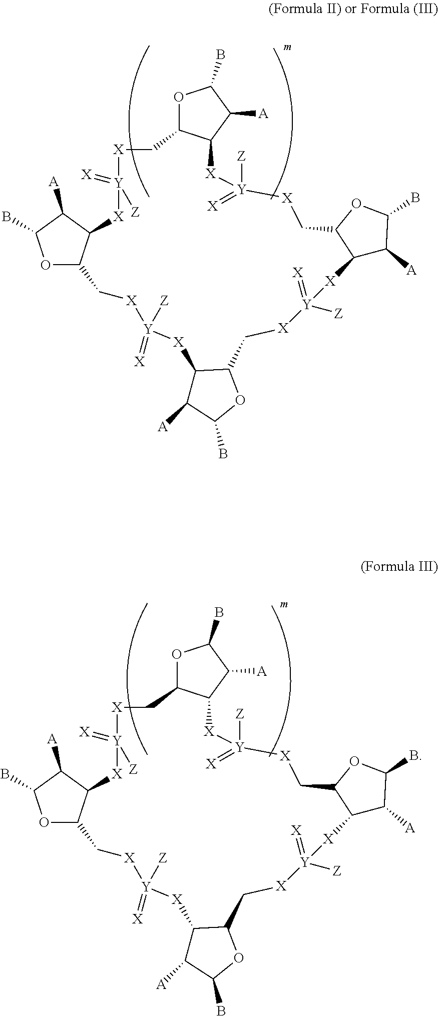

In some embodiments, engineered guide polynucleotide disclosed herein can comprise or encode for the structure of Formula (IV).

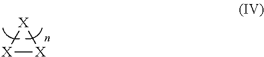

In some cases, each X can be a nucleotide comprising a base that can be independently uracil, thymine, adenine, cytosine, guanine, a salt of any of these, or a derivative of any of these. In some cases, each nucleotide can be connected to two adjacent nucleotides by, independently for each connection a phosphoester, phosphothioester, phosphothioate, or phosphoramidite linkage. In some instances, n can be independently an integer from 0-1,000. In some cases, n can be an independent integer from about: 5 to about 250, 100 to about 500, 400 to about 800, 600 to about 1200, 800 to about 2000, 1500 to about 4000 or about 300 to about 10000. In some cases, Formula (IV) can comprise a targeting domain that can be at least partially complementary to a target RNA and Formula (IV) can comprise a spacer domain. In some cases, the targeting domain does not physically comprise the spacer domain. For example, the targeting domain can be at least partially complementary to the target RNA and the spacer domain may not be complementary to the target RNA. In some cases, at least partially complementary can comprise a targeting domain (in an engineered polynucleotide or a precursor engineered polynucleotide) that can comprise a polynucleotide sequence with at least about: 70%, 75%, 80%, 85%, 90%, 95%, or 100% sequence homology to a reverse complement to the target RNA. In some instances, at least partially complementary can comprise a targeting domain comprising a polynucleotide sequence with at least about 70%, at least about 75%, at least about 80%, at least about 85%, at least about 90%, or at least about 95% sequence homology to the reverse complement of the target RNA. In some cases, the targeting domain can base-pair with at least about 80% of the target RNA. In some cases, the spacer domain can base-pair with less than about: 60%, 50%, 40%, 30%, 20%, or 10% of the target RNA.

In some embodiments, an engineered polynucleotide can comprise chirality. In some embodiments, any center atom, which can be chiral, can be independently in the (R) or (S) configuration. In some cases, chiral can comprise an atom in a molecule that can be bonded to four different types of atoms or chains of atoms. In some instances, an engineered polynucleotide, such as a guide RNA can be a single diastereomer or can be predominantly one diastereomer. In some instances, an engineered polynucleotide can have a diastereomeric excess of about: 51% to about 100%, 51% to about 60%, 60% to about 75%, 70% to about 90% or about 80% to about 99%. Diastereomeric excess can be a measurement of purity used for chiral substances. In some cases, it can reflect the degree to which a sample contains one diastereomer in greater amounts than another diastereomer. In some cases, a single pure diastereomer can have a diastereomeric excess of 100%. A sample with 70% of one diastereomer and 30% of the other can have a diastereomeric excess of 40% (70%-30%).

In some embodiments, an engineered polynucleotide can comprise: a targeting domain that is at least partially complementary to a target RNA; an RNA editing entity recruiting domain, wherein the RNA editing entity recruiting domain is configured to at least transiently associate with an RNA editing entity; and a spacer domain. In some cases, the targeting domain or the RNA editing recruiting domain may not comprise the spacer domain. In some cases, the engineered polynucleotide comprising the spacer domain can have a lower Gibbs free energy (ΔG) of binding of the engineered polynucleotide to the target RNA, relative to a ΔG of binding of a corresponding polynucleotide that lacks the spacer domain, as determined by Kelvin Probe Force Microscopy. In some instances, the engineered polynucleotide can comprise a backbone comprising a plurality of sugar and phosphate moieties covalently linked together. In some cases, backbone may not comprise a 5′ reducing hydroxyl, a 3′ reducing hydroxyl, or both, capable of being exposed to a solvent. In some cases, the targeting domain can be configured to at least partially associate with a coding region of the target RNA. In some cases, the association of the targeting domain with the coding region of the target RNA can facilitate an edit of a base of a nucleotide of the target RNA by an RNA editing entity.

### Forming Circular Guide RNAs

As discussed above, a circular or looped engineered guide polynucleotide, such as an engineered guide RNA can be formed directly or indirectly by forming a linkage (such as a covalent linkage) between more than one end of a RNA sequence, such as a 5′ end and a 3′ end. An RNA sequence can comprise an engineered guide RNA (such as a recruiting domain, targeting domain, or both). A linkage can be formed by employing an enzyme, such as a ligase. A suitable ligase (or synthetase) can include a ligase that forms a covalent bond. A covalent bond can include a carbon-oxygen bond, a carbon-sulfur bond, a carbon-nitrogen bond, a carbon-carbon bond, a phosphoric ester bond, or any combination thereof. A linkage can also be formed by employing a recombinase. An enzyme can be recruited to an RNA sequence to form a linkage. A circular or looped RNA can be formed by ligating more than one end of an RNA sequence using a linkage element. In some embodiments, a linkage can be formed by a ligation reaction. In some instances, a linkage can be formed by a homologous recombination reaction. A linkage element can employ click chemistry to form a circular or looped RNA. A linkage element can be an azide-based linkage. A circular or looped RNA can be formed by genetically encoding or chemically synthesizing the circular or looped RNA.

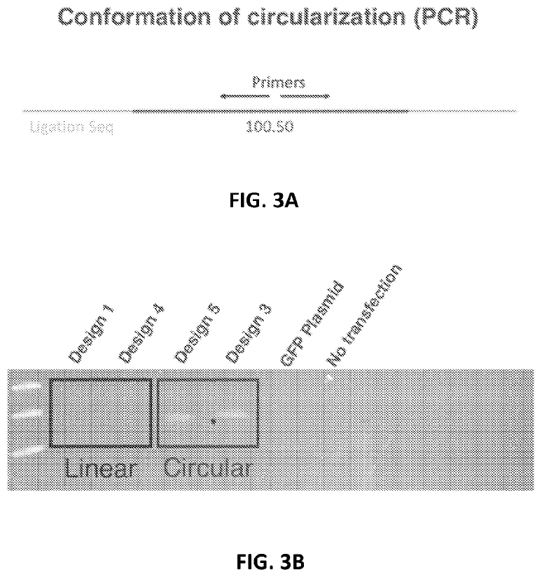

A circular or looped RNA can be formed by employing a self-cleaving entity, such as a ribozyme, tRNA, aptamer, catalytically active fragment of any of these, or any combination thereof. For example, a ribozyme, a tRNA, an aptamer, a catalytically active fragment of any of these, or any combination thereof can be added to a 3′ end, a 5′ end, or both of a precursor engineered RNA. In another example, a ribozyme, a tRNA, an aptamer, a catalytically active fragment of any of these, or any combination thereof can be added to a 3′ terminal end, a 5′ terminal end, or both of a precursor engineered RNA. A self-cleaving ribozyme can comprise, for example, an RNase P RNA a Hammerhead ribozyme (e.g. a Schistosoma mansoni ribozyme), a glmS ribozyme, an HDV-like ribozyme, an R2 element, a peptidyl transferase 23 S rRNA, a GIR1 branching ribozyme, a leadzyme, a group II intron, a hairpin ribozyme, a VS ribozyme, a CPEB3 ribozyme, a CoTC ribozyme, or a group I intron. In some cases, the self-cleaving ribozyme can be a trans-acting ribozyme that joins one RNA end on which it is present to a separate RNA end. In some embodiments, an aptamer can be added to each end of the engineered guide RNA. A ligase can be contacted with the aptamers at each end of the engineered guide RNA to form a covalent linkage between the aptamers thereby forming a circular engineered guide RNA. In some cases, a self-cleaving element or an aptamer can be configured to facilitate self-circularization of an engineered polynucleotide or a pro-polynucleotide (e.g. from a precursor engineered polypeptide) after transcription in a cell. For example, FIG. 3B shows confirmation of the circularization of the engineered guide RNAs Design 3 and Design 5 by a PCR assay. In some instances, circularization of a guide RNA can be shown by PCR. For example, primers can by developed that bind to the end of a guide RNA and are directed outward such that a product is only formed when guides are circularized.

In some cases, circularization can occur by back-slicing and ligation of an exon. For example, an RNA can be engineered from 5′ to 3′ to comprise a forward complementary sequence intron, an exon (which can comprise the guide sequence), followed by a reverse complementary sequence intron. Once transcribed, the complementary sequence introns can hybridize and form dsRNA. The internal exon containing the guide sequence can be removed by splicing and ligated by an endogenous ligase to form a circular guide. In one example, an engineered guide RNA can initiate circularization in a cell by autocatalytic reactions of encoded ribozymes. After cleavage by one or more ribozymes, the linear polynucleotide will undergo intracellular RNA ligation of the 5′ and the 3′ end of ligation sequences by an endogenous ligase to circularize the guide RNA.

A suitable self-cleaving molecule can include a ribozyme. For example, a ribozyme domain can create an autocatalytic RNA. A ribozyme can comprise an RNase P, an rRNA (such as a Peptidyl transferase 23 S rRNA), Leadzyme, Group I intron ribozyme, Group II intron ribozyme, a GIR1 branching ribozyme, a glmS ribozyme, a hairpin ribozyme, a Hammerhead ribozyme, an HDV ribozyme, a Twister ribozyme, a Twister sister ribozyme, a VS ribozyme, a Pistol ribozyme, a Hatchet ribozyme, a viroid, or any combination thereof. A ribozyme can include a P3 twister U2A ribozyme. A ribozyme can comprise 5′ GCCATCAGTCGCCGGTCCCAAGCCCGGATAAAATGGGAGGGGGCGGGAAACCGCCT 3′ (SEQ ID NO: 14). A ribozyme can comprise 5′ GCCAUCAGUCGCCGGUCCCAAGCCCGGAUAAAAUGGGAGGGGGCGGGAAACCGCC U 3′ (SEQ ID NO: 15). A ribozyme can comprise at least about: 70%, 75%, 80%, 85%, 90%, 95%, or 100% sequence homology to 5′ GCCATCAGTCGCCGGTCCCAAGCCCGGATAAAATGGGAGGGGGCGGGAAACCGCCT 3′ (SEQ ID NO: 14). A ribozyme can comprise at least about: 70%, 75%, 80%, 85%, 90%, 95%, or 100% sequence homology to 5′ GCCAUCAGUCGCCGGUCCCAAGCCCGGAUAAAAUGGGAGGGGGCGGGAAACCGCC U 3′ (SEQ ID NO: 15). A ribozyme can include a P1 Twister Ribozyme. A ribozyme can include 5′ AACACTGCCAATGCCGGTCCCAAGCCCGGATAAAAGTGGAGGGTACAGTCCACGC 3′ (SEQ ID NO: 16). A ribozyme can include 5′ AACACUGCCAAUGCCGGUCCCAAGCCCGGAUAAAAGUGGAGGGUACAGUCCACGC 3′ (SEQ ID NO: 17). A ribozyme can comprise at least about: 70%, 75%, 80%, 85%, 90%, 95%, or 100% sequence homology to 5′ AACACTGCCAATGCCGGTCCCAAGCCCGGATAAAAGTGGAGGGTACAGTCCACGC 3′ (SEQ ID NO: 16). A ribozyme can comprise at least about: 70%, 75%, 80%, 85%, 90%, 95%, or 100% sequence homology to 5′ AACACUGCCAAUGCCGGUCCCAAGCCCGGAUAAAAGUGGAGGGUACAGUCCACGC 3′ (SEQ ID NO: 17).

A ligation domain can facilitate a linkage, covalent or non-covalent, of a first nucleotide to a second nucleotide. In some embodiments, a ligation domain can recruit a ligating entity to facilitate a ligation reaction. In some cases, a ligation domain can recruit a recombining entity to facilitate a homologous recombination. In some instances, a first ligation domain can facilitate a linkage, covalent or non-covalent, to a second ligation domain. In some embodiments, a first ligation domain can facilitate the complementary pairing of a second ligation domain. In some cases, a ligation domain can comprise 5′ AACCATGCCGACTGATGGCAG 3′ (SEQ ID NO: 10). In some embodiments, a ligation domain can comprise 5′ GATGTCAGGTGCGGCTGACTACCGTC 3′ (SEQ ID NO: 11). In some cases, a ligation domain can comprise 5′ AACCAUGCCGACUGAUGGCAG 3′ (SEQ ID NO: 12). In some cases, a ligation domain can comprise 5′ GAUGUCAGGUGCGGCUGACUACCGUC 3′ (SEQ ID NO: 13). In some cases, a ligation domain can comprise at least about: 70%, 75%, 80%, 85%, 90%, 95%, or 100% sequence homology to 5′AACCATGCCGACTGATGGCAG 3′ (SEQ ID NO: 10). In some cases, a ligation domain can comprise at least about: 70%, 75%, 80%, 85%, 90%, 95%, or 100% sequence homology to 5′ GATGTCAGGTGCGGCTGACTACCGTC 3′ (SEQ ID NO: 11). In some cases, a ligation domain can comprise at least about: 70%, 75%, 80%, 85%, 90%, 95%, or 100% sequence homology to 5′ AACCAUGCCGACUGAUGGCAG 3′ (SEQ ID NO: 12). In some cases, a ligation domain can comprise at least about: 70%, 75%, 80%, 85%, 90%, 95%, or 100% sequence homology to 5′ GAUGUCAGGUGCGGCUGACUACCGUC 3′ (SEQ ID NO: 13).

In some embodiments, a precursor engineered linear polynucleotide, a precursor engineered polynucleotide or a circular engineered guide, can comprises a sequence with at least about 70%, at least about 75%, at least about 80%, at least about 85%, at least about 90%, at least about 92%, at least about 95%, at least about 97%, at least about 99%, or 100% sequence homology to a U7 or a U1 promoter sequence

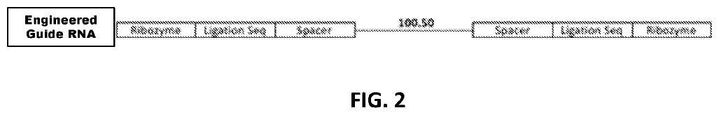

In some embodiments, a precursor engineered polynucleotide or a circular engineered guide, can comprise, in order of 5′ to 3′: a first ribozyme domain; a first ligation domain; a first spacer domain; a targeting domain that can be at least partially complementary to a target RNA, a second spacer domain, a second ligation domain, and a second ribozyme domain. In some cases, the first spacer domain, the second spacer domain, or both are configured to not bind to the target RNA when the targeting domain binds to the target RNA. In some instances, a precursor engineered polynucleotide can comprise a precursor engineered linear polynucleotide. In some cases, a precursor engineered linear polynucleotide can comprise, in order of 5′ to 3′: a first spacer domain; a targeting domain that can be at least partially complementary to a target RNA, and a second spacer domain. In some cases, the targeting domain can comprise a polynucleotide sequence with at least about at least about 80%, at least about 85%, at least about 90%, at least about 92%, at least about 95%, at least about 97%, at least about 99%, or 100% sequence homology to a reverse complement to the target RNA. In some cases, the pro-polynucleotide from the a precursor engineered linear polynucleotide, is configured to self-circularize after transcription in a mammalian cell. In some cases, a precursor engineered linear polynucleotide, can comprise a ribozyme domain 5′ to the first spacer domain or 3′ to the second spacer domain. In some cases, a precursor engineered linear polynucleotide, can comprise a ligation domain between the ribozyme domain and the first spacer domain or between the ribozyme domain and the second spacer domain. In some cases, a spacer domain of a precursor engineered linear polynucleotide, can be configured to not bind to a target RNA when a targeting domain of a precursor engineered linear polynucleotide, binds to the target RNA. Referring to FIG. 2, an engineered guide RNA is shown comprising 2 ribozyme domains, 2 ligation sequence domains, 2 spacer domains, and a targeting domain (100.50).

In some cases, a method of forming an engineered polynucleotide can comprise directly or indirectly ligating a nucleotide on a 3′ end of a precursor engineered linear polynucleotide, to a nucleotide on a 5′ end of the precursor engineered linear polynucleotide, with a ligating entity, thereby forming the engineered polynucleotide. In some cases, an engineered polynucleotide, can comprise (a) a targeting domain that can be at least partially complementary to a target RNA and, (b) a spacer domain, wherein the engineered polynucleotide does not comprise a 5′ reducing hydroxyl capable of being exposed to a solvent, wherein the engineered polynucleotide comprising the spacer domain has a lower Gibbs free energy (ΔG) of binding of the engineered polynucleotide to the target RNA, relative to a ΔG of binding of a corresponding polynucleotide that lacks the spacer domain, as determined by Kelvin Probe Force Microscopy (KPFM). In some cases, at least one of the following applies: (i) the spacer domain can be configured to not bind to the target RNA when the targeting domain at least partially binds to the target RNA; (ii) when the targeting domain at least partially binds to the target RNA, the spacer domain can be separated from the targeting domain by at least 1 nucleotide, and if the spacer domain binds to the target RNA, the binding of the spacer domain does not produce an edit of the target RNA at the portion of the target RNA that binds to the spacer domain; or (iii) when the spacer domain can be adjacent to a 5′ end or a 3′ end of the targeting domain, the spacer domain may not be complementary to the target RNA. In some instances, when the spacer domain may not be complementary to the target RNA this can mean the spacer domain may not be 100% homologous to the target RNA. In some cases, when the spacer domain may not be complementary to the target RNA, the spacer domain can comprise one or more non-complementary bases with the target RNA.

In some cases, a viral genome can comprise an engineered polynucleotide or a precursor engineered polynucleotide described herein. In some cases, a viral genome can comprise an engineered guide RNA. In some cases, a viral genome can comprise scAAV. TABLE 2 provides sequences of genes and sequences of guide RNA components described herein.

TABLE 2
Sequences of Genes and Guide RNA Components

| SEQ ID | | |
|---|---|---|
| NO | SOURCE | SEQUENCE (5′ to 3′) |
| 1 | OTC pre- | CTCACAGACACCGCTCGGTTTGTAAAACTTTTC |
| | mRNA (wild | TTC |
| | type) | |
| 2 | OTC pre- | CTCACAGACACCGCTCAGTTTGTAAAACTTTTC |
| | mRNA | TTC |
| | (mutant) | |
| 3 | OTC mRNA | CTCACAGACACCGCTCAGTTTGTAAAACTTTTC |
| | (incorrectly | TTC |
| | spliced, | |
| | mutant) | |
| 4 | OTC mRNA | CTCACAGACACCGCTCATGTCTTATCTAGCATG |
| | (correctly | ACA |
| | spliced, | |
| | mutant) | |
| 5 | OTC mRNA | CTCACAGACACCGCTCGTGTCTTATCTAGCATG |
| | (correctly | ACA |
| | spliced, wild | |
| | type) | |
| 6 | Spacer domain | ATATA |
| | 1 | |
| 7 | Spacer domain | ATAAT |
| | 2 | |
| 8 | Spacer domain | AUAAU |
| | 1 RNA | |
| | sequence | |
| 9 | Spacer domain | AUAUA |
| | 2 RNA | |
| | sequence | |
| 10 | Ligation | AACCATGCCGACTGATGGCAG |
| | domain 1 | |
| 11 | Ligation | GATGTCAGGTGCGGCTGACTACCGTC |
| | domain 2 | |
| 12 | Ligation | AACCAUGCCGACUGAUGGCAG |
| | domain 1 RNA | |
| | sequence | |
| 13 | Ligation | GAUGUCAGGUGCGGCUGACUACCGUC |
| | domain 2 RNA | |
| | sequence | |
| 14 | Ribozyme | GCCATCAGTCGCCGGTCCCAAGCCCGGATAAAA |
| | domain 1 | TGGGAGGGGGCGGGAAACCGCCT |
| 15 | Ribozyme | GCCAUCAGUCGCCGGUCCCAAGCCCGGAUAAA |
| | domain 1 RNA | AUGGGAGGGGGCGGGAAACCGCCU |
| | sequence | |
| 16 | Twister | AACACTGCCAATGCCGGTCCCAAGCCCGGATAA |
| | ribozyme | AAGTGGAGGGTACAGTCCACGC |
| | domain | |
| 17 | Twister | AACACUGCCAAUGCCGGUCCCAAGCCCGGAUA |
| | ribozyme | AAAGUGGAGGGUACAGUCCACGC |
| | domain RNA | |
| | sequence | |
| 18 | scITR | TCTGCGCGCTCGCTCGCTCACTGAGGCCGGGCG |
| | sequence | ACCAAAGGTCGCCCGACGCCCGGGCTTTGCCCG |
| | | GGCGGCCTCAGTGAGCGAGCGAGCGCGCAGA |
| 19 | Strawberry 1 | TCTACGCCATATTATCCACAGTCCAACGGCCAG |
| | sequence | GCGGAGGCTAGTAACAAGGTTATCCTCGGCATC |
| | | CTCCGCA |
| 20 | U6 promoter | GAGGGCCTATTTCCCATGATTCCTTCATATTTGC |
| | sequence | ATATACGATACAAGGCTGTTAGAGAGATAATTA |
| | | GAATTAATTTGACTGTAAACACAAAGATATTAG |
| | | TACAAAATACGTGACGTAGAAAGTAATAATTTC |
| | | TTGGGTAGTTTGCAGTTTTAAAATTATGTTTTAA |
| | | AATGGACTATCATATGCTTACCGTAACTTGAAA |
| | | GTATTTCGATTTCTTGGCTTTATATATCTTGTGG |
| | | AAAGGACGAAACACC |
| 21 | P3 twister | GGCCATCAGTCGCCGGTCCCAAGCCCGGATAAA |
| | U2A ribozyme | ATGGGAGGGGGCGGGAAACCGCCT |
| | sequence | |
| 22 | Left linker | AACCATGCCGACTGATGGCAGATAATATAAT |
| | and spacer | |
| | sequence | |
| 23 | Rab7a 3′UTR | GATAAAAGGCGTACATAATTCTTGTGTCTACTG |
| | 0.100.50 guide | TACAGAATACTGCCGCCAGCTGGATTTCCCAAT |
| | sequence | TCTGAGTAACACTCTGCAATCCAAACAGGGTTC |
| | | A |
| 24 | Right linker | ATATAAATATCTGCCATCAGTCGGCGTGGACTG |
| | and spacer | TAG |
| | sequence | |
| 25 | Pl twister | AACACTGCCAATGCCGGTCCCAAGCCCGGATAA |
| | ribozyme | AAGTGGAGGGTACAGTCCACGC |
| 26 | CMV | ATACGCGTTGACATTGATTATTGACTAGTTATT |
| | promoter | AATAGTAATCAATTACGGGGTCATTAGTTCATA |
| | | GCCCATATATGGAGTTCCGCGTTACATAACTTA |
| | | CGGTAAATGGCCCGCCTGGCTGACCGCCCAACG |
| | | ACCCCCGCCCATTGACGTCAATAATGACGTATG |
| | | TTCCCATAGTAACGCCAATAGGGACTTTCCATT |
| | | GACGTCAATGGGTGGAGTATTTACGGTAAACTG |
| | | CCCACTTGGCAGTACATCAAGTGTATCATATGC |
| | | CAAGTACGCCCCCTATTGACGTCAATGACGGTA |
| | | AATGGCCCGCCTGGCATTATGCCCAGTACATGA |
| | | CCTTATGGGACTTTCCTACTTGGCAGTACATCTA |
| | | CGTATTAGTCATCGCTATTACCATGGTGATGCG |
| | | GTTTTGGCAGTACATCAATGGGCGTGGATAGCG |
| | | GTTTGACTCACGGGGATTTCCAAGTCTCCACCC |
| | | CATTGACGTCAATGGGAGTTTGTTTTGGCACCA |
| | | AAATCAACGGGACTTTCCAAAATGTCGTAACAA |
| | | CTCCGCCCCATTGACGCAAATGGGCGGTAGGCG |
| | | TGTACGGTGGGAGGTCTATATAAGCAGAGCTCG |
| | | TTTAGTGAACCGTCAGATCGCCTGGAGACGCCA |
| | | TCCACGCTGTTTTGACCTCCATAGTAGACACCG |
| | | GGACCGATCCAGCCTCCGGACTCTAGAGGATCG |
| | | AACC |
| 27 | eGFP | ATGGTGAGCAAGGGCGAGGAGCTGTTCACCGG |
| | seqeuence | GGTGGTGCCCATCCTGGTCGAGCTGGACGGCGA |
| | | CGTAAACGGCCACAAGTTCAGCGTGTCCGGCGA |
| | | GGGCGAGGGCGATGCCACCTACGGCAAGCTGA |
| | | CCCTGAAGTTCATCTGCACCACCGGCAAGCTGC |
| | | CCGTGCCCTGGCCCACCCTCGTGACCACCCTGA |
| | | CCTACGGCGTGCAGTGCTTCAGCCGCTACCCCG |
| | | ACCACATGAAGCAGCACGACTTCTTCAAGTCCG |
| | | CCATGCCCGAAGGCTACGTCCAGGAGCGCACC |
| | | ATCTTCTTCAAGGACGACGGCAACTACAAGACC |
| | | CGCGCCGAGGTGAAGTTCGAGGGCGACACCCT |
| | | GGTGAACCGCATCGAGCTGAAGGGCATCGACTT |
| | | CAAGGAGGACGGCAACATCCTGGGGCACAAGC |
| | | TGGAGTACAACTACAACAGCCACAACGTCTATA |
| | | TCATGGCCGACAAGCAGAAGAACGGCATCAAG |
| | | GTGAACTTCAAGATCCGCCACAACATCGAGGAC |
| | | GGCAGCGTGCAGCTCGCCGACCACTACCAGCA |
| | | GAACACCCCCATCGGCGACGGCCCCGTGCTGCT |
| | | GCCCGACAACCACTACCTGAGCACCCAGTCCGC |
| | | CCTGAGCAAAGACCCCAACGAGAAGCGCGATC |
| | | ACATGGTCCTGCTGGAGTTCGTGACCGCCGCCG |
| | | GGATCACTCTCGGCATGGACGAGCTGTACAAGT |
| | | ACTCAGATCTCGAGCTCAAGTGA |
| 28 | SV40STOP | CAGACATGATAAGATACATTGATGAGTTTGGAC |
| | sequence | AAACCACAACTAGAATGCAGTGAAAAAAATGC |
| | | TTTATTTGTGAAATTTGTGATGCTATTGCTTTAT |
| | | TTGTAACCATTATAAGCTGCAATAAACAAGTTA |
| | | ACAACAACAATTGCATTCATTTTATGTTTCAGG |
| | | TTCAGGGGGAGGTGTGGGAGGTTTTTTAAA |
| 29 | Strawberry 2 | CTCACGGACCAGTGCAACATATTCCCAACATCC |
| | | CGTTGCAGCCTATCATTAAACCTTGGCCGGTCG |
| | | CGG |
| 30 | AAV2 ITR | AGGAACCCCTAGTGATGGAGTTGGCCACTCCCT |
| | | CTCTGCGCGCTCGCTCGCTCACTGAGGCCGCCC |
| | | GGGCAAAGCCCGGGCGTCGGGCGACCTTTGGTC |
| | | GCCCGGCCTCAGTGAGCGAGCGAGCGCGCAGA |
| | | GAGGGAGTGGCCAA |
| 31 | Full Viral | TCTGCGCGCTCGCTCGCTCACTGAGGCCGGGCG |
| | Genome | ACCAAAGGTCGCCCGACGCCCGGGCTTTGCCCG |
| | | GGCGGCCTCAGTGAGCGAGCGAGCGCGCAGAG |
| | | GATCCTCTACGCCATATTATCCACAGTCCAACG |
| | | GCCAGGCGGAGGCTAGTAACAAGGTTATCCTCG |
| | | GCATCCTCCGCAGGTACCGAGGGCCTATTTCCC |
| | | ATGATTCCTTCATATTTGCATATACGATACAAG |
| | | GCTGTTAGAGAGATAATTAGAATTAATTTGACT |
| | | GTAAACACAAAGATATTAGTACAAAATACGTG |
| | | ACGTAGAAAGTAATAATTTCTTGGGTAGTTTGC |
| | | AGTTTTAAAATTATGTTTTAAAATGGACTATCA |
| | | TATGCTTACCGTAACTTGAAAGTATTTCGATTTC |
| | | TTGGCTTTATATATCTTGTGGAAAGGACGAAAC |
| | | ACCGGCCATCAGTCGCCGGTCCCAAGCCCGGAT |
| | | AAAATGGGAGGGGGCGGGAAACCGCCTAACCA |
| | | TGCCGACTGATGGCAGATAATATAATGATAAAA |
| | | GGCGTACATAATTCTTGTGTCTACTGTACAGAA |
| | | TACTGCCGCCAGCTGGATTTCCCAATTCTGAGT |
| | | AACACTCTGCAATCCAAACAGGGTTCAATATAA |
| | | ATATCTGCCATCAGTCGGCGTGGACTGTAGAAC |
| | | ACTGCCAATGCCGGTCCCAAGCCCGGATAAAA |
| | | GTGGAGGGTACAGTCCACGCTTTTTTTACATAC |
| | | GCGTTGACATTGATTATTGACTAGTTATTAATA |
| | | GTAATCAATTACGGGGTCATTAGTTCATAGCCC |
| | | ATATATGGAGTTCCGCGTTACATAACTTACGGT |
| | | AAATGGCCCGCCTGGCTGACCGCCCAACGACCC |
| | | CCGCCCATTGACGTCAATAATGACGTATGTTCC |
| | | CATAGTAACGCCAATAGGGACTTTCCATTGACG |
| | | TCAATGGGTGGAGTATTTACGGTAAACTGCCCA |
| | | CTTGGCAGTACATCAAGTGTATCATATGCCAAG |
| | | TACGCCCCCTATTGACGTCAATGACGGTAAATG |
| | | GCCCGCCTGGCATTATGCCCAGTACATGACCTT |
| | | ATGGGACTTTCCTACTTGGCAGTACATCTACGT |
| | | ATTAGTCATCGCTATTACCATGGTGATGCGGTT |
| | | TTGGCAGTACATCAATGGGCGTGGATAGCGGTT |
| | | TGACTCACGGGGATTTCCAAGTCTCCACCCCAT |
| | | TGACGTCAATGGGAGTTTGTTTTGGCACCAAAA |
| | | TCAACGGGACTTTCCAAAATGTCGTAACAACTC |
| | | CGCCCCATTGACGCAAATGGGCGGTAGGCGTGT |
| | | ACGGTGGGAGGTCTATATAAGCAGAGCTCGTTT |
| | | AGTGAACCGTCAGATCGCCTGGAGACGCCATCC |
| | | ACGCTGTTTTGACCTCCATAGTAGACACCGGGA |
| | | CCGATCCAGCCTCCGGACTCTAGAGGATCGAAC |
| | | CCTTAAGCCGCCACCATGGTGAGCAAGGGCGA |
| | | GGAGCTGTTCACCGGGGTGGTGCCCATCCTGGT |
| | | CGAGCTGGACGGCGACGTAAACGGCCACAAGT |
| | | TCAGCGTGTCCGGCGAGGGCGAGGGCGATGCC |
| | | ACCTACGGCAAGCTGACCCTGAAGTTCATCTGC |
| | | ACCACCGGCAAGCTGCCCGTGCCCTGGCCCACC |
| | | CTCGTGACCACCCTGACCTACGGCGTGCAGTGC |
| | | TTCAGCCGCTACCCCGACCACATGAAGCAGCAC |
| | | GACTTCTTCAAGTCCGCCATGCCCGAAGGCTAC |
| | | GTCCAGGAGCGCACCATCTTCTTCAAGGACGAC |
| | | GGCAACTACAAGACCCGCGCCGAGGTGAAGTT |
| | | CGAGGGCGACACCCTGGTGAACCGCATCGAGC |
| | | TGAAGGGCATCGACTTCAAGGAGGACGGCAAC |
| | | ATCCTGGGGCACAAGCTGGAGTACAACTACAA |
| | | CAGCCACAACGTCTATATCATGGCCGACAAGCA |
| | | GAAGAACGGCATCAAGGTGAACTTCAAGATCC |
| | | GCCACAACATCGAGGACGGCAGCGTGCAGCTC |
| | | GCCGACCACTACCAGCAGAACACCCCCATCGGC |
| | | GACGGCCCCGTGCTGCTGCCCGACAACCACTAC |
| | | CTGAGCACCCAGTCCGCCCTGAGCAAAGACCCC |
| | | AACGAGAAGCGCGATCACATGGTCCTGCTGGA |
| | | GTTCGTGACCGCCGCCGGGATCACTCTCGGCAT |
| | | GGACGAGCTGTACAAGTACTCAGATCTCGAGCT |
| | | CAAGTGAACCGGTCAGACATGATAAGATACATT |
| | | GATGAGTTTGGACAAACCACAACTAGAATGCA |
| | | GTGAAAAAAATGCTTTATTTGTGAAATTTGTGA |
| | | TGCTATTGCTTTATTTGTAACCATTATAAGCTGC |
| | | AATAAACAAGTTAACAACAACAATTGCATTCAT |
| | | TTTATGTTTCAGGTTCAGGGGGAGGTGTGGGAG |
| | | _{GTTTTTTAAAGAGCTCCTCACGGACCAGTGCAA} |
| | | CATATTCCCAACATCCCGTTGCAGCCTATCATT |
| | | AAACCTTGGCCGGTCGCGGCTGCAGAGGAACC |
| | | CCTAGTGATGGAGTTGGCCACTCCCTCTCTGCG |
| | | CGCTCGCTCGCTCACTGAGGCCGCCCGGGCAAA |
| | | GCCCGGGCGTCGGGCGACCTTTGGTCGCCCGGC |
| | | CTCAGTGAGCGAGCGAGCGCGCAGAGAGGGAG |
| | | TGGCCAA |
| 56 | DMD exon 71 | AAGTACTCACGCAGAATCTACTGGCCAGAAGTT |
| | antisense | GATCAGAGTAACGGGACCGCAAAACAAAAAAT |
| | guide | GAGGTGGTGAAGGAGACACACGCAAACTCAGC |
| | | CGC |
| 57 | DMD exon 74 | CTGGTTCAAACTTTGGCAGTAATGCTGGATTAA |
| | antisense | CAAATGTTCATCATCTCCGGAAAATAAAATCAA |
| | guide | AGGTTGTGGTTTGTTCCCCCCCTTATGTTGCTTT |
| 58 | ABCA4 guide | GCCGGCACCATTCACTCCCAGGAGGCCAAAGC |
| | | ACTCTCCAGTGAGAACTCGGACCACAGCCTCCC |
| | | GCTGCTGGGCTGGAGGTGCCTGGATAAATCTTG |
| | | GT |
| 59 | ABCA4 guide | CCCCAGTGAGCATCTTGAATGTGGTTGTATTGC |
| | | CGGCACCATTCACTCCCAGGAGGCCAAAGCACT |
| | | CTCCAGTGAGAACTCGGACCACAGCCTCCCGCT |
| | | G |
| 60 | ABCA4 guide | CCCCAGTGAGCATCTTGAATGTGGTTGTATTGC |
| | | CGGCACCATTCACTCCCAGGAGGCCAAAGCACT |
| | | CTCCAGTGAGAACTCGGACCACAGCCTCCCGCT |
| | | GGTGGAATTTTTGGAGCAGGTTTTCTGACTTCG |
| | | GTCGGAAAACCCCT |
| 61 | APP guide | TCTGCACCCATCTTCACTTC |
| 62 | APP guide | TCATATCCTGAGTCATGTCGGAATTCTGCACCC |
| | | ATCTTCACTTCAGAGATCTCCTCCGTC |
| 63 | APP guide | TCATATCCTGAGTCATGCCGGAATTCTGCACCC |
| | | ATCTTCACTTCAGAGACCTCCTCCGTC |
| 64 | SERPINA1 | ACCGAUGGGUAUGGCCUCUAAAAACAUGGCCC |
| | guide | CAGCAGCUUCAGUCCCUUUCUCGUCGAUGGUC |
| | | AGCACAGCCUUAUGCACGGCCUGGAGGGGAGA |
| | | GAAGCAGAGUGGAAUUUUUGGAG |
| | | CAGGUUUUCUGACUUCGGUCGGAAAACCCCU |
| 65 | SERPINAI | ACCGAUGGGUAUGGCCUCUAAAAACAUGGCCC |
| | guide | CAGCAGCUUCAGUCCCUUACUCGUCGAUGGUC |
| | | AGCACAGCCUUAUGCACGGCCUGGAGGGGAGA |
| | | GAAGCAGAGUGGAAUUUUUGGAG |
| | | CAGGUUUUCUGACUUCGGUCGGAAAACCCCU |

In some cases, a viral genome can comprise or encode a scITR sequence, which can comprise at least about: 70%, 75%, 80%, 85%, 90%, 95%, or 100% sequence homology to 5′ TCTGCGCGCTCGCTCGCTCACTGAGGCCGGGCGACCAAAGGTCGCCCGACGCCCGG GCTTTGCCCGGGCGGCCTCAGTGAGCGAGCGAGCGCGCAGA 3′ (SEQ ID NO: 18). In some cases, a viral genome can comprise or encode a strawberry 1 sequence, which can comprise at least about: 70%, 75%, 80%, 85%, 90%, 95%, or 100% sequence homology to 5′ TCTACGCCATATTATCCACAGTCCAACGGCCAGGCGGAGGCTAGTAACAAGGTTATC CTCGGCATCCTCCGCA 3′ (SEQ ID NO: 19). In some cases, a viral genome can comprise or encode a U6 promoter sequence, which can comprise at least about: 70%, 75%, 80%, 85%, 90%, 95%, or 100% sequence homology to 5′ GAGGGCCTATTTCCCATGATTCCTTCATATTTGCATATACGATACAAGGCTGTTAGA GAGATAATTAGAATTAATTTGACTGTAAACACAAAGATATTAGTACAAAATACGTGA CGTAGAAAGTAATAATTTCTTGGGTAGTTTGCAGTTTTAAAATTATGTTTTAAAATGG ACTATCATATGCTTACCGTAACTTGAAAGTATTTCGATTTCTTGGCTTTATATATCTT GTGGAAAGGACGAAACACC 3′ (SEQ ID NO: 20). In some cases, a viral genome can comprise or encode a P3 twister U2A ribozyme sequence, which can comprise at least about: 70%, 75%, 80%, 85%, 90%, 95%, or 100% sequence homology to 5′ GGCCATCAGTCGCCGGTCCCAAGCCCGGATAAAATGGGAGGGGGCGGGAAACCGCC T 3′ (SEQ ID NO: 21). In some cases, a viral genome can comprise or encode a left linker and spacer sequence which can comprise at least about: 70%, 75%, 80%, 85%, 90%, 95%, or 100% sequence homology to 5′ AACCATGCCGACTGATGGCAGATAATATAAT 3′ (SEQ ID NO: 22). In some cases, a viral genome can comprise or encode a Rab7a 3′UTR 0.100.50 guide sequence, which can comprise at least about: 70%, 75%, 80%, 85%, 90%, 95%, or 100% sequence homology to 5′ GATAAAAGGCGTACATAATTCTTGTGTCTACTGTACAGAATACTGCCGCCAGCTGGA TTTCCCAATTCTGAGTAACACTCTGCAATCCAAACAGGGTTCA 3′ (SEQ ID NO: 23). In some cases, a viral genome can comprise or encode a right linker and spacer sequence, which can comprise at least about: 70%, 75%, 80%, 85%, 90%, 95%, or 100% sequence homology to 5′ ATATAAATATCTGCCATCAGTCGGCGTGGACTGTAG 3′ (SEQ ID NO: 24). In some cases, a viral genome can comprise or encode a P1 twister ribozyme, which can comprise at least about: 70%, 75%, 80%, 85%, 90%, 95%, or 100% sequence homology to 5′ AACACTGCCAATGCCGGTCCCAAGCCCGGATAAAAGTGGAGGGTACAGTCCACGC 3′ (SEQ ID NO: 25). In some cases, a viral genome can comprise or encode a CMV promoter, which can comprise at least about: 70%, 75%, 80%, 85%, 90%, 95%, or 100% sequence homology to 5′ ATACGCGTTGACATTGATTATTGACTAGTTATTAATAGTAATCAATTACGGGGTCATT AGTTCATAGCCCATATATGGAGTTCCGCGTTACATAACTTACGGTAAATGGCCCGCC TGGCTGACCGCCCAACGACCCCCGCCCATTGACGTCAATAATGACGTATGTTCCCAT AGTAACGCCAATAGGGACTTTCCATTGACGTCAATGGGTGGAGTATTTACGGTAAAC TGCCCACTTGGCAGTACATCAAGTGTATCATATGCCAAGTACGCCCCCTATTGACGT CAATGACGGTAAATGGCCCGCCTGGCATTATGCCCAGTACATGACCTTATGGGACTT TCCTACTTGGCAGTACATCTACGTATTAGTCATCGCTATTACCATGGTGATGCGGTTT TGGCAGTACATCAATGGGCGTGGATAGCGGTTTGACTCACGGGGATTTCCAAGTCTC CACCCCATTGACGTCAATGGGAGTTTGTTTTGGCACCAAAATCAACGGGACTTTCCA AAATGTCGTAACAACTCCGCCCCATTGACGCAAATGGGCGGTAGGCGTGTACGGTG GGAGGTCTATATAAGCAGAGCTCGTTTAGTGAACCGTCAGATCGCCTGGAGACGCC ATCCACGCTGTTTTGACCTCCATAGTAGACACCGGGACCGATCCAGCCTCCGGACTC TAGAGGATCGAACC 3′ (SEQ ID NO: 26). In some cases, a viral genome can comprise or encode a eGFP which can comprise at least about: 70%, 75%, 80%, 85%, 90%, 95%, or 100% sequence homology to 5′ ATGGTGAGCAAGGGCGAGGAGCTGTTCACCGGGGTGGTGCCCATCCTGGTCGAGCT GGACGGCGACGTAAACGGCCACAAGTTCAGCGTGTCCGGCGAGGGCGAGGGCGATG CCACCTACGGCAAGCTGACCCTGAAGTTCATCTGCACCACCGGCAAGCTGCCCGTGC CCTGGCCCACCCTCGTGACCACCCTGACCTACGGCGTGCAGTGCTTCAGCCGCTACC CCGACCACATGAAGCAGCACGACTTCTTCAAGTCCGCCATGCCCGAAGGCTACGTCC AGGAGCGCACCATCTTCTTCAAGGACGACGGCAACTACAAGACCCGCGCCGAGGTG AAGTTCGAGGGCGACACCCTGGTGAACCGCATCGAGCTGAAGGGCATCGACTTCAA GGAGGACGGCAACATCCTGGGGCACAAGCTGGAGTACAACTACAACAGCCACAACG TCTATATCATGGCCGACAAGCAGAAGAACGGCATCAAGGTGAACTTCAAGATCCGC CACAACATCGAGGACGGCAGCGTGCAGCTCGCCGACCACTACCAGCAGAACACCCC CATCGGCGACGGCCCCGTGCTGCTGCCCGACAACCACTACCTGAGCACCCAGTCCGC CCTGAGCAAAGACCCCAACGAGAAGCGCGATCACATGGTCCTGCTGGAGTTCGTGA CCGCCGCCGGGATCACTCTCGGCATGGACGAGCTGTACAAGTACTCAGATCTCGAGC TCAAGTGA 3′ (SEQ ID NO: 27). In some cases, a viral genome can comprise or encode a SV40STOP, which can comprise at least about: 70%, 75%, 80%, 85%, 90%, 95%, or 100% homology to 5′ CAGACATGATAAGATACATTGATGAGTTTGGACAAACCACAACTAGAATGCAGTGA AAAAAATGCTTTATTTGTGAAATTTGTGATGCTATTGCTTTATTTGTAACCATTATAA GC TGCAATAAACAAGTTAACAACAACAATTGCATTCATTTTATGTTTCAGGTTCAGG GGGAGGTGTGGGAGGTTTTTTAAA 3′ (SEQ ID NO: 28). In some cases, a viral genome can comprise or encode a Strawberry 2, which can comprise at least about: 70%, 75%, 80%, 85%, 90%, 95%, or 100% sequence homology to 5′ CTCACGGACCAGTGCAACATATTCCCAACATCCCGTTGCAGCCTATCATTAAACCTT GGCCGGTCGCGG 3′ (SEQ ID NO: 29). In some cases, a viral genome can comprise or encode a AAV2 ITR, which can comprise at least about: 70%, 75%, 80%, 85%, 90%, 95%, or 100% sequence homology to 5′ AGGAACCCCTAGTGATGGAGTTGGCCACTCCCTCTCTGCGCGCTCGCTCGCTCACTG AGGCCGCCCGGGCAAAGCCCGGGCGTCGGGCGACCTTTGGTCGCCCGGCCTCAGTG AGCGAGCGAGCGCGCAGAGAGGGAGTGGCCAA 3′ (SEQ ID NO: 30). In some cases, a viral genome can comprise or encode a full genome sequence with at least about: 70%, 75%, 80%, 85%, 90%, 95%, or 100% sequence homology to

(SEQ ID NO: 31)
TCTGCGCGCTCGCTCGCTCACTGAGGCCGGGCGAC
CAAAGGTCGCCCGACGCCCGGGCTTTGCCCGGGCG
GCCTCAGTGAGCGAGCGAGCGCGCAGAGGATCCTC
TACGCCATATTATCCACAGTCCAACGGCCAGGCGG
AGGCTAGTAACAAGGTTATCCTCGGCATCCTCCGC
AGGTACCGAGGGCCTATTTCCCATGATTCCTTCAT
ATTTGCATATACGATACAAGGCTGTTAGAGAGATA
ATTAGAATTAATTTGACTGTAAACACAAAGATATT
AGTACAAAATACGTGACGTAGAAAGTAATAATTTC
TTGGGTAGTTTGCAGTTTTAAAATTATGTTTTAAA
ATGGACTATCATATGCTTACCGTAACTTGAAAGTA
TTTCGATTTCTTGGCTTTATATATCTTGTGGAAAG
GACGAAACACCGGCCATCAGTCGCCGGTCCCAAGC
CCGGATAAAATGGGAGGGGGCGGGAAACCGCCTAA
CCATGCCGACTGATGGCAGATAATATAATGATAAA
AGGCGTACATAATTCTTGTGTCTACTGTACAGAAT
ACTGCCGCCAGCTGGATTTCCCAATTCTGAGTAAC
ACTCTGCAATCCAAACAGGGTTCAATATAAATATC
TGCCATCAGTCGGCGTGGACTGTAGAACACTGCCA
ATGCCGGTCCCAAGCCCGGATAAAAGTGGAGGGTA
CAGTCCACGCTTTTTTTACATACGCGTTGACATTG
ATTATTGACTAGTTATTAATAGTAATCAATTACGG
GGTCATTAGTTCATAGCCCATATATGGAGTTCCGC
GTTACATAACTTACGGTAAATGGCCCGCCTGGCTG
ACCGCCCAACGACCCCCGCCCATTGACGTCAATAA
TGACGTATGTTCCCATAGTAACGCCAATAGGGACT
TTCCATTGACGTCAATGGGTGGAGTATTTACGGTA
AACTGCCCACTTGGCAGTACATCAAGTGTATCATA
TGCCAAGTACGCCCCCTATTGACGTCAATGACGGT
AAATGGCCCGCCTGGCATTATGCCCAGTACATGAC
CTTATGGGACTTTCCTACTTGGCAGTACATCTACG
TATTAGTCATCGCTATTACCATGGTGATGCGGTTT
TGGCAGTACATCAATGGGCGTGGATAGCGGTTTGA
CTCACGGGGATTTCCAAGTCTCCACCCCATTGACG
TCAATGGGAGTTTGTTTTGGCACCAAAATCAACGG
GACTTTCCAAAATGTCGTAACAACTCCGCCCCATT
GACGCAAATGGGCGGTAGGCGTGTACGGTGGGAGG
TCTATATAAGCAGAGCTCGTTTAGTGAACCGTCAG
ATCGCCTGGAGACGCCATCCACGCTGTTTTGACCT
CCATAGTAGACACCGGGACCGATCCAGCCTCCGGA
CTCTAGAGGATCGAACCCTTAAGCCGCCACCATGG
TGAGCAAGGGCGAGGAGCTGTTCACCGGGGTGGTG
CCCATCCTGGTCGAGCTGGACGGCGACGTAAACGG
CCACAAGTTCAGCGTGTCCGGCGAGGGCGAGGGCG
ATGCCACCTACGGCAAGCTGACCCTGAAGTTCATC
TGCACCACCGGCAAGCTGCCCGTGCCCTGGCCCAC
CCTCGTGACCACCCTGACCTACGGCGTGCAGTGCT
TCAGCCGCTACCCCGACCACATGAAGCAGCACGAC
TTCTTCAAGTCCGCCATGCCCGAAGG5′CTACGTC
CAGGAGCGCACCATCTTCTTCAAGGACGACGGCAA
CTACAAGACCCGCGCCGAGGTGAAGTTCGAGGGCG
ACACCCTGGTGAACCGCATCGAGCTGAAGGGCATC
GACTTCAAGGAGGACGGCAACATCCTGGGGCACAA
GCTGGAGTACAACTACAACAGCCACAACGTCTATA
TCATGGCCGACAAGCAGAAGAACGGCATCAAGGTG
AACTTCAAGATCCGCCACAACATCGAGGACGGCAG
CGTGCAGCTCGCCGACCACTACCAGCAGAACACCC
CCATCGGCGACGGCCCCGTGCTGCTGCCCGACAAC
CACTACCTGAGCACCCAGTCCGCCCTGAGCAAAGA
CCCCAACGAGAAGCGCGATCACATGGTCCTGCTGG
AGTTCGTGACCGCCGCCGGGATCACTCTCGGCATG
GACGAGCTGTACAAGTACTCAGATCTCGAGCTCAA
GTGAACCGGTCAGACATGATAAGATACATTGATGA
GTTTGGACAAACCACAACTAGAATGCAGTGAAAAA
AATGCTTTATTTGTGAAATTTGTGATGCTATTGCT
TTATTTGTAACCATTATAAGCTGCAATAAACAAGT
TAACAACAACAATTGCATTCATTTTATGTTTCAGG
TTCAGGGGGAGGTGTGGGAGGTTTTTTAAAGAGCT
CCTCACGGACCAGTGCAACATATTCCCAACATCCC
GTTGCAGCCTATCATTAAACCTTGGCCGGTCGCGG
CTGCAGAGGAACCCCTAGTGATGGAGTTGGCCACT
CCCTCTCTGCGCGCTCGCTCGCTCACTGAGGCCGC
CCGGGCAAAGCCCGGGCGTCGGGCGACCTTTGGTC
GCCCGGCCTCAGTGAGCGAGCGAGCGCGCAGAGAG
GGAGTGGCCAA 3′.

### Engineered Polynucleotide Domains

A. Circular Chassis

The present disclosure provides compositions of circularized engineered polynucleotides that facilitate RNA editing of a base in a target RNA of interest. As described herein, circularized polynucleotides can include domains for associating with a target RNA and facilitating editing of a target polynucleotide. For example, engineered polynucleotides of the present disclosure facilitate an RNA edit comprising a chemical modification of a base, such as deamination of an adenosine (A) to an inosine (I). Inosines are read as guanosines (G). As such, the engineered polynucleotides and methods of use thereof disclosed herein of RNA editing can be used for correction of a G to A point mutation in a gene that may be implicated in a disease or disease pathway. Thus, engineered polynucleotides disclosed herein can be used as a therapeutic in a method of treatment.

Non-functional regions (e.g. regions of the circularized guides that do not directly associate with a target polynucleotide or do not directly facilitate editing of the target polynucleotide) can form a circularized chassis. Such a chassis can include spacer domains as described herein. A circularized chassis can be used as a vehicle to incorporate functional portions of any design to target any engineered polynucleotide. For example, a circularized chassis can comprise an engineered linear polynucleotide. Accordingly, engineered polynucleotides configured to associate with different target polynucleotides may share a single chassis or can have different chasses.

B. Targeting Domain

As discussed above, the circularized engineered polynucleotide can include a “functional” portion that is responsible for association with the target polynucleotide and facilitating editing of the engineered polynucleotide. In some cases, this portion can include a targeting domain or other domains described below, which can comprise structural features for association with an RNA editing entity.

An engineered guide RNA, an engineered polynucleotide, a precursor engineered linear polynucleotide, or a precursor engineered polynucleotide can comprise one or more domains, such as 1, 2, 3, 4, 5 or more domains that are responsible for the binding of the target polynucleotide and/or facilitating editing of the target polynucleotide. In some instances, an engineered guide RNA, an engineered polynucleotide, a precursor engineered linear polynucleotide, or a precursor engineered polynucleotide can comprise an RNA editing entity recruiting domain, a targeting domain, more than one of either, or a combination thereof. In some embodiments, an engineered guide RNA, an engineered polynucleotide, a precursor engineered linear polynucleotide, or a precursor engineered polynucleotide can comprise a targeting domain and an RNA editing entity recruiting domain. In some instances, an engineered guide RNA, an engineered polynucleotide, a precursor engineered linear polynucleotide, or a precursor engineered polynucleotide can comprise a targeting domain and two recruiting domains.

A targeting domain can be positioned adjacent to an RNA editing entity recruiting domain, including immediately adjacent or adjacent to but separated by a number of nucleotides. In some embodiments, a targeting domain can be flanked by two RNA editing entity recruiting domains. In some cases, two or more RNA editing entity recruiting domains can be adjacent to one another.

An engineered guide RNA, an engineered polynucleotide, a precursor engineered linear polynucleotide, or a precursor engineered polynucleotide can comprise a targeting domain for targeting a specific sequence region or base in a nucleic acid sequence for an RNA editing entity to perform a chemical transformation. In some cases, a targeting domain can comprise at least a single nucleotide that can be mismatched to the target RNA. In some cases, a mismatched nucleotide in a targeting domain can be adjacent to two nucleotides that can be complementary to the target RNA. In some cases, the mismatched nucleotide in the targeting domain can be adjacent to two nucleotides, one on each side of the mismatched nucleotide, that can be complementary to the target RNA. A targeting domain can comprise a sequence length that can be longer than an antisense RNA, a short hairpin RNA, a siRNA, miRNA, or snoRNA. A targeting domain can comprise a sequence length sufficient for an engineered guide RNA, an engineered polynucleotide, a precursor engineered linear polynucleotide, or a precursor engineered polynucleotide to form a secondary structure. A targeting domain can comprise a sequence length from about 20 bases to about 1,000 bases in length. A targeting domain can comprise a sequence length from about 50 bases to about 1,000 bases in length. A targeting domain can comprise a sequence length from about 100 bases to about 1,000 bases in length. A targeting domain can comprise a sequence length from about 200 bases to about 1,000 bases in length. A targeting domain can comprise a sequence length from about 500 bases to about 1,000 bases in length. A targeting domain can comprise a sequence length of at least about: 50, 60, 70, 80, 90, 100, 110, 120, 130, 140, 150, 160, 170, 180, 190, 200 bases in length.

In some instances, an engineered polynucleotide, or a precursor engineered polynucleotide can comprise one targeting domain. In some cases, an engineered polynucleotide, or a precursor engineered polynucleotide can comprise more than one targeting domain. In some embodiments, an engineered polynucleotide, or a precursor engineered polynucleotide can comprise two targeting domains. In some instances, an engineered polynucleotide, or a precursor engineered polynucleotide can comprise two targeting domains that target a same target RNA. In some instances, an engineered polynucleotide, or a precursor engineered polynucleotide can comprise two targeting domains that target different target RNAs. In some instances, an engineered polynucleotide, or a precursor engineered polynucleotide can comprise two targeting domains that comprise a same polynucleotide sequence identity. In some instances, an engineered polynucleotide, or a precursor engineered polynucleotide can comprise two targeting domains that comprise different polynucleotide sequence identities.

C. RNA Editing Entity Recruiting Domains

A functional portion of an engineered polynucleotide as described herein can include an RNA editing entity recruiting domain. An RNA editing entity recruiting domain can be a domain that comprises a secondary structure recognized by an RNA editing entity. Such structure when present on an RNA editing entity recruiting domain can be present in the absence of association with a target polynucleotide. Alternatively, such structures can be absent in the absence of association with the target polynucleotide and can form upon hybridization with the target polynucleotide. In such a case, the engineered polynucleotide may not comprise an RNA editing entity recruiting domain that is distinct from the targeting domain. Rather, the RNA editing entity recruiting domain may be the targeting domain itself when in association with the target polynucleotide.

An RNA editing entity recruiting domain can comprise at least about: 70%, 75%, 80%, 85%, 90%, 95%, 99% or 100% sequence homology to at least about 20 nucleotides of: an Alu domain, an APOBEC recruiting domain, a GluR2 domain, a TALEN recruiting domain, a Zn-finger polypeptide recruiting domain, a mega-TAL recruiting domain, or a Cas13 recruiting domain. In some embodiments, an RNA editing entity recruiting domain can comprise at least about 80% sequence homology to at least about 20 nucleotides of an Alu domain. In some cases, an RNA editing entity recruiting domain can comprise at least about 80% sequence homology to an Alu-recruiting domain. In some embodiments, an RNA editing entity recruiting domain can comprise at least about: 70%, 75%, 80%, 85%, 90%, or 95% sequence homology to at least about: 15, 20, 25, 30, or 35 nucleic acids of an Alu domain. In some cases, at least a portion of an RNA editing entity recruiting domain can comprise at least about 80% sequence homology to an Alu domain encoding sequence. In some embodiments, at least a portion of an RNA editing entity recruiting domain can comprise at least about 85% sequence homology to an Alu domain encoding sequence. In some cases, at least a portion of an RNA editing entity recruiting domain can comprise at least about 90% sequence homology to an Alu domain encoding sequence. In some embodiments, at least a portion of an RNA editing entity recruiting domain can comprise at least about 95% sequence homology to an Alu domain encoding sequence. In some cases, an Alu-domain-encoding sequence can be a non-naturally occurring sequence. In some embodiments, an Alu-domain-encoding sequence can comprise a modified portion. In some cases, an Alu-domain-encoding sequence can comprise a portion of a naturally occurring Alu-domain-encoding-sequence.

In some embodiments, an RNA editing entity recruiting domain can comprise at least about: 70%, 75%, 80%, 85%, 90%, 95%, or 100% sequence homology to at least about 20 nucleotides of an APOBEC domain. In some cases, an RNA editing entity recruiting domain can comprise at least about: 70%, 75%, 80%, 85%, 90%, 95%, or 100% sequence homology to an APOBEC-recruiting domain. In some embodiments, an RNA editing entity recruiting domain can comprise at least about: 70%, 75%, 80%, 85%, 90%, or 95% sequence homology to at least about: 15, 20, 25, 30, or 35 nucleic acids of an APOBEC domain. In some cases, at least a portion of an RNA editing entity recruiting domain can comprise at least about 80% sequence homology to an APOBEC domain encoding sequence. In some embodiments, at least a portion of an RNA editing entity recruiting domain can comprise at least about 85% sequence homology to an APOBEC domain encoding sequence. In some cases, at least a portion of an RNA editing entity recruiting domain can comprise at least about 90% sequence homology to an APOBEC domain encoding sequence. In some embodiments, at least a portion of an RNA editing entity recruiting domain can comprise at least about 95% sequence homology to an APOBEC domain encoding sequence. In some cases, an APOBEC-domain-encoding sequence can be a non-naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence.

In some embodiments, a recruiting domain comprises a CRISPR associated recruiting domain sequence. For example, a CRISPR associated recruiting sequence can comprise a Cas protein sequence. In some cases, a Cas13 recruiting domain can comprise a Cas13a recruiting domain, a Cas13b recruiting domain, a Cas13c recruiting domain, or a Cas13d recruiting domain. In some cases, an RNA editing entity recruiting domain can comprise at least about: 70%, 75%, 80%, 85%, 90%, 95%, or 100% sequence homology to at least about 20 nucleic acids of a Cas13b recruiting domain. In some embodiments, an RNA editing entity recruiting domain can comprise at least about 80% sequence homology to a Cas13b recruiting domain. In some cases, an RNA editing entity recruiting domain can comprise at least about: 70%, 75%, 80%, 85%, 90%, 95%, or 100% sequence homology to at least about: 15, 20, 25, 30, or 35 nucleic acids of a Cas13b domain. In some embodiments, at least a portion of an RNA editing entity recruiting domain can comprise at least about 80% sequence homology to a Cas13b domain encoding sequence. In some cases, at least a portion of an RNA editing entity recruiting domain can comprise at least about 85% sequence homology to a Cas13b domain encoding sequence. In some embodiments, at least a portion of an RNA editing entity recruiting domain can comprise at least about 90% sequence homology to a Cas13b domain encoding sequence. In some cases, at least a portion of an RNA editing entity recruiting domain can comprise at least about 95% sequence homology to a Cas13b domain encoding sequence. In some embodiments, a Cas13b-domain-encoding sequence can be a non-naturally occurring sequence. In some cases, a Cas13b-domain-encoding sequence can comprise a modified portion. In some embodiments, a Cas13b-domain-encoding sequence can comprise a portion of a naturally occurring Cas13b-domain-encoding-sequence.

In some cases, an RNA editing entity recruiting domain can comprise at least about: 70%, 75%, 80%, 85%, 90%, 95%, or 100% sequence homology to at least about 20 nucleotides of a GluR2 domain. In some embodiments, an RNA editing entity recruiting domain can comprise at least about: 70%, 75%, 80%, 85%, 90%, 95%, or 100% sequence homology to a GluR2-recruiting domain. In some cases, an RNA editing entity recruiting domain can comprise at least about: 70%, 75%, 80%, 85%, 90%, 95%, or 100% sequence homology to at least about: 15, 20, 25, 30, or 35 nucleic acids of a GluR2 domain. In some embodiments, at least a portion of an RNA editing entity recruiting domain can comprise at least about 80% sequence homology to a GluR2 domain encoding sequence. In some cases, at least a portion of an RNA editing entity recruiting domain can comprise at least about 85% sequence homology to a GluR2 domain encoding sequence. In some embodiments, at least a portion of an RNA editing entity recruiting domain can comprise at least about 90% sequence homology to a GluR2 domain encoding sequence. In some cases, at least a portion of an RNA editing entity recruiting domain can comprise at least about 95% sequence homology to a GluR2 domain encoding sequence. In some embodiments, a GluR2-domain-encoding sequence can be a non-naturally occurring sequence. In some cases, a GluR2-domain-encoding sequence can comprise a modified portion. In some embodiments, a GluR2-domain-encoding sequence can comprise a portion of a naturally occurring GluR2-domain-encoding sequence. In some cases, at least a portion of a recruiting domain can comprise at least about: 70%, 75%, 80%, 85%, 90%, 95%, or 100% sequence homology to an encoding sequence that recruits an ADAR. In some instances, an RNA editing entity recruiting domain can comprise at least about: 70%, 75%, 80%, 85%, 90%, 95%, or 100% sequence homology to an MS2-bacteriophage-coat-protein-recruiting domain.

In cases where a recruiting domain can be absent, an engineered polynucleotide can be still capable of associating with a subject RNA editing entity (e.g., ADAR) to facilitate editing of a target RNA and/or modulate expression of a polypeptide encoded by a subject target RNA. This may be achieved through structural features. Structural features may comprise any one of a: mismatch, symmetrical bulge, asymmetrical bulge, symmetrical internal loop, asymmetrical internal loop, hairpins, wobble base pairs, a structured motif, circularized RNA, chemical modification, or any combination thereof. In an aspect, a double stranded RNA (dsRNA) substrate, for example hybridized polynucleotide strands, can be formed upon hybridization of an engineered polynucleotide of the present disclosure to a target RNA. Described herein can be a feature, which corresponds to one of several structural features that may be present in a dsRNA substrate of the present disclosure. Examples of features include a mismatch, a bulge (symmetrical bulge or asymmetrical bulge), an internal loop (symmetrical internal loop or asymmetrical internal loop), or a hairpin (a hairpin comprising a non-targeting domain). Engineered polynucleotides of the present disclosure may have from 1 to 50 features. Engineered polynucleotides of the present disclosure may have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features.

As disclosed herein, a structured motif comprises two or more features in a dsRNA substrate.

A double stranded RNA (dsRNA) substrate can be formed upon hybridization of an engineered guide RNA of the present disclosure to a target RNA. As disclosed herein, a mismatch refers to a nucleotide in a guide RNA that can be unpaired to an opposing nucleotide in a target RNA within the dsRNA. A mismatch can comprise any two nucleotides that do not base pair, are not complementary, or both. In some embodiments, a mismatch can be an A/C mismatch. An A/C mismatch may comprise a C in an engineered guide RNA of the present disclosure opposite an A in a target RNA. An A/C mismatch may comprise a A in an engineered guide RNA of the present disclosure opposite an C in a target RNA. In an embodiment, a G/G mismatch may comprise a Gin an engineered guide RNA of the present disclosure opposite a G in a target RNA. In some embodiments, a mismatch positioned 5′ of the edit site may facilitate base-flipping of the target A to be edited. A mismatch may also help confer sequence specificity. In an embodiment, a mismatch comprises a G/G mismatch. In an embodiment, a mismatch comprises an A/C mismatch, wherein the A can be in the target RNA and the C can be in the targeting sequence of the engineered polynucleotide. In another embodiment, the A in the A/C mismatch can be the base of the nucleotide in the target RNA edited by a subject RNA editing entity.

In some cases, a subject targeting sequence comprises at least partial sequence complementarity to a region of a target RNA that at least partially encodes a subject polypeptide. In some cases, a targeting sequence can comprise a targeting domain. In some cases, a targeting sequence comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence may have a single base mismatch. In other cases, the targeting sequence of a subject engineered polynucleotide comprises at least about 1, 2, 3, 4, 5, 6, 7, 8, 9, 10, 11, 12, 13, 14, 15, 16, 20, 30, 40 or up to about 50 base mismatches. In some aspects, nucleotide mismatches can be associated with structural features provided herein. In some aspects, a targeting sequence comprises at least about 1, 2, 3, 4, 5, 6, 7, 8, 9, 10, 11, 12, 13, 14, or up to about 15 nucleotides that differ in complementarity from a wildtype RNA of a subject target RNA. In some cases, a targeting sequence comprises at least 50 nucleotides having complementarity to a target RNA. In some cases, a targeting sequence comprises from 50 to 150 nucleotides having complementarity to a target RNA. In some cases, a targeting sequence comprises from 50 to 200 nucleotides having complementarity to a target RNA. In some cases, a targeting sequence comprises from 50 to 250 nucleotides having complementarity to a target RNA. In some cases, a targeting sequence comprises from 50 to 300 nucleotides having complementarity to a target RNA. In some cases, a targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to a target RNA. In some cases, a targeting sequence comprises more than 50 nucleotides total and has at least 50 nucleotides having complementarity to a target RNA. In some cases, a targeting sequence comprises from 50 to 400 nucleotides total and has from 50 to 150 nucleotides having complementarity to a target RNA. In some cases, a targeting sequence comprises from 50 to 400 nucleotides total and has from 50 to 200 nucleotides having complementarity to a target RNA. In some cases, a targeting sequence comprises from 50 to 400 nucleotides total and has from 50 to 250 nucleotides having complementarity to a target RNA. In some cases, a targeting sequence comprises from 50 to 400 nucleotides total and has from 50 to 300 nucleotides having complementarity to a target RNA. In some cases, the at least 50 nucleotides having complementarity to a target RNA are separated by one or more mismatches, one or more bulges, or one or more loops, or any combination thereof. In some cases, the from 50 to 150 nucleotides having complementarity to a target RNA are separated by one or more mismatches, one or more bulges, or one or more loops, or any combination thereof. In some cases, the from 50 to 200 nucleotides having complementarity to a target RNA are separated by one or more mismatches, one or more bulges, or one or more loops, or any combination thereof. In some cases, the from 50 to 250 nucleotides having complementarity to a target RNA are separated by one or more mismatches, one or more bulges, or one or more loops, or any combination thereof. In some cases, the from 50 to 300 nucleotides having complementarity to a target RNA are separated by one or more mismatches, one or more bulges, or one or more loops, or any combination thereof. For example, a targeting sequence comprises a total of 54 nucleotides wherein, sequentially, 25 nucleotides are complementarity to a target RNA, 4 nucleotides form a bulge, and 25 nucleotides are complementarity to a target RNA. As another example, a targeting sequence comprises a total of 118 nucleotides wherein, sequentially, 25 nucleotides are complementarity to a target RNA, 4 nucleotides form a bulge, 25 nucleotides are complementarity to a target RNA, 14 nucleotides form a loop, and 50 nucleotides are complementary to a target RNA.

In an aspect, a structural feature can form in an engineered polynucleotide independently. In other cases, a structural feature can form when an engineered polynucleotide binds to a target RNA. A structural feature can also form when an engineered polynucleotide associates with other molecules such as a peptide, a nucleotide, or a small molecule. In certain embodiments, a structural feature of an engineered polynucleotide can be formed independent of a target RNA, and its structure can change as a result of the engineered polypeptide hybridization with a target RNA region. In certain embodiments, a structural feature can be present when an engineered polynucleotide can be in association with a target RNA.

In some cases, a structural feature can be a hairpin. In some cases, an engineered polynucleotide can lack a hairpin domain. In other cases, an engineered polynucleotide can contain a hairpin domain or more than one hairpin domain. A hairpin can be located anywhere in a polynucleotide. As disclosed herein, a hairpin can be an RNA duplex wherein a single RNA strand has folded in upon itself to form the RNA duplex. The single RNA strand folds upon itself due to having nucleotide sequences upstream and downstream of the folding region base pairs to each other. A hairpin may have from 10 to 500 nucleotides in length of the entire duplex structure. The stem-loop structure of a hairpin may be from 3 to 15 nucleotides long. A hairpin may be present in any of the engineered polynucleotides disclosed herein. The engineered polynucleotides disclosed herein may have from 1 to 10 hairpins. In some embodiments, the engineered polynucleotides disclosed herein have 1 hairpin. In some embodiments, the engineered polynucleotides disclosed herein have 2 hairpins. As disclosed herein, a hairpin may refer to a recruitment hairpin or a hairpin or a non-recruitment hairpin. A hairpin can be located anywhere within the engineered polynucleotides of the present disclosure. In some embodiments, one or more hairpins can be present at the 3′ end of an engineered polynucleotide of the present disclosure, at the 5′ end of an engineered polynucleotide of the present disclosure or within the targeting sequence of an engineered polynucleotide of the present disclosure, or any combination thereof.

In aspect, a structural feature comprises a recruitment hairpin, as disclosed herein. A recruitment hairpin may recruit an RNA editing entity, such as ADAR. In some embodiments, a recruitment hairpin comprises a GluR2 domain. In some embodiments, a recruitment hairpin comprises an Alu domain.

In yet another aspect, a structural feature comprises a non-recruitment hairpin. A non-recruitment hairpin, as disclosed herein, may exhibit functionality that improves localization of the engineered polynucleotide to the target RNA. In some embodiments, the non-recruitment hairpin improves nuclear retention. In some embodiments, the non-recruitment hairpin comprises a hairpin from U7 snRNA.

In another aspect, a structural feature comprises a wobble base. A wobble base pair refers to two bases that weakly pair. For example, a wobble base pair of the present disclosure may refer to a G paired with a U.

A hairpin of the present disclosure can be of any length. In an aspect, a hairpin can be from about 5-200 or more nucleotides. In some cases, a hairpin can comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 or more nucleotides. In other cases, a hairpin can also comprise 5 to 10, 5 to 20, 5 to 30, 5 to 40, 5 to 50, 5 to 60, 5 to 70, 5 to 80, 5 to 90, 5 to 100, 5 to 110, 5 to 120, 5 to 130, 5 to 140, 5 to 150, 5 to 160, 5 to 170, 5 to 180, 5 to 190, 5 to 200, 5 to 210, 5 to 220, 5 to 230, 5 to 240, 5 to 250, 5 to 260, 5 to 270, 5 to 280, 5 to 290, 5 to 300, 5 to 310, 5 to 320, 5 to 330, 5 to 340, 5 to 350, 5 to 360, 5 to 370, 5 to 380, 5 to 390, or 5 to 400 nucleotides. A hairpin can be a structural feature formed from a single strand of RNA with sufficient complementarity to itself to hybridize into a double stranded RNA motif/structure consisting of double-stranded hybridized RNA separated by a nucleotide loop.

In some cases, a structural feature can be a bulge. A bulge can comprise a single (intentional) nucleic acid mismatch between the target strand and an engineered polynucleotide strand. In other cases, more than one consecutive mismatch between strands constitutes a bulge as long as the bulge region, mismatched stretch of bases, can be flanked on both sides with hybridized, complementary dsRNA regions. A bulge can be located at any location of a polynucleotide. In some cases, a bulge can be located from about 30 to about 70 nucleotides from a 5′ hydroxyl or the 3′ hydroxyl.

In an embodiment, a double stranded RNA (dsRNA) substrate can be formed upon hybridization of an engineered polynucleotide of the present disclosure to a target RNA. As disclosed herein, a bulge refers to the structure formed upon formation of the dsRNA substrate, where nucleotides in either the engineered polynucleotide or the target RNA are not complementary to their positional counterparts on the opposite strand. A bulge may change the secondary or tertiary structure of the dsRNA substrate. A bulge may have from 1 to 4 nucleotides on the engineered polynucleotide side of the dsRNA substrate or the target RNA side of the dsRNA substrate. In some embodiments, the engineered polynucleotides of the present disclosure have 2 bulges. In some embodiments, the engineered polynucleotides of the present disclosure have 3 bulges. In some embodiments, the engineered polynucleotides of the present disclosure have 4 bulges. In some embodiments, the presence of a bulge in a dsRNA substrate may position ADAR to selectively edit the target A in the target RNA and reduce off-target editing of non-targets. In some embodiments, the presence of a bulge in a dsRNA substrate may recruit additional ADAR. Bulges in dsRNA substrates disclosed herein may recruit other proteins, such as other RNA editing entities. In some embodiments, a bulge positioned 5′ of the edit site may facilitate base-flipping of the target A to be edited. A bulge may also help confer sequence specificity. A bulge may help direct ADAR editing by constraining it in an orientation that yield selective editing of the target A. In some embodiments, selective editing of the target A is achieved by positioning the target A between two bulges (e.g., positioned between a 5′ end bulge and a 3′ end bulge, based on the engineered polynucleotide). In some embodiments, the two bulges are both symmetrical bulges. In some embodiments, the two bulges each are formed by 2 nucleotides on the engineered polynucleotide side of the dsRNA target and 2 nucleotides on the target RNA side of the dsRNA substrate. In some embodiments, the two bulges each are formed by 3 nucleotides on the engineered polynucleotide side of the dsRNA target and 3 nucleotides on the target RNA side of the dsRNA substrate. In some embodiments, the two bulges each are formed by 4 nucleotides on the engineered polynucleotide side of the dsRNA target and 4 nucleotides on the target RNA side of the dsRNA substrate. In some embodiments, the target A is position between the two bulges, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a bulge (e.g., from a 5′end bulge or a 3′ end bulge). In some embodiments, additional structural features are located between the bulges (e.g., between the 5′end bulge and the 3′ end bulge). In some embodiments, a mismatch in a bulge comprises a nucleotide base for editing in the target RNA (e.g., an A/C mismatch in the bulge, wherein part of the bulge in the engineered polynucleotide comprises a C mismatched to an A in the part of the bulge in the target RNA, and the A is edited).

In an aspect, a double stranded RNA (dsRNA) substrate can be formed upon hybridization of an engineered polynucleotide of the present disclosure to a target RNA. A bulge may be a symmetrical bulge or an asymmetrical bulge. A bulge may be formed by 1 to 4 participating nucleotides on either the guide RNA side or the target RNA side of the dsRNA substrate. A symmetrical bulge can be formed when the same number of nucleotides can be present on each side of the bulge. A symmetrical bulge may have from 2 to 4 nucleotides on the engineered polynucleotide side of the dsRNA substrate or the target RNA side of the dsRNA substrate. For example, a symmetrical bulge in a dsRNA substrate of the present disclosure may have the same number of nucleotides on the engineered polynucleotide side and the target RNA side of the dsRNA substrate. A symmetrical bulge of the present disclosure may be formed by 2 nucleotides on the engineered polynucleotide side of the dsRNA target and 2 nucleotides on the target RNA side of the dsRNA substrate. A symmetrical bulge of the present disclosure may be formed by 3 nucleotides on the engineered polynucleotide side of the dsRNA target and 3 nucleotides on the target RNA side of the dsRNA substrate. A symmetrical bulge of the present disclosure may be formed by 4 nucleotides on the engineered polynucleotide side of the dsRNA target and 4 nucleotides on the target RNA side of the dsRNA substrate.

A double stranded RNA (dsRNA) substrate can be formed upon hybridization of an engineered guide RNA of the present disclosure to a target RNA. A bulge may be a symmetrical bulge or an asymmetrical bulge. An asymmetrical bulge can be formed when a different number of nucleotides can be present on each side of the bulge. An asymmetrical bulge may have from 1 to 4 participating nucleotides on either the guide RNA side or the target RNA side of the dsRNA substrate. For example, an asymmetrical bulge in a dsRNA substrate of the present disclosure may have different numbers of nucleotides on the engineered guide RNA side and the target RNA side of the dsRNA substrate. An asymmetrical bulge of the present disclosure may be formed by 0 nucleotides on the engineered guide RNA side of the dsRNA substrate and 1 nucleotide on the target RNA side of the dsRNA substrate. An asymmetrical bulge of the present disclosure may be formed by 0 nucleotides on the target RNA side of the dsRNA substrate and 1 nucleotide on the engineered guide RNA side of the dsRNA substrate. An asymmetrical bulge of the present disclosure may be formed by 0 nucleotides on the engineered guide RNA side of the dsRNA substrate and 2 nucleotides on the target RNA side of the dsRNA substrate. An asymmetrical bulge of the present disclosure may be formed by 0 nucleotides on the target RNA side of the dsRNA substrate and 2 nucleotides on the engineered guide RNA side of the dsRNA substrate. An asymmetrical bulge of the present disclosure may be formed by 0 nucleotides on the engineered guide RNA side of the dsRNA substrate and 3 nucleotides on the target RNA side of the dsRNA substrate. An asymmetrical bulge of the present disclosure may be formed by 0 nucleotides on the target RNA side of the dsRNA substrate and 3 nucleotides on the engineered guide RNA side of the dsRNA substrate. An asymmetrical bulge of the present disclosure may be formed by 0 nucleotides on the engineered guide RNA side of the dsRNA substrate and 4 nucleotides on the target RNA side of the dsRNA substrate. An asymmetrical bulge of the present disclosure may be formed by 0 nucleotides on the target RNA side of the dsRNA substrate and 4 nucleotides on the engineered guide RNA side of the dsRNA substrate. An asymmetrical bulge of the present disclosure may be formed by 1 nucleotide on the engineered guide RNA side of the dsRNA substrate and 2 nucleotides on the target RNA side of the dsRNA substrate. An asymmetrical bulge of the present disclosure may be formed by 1 nucleotide on the target RNA side of the dsRNA substrate and 2 nucleotides on the engineered guide RNA side of the dsRNA substrate. An asymmetrical bulge of the present disclosure may be formed by 1 nucleotide on the engineered guide RNA side of the dsRNA substrate and 3 nucleotides on the target RNA side of the dsRNA substrate. An asymmetrical bulge of the present disclosure may be formed by 1 nucleotide on the target RNA side of the dsRNA substrate and 3 nucleotides on the engineered guide RNA side of the dsRNA substrate. An asymmetrical bulge of the present disclosure may be formed by 1 nucleotides on the engineered guide RNA side of the dsRNA substrate and 4 nucleotides on the target RNA side of the dsRNA substrate. An asymmetrical bulge of the present disclosure may be formed by 1 nucleotide on the target RNA side of the dsRNA substrate and 4 nucleotides on the engineered guide RNA side of the dsRNA substrate. An asymmetrical bulge of the present disclosure may be formed by 2 nucleotides on the engineered guide RNA side of the dsRNA substrate and 3 nucleotides on the target RNA side of the dsRNA substrate. An asymmetrical bulge of the present disclosure may be formed by 2 nucleotides on the target RNA side of the dsRNA substrate and 3 nucleotides on the engineered guide RNA side of the dsRNA substrate. An asymmetrical bulge of the present disclosure may be formed by 2 nucleotides on the engineered guide RNA side of the dsRNA substrate and 4 nucleotides on the target RNA side of the dsRNA substrate. An asymmetrical bulge of the present disclosure may be formed by 2 nucleotides on the target RNA side of the dsRNA substrate and 4 nucleotides on the engineered guide RNA side of the dsRNA substrate. An asymmetrical bulge of the present disclosure may be formed by 3 nucleotides on the engineered guide RNA side of the dsRNA substrate and 4 nucleotides on the target RNA side of the dsRNA substrate. An asymmetrical bulge of the present disclosure may be formed by 3 nucleotides on the target RNA side of the dsRNA substrate and 4 nucleotides on the engineered guide RNA side of the dsRNA substrate. In some embodiments, an asymmetrical bulge increases efficiency of editing a target A. In some embodiments, an asymmetrical bulge that increases efficiency of editing a target A is an asymmetrical bulge that is formed to reduce the number of adenosines in the sequence of the engineered polynucleotide. Non-limiting examples of an asymmetrical bulge that increases efficiency of editing a target A are an asymmetrical bulge formed by 0 nucleotides on the engineered polynucleotide side of the dsRNA substrate and 1 nucleotide on the target RNA side of the dsRNA substrate; an asymmetrical bulge of formed by 0 nucleotides on the engineered polynucleotide side of the dsRNA substrate and 2 nucleotides on the target RNA side of the dsRNA substrate; an asymmetrical bulge of formed by 0 nucleotides on the engineered polynucleotide side of the dsRNA substrate and 3 nucleotides on the target RNA side of the dsRNA substrate; an asymmetrical bulge of formed by 0 nucleotides on the engineered polynucleotide side of the dsRNA substrate and 4 nucleotides on the target RNA side of the dsRNA substrate; an asymmetrical bulge of formed by 1 nucleotide on the engineered polynucleotide side of the dsRNA substrate and 2 nucleotides on the target RNA side of the dsRNA substrate; an asymmetrical bulge of formed by 1 nucleotide on the engineered polynucleotide side of the dsRNA substrate and 3 nucleotides on the target RNA side of the dsRNA substrate; an asymmetrical bulge of formed by 1 nucleotide on the engineered polynucleotide side of the dsRNA substrate and 4 nucleotides on the target RNA side of the dsRNA substrate; an asymmetrical bulge of formed by 2 nucleotides on the engineered polynucleotide side of the dsRNA substrate and 3 nucleotides on the target RNA side of the dsRNA substrate; an asymmetrical bulge of formed by 2 nucleotides on the engineered polynucleotide side of the dsRNA substrate and 4 nucleotides on the target RNA side of the dsRNA substrate; and an asymmetrical bulge of formed by 3 nucleotides on the engineered polynucleotide side of the dsRNA substrate and 4 nucleotides on the target RNA side of the dsRNA substrate.

In an aspect, a double stranded RNA (dsRNA) substrate can be formed upon hybridization of an engineered polynucleotide of the present disclosure to a target RNA. In some cases, a structural feature can be a loop. As disclosed herein, an internal loop refers to the structure formed upon formation of the dsRNA substrate, where nucleotides in either the engineered polynucleotide or the target RNA are not complementary to their positional counterparts on the opposite strand and where one side of the internal loop, either on the target RNA side or the engineered polynucleotide side of the dsRNA substrate, has greater than 5 nucleotides. An internal loop may be a symmetrical internal loop or an asymmetrical internal loop. Internal loops present in the vicinity of the edit site may help with base flipping of the target A in the target RNA to be edited. A double stranded RNA (dsRNA) substrate can be formed upon hybridization of an engineered polynucleotide of the present disclosure to a target RNA. An internal loop may be a symmetrical internal loop or an asymmetrical internal loop. In some embodiments, selective editing of the target A is achieved by positioning the target A between two loops (e.g., positioned between a 5′ end loop and a 3′ end loop, based on the engineered polynucleotide). In some embodiments, the two loops are both symmetrical loops. In some embodiments, the two loops each are formed by 5 nucleotides on the engineered polynucleotide side of the dsRNA target and 5 nucleotides on the target RNA side of the dsRNA substrate. In some embodiments, the two loops each are formed by 6 nucleotides on the engineered polynucleotide side of the dsRNA target and 6 nucleotides on the target RNA side of the dsRNA substrate. In some embodiments, the two loops each are formed by 7 nucleotides on the engineered polynucleotide side of the dsRNA target and 7 nucleotides on the target RNA side of the dsRNA substrate. In some embodiments, the two loops each are formed by 8 nucleotides on the engineered polynucleotide side of the dsRNA target and 8 nucleotides on the target RNA side of the dsRNA substrate. In some embodiments, the two loops each are formed by 9 nucleotides on the engineered polynucleotide side of the dsRNA target and 9 nucleotides on the target RNA side of the dsRNA substrate. In some embodiments, the two loops each are formed by 10 nucleotides on the engineered polynucleotide side of the dsRNA target and 10 nucleotides on the target RNA side of the dsRNA substrate. In some embodiments, the target A is position between the two loops, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a loop (e.g., from a 5′end loop or a 3′ end loop). In some embodiments, additional structural features are located between the loops (e.g., between the 5′end loop and the 3′ end loop). In some embodiments, a mismatch in a loop comprises a nucleotide base for editing in the target RNA (e.g., an A/C mismatch in the loop, wherein part of the bulge in the engineered polynucleotide comprises a C mismatched to an A in the part of the loop in the target RNA, and the A is edited).

A symmetrical internal loop can be formed when the same number of nucleotides can be present on each side of the internal loop. For example, a symmetrical internal loop in a dsRNA substrate of the present disclosure may have the same number of nucleotides on the engineered polynucleotide side and the target RNA side of the dsRNA substrate. A symmetrical internal loop of the present disclosure may be formed by 5 nucleotides on the engineered polynucleotide side of the dsRNA target and 5 nucleotides on the target RNA side of the dsRNA substrate. A symmetrical internal loop of the present disclosure may be formed by 6 nucleotides on the engineered polynucleotide side of the dsRNA target and 6 nucleotides on the target RNA side of the dsRNA substrate. A symmetrical internal loop of the present disclosure may be formed by 7 nucleotides on the engineered polynucleotide side of the dsRNA target and 7 nucleotides on the target RNA side of the dsRNA substrate. A symmetrical internal loop of the present disclosure may be formed by 8 nucleotides on the engineered polynucleotide side of the dsRNA target and 8 nucleotides on the target RNA side of the dsRNA substrate. A symmetrical internal loop of the present disclosure may be formed by 9 nucleotides on the engineered polynucleotide side of the dsRNA target and 9 nucleotides on the target RNA side of the dsRNA substrate. A symmetrical internal loop of the present disclosure may be formed by 10 nucleotides on the engineered polynucleotide side of the dsRNA target and 10 nucleotides on the target RNA side of the dsRNA substrate.

In an aspect, a double stranded RNA (dsRNA) substrate can be formed upon hybridization of an engineered polynucleotide of the present disclosure to a target RNA. An internal loop may be a symmetrical internal loop or an asymmetrical internal loop. Internal loops present in the vicinity of the edit site may help with base flipping of the target A in the target RNA to be edited. A double stranded RNA (dsRNA) substrate is formed upon hybridization of an engineered polynucleotide of the present disclosure to a target RNA. An internal loop may be a symmetrical internal loop or an asymmetrical internal loop. A symmetrical internal loop is formed when the same number of nucleotides is present on each side of the internal loop. For example, a symmetrical internal loop in a dsRNA substrate of the present disclosure may have the same number of nucleotides on the engineered polynucleotide side and the target RNA side of the dsRNA substrate. A symmetrical internal loop of the present disclosure may be formed by 5 nucleotides on the engineered polynucleotide side of the dsRNA target and 5 nucleotides on the target RNA side of the dsRNA substrate. A symmetrical internal loop of the present disclosure may be formed by 6 nucleotides on the engineered polynucleotide side of the dsRNA target and 6 nucleotides on the target RNA side of the dsRNA substrate. A symmetrical internal loop of the present disclosure may be formed by 7 nucleotides on the engineered polynucleotide side of the dsRNA target and 7 nucleotides on the target RNA side of the dsRNA substrate. A symmetrical internal loop of the present disclosure may be formed by 8 nucleotides on the engineered polynucleotide side of the dsRNA target and 8 nucleotides on the target RNA side of the dsRNA substrate. A symmetrical internal loop of the present disclosure may be formed by 9 nucleotides on the engineered polynucleotide side of the dsRNA target and 9 nucleotides on the target RNA side of the dsRNA substrate. A symmetrical internal loop of the present disclosure may be formed by 10 nucleotides on the engineered polynucleotide side of the dsRNA target and 10 nucleotides on the target RNA side of the dsRNA substrate. One side of the internal loop, either on the target RNA side or the engineered polynucleotide side of the dsRNA substrate, may be formed by from 5 to 150 nucleotides. One side of the internal loop may be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop may be formed by 5 nucleotides. One side of the internal loop may be formed by 10 nucleotides. One side of the internal loop may be formed by 15 nucleotides. One side of the internal loop may be formed by 20 nucleotides. One side of the internal loop may be formed by 25 nucleotides. One side of the internal loop may be formed by 30 nucleotides. One side of the internal loop may be formed by 35 nucleotides. One side of the internal loop may be formed by 40 nucleotides. One side of the internal loop may be formed by 45 nucleotides. One side of the internal loop may be formed by 50 nucleotides. One side of the internal loop may be formed by 55 nucleotides. One side of the internal loop may be formed by 60 nucleotides. One side of the internal loop may be formed by 65 nucleotides. One side of the internal loop may be formed by 70 nucleotides. One side of the internal loop may be formed by 75 nucleotides. One side of the internal loop may be formed by 80 nucleotides. One side of the internal loop may be formed by 85 nucleotides. One side of the internal loop may be formed by 90 nucleotides. One side of the internal loop may be formed by 95 nucleotides. One side of the internal loop may be formed by 100 nucleotides. One side of the internal loop may be formed by 110 nucleotides. One side of the internal loop may be formed by 120 nucleotides. One side of the internal loop may be formed by 130 nucleotides. One side of the internal loop may be formed by 140 nucleotides. One side of the internal loop may be formed by 150 nucleotides. One side of the internal loop may be formed by 200 nucleotides. One side of the internal loop may be formed by 250 nucleotides. One side of the internal loop may be formed by 300 nucleotides. One side of the internal loop may be formed by 350 nucleotides. One side of the internal loop may be formed by 400 nucleotides. One side of the internal loop may be formed by 450 nucleotides. One side of the internal loop may be formed by 500 nucleotides. One side of the internal loop may be formed by 600 nucleotides. One side of the internal loop may be formed by 700 nucleotides. One side of the internal loop may be formed by 800 nucleotides. One side of the internal loop may be formed by 900 nucleotides. One side of the internal loop may be formed by 1000 nucleotides. An internal loop may be a symmetrical internal loop or an asymmetrical internal loop. Internal loops present in the vicinity of the edit site may help with base flipping of the target A in the target RNA to be edited. A double stranded RNA (dsRNA) substrate is formed upon hybridization of an engineered polynucleotide of the present disclosure to a target RNA. An internal loop may be a symmetrical internal loop or an asymmetrical internal loop. A symmetrical internal loop is formed when the same number of nucleotides is present on each side of the internal loop. For example, a symmetrical internal loop in a dsRNA substrate of the present disclosure may have the same number of nucleotides on the engineered polynucleotide side and the target RNA side of the dsRNA substrate. A symmetrical internal loop of the present disclosure may be formed by from 5 to 150 nucleotides on the engineered polynucleotide side of the dsRNA target and from 5 to 150 nucleotides on the target RNA side of the dsRNA substrate, wherein the number of nucleotides is the same on the engineered side of the dsRNA target and the target RNA side of the dsRNA substrate. A symmetrical internal loop of the present disclosure may be formed by from 5 to 1000 nucleotides on the engineered polynucleotide side of the dsRNA target and from 5 to 1000 nucleotides on the target RNA side of the dsRNA substrate, wherein the number of nucleotides is the same on the engineered side of the dsRNA target and the target RNA side of the dsRNA substrate. A symmetrical internal loop of the present disclosure may be formed by 5 nucleotides on the engineered polynucleotide side of the dsRNA target and 5 nucleotides on the target RNA side of the dsRNA substrate. A symmetrical internal loop of the present disclosure may be formed by 6 nucleotides on the engineered polynucleotide side of the dsRNA target and 6 nucleotides on the target RNA side of the dsRNA substrate. A symmetrical internal loop of the present disclosure may be formed by 7 nucleotides on the engineered polynucleotide side of the dsRNA target and 7 nucleotides on the target RNA side of the dsRNA substrate. A symmetrical internal loop of the present disclosure may be formed by 8 nucleotides on the engineered polynucleotide side of the dsRNA target and 8 nucleotides on the target RNA side of the dsRNA substrate. A symmetrical internal loop of the present disclosure may be formed by 9 nucleotides on the engineered polynucleotide side of the dsRNA target and 9 nucleotides on the target RNA side of the dsRNA substrate. A symmetrical internal loop of the present disclosure may be formed by 10 nucleotides on the engineered polynucleotide side of the dsRNA target and 10 nucleotides on the target RNA side of the dsRNA substrate. A symmetrical internal loop of the present disclosure may be formed by 15 nucleotides on the engineered polynucleotide side of the dsRNA target and 15 nucleotides on the target RNA side of the dsRNA substrate. A symmetrical internal loop of the present disclosure may be formed by 20 nucleotides on the engineered polynucleotide side of the dsRNA target and 20 nucleotides on the target RNA side of the dsRNA substrate. A symmetrical internal loop of the present disclosure may be formed by 30 nucleotides on the engineered polynucleotide side of the dsRNA target and 30 nucleotides on the target RNA side of the dsRNA substrate. A symmetrical internal loop of the present disclosure may be formed by 40 nucleotides on the engineered polynucleotide side of the dsRNA target and 40 nucleotides on the target RNA side of the dsRNA substrate. A symmetrical internal loop of the present disclosure may be formed by 50 nucleotides on the engineered polynucleotide side of the dsRNA target and 50 nucleotides on the target RNA side of the dsRNA substrate. A symmetrical internal loop of the present disclosure may be formed by 60 nucleotides on the engineered polynucleotide side of the dsRNA target and 60 nucleotides on the target RNA side of the dsRNA substrate. A symmetrical internal loop of the present disclosure may be formed by 70 nucleotides on the engineered polynucleotide side of the dsRNA target and 70 nucleotides on the target RNA side of the dsRNA substrate. A symmetrical internal loop of the present disclosure may be formed by 80 nucleotides on the engineered polynucleotide side of the dsRNA target and 80 nucleotides on the target RNA side of the dsRNA substrate. A symmetrical internal loop of the present disclosure may be formed by 90 nucleotides on the engineered polynucleotide side of the dsRNA target and 90 nucleotides on the target RNA side of the dsRNA substrate. A symmetrical internal loop of the present disclosure may be formed by 100 nucleotides on the engineered polynucleotide side of the dsRNA target and 100 nucleotides on the target RNA side of the dsRNA substrate. A symmetrical internal loop of the present disclosure may be formed by 110 nucleotides on the engineered polynucleotide side of the dsRNA target and 110 nucleotides on the target RNA side of the dsRNA substrate. A symmetrical internal loop of the present disclosure may be formed by 120 nucleotides on the engineered polynucleotide side of the dsRNA target and 120 nucleotides on the target RNA side of the dsRNA substrate. A symmetrical internal loop of the present disclosure may be formed by 130 nucleotides on the engineered polynucleotide side of the dsRNA target and 130 nucleotides on the target RNA side of the dsRNA substrate. A symmetrical internal loop of the present disclosure may be formed by 140 nucleotides on the engineered polynucleotide side of the dsRNA target and 140 nucleotides on the target RNA side of the dsRNA substrate. A symmetrical internal loop of the present disclosure may be formed by 150 nucleotides on the engineered polynucleotide side of the dsRNA target and 150 nucleotides on the target RNA side of the dsRNA substrate. A symmetrical internal loop of the present disclosure may be formed by 200 nucleotides on the engineered polynucleotide side of the dsRNA target and 200 nucleotides on the target RNA side of the dsRNA substrate. A symmetrical internal loop of the present disclosure may be formed by 250 nucleotides on the engineered polynucleotide side of the dsRNA target and 250 nucleotides on the target RNA side of the dsRNA substrate. A symmetrical internal loop of the present disclosure may be formed by 300 nucleotides on the engineered polynucleotide side of the dsRNA target and 300 nucleotides on the target RNA side of the dsRNA substrate. A symmetrical internal loop of the present disclosure may be formed by 350 nucleotides on the engineered polynucleotide side of the dsRNA target and 350 nucleotides on the target RNA side of the dsRNA substrate. A symmetrical internal loop of the present disclosure may be formed by 400 nucleotides on the engineered polynucleotide side of the dsRNA target and 400 nucleotides on the target RNA side of the dsRNA substrate. A symmetrical internal loop of the present disclosure may be formed by 450 nucleotides on the engineered polynucleotide side of the dsRNA target and 450 nucleotides on the target RNA side of the dsRNA substrate. A symmetrical internal loop of the present disclosure may be formed by 500 nucleotides on the engineered polynucleotide side of the dsRNA target and 500 nucleotides on the target RNA side of the dsRNA substrate. A symmetrical internal loop of the present disclosure may be formed by 600 nucleotides on the engineered polynucleotide side of the dsRNA target and 600 nucleotides on the target RNA side of the dsRNA substrate. A symmetrical internal loop of the present disclosure may be formed by 700 nucleotides on the engineered polynucleotide side of the dsRNA target and 700 nucleotides on the target RNA side of the dsRNA substrate. A symmetrical internal loop of the present disclosure may be formed by 800 nucleotides on the engineered polynucleotide side of the dsRNA target and 800 nucleotides on the target RNA side of the dsRNA substrate. A symmetrical internal loop of the present disclosure may be formed by 900 nucleotides on the engineered polynucleotide side of the dsRNA target and 900 nucleotides on the target RNA side of the dsRNA substrate. A symmetrical internal loop of the present disclosure may be formed by 1000 nucleotides on the engineered polynucleotide side of the dsRNA target and 1000 nucleotides on the target RNA side of the dsRNA substrate.

In an aspect, a double stranded RNA (dsRNA) substrate is formed upon hybridization of an engineered polynucleotide of the present disclosure to a target RNA. An internal loop may be a symmetrical internal loop or an asymmetrical internal loop. An asymmetrical internal loop is formed when a different number of nucleotides is present on each side of the internal loop. For example, an asymmetrical internal loop in a dsRNA substrate of the present disclosure may have different numbers of nucleotides on the engineered polynucleotide side and the target RNA side of the dsRNA substrate. An asymmetrical internal loop of the present disclosure may be formed by from 5 to 150 nucleotides on the engineered polynucleotide side of the dsRNA substrate and from 5 to 150 nucleotides on the target RNA side of the dsRNA substrate, wherein the number of nucleotides is the different on the engineered side of the dsRNA target than the number of nucleotides on the target RNA side of the dsRNA substrate. An asymmetrical internal loop of the present disclosure may be formed by from 5 to 1000 nucleotides on the engineered polynucleotide side of the dsRNA substrate and from 5 to 1000 nucleotides on the target RNA side of the dsRNA substrate, wherein the number of nucleotides is the different on the engineered side of the dsRNA target than the number of nucleotides on the target RNA side of the dsRNA substrate. An asymmetrical internal loop of the present disclosure may be formed by 5 nucleotides on the engineered polynucleotide side of the dsRNA substrate and 6 nucleotides on the target RNA side of the dsRNA substrate. An asymmetrical internal loop of the present disclosure may be formed by 5 nucleotides on the target RNA side of the dsRNA substrate and 6 nucleotides on the engineered polynucleotide side of the dsRNA substrate. An asymmetrical internal loop of the present disclosure may be formed by 5 nucleotides on the engineered polynucleotide side of the dsRNA substrate and 7 nucleotides on the target RNA side of the dsRNA substrate. An asymmetrical internal loop of the present disclosure may be formed by 5 nucleotides on the target RNA side of the dsRNA substrate and 7 nucleotides on the engineered polynucleotide side of the dsRNA substrate. An asymmetrical internal loop of the present disclosure may be formed by 5 nucleotides on the engineered polynucleotide side of the dsRNA substrate and 8 nucleotides internal loop the target RNA side of the dsRNA substrate. An asymmetrical internal loop of the present disclosure may be formed by 5 nucleotides on the target RNA side of the dsRNA substrate and 8 nucleotides on the engineered polynucleotide side of the dsRNA substrate. An asymmetrical internal loop of the present disclosure may be formed by 5 nucleotides on the engineered polynucleotide side of the dsRNA substrate and 9 nucleotides internal loop the target RNA side of the dsRNA substrate. An asymmetrical internal loop of the present disclosure may be formed by 5 nucleotides on the target RNA side of the dsRNA substrate and 9 nucleotides on the engineered polynucleotide side of the dsRNA substrate. An asymmetrical internal loop of the present disclosure may be formed by 5 nucleotides on the engineered polynucleotide side of the dsRNA substrate and 10 nucleotides internal loop the target RNA side of the dsRNA substrate. An asymmetrical internal loop of the present disclosure may be formed by 5 nucleotides on the target RNA side of the dsRNA substrate and 10 nucleotides on the engineered polynucleotide side of the dsRNA substrate. An asymmetrical internal loop of the present disclosure may be formed by 6 nucleotides on the engineered polynucleotide side of the dsRNA substrate and 7 nucleotides internal loop the target RNA side of the dsRNA substrate. An asymmetrical internal loop of the present disclosure may be formed by 6 nucleotides on the target RNA side of the dsRNA substrate and 7 nucleotides on the engineered polynucleotide side of the dsRNA substrate. An asymmetrical internal loop of the present disclosure may be formed by 6 nucleotides on the engineered polynucleotide side of the dsRNA substrate and 8 nucleotides internal loop the target RNA side of the dsRNA substrate. An asymmetrical internal loop of the present disclosure may be formed by 6 nucleotides on the target RNA side of the dsRNA substrate and 8 nucleotides on the engineered polynucleotide side of the dsRNA substrate. An asymmetrical internal loop of the present disclosure may be formed by 6 nucleotides on the engineered polynucleotide side of the dsRNA substrate and 9 nucleotides internal loop the target RNA side of the dsRNA substrate. An asymmetrical internal loop of the present disclosure may be formed by 6 nucleotides on the target RNA side of the dsRNA substrate and 9 nucleotides on the engineered polynucleotide side of the dsRNA substrate. An asymmetrical internal loop of the present disclosure may be formed by 6 nucleotides on the engineered polynucleotide side of the dsRNA substrate and 10 nucleotides internal loop the target RNA side of the dsRNA substrate. An asymmetrical internal loop of the present disclosure may be formed by 6 nucleotides on the target RNA side of the dsRNA substrate and 10 nucleotides on the engineered polynucleotide side of the dsRNA substrate. An asymmetrical internal loop of the present disclosure may be formed by 7 nucleotides on the engineered polynucleotide side of the dsRNA substrate and 8 nucleotides internal loop the target RNA side of the dsRNA substrate. An asymmetrical internal loop of the present disclosure may be formed by 7 nucleotides on the target RNA side of the dsRNA substrate and 8 nucleotides on the engineered polynucleotide side of the dsRNA substrate. An asymmetrical internal loop of the present disclosure may be formed by 7 nucleotides on the engineered polynucleotide side of the dsRNA substrate and 9 nucleotides internal loop the target RNA side of the dsRNA substrate. An asymmetrical internal loop of the present disclosure may be formed by 7 nucleotides on the target RNA side of the dsRNA substrate and 9 nucleotides on the engineered polynucleotide side of the dsRNA substrate. An asymmetrical internal loop of the present disclosure may be formed by 7 nucleotides on the engineered polynucleotide side of the dsRNA substrate and 10 nucleotides internal loop the target RNA side of the dsRNA substrate. An asymmetrical internal loop of the present disclosure may be formed by 7 nucleotides on the target RNA side of the dsRNA substrate and 10 nucleotides on the engineered polynucleotide side of the dsRNA substrate. An asymmetrical internal loop of the present disclosure may be formed by 8 nucleotides on the engineered polynucleotide side of the dsRNA substrate and 9 nucleotides internal loop the target RNA side of the dsRNA substrate. An asymmetrical internal loop of the present disclosure may be formed by 8 nucleotides on the target RNA side of the dsRNA substrate and 9 nucleotides on the engineered polynucleotide side of the dsRNA substrate. An asymmetrical internal loop of the present disclosure may be formed by 8 nucleotides on the engineered polynucleotide side of the dsRNA substrate and 10 nucleotides internal loop the target RNA side of the dsRNA substrate. An asymmetrical internal loop of the present disclosure may be formed by 8 nucleotides on the target RNA side of the dsRNA substrate and 10 nucleotides on the engineered polynucleotide side of the dsRNA substrate. An asymmetrical internal loop of the present disclosure may be formed by 9 nucleotides on the engineered polynucleotide side of the dsRNA substrate and 10 nucleotides internal loop the target RNA side of the dsRNA substrate. An asymmetrical internal loop of the present disclosure may be formed by 9 nucleotides on the target RNA side of the dsRNA substrate and 10 nucleotides on the engineered polynucleotide side of the dsRNA substrate. An asymmetrical internal loop of the present disclosure may be formed by 5 nucleotides on the target RNA side of the dsRNA substrate and 50 nucleotides on the engineered polynucleotide side of the dsRNA substrate. An asymmetrical internal loop of the present disclosure may be formed by 5 nucleotides on the target RNA side of the dsRNA substrate and 100 nucleotides on the engineered polynucleotide side of the dsRNA substrate. An asymmetrical internal loop of the present disclosure may be formed by 5 nucleotides on the target RNA side of the dsRNA substrate and 150 nucleotides on the engineered polynucleotide side of the dsRNA substrate. An asymmetrical internal loop of the present disclosure may be formed by 5 nucleotides on the target RNA side of the dsRNA substrate and 200 nucleotides on the engineered polynucleotide side of the dsRNA substrate. An asymmetrical internal loop of the present disclosure may be formed by 5 nucleotides on the target RNA side of the dsRNA substrate and 300 nucleotides on the engineered polynucleotide side of the dsRNA substrate. An asymmetrical internal loop of the present disclosure may be formed by 5 nucleotides on the target RNA side of the dsRNA substrate and 400 nucleotides on the engineered polynucleotide side of the dsRNA substrate. An asymmetrical internal loop of the present disclosure may be formed by 5 nucleotides on the target RNA side of the dsRNA substrate and 500 nucleotides on the engineered polynucleotide side of the dsRNA substrate. An asymmetrical internal loop of the present disclosure may be formed by 5 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 5 nucleotides on the engineered polynucleotide side of the dsRNA substrate. An asymmetrical internal loop of the present disclosure may be formed by 500 nucleotides on the target RNA side of the dsRNA substrate and 5 nucleotides on the engineered polynucleotide side of the dsRNA substrate. An asymmetrical internal loop of the present disclosure may be formed by 400 nucleotides on the target RNA side of the dsRNA substrate and 5 nucleotides on the engineered polynucleotide side of the dsRNA substrate. An asymmetrical internal loop of the present disclosure may be formed by 300 nucleotides on the target RNA side of the dsRNA substrate and 5 nucleotides on the engineered polynucleotide side of the dsRNA substrate. An asymmetrical internal loop of the present disclosure may be formed by 200 nucleotides on the target RNA side of the dsRNA substrate and 5 nucleotides on the engineered polynucleotide side of the dsRNA substrate. An asymmetrical internal loop of the present disclosure may be formed by 150 nucleotides on the target RNA side of the dsRNA substrate and 5 nucleotides on the engineered polynucleotide side of the dsRNA substrate. An asymmetrical internal loop of the present disclosure may be formed by 100 nucleotides on the target RNA side of the dsRNA substrate and 5 nucleotides on the engineered polynucleotide side of the dsRNA substrate. An asymmetrical internal loop of the present disclosure may be formed by 50 nucleotides on the target RNA side of the dsRNA substrate and 5 nucleotides on the engineered polynucleotide side of the dsRNA substrate. An asymmetrical internal loop of the present disclosure may be formed by 50 nucleotides on the target RNA side of the dsRNA substrate and 100 nucleotides on the engineered polynucleotide side of the dsRNA substrate. An asymmetrical internal loop of the present disclosure may be formed by 50 nucleotides on the target RNA side of the dsRNA substrate and 150 nucleotides on the engineered polynucleotide side of the dsRNA substrate. An asymmetrical internal loop of the present disclosure may be formed by 50 nucleotides on the target RNA side of the dsRNA substrate and 200 nucleotides on the engineered polynucleotide side of the dsRNA substrate. An asymmetrical internal loop of the present disclosure may be formed by 50 nucleotides on the target RNA side of the dsRNA substrate and 300 nucleotides on the engineered polynucleotide side of the dsRNA substrate. An asymmetrical internal loop of the present disclosure may be formed by 50 nucleotides on the target RNA side of the dsRNA substrate and 400 nucleotides on the engineered polynucleotide side of the dsRNA substrate. An asymmetrical internal loop of the present disclosure may be formed by 50 nucleotides on the target RNA side of the dsRNA substrate and 500 nucleotides on the engineered polynucleotide side of the dsRNA substrate. An asymmetrical internal loop of the present disclosure may be formed by 50 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 50 nucleotides on the engineered polynucleotide side of the dsRNA substrate. An asymmetrical internal loop of the present disclosure may be formed by 500 nucleotides on the target RNA side of the dsRNA substrate and 50 nucleotides on the engineered polynucleotide side of the dsRNA substrate. An asymmetrical internal loop of the present disclosure may be formed by 400 nucleotides on the target RNA side of the dsRNA substrate and 50 nucleotides on the engineered polynucleotide side of the dsRNA substrate. An asymmetrical internal loop of the present disclosure may be formed by 300 nucleotides on the target RNA side of the dsRNA substrate and 50 nucleotides on the engineered polynucleotide side of the dsRNA substrate. An asymmetrical internal loop of the present disclosure may be formed by 200 nucleotides on the target RNA side of the dsRNA substrate and 50 nucleotides on the engineered polynucleotide side of the dsRNA substrate. An asymmetrical internal loop of the present disclosure may be formed by 150 nucleotides on the target RNA side of the dsRNA substrate and 50 nucleotides on the engineered polynucleotide side of the dsRNA substrate. An asymmetrical internal loop of the present disclosure may be formed by 100 nucleotides on the target RNA side of the dsRNA substrate and 50 nucleotides on the engineered polynucleotide side of the dsRNA substrate. An asymmetrical internal loop of the present disclosure may be formed by 100 nucleotides on the target RNA side of the dsRNA substrate and 150 nucleotides on the engineered polynucleotide side of the dsRNA substrate. An asymmetrical internal loop of the present disclosure may be formed by 100 nucleotides on the target RNA side of the dsRNA substrate and 200 nucleotides on the engineered polynucleotide side of the dsRNA substrate. An asymmetrical internal loop of the present disclosure may be formed by 100 nucleotides on the target RNA side of the dsRNA substrate and 300 nucleotides on the engineered polynucleotide side of the dsRNA substrate. An asymmetrical internal loop of the present disclosure may be formed by 100 nucleotides on the target RNA side of the dsRNA substrate and 400 nucleotides on the engineered polynucleotide side of the dsRNA substrate. An asymmetrical internal loop of the present disclosure may be formed by 100 nucleotides on the target RNA side of the dsRNA substrate and 500 nucleotides on the engineered polynucleotide side of the dsRNA substrate. An asymmetrical internal loop of the present disclosure may be formed by 100 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 100 nucleotides on the engineered polynucleotide side of the dsRNA substrate. An asymmetrical internal loop of the present disclosure may be formed by 500 nucleotides on the target RNA side of the dsRNA substrate and 100 nucleotides on the engineered polynucleotide side of the dsRNA substrate. An asymmetrical internal loop of the present disclosure may be formed by 400 nucleotides on the target RNA side of the dsRNA substrate and 100 nucleotides on the engineered polynucleotide side of the dsRNA substrate. An asymmetrical internal loop of the present disclosure may be formed by 300 nucleotides on the target RNA side of the dsRNA substrate and 100 nucleotides on the engineered polynucleotide side of the dsRNA substrate. An asymmetrical internal loop of the present disclosure may be formed by 200 nucleotides on the target RNA side of the dsRNA substrate and 100 nucleotides on the engineered polynucleotide side of the dsRNA substrate. An asymmetrical internal loop of the present disclosure may be formed by 150 nucleotides on the target RNA side of the dsRNA substrate and 100 nucleotides on the engineered polynucleotide side of the dsRNA substrate. An asymmetrical internal loop of the present disclosure may be formed by 150 nucleotides on the target RNA side of the dsRNA substrate and 200 nucleotides on the engineered polynucleotide side of the dsRNA substrate. An asymmetrical internal loop of the present disclosure may be formed by 150 nucleotides on the target RNA side of the dsRNA substrate and 300 nucleotides on the engineered polynucleotide side of the dsRNA substrate. An asymmetrical internal loop of the present disclosure may be formed by 150 nucleotides on the target RNA side of the dsRNA substrate and 400 nucleotides on the engineered polynucleotide side of the dsRNA substrate. An asymmetrical internal loop of the present disclosure may be formed by 150 nucleotides on the target RNA side of the dsRNA substrate and 500 nucleotides on the engineered polynucleotide side of the dsRNA substrate. An asymmetrical internal loop of the present disclosure may be formed by 150 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 150 nucleotides on the engineered polynucleotide side of the dsRNA substrate. An asymmetrical internal loop of the present disclosure may be formed by 500 nucleotides on the target RNA side of the dsRNA substrate and 5 nucleotides on the engineered polynucleotide side of the dsRNA substrate. An asymmetrical internal loop of the present disclosure may be formed by 400 nucleotides on the target RNA side of the dsRNA substrate and 150 nucleotides on the engineered polynucleotide side of the dsRNA substrate. An asymmetrical internal loop of the present disclosure may be formed by 300 nucleotides on the target RNA side of the dsRNA substrate and 150 nucleotides on the engineered polynucleotide side of the dsRNA substrate. An asymmetrical internal loop of the present disclosure may be formed by 200 nucleotides on the target RNA side of the dsRNA substrate and 300 nucleotides on the engineered polynucleotide side of the dsRNA substrate. An asymmetrical internal loop of the present disclosure may be formed by 200 nucleotides on the target RNA side of the dsRNA substrate and 400 nucleotides on the engineered polynucleotide side of the dsRNA substrate. An asymmetrical internal loop of the present disclosure may be formed by 200 nucleotides on the target RNA side of the dsRNA substrate and 500 nucleotides on the engineered polynucleotide side of the dsRNA substrate. An asymmetrical internal loop of the present disclosure may be formed by 200 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 200 nucleotides on the engineered polynucleotide side of the dsRNA substrate. An asymmetrical internal loop of the present disclosure may be formed by 500 nucleotides on the target RNA side of the dsRNA substrate and 200 nucleotides on the engineered polynucleotide side of the dsRNA substrate. An asymmetrical internal loop of the present disclosure may be formed by 400 nucleotides on the target RNA side of the dsRNA substrate and 200 nucleotides on the engineered polynucleotide side of the dsRNA substrate. An asymmetrical internal loop of the present disclosure may be formed by 300 nucleotides on the target RNA side of the dsRNA substrate and 200 nucleotides on the engineered polynucleotide side of the dsRNA substrate. An asymmetrical internal loop of the present disclosure may be formed by 300 nucleotides on the target RNA side of the dsRNA substrate and 400 nucleotides on the engineered polynucleotide side of the dsRNA substrate. An asymmetrical internal loop of the present disclosure may be formed by 300 nucleotides on the target RNA side of the dsRNA substrate and 500 nucleotides on the engineered polynucleotide side of the dsRNA substrate. An asymmetrical internal loop of the present disclosure may be formed by 300 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 300 nucleotides on the engineered polynucleotide side of the dsRNA substrate. An asymmetrical internal loop of the present disclosure may be formed by 500 nucleotides on the target RNA side of the dsRNA substrate and 300 nucleotides on the engineered polynucleotide side of the dsRNA substrate. An asymmetrical internal loop of the present disclosure may be formed by 400 nucleotides on the target RNA side of the dsRNA substrate and 300 nucleotides on the engineered polynucleotide side of the dsRNA substrate. An asymmetrical internal loop of the present disclosure may be formed by 400 nucleotides on the target RNA side of the dsRNA substrate and 500 nucleotides on the engineered polynucleotide side of the dsRNA substrate. An asymmetrical internal loop of the present disclosure may be formed by 400 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 400 nucleotides on the engineered polynucleotide side of the dsRNA substrate. An asymmetrical internal loop of the present disclosure may be formed by 500 nucleotides on the target RNA side of the dsRNA substrate and 400 nucleotides on the engineered polynucleotide side of the dsRNA substrate. An asymmetrical internal loop of the present disclosure may be formed by 500 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 500 nucleotides on the engineered polynucleotide side of the dsRNA substrate. In some embodiments, an asymmetrical loop increases efficiency of editing a target A. In some embodiments, an asymmetrical loop that increases efficiency of editing a target A is an asymmetrical bulge that is formed to reduce the number of adenosines in the sequence of the engineered polynucleotide. Non-limiting examples of an asymmetrical loop that increases efficiency of editing a target A are an asymmetrical loop formed by 5 nucleotides on the engineered polynucleotide side of the dsRNA substrate and 20 nucleotide on the target RNA side of the dsRNA substrate; an asymmetrical bulge of formed by 10 nucleotides on the engineered polynucleotide side of the dsRNA substrate and 50 nucleotides on the target RNA side of the dsRNA substrate; an asymmetrical bulge of formed by 60 nucleotides on the engineered polynucleotide side of the dsRNA substrate and 80 nucleotides on the target RNA side of the dsRNA substrate; an asymmetrical bulge of formed by 18 nucleotides on the engineered polynucleotide side of the dsRNA substrate and 24 nucleotides on the target RNA side of the dsRNA substrate; an asymmetrical bulge of formed by 100 nucleotide on the engineered polynucleotide side of the dsRNA substrate and 150 nucleotides on the target RNA side of the dsRNA substrate; an asymmetrical bulge of formed by 70 nucleotide on the engineered polynucleotide side of the dsRNA substrate and 75 nucleotides on the target RNA side of the dsRNA substrate; an asymmetrical bulge of formed by 8 nucleotide on the engineered polynucleotide side of the dsRNA substrate and 15 nucleotides on the target RNA side of the dsRNA substrate; an asymmetrical bulge of formed by 45 nucleotides on the engineered polynucleotide side of the dsRNA substrate and 46 nucleotides on the target RNA side of the dsRNA substrate; an asymmetrical bulge of formed by 45 nucleotides on the engineered polynucleotide side of the dsRNA substrate and 50 nucleotides on the target RNA side of the dsRNA substrate; and an asymmetrical bulge of formed by 7 nucleotides on the engineered polynucleotide side of the dsRNA substrate and 15 nucleotides on the target RNA side of the dsRNA substrate.

Structural features that comprise a bulge or loop can be of any size. In some cases, a bulge or loop comprise at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 600, 700, 800, 900, or 1000 bases. In some cases, a bulge or loop comprise at least about 1-10, 5-15, 10-20, 15-25, 20-30, 1-30, 1-40, 1-50, 1-60, 1-70, 1-80, 1-90, 1-100, 1-110, 1-120, 1-130, 1-140, 1-150, 1-200, 1-250, 1-300, 1-350, 1-400, 1-450, 1-500, 1-600, 1-700, 1-800, 1-900, 1-1000, 20-50, 20-60, 20-70, 20-80, 20-90, 20-100, 20-110, 20-120, 20-130, 20-140, 20-150, 1-200, 1-250, 1-300, 1-350, 1-400, 1-450, 1-500, 1-600, 1-700, 1-800, 1-900, 1-1000, 30-40, 30-50, 30-60, 30-70, 30-80, 30-90, 30-100, 30-110, 30-120, 30-130, 30-140, 30-150, 30-200, 30-250, 30-300, 30-350, 30-400, 30-450, 30-500, 30-600, 30-700, 30-800, 30-900, 30-1000, 40-50, 40-60, 40-70, 40-80, 40-90, 40-100, 40-110, 40-120, 40-130, 40-140, 40-150, 40-200, 40-250, 40-300, 40-350, 40-400, 40-450, 40-500, 40-600, 40-700, 40-800, 40-900, 40-1000, 50-60, 50-70, 50-80, 50-90, 50-100, 50-110, 50-120, 50-130, 50-140, 50-150, 50-200, 50-250, 50-300, 50-350, 50-400, 50-450, 50-500, 50-600, 50-700, 50-800, 50-900, 50-1000, 60-70, 60-80, 60-90, 60-100, 60-110, 60-120, 60-130, 60-140, 60-150, 60-200, 60-250, 60-300, 60-350, 60-400, 60-450, 60-500, 60-600, 60-700, 60-800, 60-900, 60-1000, 70-80, 70-90, 70-100, 70-110, 70-120, 70-130, 70-140, 70-150, 70-200, 70-250, 70-300, 70-350, 70-400, 70-450, 70-500, 70-600, 70-700, 70-800, 70-900, 70-1000, 80-90, 80-100, 80-110, 80-120, 80-130, 80-140, 80-150, 80-200, 80-250, 80-300, 80-350, 80-400, 80-450, 80-500, 80-600, 80-700, 80-800, 80-900, 80-1000, 90-100, 90-110, 90-120, 90-130, 90-140, 90-150, 90-200, 90-250, 90-300, 90-350, 90-400, 90-450, 90-500, 90-600, 90-700, 90-800, 90-900, 90-1000, 100-110, 100-120, 100-130, 100-140, 100-150, 100-200, 100-250, 100-300, 100-350, 100-400, 100-450, 100-500, 100-600, 100-700, 100-800, 100-900, 100-1000, 110-120, 110-130, 110-140, 110-150, 110-200, 110-250, 110-300, 110-350, 110-400, 110-450, 110-500, 110-600, 110-700, 110-800, 110-900, 110-1000, 120-130, 120-140, 120-150, 120-200, 120-250, 120-300, 120-350, 120-400, 120-450, 120-500, 120-600, 120-700, 120-800, 120-900, 120-1000, 130-140, 130-150, 130-200, 130-250, 130-300, 130-350, 130-400, 130-450, 130-500, 130-600, 130-700, 130-800, 130-900, 130-1000, 140-150, 140-200, 140-250, 140-300, 140-350, 140-400, 140-450, 140-500, 140-600, 140-700, 140-800, 140-900, 140-1000, 150-200, 150-250, 150-300, 150-350, 150-400, 150-450, 150-500, 150-600, 150-700, 150-800, 150-900, 150-1000, 200-250, 200-300, 200-350, 200-400, 200-450, 200-500, 200-600, 200-700, 200-800, 200-900, 200-1000, 250-300, 250-350, 250-400, 250-450, 250-500, 250-600, 250-700, 250-800, 250-900, 250-1000, 300-350, 300-400, 300-450, 300-500, 300-600, 300-700, 300-800, 300-900, 300-1000, 350-400, 350-450, 350-500, 350-600, 350-700, 350-800, 350-900, 350-1000, 400-450, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, or 900-1000 bases in total.

In some cases, a structural feature can be a structured motif. As disclosed herein, a structured motif comprises two or more structural features in a dsRNA substrate. A structured motif can comprise of any combination of structural features, such as in the above claims, to generate an ideal substrate for ADAR editing at a precise location(s). These structural motifs could be artificially engineered to maximized ADAR editing, and/or these structural motifs can be modeled to recapitulate known ADAR substrates.

In some cases, a polynucleotide provided herein can be circularized or in a circular configuration. In some aspects, an at least partially circular polynucleotide lacks a 5′ hydroxyl or a 3′ hydroxyl.

In some cases, the target RNA can comprise a nuclear RNA, a cytoplasmic RNA, or a mitochondrial RNA. In some embodiments, the target RNA can comprise an intergenic DNA (including, without limitation, heterochromatic DNA), a messenger RNA (mRNA), a pre-messenger RNA, a transfer RNA (tRNA), a ribosomal RNA (rRNA), a ribozyme, cDNA, a recombinant polynucleotide, a branched polynucleotide, a plasmid, a vector, an isolated DNA of a sequence, an isolated RNA of a sequence, a sgRNA, a guide RNA, a nucleic acid probe, a primer, an snRNA, a long non-coding RNA, a small RNA, a snoRNA, a siRNA, a miRNA, a tRNA-derived small RNA (tsRNA), an antisense RNA, an shRNA, or a small rDNA-derived RNA (srRNA).

In some embodiments, an engineered guide RNA, an engineered polynucleotide, or a precursor engineered polynucleotide can form a secondary structure comprising a stem-loop, a cruciform, a toe hold, a mismatch bulge, or any combination thereof. In some cases, a secondary structure can comprise a stem, a hairpin loop, a pseudoknot, a bulge, an internal loop, a multiloop, a G-quadruplex, or any combination thereof. In some embodiments, an engineered guide RNA, an engineered polynucleotide, or a precursor engineered polynucleotide can adopt an A-form, a B-form, a Z-form, or any combination thereof.

An engineered guide RNA, an engineered polynucleotide, or a precursor engineered polynucleotide comprising a secondary structure can significantly enhance affinity of binding to a target RNA, enhance specificity of binding to a target RNA, enhance efficiency of editing of a target RNA, reduce off-target editing, reduce editing of a non-target RNA, enhance efficiency of recruiting an RNA editing entity, or a combination thereof, as compared to a comparable guide RNA or a comparable polynucleotide that lacks the secondary structure. An engineered guide RNA, an engineered polynucleotide, or a precursor engineered polynucleotide comprising a bulge can significantly enhance affinity of binding to a target RNA, enhance specificity of binding to a target RNA, enhance efficiency of editing of a target RNA, reduce off-target editing, reduce editing of a non-target RNA, enhance efficiency of recruiting an RNA editing entity, or a combination thereof, as compared to a comparable guide RNA or comparable polynucleotide that lacks the bulge. An engineered guide RNA, an engineered polynucleotide, or a precursor engineered polynucleotide comprising a stem-loop, a cruciform, a toe hold, a mismatch bulge, a stem, a hairpin loop, a pseudoknot, an internal loop, a multiloop, a G-quadruplex, or any combination thereof can significantly enhance affinity of binding to a target RNA, enhance specificity of binding to a target RNA, enhance efficiency of editing of a target RNA, reduce off-target editing, reduce editing of a non-target RNA, enhance efficiency of recruiting an RNA editing entity, or a combination thereof, as compared to a comparable guide RNA or comparable polynucleotide that lacks the stem-loop, the cruciform, the toe hold, the mismatch bulge, the stem, the hairpin loop, the pseudoknot, the internal loop, the multiloop, the G-quadruplex, or any combination thereof.

An engineered guide RNA, an engineered polynucleotide, or a precursor engineered polynucleotide can comprise a sequence of an RNA Polymerase III promoter. In some cases, an engineered polynucleotide, or a precursor engineered polynucleotide can comprise a sequence with at least about: 70%, 75%, 80%, 85%, 90%, 95% or 100% homology to a U7, a U1, a U6, an H1, a 7SK promoter sequence, or any combination thereof. In some cases, an engineered polynucleotide, or a precursor engineered polynucleotide can comprise a Pol II promoter, such as a CMV promoter, a EF1alpha promoter, a MCK promoter, or a Spc5-12 promotor. In some instances, an engineered guide RNA, an engineered polynucleotide, or a precursor engineered polynucleotide can comprise an RNA sequence motif. In some cases, an engineered polynucleotide, or a precursor engineered polynucleotide can comprise a sequence with at least about: 70%, 75%, 80%, 85%, 90%, 95% or 100% homology to an RNA sequence motif described herein. In some instances, an RNA sequence motif can comprise a SmOPT sequence, a hnRNPA1 sequence, a MALAT1 sequence, a SIRLOIN sequence, a GluR2 sequence, G-quadruplex capping, a Alu sequence, a Cas9 gRNA, or combination thereof. In some cases, an RNA sequence motif can comprise a secondary RNA structure, such as a hairpin structure. In some cases, an RNA sequence motif can comprise one or more RNA sequence motifs, one or more promoter sequences, or both. For example, an engineered polynucleotide can comprise a U7 promoter sequence fused with a SmOPT sequence.

A domain can form a two-dimensional shape or secondary structure. For example, a targeting domain, an RNA editing entity recruiting domain, or a combination thereof can form a secondary structure that can comprise a linear region, a cruciform, a toe hold, a stem-loop, or portion thereof, or any combination thereof. A domain itself can form a substantially linear two-dimensional structure. A domain can form a secondary structure that can comprise a cruciform. A domain can form a secondary structure that can comprise a stem-loop. A domain can form a secondary structure that can comprise a toehold. In some instances, a domain can be a single nucleotide. In some cases, a domain can be at least a single nucleotide. In some cases, a domain can comprise a sequence of polynucleotide. In some embodiments, a domain can comprise a specific sequence of polynucleotide. In some cases, a domain can comprise a non-specific sequence of polynucleotide. In some embodiments, the sequence domain can overlap with the sequence of another domain. In other cases, the sequence of a domain may not overlap with the sequence of another domain.

An engineered guide RNA, an engineered polynucleotide, or a precursor engineered polynucleotide can further comprise an RNA editing entity recruiting domain. In some embodiments, a targeting domain can be within about 5, 10, 15, 20, 25, 30, 35, 40, 45, or 50 nucleotides of an RNA editing entity recruiting domain.

The association of the targeting domain of the engineered guide polynucleotide and the coding region of a target RNA can facilitate an edit of a base in the target RNA by an RNA editing entity such as ADAR1, ADAR2, APOBEC or any combination thereof. In some cases, an edit of a base can be a chemical transformation of a base. In some cases, the targeting domain can at least partially bind or is capable of at least partially binding to a target RNA that is implemented in a disease or condition. In some cases, the RNA editing can be determined in an in vitro assay by directly or indirectly introducing (e.g. transfecting) a target RNA and an engineered guide polynucleotide designed to target the target RNA into the same cell. In some cases, an engineered guide can be introduced into a cell by a vector, such as a AAV2 vector. In some cases, an engineered guide can be introduced into a cell by transfection, such as transfection of a plasmid encoding an engineered guide RNA. The target RNA may be sequenced to identify editing by the engineered guide polynucleotide. In some cases, a cell can be a primary cell. In some cases, a primary cell or a cell can be a neuron, a photoreceptor cell (e.g. a S cone cell, a L cone cell, a M cone cell, a rod cell) , a retinal pigment epithelium cell, a glia cell (e.g. an astrocyte, an oligodendrocyte, a microglia), a muscle cell (e.g. a myoblast, a myotube), a hepatocyte, a lung epithelial cell, or a fibroblast (e.g. dermal fibroblast). In some cases, a cell can be a horizontal cell, a ganglion cell, or a bipolar cell. In some cases, a cell line can be a mammalian cell line, such as HEK293T, NCI-60, MCF-7, HL-60, 293T (human embryonic kidney), RD (human embryo rhabdomyosarcoma), LHCN (human skeletal myoblast) differentiated, LHCN undifferentiated, Saos-2, CHO, or HeLa cells. In some cases, a cell can be differentiated. In some cases, a cell can be undifferentiated. In some cases, a cell line can be an insect cell line, such as Sf9.

An engineered guide RNA, an engineered polynucleotide, or a precursor engineered linear polynucleotide encoding an engineered polynucleotide, can facilitate an edit of a target RNA, for example, via an RNA editing entity. In some cases, an engineered polynucleotide may not facilitate an edit of a target RNA. In some instances, an engineered polynucleotide can have an increased editing efficiency to a target RNA by at least about 90%, relative to an otherwise comparable polynucleotide that can comprise a 5′ reducing hydroxyl, a 3′ reducing hydroxyl, or both. In some embodiments, an editing efficiency can be determined by (i) transfecting a target RNA into a primary cell line, (ii) transfecting an engineered polynucleotide and an otherwise comparable polynucleotide that can comprise a 5′ reducing hydroxyl, a 3′ reducing hydroxyl or both, into a primary cell line, and (iii) sequencing the target RNA. In some embodiments, an editing efficiency can be determined by (i) transfecting a target RNA into a primary cell line, (ii) transfecting an engineered polynucleotide and an otherwise comparable polynucleotide that can comprise the 5′ reducing hydroxyl, the 3′ reducing hydroxyl or both, into a primary cell line, and (iii) mass spectroscopy of the target RNA. In some embodiments, an edit of a base of a nucleotide of a target RNA by an RNA editing entity can be determined in an in vitro assay comprising: (i) directly or indirectly introducing (e.g. transfecting) the target RNA into a primary cell line, (ii) directly or indirectly introducing (e.g. transfecting) the engineered polynucleotide into a primary cell line, and (iii) sequencing the target RNA. In some cases, transfecting the target RNA into a primary cell line can comprise transfecting a plasmid encoding for the target RNA into a primary cell line. In some instances, transfecting an engineered polynucleotide into a primary cell line can comprise transfecting a precursor engineered polynucleotide, or a polynucleotide (e.g. plasmid) that encodes for a precursor engineered linear polynucleotide, into a primary cell line. In some cases, sequencing can comprise Sanger sequencing of a target RNA after the target RNA has been converted to cDNA by reverse transcriptase. In some instances, a primary cell line can comprise a neuron, a photoreceptor cell, a retinal pigment epithelium cell, a glia cell, a myoblast cell, a myotube cell, a hepatocyte, a lung epithelial cell, or a fibroblast cell.

An RNA editing entity can comprise an endogenous enzyme. In some instances, an RNA editing entity can comprise a recombinant enzyme. In some cases, an RNA editing entity can comprise a fusion polypeptide. In some embodiments, an RNA editing entity can comprise APOBEC1, APOBEC2, APOBEC3A, APOBEC3B, APOBEC3C, APOBEC3D (“APOBEC3E” now can refer to this), APOBEC3F, APOBEC3G, APOBEC3H, APOBEC4, Activation-induced (cytidine) deaminase (AID), ADAR1, ADAR1p110, ADAR1p150, ADAR2, ADAR3, or any combination thereof. In some cases, an RNA editing entity can comprise at least about: 70%, 75%, 80%, 85%, 90%, 95%, or 100% sequence homology to APOBEC1, APOBEC2, APOBEC3A, APOBEC3B, APOBEC3C, APOBEC3D (“APOBEC3E” now can refer to this), APOBEC3F, APOBEC3G, APOBEC3H, APOBEC4, Activation-induced (cytidine) deaminase (AID), ADAR1, ADAR1p110, ADAR1p150, ADAR2, ADAR3, or any combination thereof. In some cases, an RNA editing entity can comprise a human enzyme such as hADAR1, hADAR2, or hADAR3. In some cases, an RNA editing entity can be a virus-encoded RNA-dependent RNA polymerase. In some cases, an RNA editing entity can be a virus-encoded RNA-dependent RNA polymerase from measles, mumps, or parainfluenza. In some instances, an RNA editing entity can be an enzyme from Trypanosoma brucei capable of adding or deleting a nucleotide or nucleotides in a target RNA. In some instances, an RNA editing entity can be an enzyme from Trypanosoma brucei capable of adding or deleting an Uracil or more than one Uracil in a target RNA.

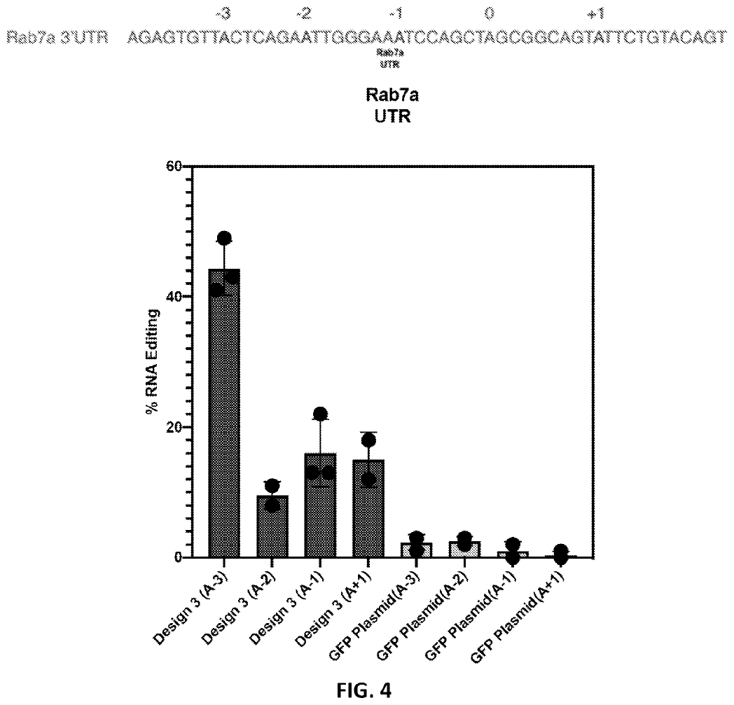

An engineered guide RNA, an engineered polynucleotide, a precursor engineered linear polynucleotide, or a precursor engineered polynucleotide can recruit an RNA editing entity that, when associated with an engineered guide RNA and a target RNA, performs a chemical transformation on a base of a nucleotide in the target RNA. In some cases, a chemical transformation can comprise an edit of a base. In some embodiments, a chemical transformation, such as an edit of a base can result in an increased level of a protein or fragment thereof after translation of a target RNA with the chemical transformation, relative to an otherwise comparable target RNA lacking the chemical transformation. In some cases, an increased level can be from about: 5% to about 100%, 10% to about 50%, 25% to about 75%, or from about 40% to about 90%. In some embodiments, a chemical transformation can result in a decreased level of a protein or fragment thereof after translation of a target RNA with the chemical transformation, relative to an otherwise comparable target RNA lacking the chemical transformation. In some cases, a decreased level can be from about: 5% to about 99%, 10% to about 50%, 25% to about 75%, or from about 40% to about 90%. In some embodiments, a chemical transformation can result in an increased length of a protein or fragment thereof, an increased functionality of a protein or fragment thereof, increased stability of a protein or fragment thereof, or any combination thereof after translation of the target RNA with the edit of the base, relative to a translated protein of an otherwise comparable target RNA lacking the edit. In some cases, an increased length can be from about: 5% to about 100%, 2% to about 10%, 10% to about 25%, 25% to about 50%, 40% to about 80%, or about 75% to about 150%. In some cases, the increased length of a protein can be over 100%. In some cases, the increased stability can be an increased half-life of the protein or fragment thereof. In some cases, the increased half-life can be at least about: 2×, 3×, 4×, 5×, 6×, 7×, 8×, 9×, or 10× greater than to a translated protein of an otherwise comparable target RNA lacking the edit. In some cases, increased functionality can comprise a protein or fragment thereof, such as an enzyme, that can increase the speed of a reaction, increase the Vₘₐₓ, or both. In some cases, increased functionality can comprise a protein (e.g. enzyme) or fragment thereof, encoded by a target RNA with the edit of the base, comprising a lower energy of activation as compared to a translated protein of an otherwise comparable target RNA lacking the edit. In some cases, an edit of a base of a polynucleotide can comprise a chemical transformation. In some embodiments, a chemical transformation can convert a sense codon into a stop codon. In some cases, the stop codon can be implicated in a disease pathogenic pathway and converting the stop codon to the sense codon can reduce the disease pathogenic pathway. In some cases, a chemical transformation can convert a stop codon into a sense codon. In some embodiments, a chemical transformation can convert a first sense codon into a second sense codon. For example, in a missense mutation. In some cases, the first sense codon can be implicated in a disease pathogenic pathway and converting the first sense codon to the second sense codon can reduce the disease pathogenic pathway In some instances, a chemical transformation can convert a first stop codon into a second stop codon. In some instances, a chemical transformation can convert a sense codon specifying a first amino acid into a second sense codon specifying a second amino acid. In some cases, the first amino acid can be a protease cleavage site. In some embodiments, a chemical transformation can alter the localization, folding, or synthesis of a protein or fragment thereof after translation of a target RNA with the chemical transformation, relative to an otherwise comparable target RNA lacking the chemical transformation. In some cases, a chemical transformation can alter the localization, folding, or synthesis of a target RNA with the chemical transformation, relative to an otherwise comparable target RNA lacking the chemical transformation. In some embodiments, a target RNA can comprise a coding or a non-coding RNA. In some cases, RNA editing can occur at multiple positions of a target RNA as shown in FIG. 4. FIG. 4 shows the percent RNA editing of different adenosine nucleotides in the RAB7A 3′UTR from a circular guide RNA compared to a control. The circular guide RNA was shown to edit multiple nucleotides at different positions in the Rab7a 3′ UTR sequence. The highest percent RNA editing occurred at the −3 position in the Rab7a 3′UTR sequence.

In some embodiments, RNA editing can be evaluated by determining by the percent RNA editing of a target RNA. In some cases, RNA editing can be determined by changes in a level of protein. In some cases, a level of a protein can be measured by a Western blot. In some cases, a level of a protein can be measured by densitometry with a quantitative protein gel. In some cases, the percent RNA editing of a target RNA can be determined at different time points (e.g. 24 hours, 48 hours, 96 hours) after transfection with an engineered polynucleotide by reverse transcribing the target RNA to cDNA then using Sanger sequencing to determine the percent RNA editing of a target RNA. In some cases, the cDNA can be amplified prior to sequencing by polymerase chain reaction. Sanger traces from Sanger sequencing can be analyzed to assess the editing efficiency of guide RNAs. In some cases, droplet digital PCR can be used to assess the editing efficiency of guide RNAs. In some cases, quantitative real-time PCR can be used to assess the editing efficiency of guide RNAs. In some cases, next generation sequencing technologies (e.g. sequencing by synthesis) can be used to determine percent RNA editing of a target RNA. For example, RNA sequencing can be used to determine the percent RNA editing of a target RNA after transfection with a guide RNA or guide polynucleotide. In some instances, the individual sequencing reads can be analyzed to determine the percent RNA editing.

In some embodiments, the association of the targeting domain of the engineered guide polynucleotide and an untranslated region of the target RNA can facilitate a reduction in a level of a polypeptide encoded for by a target RNA. In some cases, the reduction in an expression level of a polypeptide encoded by a target RNA may not be dependent on an RNA-directed RNA endonuclease. In some cases, a reduction in the expression level of a polypeptide encoded by a target RNA may not be Dicer-dependent, RISC-dependent or Argonaute-dependent. In some cases, a reduction in the expression level of a polypeptide encoded by a target RNA may be at least partially independent of Dicer, RISC, Argonaute, or any combination thereof. In some cases, a reduction in the level of a polypeptide encoded for by a target RNA can be determined in an in vitro assay by directly or indirectly introducing (e.g. transfecting) a target RNA into a first cell and a second cell of a cell line, then directly or indirectly introducing (e.g. transfecting) the engineered polynucleotide targeting the target RNA into the first cell of the cell line and comparing the amount of the polypeptide encoded for by the target RNA in the first cell and the second cell. In some cases, a cell can be a primary cell. In some cases, a primary cell or a cell can be a neuron, a photoreceptor cell (e.g. a S cone cell, a L cone cell, a M cone cell, a rod cell), a retinal pigment epithelium cell, a glia cell (e.g. an astrocyte, an oligodendrocyte, a microglia), a muscle cell (e.g. a myoblast, a myotube), a hepatocyte, a lung epithelial cell, or a fibroblast (e.g. dermal fibroblast). In some cases, a cell can be a horizontal cell, a ganglion cell, or a bipolar cell. In some cases, a cell line can be a mammalian cell line, such as HEK293T, NCI-60, MCF-7, HL-60, RD, LHCN differentiated, LHCN undifferentiated, Saos-2, CHO, or HeLa cells. In some cases, a cell line can be an insect cell line, such as Sf9.

In some cases, the targeting domain can associate with a coding region, a non-coding region, or both in a target RNA. In some instances, a non-coding region can comprise an intronic region of the target RNA. In some cases, the targeting domain can be configured to at least partially associate with at least a portion of an intronic region of the target RNA. In some instances, a non-coding region can comprise an untranslated region (UTR) such as a 3′ UTR, a 5′ UTR, or both. In some cases, a targeting domain can cause an edit of a base by targeting a UTR. In some instances, a targeting domain may cause substantially little to no editing of an mRNA by targeting an UTR. In some cases, a targeting domain may cause a knockdown in mRNA levels, protein levels or both by targeting a non-coding region. In some cases, a targeting domain can be configured to at least partially associate with at least a portion of an upstream open reading frame (uORF) of a target RNA. In some cases, a targeting domain may not be configured to at least partially associate with an upstream open reading frame (uORF) of a target RNA.

In some cases, a targeting domain may not comprise an aptamer. An aptamer can be a polynucleotide sequence that binds through shape complementarity rather than nucleotide base complementarity to a target, such as a target RNA. In some embodiments, an aptamer is not include a recruiting domain. In some embodiments, an aptamer is a recruiting domain.

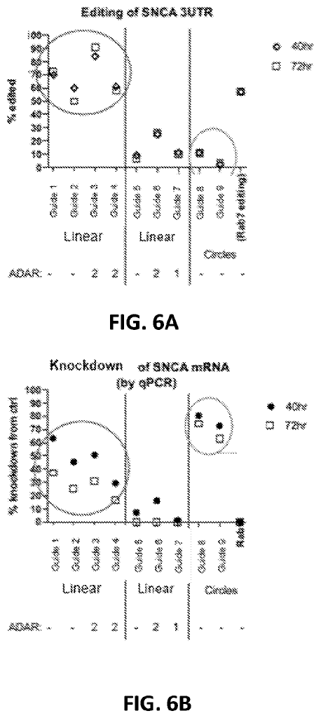

In some cases, an engineered guide described herein can facilitate a knockdown. A knockdown can reduce the expression of a target RNA, reduce protein expression or both. In some cases, a knockdown can be accompanied by editing of an mRNA. In some instances, a knockdown can be mediated by an RNA editing enzyme (e.g. ADAR). In some instances, an RNA editing enzyme can cause a knockdown by hydrolytic deamination of multiple adenosines in an RNA. Hydrolytic deamination of multiple adenosines in an RNA can be referred to as hyper-editing. In some cases, hyper-editing can occur in cis (e.g. in an Alu element) or in trans (e.g. in a target RNA by an engineered polynucleotide). In some cases, a knockdown can occur with substantially little to no editing of an mRNA. In some instances, a knockdown can occur by targeting an untranslated region of the target RNA, such as a 3′ UTR, a 5′ UTR or both. In some cases, a knockdown can occur by targeting a coding region of the target RNA. FIG. 6A shows the percent RNA editing of nucleotides and the percent knockdown of 3′ UTR alpha synuclein (SNCA) mRNA from linear guide RNAs and circular guide RNAs in FIG. 6B. The circular guide RNA showed lower editing levels compared to the linear guides for editing the SNCA 3′UTR. However, the circular guide RNA showed increased knockdown of the SNCA mRNA compared to the linear guide RNAs. The increased knockdown efficiency of circular guides may be independent of ADAR. The editing of the SNCA mRNA may be dependent of ADAR.

### Chemical Modifications

In some instances, an engineered guide RNA, an engineered polynucleotide, and a precursor engineered polynucleotide can comprise a modification. An engineered guide RNA, an engineered polynucleotide, and a precursor engineered polynucleotide can comprise more than one modification. A modification can include a modified base. A modification can include a sugar modification, such as adding a glucose or other sugar-based moiety to one or more bases of an engineered guide RNA, an engineered polynucleotide, or a precursor engineered polynucleotide. A modification can comprise a protein coating over at least a portion of an engineered guide RNA, an engineered polynucleotide, or a precursor engineered polynucleotide. In some cases, an engineered guide RNA, an engineered polynucleotide, or a precursor engineered polynucleotide can comprise a sugar modification on a nucleotide. In some instances, a sugar modification of a nucleotide of an engineered guide RNA, an engineered polynucleotide, or a precursor engineered polynucleotide can comprise a methyl group, a fluoro group, a methoxyethyl group, an ethyl group, a phosphate group, an amide group, an ester group, or any combination thereof. A sugar modification of a nucleotide of an engineered guide RNA, an engineered polynucleotide, or a precursor engineered polynucleotide can comprise a 2′-O-methylation. A modification can increase stability or half-life, enhance the efficiency of editing, significantly reduce off-target editing, enhance efficiency of recruiting an RNA editing entity, or a combination thereof of an engineered guide RNA, an engineered polynucleotide, or a precursor engineered polynucleotide, as compared to a substantially similar engineered guide RNA, or an engineered polynucleotide, without the modification.

In some instances, chemical modifications to enhance guide stability, synthesis, localization, intracellular retention, or lengthen half-lives may not be genetically encodable. An engineered polynucleotide can be circular, substantially circular, or otherwise linked in a contiguous fashion (e.g. can be arranged as a loop) and can also retain a substantially similar secondary structure as a substantially similar engineered polynucleotide that may not be circular or may not be a loop. A circular or looped engineered polynucleotide can be pre-strained.

An aspect of the disclosure provides for an engineered guide RNA, an engineered polynucleotide, a precursor engineered linear polynucleotide, a precursor engineered polynucleotide, vectors comprising engineered polynucleotides, compositions, and pharmaceutical compositions for RNA editing. Any of the above or as described herein can be configured for an A (adenosine) to I (inosine) edit, a C (cytosine) to U (uracil) edit, a U to C edit, or a combination thereof. In some cases, an RNA edit can be an addition and deletion of a nucleotide and/or nucleotides in a target RNA. In some instances, an RNA edit can be an addition and deletion of a U nucleotide in a target RNA. In some cases, an A to I edit can be interpreted or read as a C to U mutation. In some embodiments, an A to I edit can be interpreted or read as an A to G mutation. In some cases, a target RNA can comprise a mutation, such as a missense or a nonsense mutation. In some cases, a target RNA can comprise a missense mutation. In some cases, a target RNA can comprise a nonsense mutation. In some cases, a targeting domain can at least partially bind to a target RNA. In some instances, a target RNA can be implemented in a disease or condition, such as Rett syndrome, Huntington's disease, a muscular dystrophy, sickle cell anemia, or Tay-Sachs Disease. Engineered polynucleotides, vectors comprising engineered polynucleotides, compositions, and pharmaceutical compositions as described herein can provide enhanced editing efficiencies as compared to native systems, reduced off-target editing, enhanced stability, enhanced synthesis, enhanced intracellular retention, or enhanced half-lives, or any combination thereof.

### Delivery

Any engineered polynucleotide described herein (e.g. a circularized polynucleotide) or an engineered precursor polynucleotide as described herein capable of circularization in a cell can be delivered to a cell via a vector. Such vectors can include genetically encodable vectors as described below (e.g. viral vectors) as well as other vehicles such as liposomes or nanoparticles described below. Such vectors can be designed to perform targeted delivery of an engineered polynucleotide described herein (e.g. a circularized polynucleotide) or an engineered precursor polynucleotide as described herein to a cell. Specifically, such vectors can deliver an engineered polynucleotide described herein (e.g. a circularized polynucleotide) or an engineered precursor polynucleotide as described herein to a cell of a subject suffering from a disease or condition as described herein. Such disease or condition can include diseases or conditions where a mutation of an engineered polynucleotide (e.g. a pre-RNA or an mRNA) is implicated in the progression of the disease or condition. Accordingly, delivery of an engineered polynucleotide described herein (e.g. a circularized polynucleotide) or an engineered precursor polynucleotide as described herein to the cell of the subject can be used to treat the disease or condition, or even prevent the disease or condition where the administering is performed prophylactically.

An aspect of the disclosure provides vectors and methods of administration for engineered guide RNAs, engineered polynucleotides, precursor engineered linear polynucleotides, precursor engineered polynucleotides and pharmaceutical compositions containing polynucleotides. The circular or looped guide RNA described herein can be delivered to a cell or a subject using a variety of methods such as an AAV vector.

A vector can be employed to deliver a nucleic acid such as an engineered guide RNA, an engineered polynucleotide, a precursor engineered linear polynucleotide, or a precursor engineered polynucleotide. In some cases, a vector can comprise a polypeptide coat, wherein at least a portion of an engineered guide RNA, an engineered polynucleotide, or a precursor engineered polynucleotide can be present inside of the polypeptide coat. In some cases, a vector can comprise a precursor engineered linear polynucleotide, an engineered polynucleotide, or both. A vector can comprise DNA, such as double-stranded DNA or single-stranded DNA. A vector can comprise RNA. In some embodiments, an RNA can comprise a base modification. A vector can comprise a recombinant vector. A vector can be modified from a naturally occurring vector. A vector can comprise at least a portion of a non-naturally occurring vector. Any vector can be utilized. In some embodiments, a vector can comprise a viral vector, a liposome, a nanoparticle, an exosome, an extracellular vesicle, a nanomesh, or any combination thereof. In some instances, a viral vector can comprise an adenoviral vector, an adeno-associated viral vector (AAV), a lentiviral vector, a retroviral vector, a portion of any of these, or any combination thereof. In some embodiments, a nanoparticle vector can comprise a polymeric-based nanoparticle, an aminolipid-based nanoparticle, a metallic nanoparticle (such as gold-based nanoparticle), a portion of any of these, or any combination thereof. In some cases, a vector can comprise an AAV vector. In some instances, a vector can be a single-stranded AAV vector. A vector can be modified to include a modified VP1 protein (such as an AAV vector modified to include a VP1 protein). An AAV can comprise a serotype—such as an AAV1 serotype, an AAV2 serotype, AAV3 serotype, an AAV4 serotype, AAV5 serotype, an AAV6 serotype, AAV7 serotype, an AAV8 serotype, an AAV9 serotype, a derivative of any of these, or any combination thereof. In some cases, an AAV vector can be a chimera of one or more serotypes (e.g., an AAV2/5 virus having Rep and ITRs from AAV2 and capsid polypeptides from AAV5). In some instances, an AAV vector can be a variant of a serotype. In some cases, the viral vector can be a self-complementary adeno-associated viral (scAAV) vector.

A nucleic acid can at least partially encode an engineered guide RNA, an engineered polynucleotide, or a precursor engineered guide RNA. A nucleic acid can be double-stranded, single stranded, or a combination thereof. A nucleic acid can be DNA, RNA, locked nucleic acid (LNA), or peptide nucleic acid (PNA). In some cases, a nucleic acid can comprise a targeting tag. In some instances, a targeting tag can at least partially direct a nucleic acid to a specific organ or area in the body. In some cases, a targeting tag can comprise N-Acetylgalactosamine (GlaNac).

In some embodiments, engineered guide RNAs, engineered polynucleotides, precursor engineered linear polynucleotides, precursor engineered polynucleotides, can be comprised in a pharmaceutical composition. In some cases, a circular or looped engineered guide or a precursor thereof can be comprised in a pharmaceutical composition. In some embodiments, a pharmaceutical composition in unit dose form can comprise an engineered guide RNA, an engineered polynucleotide (e.g. engineered guide polynucleotides), precursor engineered polynucleotides, vectors comprising the engineered polynucleotides, or the precursor engineered polynucleotides, or nucleic acids of the engineered polynucleotides or the precursor engineered polynucleotides; and a pharmaceutically acceptable excipient, diluent, or a carrier. A pharmaceutical composition can comprise a first active ingredient. The first active ingredient can comprise an engineered polynucleotide, a precursor engineered polynucleotide, a vector comprising the engineered polynucleotides, or a precursor engineered polynucleotide, or s nucleic acid of the engineered polynucleotides or the precursor engineered polynucleotides. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient. In some cases, a pharmaceutical composition can comprise a pharmaceutical formulation.

In some cases, an engineered polynucleotide can be co-delivered with an RNA editing entity. In some embodiments, an RNA editing entity can be separately delivered to a cell or to a subject. In some instances, an engineered polynucleotide can be associated with or directly linked to an RNA editing entity and the associated or directly linked composition can be delivered to a cell or to a subject. In some cases, compositions can include a sequence encoding an editing entity. Compositions can include more than one editing entity, such as 1, 2, 3, 4, 5 or more. Compositions can comprise more than one editing entity, each that each editing entity can independently edit a target sequence.

In some embodiments, compositions disclosed herein can be in unit dose forms or multiple-dose forms. For example, a pharmaceutical composition described herein can be in unit dose form. Unit dose forms, as used herein, can refer to physically discrete units suitable for administration to human or non-human subjects (e.g., animals). In some cases, unit dose forms can be packaged individually. Each unit dose can contain a predetermined quantity of an active ingredient(s) that can be sufficient to produce the desired therapeutic effect in association with pharmaceutical carriers, diluents, excipients, or any combination thereof. Examples of unit dose forms can include, ampules, syringes, and individually packaged tablets and capsules. In some instances, a unit dose form can be comprised in a disposable syringe. In some instances, unit-dosage forms can be administered in fractions or multiples thereof. A multiple-dose form can be a plurality of identical unit dose forms packaged in a single container, which can be administered in segregated a unit dose form. Examples of a multiple-dose form can include vials, bottles of tablets or capsules, or bottles of pints or gallons. In some instances, a multiple-dose form can comprise the same pharmaceutically active agents. In some instances, a multiple-dose form can comprise different pharmaceutically active agents.

A composition can comprise a combination of the active agent, e.g., an engineered guide RNA, an engineered polynucleotide, a precursor engineered polynucleotide, or a vector or a nucleic acid containing the engineered polynucleotide or the precursor engineered polynucleotide of this disclosure, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), which can be present singly or in combination, comprising alone or in combination 1-99.99% by weight or volume. Exemplary protein excipients include serum albumins such as human serum albumin (HSA), recombinant human albumin (rHA), gelatin, casein, and the like. Representative amino acid components, antibody components, or both, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients can be also intended within the scope of this technology, examples of which include but can be not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), and myoinositol.

A composition described herein can compromise an excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent. In some cases, an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts, or combinations thereof can be used in a pharmaceutical formulation.

An excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitors.

A pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The binders that can be used in a pharmaceutical formulation can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol, water, or a combination thereof.

A pharmaceutical formulation can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof

In some cases, a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isomorphous silicate, and microcrystalline cellulose as high HLB emulsifier surfactants.

In some embodiments, a pharmaceutical formulation can comprise a disintegrant as an excipient. In some instances, a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments, a disintegrant can be an effervescent disintegrant. Non-limiting examples of suitable effervescent disintegrants can include sodium bicarbonate in combination with citric acid, and sodium bicarbonate in combination with tartaric acid.

In some cases, an excipient can comprise a sweetener, a flavoring agent or both. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. In some cases, flavoring agents incorporated into a composition can be chosen from synthetic flavor oils and flavoring aromatics; natural oils; extracts from plants, leaves, flowers, and fruits; and combinations thereof. In some embodiments, a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

A pharmaceutical composition can comprise a diluent. Non-limiting examples of diluents can include water, glycerol, methanol, ethanol, and other similar biocompatible diluents. In some cases, a diluent can be an aqueous acid such as acetic acid, citric acid, maleic acid, hydrochloric acid, phosphoric acid, nitric acid, sulfuric acid, or similar. In other cases, a diluent can be selected from a group comprising alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

### Kits

In some embodiments, a kit can comprise an engineered guide RNA, an engineered polynucleotide, a precursor engineered polynucleotide, a vector comprising the engineered polynucleotide, or the precursor engineered polynucleotide, a nucleic acid of the engineered polynucleotide or the precursor engineered polynucleotide, or a pharmaceutical composition and a container. In some instances, a container can be plastic, glass, metal, or any combination thereof. In some cases, a kit can comprise instructions for use, such as instructions for administration to a subject in need thereof.

In some instances, a packaged product comprising a composition described herein can be properly labeled. In some instances, the pharmaceutical composition described herein can be manufactured according to good manufacturing practice (cGMP) and labeling regulations. In some cases, a pharmaceutical composition disclosed herein can be aseptic.

### Methods of Treatment

In some embodiments, a method of treating or preventing, a disease or condition in a subject in need thereof can comprise administering an engineered guide RNA, an engineered polynucleotide, a precursor engineered linear polynucleotide, a precursor engineered polynucleotide, a vector comprising an engineered polynucleotide, a precursor engineered linear polynucleotide, or the precursor engineered polynucleotide, a nucleic acid of an engineered polynucleotide, a precursor engineered linear polynucleotide, or the precursor engineered polynucleotide, a pharmaceutical composition comprising an engineered polynucleotide, a precursor engineered linear polynucleotide, or the precursor engineered polynucleotide, and a pharmaceutically acceptable excipient, diluent, or carrier. In some cases, the method can comprise administering a second therapy. In some cases, a second therapy can comprise an antibiotic, an antiviral, a cancer treatment (e.g. radiation, chemotherapy, a checkpoint inhibitor, a CAR-T cell treatment), a neurological treatment, a steroid, an anti-inflammatory treatment, or any combination thereof. In some cases, a second therapy can comprise an antibody. In some instances, a second therapy can comprise bapineuzumab, solanezumab, gantenerumab, crenezumab, ponezumab, aducanumab, BAN2401, or any combination thereof. The administration can be performed at least about: 1 time per day, 2 times per day, 3 times per day, 4 times per day, 5 times per day, or 6 times per day. The method can be performed for about: 1 day to about 8 days, 1 week to about 5 weeks, 1 month to about 12 months, 1 year to about 3 years, 3 years to about 10 years, 10 years to about 50 years, 25 years to about 100 years, or about 50 years to about 130 years. In some cases, the method can comprise diagnosing a subject as having a disease. In some cases, the subject may have been diagnosed prior to treating. In some cases, diagnosing a subject with a disease or condition can comprise diagnosing with a physical examination, a biopsy, a radiological image, a genetic test, a blood test, a urine test, an antibody test, or any combination thereof. In some instances, a radiological image can comprise the radiological image, wherein the radiological image comprises a computed tomography (CT) image, a nuclear scan, an X-Ray image, a magnetic resonance image (MRI), an ultrasound image, or any combination thereof.

In some embodiments, a method of treating a disease or condition in a subject in need thereof or preventing a disease or condition in a subject in need thereof can comprise administering or prophylactically administering, respectively, an engineered guide RNA, an engineered polynucleotide, a precursor engineered linear polynucleotide, a precursor engineered polynucleotide, a vector comprising an engineered polynucleotide or a precursor engineered polynucleotide, a precursor engineered linear polynucleotide, or a nucleic acid of an engineered polynucleotide or a precursor engineered polynucleotide, a precursor engineered linear polynucleotide, or a pharmaceutical composition comprising an engineered polynucleotide or a precursor engineered polynucleotide to a subject, a cell, or both. In some embodiments, a cell can comprise a mammalian cell. In some cases, a cell can be from any tissue or organ, for example a skin cell, a lung cell, a heart cell, an epithelial cell, a reproductive cell, an eye cell, a kidney cell, a liver cell, a pancreas cell, an intestinal cell, a muscle cell, a gland cell, an eye cell, a brain cell, or a blood cell. In some cases, a cell can comprise a neuron, a photoreceptor cell, a retinal pigment epithelium cell, a glia cell, a myoblast cell, a myotube cell, a hepatocyte, a lung epithelial cell, or a fibroblast cell. In some cases, a cell can be a stem cell, such as embryonic stem cells, pluripotent stem cells, or totipotent stem cells. In some instances, a cell can comprise a human cell. In some cases, a cell can comprise a leukocyte. In some embodiments, a cell can comprise a lymphocyte. In some instances, a cell can comprise a T-cell. In some case, a cell can comprise a helper CD4+ T-cell, a cytotoxic CD8+ T-cell, a memory T-cell, a regulatory CD4+ T-cell, a natural killer T-cell, a mucosal associated T-cell, a gamma delta T-cell, or any combination thereof. In some embodiments, a cell can comprise a B-cell. In some cases, a cell can comprise a plasmablast, a plasma cell, a lymphoplasmacytoid cell, a memory B-cell, a follicular B-cell, a marginal zone B-cell, a B-1 cell, a regulatory B cell, or any combination thereof.

Administration of an engineered guide polynucleotide, such as an engineered guide RNA can be effected in one dose, continuously or intermittently throughout the course of treatment. Methods of determining the most effective means and dosage of administration can be known to those of skill in the art and can vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be known in the art. Routes of administration can also be determined and method of determining the most effective routes of administration can be known to those of skill in the art and can vary with the composition used for treatment, the purpose of the treatment, the health condition or disease stage of the subject being treated, and target cell or tissue. Non-limiting examples of routes of administration include oral administration, nasal administration, injection, and topical application.

Administration or application of a composition disclosed herein can be performed for a duration of at least about at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000 days consecutive or nonconsecutive days. In some cases, the composition can be administered for life. In some embodiments, administration or application of the composition described herein can be from about 1 to about 30 days, from about 1 to about 60 days, from about 1 to about 90 days, from about 1 to about 300 days, from about 1 to about 3000 days, from about 30 days to about 90 days, from about 60 days to about 900 days, from about 30 days to about 900 days, or from about 90 days to about 1500 days. In some embodiments, administration or application of the composition described herein can be from about: 1 week to about 5 weeks, 1 month to about 12 months, 1 year to about 3 years, 2 years to about 8 years, 3 years to about 10 years, 10 years to about 50 years, 15 years to about 40 years, 25 years to about 100 years, 30 years to about 75 years, 60 years to about 110 years, or about 50 years to about 130 years.

Administration or application of a composition disclosed herein can be performed for a duration of at least about: 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or for life. Administration can be performed repeatedly over a lifetime of a subject, such as once a day, once a week, or once a month for the lifetime of a subject. Administration can be performed repeatedly over a substantial portion of a subject's life, such as once a day, once a week, or once a month for at least about: 1 year, 5 years, 10 years, 15 years, 20 years, 25 years, 30 years, or more.

Administration or application of composition disclosed herein can be performed at least about: 1, 2, 3, 4, 5, 6, 7, 8, 9, 10, 11, 12, 13, 14, 15, 16, 17, 18, 19, 20, 21, 22, 23, or 24 times a in a 24-hour period. In some cases, administration or application of a composition disclosed herein can be performed continuously throughout a 24-hour period. In some embodiments, administration or application of composition disclosed herein can be performed at least about: 1, 2, 3, 4, 5, 6, 7, 8, 9, 10, 11, 12, 13, 14, 15, 16, 17, 18, 19, 20, or 21 times a week. In some cases, administration or application of a composition disclosed herein can be performed at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or more times a month. In some embodiments, a composition can be administered as a single dose or as divided doses. For example, administration of a capsule, or a tablet and comprise administration of more than one capsules or tablets. In some cases, the compositions described herein can be administered at a first time point and a second time point. In some embodiments, a composition can be administered such that a first administration can be administered before the other with a difference in administration time of about: 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

### Administration

Administration can refer to methods that can be used to enable the delivery of a pharmaceutical composition described herein (e.g. an engineered polynucleotide) to the desired site of biological action. For example, an engineered guide RNA can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, disclosed herein can be a method, comprising administering a composition disclosed herein to a subject (e.g., a human) in need thereof. In some instances, the method can treat or prevent a disease in the subject.

### Disease Applications and Targets

As described herein, an engineered polynucleotide (e.g. a circularized engineered polynucleotide) or a precursor engineered polynucleotide capable circularization in a cell (e.g. a precursor engineered linear polynucleotide) can be used to treat a disease or condition in a subject. A disease or condition can comprise a neurodegenerative disease, a muscular disorder, a metabolic disorder, an ocular disorder (e.g. an ocular disease), a cancer, a liver disease (Alpha-1 antitrypsin (AAT) deficiency), or any combination thereof. The disease or condition can comprise cystic fibrosis, albinism, alpha-1 -antitrypsin deficiency, Alzheimer disease , Amyotrophic lateral sclerosis, Asthma, β-thalassemia, Cadasil syndrome, Charcot-Marie-Tooth disease, Chronic Obstructive Pulmonary Disease (COPD), dementia, Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Parkinson's disease, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, a tauopathy, a synucleinopathy, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, various forms of cancer (e.g., BRCA1 and 2 linked breast cancer and ovarian cancer). In some cases, a treatment of a disease or condition such as a neurodegenerative disease (e.g. Alzheimer's, Parkinson's) can comprise producing an edit, a knockdown or both of amyloid precursor protein (APP), tau, alpha-synuclein, or any combination thereof. In some cases, APP, tau, and alpha-synuclein can comprise a pathogenic variant. In some instances, APP can comprise a pathogenic variant such as A673V mutation or A673T mutation. In some cases, a treatment of a disease or condition such as a neurodegenerative disease (Parkinson's) can comprise producing an edit, a knockdown or both of a pathogenic variant of LRRK2. In some cases, a pathogenic variant of LRRK can comprise a G2019S mutation. The disease or condition can comprise a muscular dystrophy, an ornithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, a tauopathy, a synucleinopathy, or any combination thereof. In some cases, an engineered polynucleotide can correct a missense mutation in a patient with Rett (e.g. mutate a stop codon to encode for a Trp). In some cases, an engineered polynucleotide can correct a missense mutation or induce a knockdown in a patient with Parkinson's. In some cases, an engineered polynucleotide can induce a mutation in a patient with Alzheimer's, which can reduce cleavage by a protein at a cleavage site in APP. In some cases, an engineered polynucleotide can generate exon skipping in a patient with muscular dystrophy. In some cases, an engineered polynucleotide can correct a mutation in HexA in a patient with Tay-Sachs disease. In some cases, an engineered polynucleotide can correct a mutation in HexA in a patient with Tay-Sachs disease. In some cases, an engineered polynucleotide can correct a mutation in a patient with AAT deficiency (e.g. edit SERPINA1). In some cases, Administration of a composition can be sufficient to: (a) decrease expression of a gene relative to an expression of the gene prior to administration; (b) edit at least one point mutation in a subject, such as a subject in need thereof; (c) edit at least one stop codon in the subject to produce a readthrough of a stop codon; (d) produce an exon skip in the subject, or (e) any combination thereof. A disease or condition can comprise a muscular dystrophy. A muscular dystrophy can include myotonic, Duchenne, Becker, Limb-girdle, facioscapulohumeral, congenital, oculopharyngeal, distal, Emery-Dreifuss, or any combination thereof. A disease or condition can comprise pain, such as chronic pain. Pain can include neuropathic pain, nociceptive pain, or a combination thereof. Nociceptive pain can include visceral pain, somatic pain, or a combination thereof. Certain specification diseases or conditions and associated targets are referenced below.

APP. In some embodiments, the present disclosure provides compositions and methods of use thereof of circularized engineered polynucleotides (or precursor engineered polynucleotides configured to circularize in a cell) that are capable of facilitating RNA editing of an amyloid precursor protein (APP). For example, circularized engineered polynucleotides can facilitate editing of the cleavage site in APP, so that beta/gamma secretases exhibit reduced cleavage of APP or can no longer cut APP and, therefore, reduced levels of Abeta 40/42 or no Abetas can be produced. In some embodiments, a circularized engineered polynucleotide of the present disclosure can target any one of or any combination of the following sites in APP for RNA editing: K670E, K670R, K670G, M671V, A673V, A673T, D672G, E682G, H684R, K687R, K687E, or K687G, I712X,or T714X. Said circularized engineered polynucleotides targeting a site in APP can be encoded for by an engineered polynucleotide construct (e.g. a precursor polynucleotide) of the present disclosure. Sequences of circularized engineered polynucleotides (or precursor engineered polynucleotides configured to circularize in a cell) that target the APP gene may comprise a targeting domain with at least about: 70%, 75%, 80%, 85%, 90%, 95%, or 100% sequence identity to SEQ ID NO: 61, SEQ ID NO: 62, or SEQ ID NO: 63. Said circularized engineered polynucleotides (or precursor engineered polynucleotides configured to circularize in a cell) may be delivered via viral vector (e.g., encoded for and delivered via AAV) as disclosed herein and may be administered via any route of administration disclosed herein to a subject in need thereof. The subject may be human and may be at risk of developing or has developed Alzheimer's disease. The subject may be human and may be at risk of developing or has developed a neurological disease in which APP impacts disease pathology. Thus, the circularized engineered polynucleotides (or precursor engineered polynucleotides configured to circularize in a cell) may be used in a method of treatment of neurological diseases (e.g., Alzheimer's disease).

ABCA4. In some embodiments, the present disclosure provides compositions and methods of use thereof of circularized engineered polynucleotides (or precursor engineered polynucleotides configured to circularize in a cell) that are capable of facilitating RNA editing of an ATP Binding Cassette Subfamily A Member 4 (ABCA4). For example, circularized engineered polynucleotides can facilitate correction of a G with an A at nucleotide position 6320 in an ABCA4 gene; G with an A at nucleotide position 5714 in a ABCA4 gene; and/or a G with an A at nucleotide position 5714 in a ABCA4 gene. Said circularized engineered polynucleotides targeting a site in ABCA4 can be encoded for by an engineered polynucleotide construct (e.g. a precursor polynucleotide) of the present disclosure. Sequences of circularized engineered polynucleotides (or precursor engineered polynucleotides configured to circularize in a cell) that target the ABCA4 gene may comprise a targeting domain with at least about: 70%, 75%, 80%, 85%, 90%, 95%, or 100% sequence identity to any one of: SEQ ID NO: 58, SEQ ID NO: 59, or SEQ ID NO: 60. Said circularized engineered polynucleotides (or precursor engineered polynucleotides configured to circularize in a cell) may be delivered via viral vector (e.g., encoded for and delivered via AAV) as disclosed herein and may be administered via any route of administration disclosed herein to a subject in need thereof. The subject may be human and may be at risk of developing or has developed Stargardt macular degeneration. Such Stargardt macular degeneration may be at least partially caused by a mutation of ABCA4, for which an engineered polynucleotide sequence described herein can facilitate editing in, thus correcting the mutation in ABCA4 and reducing the incidence of Stargardt macular degeneration in the subject. Thus, the circularized engineered polynucleotides (or precursor engineered polynucleotides configured to circularize in a cell) may be used in a method of treatment of Stargardt macular degeneration.

Alpha-synuclein (SNCA). The Alpha-synuclein gene is made up of 5 exons and encodes a 140 amino-acid protein with a predicted molecular mass of ˜14.5 kDa. The encoded product is an intrinsically disordered protein with unknown functions. Usually, Alpha-synuclein is a monomer. Under certain stress conditions or other unknown causes, α-synuclein self-aggregates into oligomers. Lewy-related pathology (LRP), primarily comprised of Alpha-synuclein in more than 50% of autopsy-confirmed Alzheimer's disease patients' brains. While the molecular mechanism of how Alpha-synuclein affects the development of Alzheimer's disease is unclear, experimental evidence has shown that Alpha-synuclein interacts with Tau-p and may seed the intracellular aggregation of Tau-p. Moreover, Alpha-synuclein could regulate the activity of GSK3β, which can mediate Tau-hyperphosphorylation. Alpha-synuclein can also self-assemble into pathogenic aggregates (Lewy bodies). Both Tau and a-synuclein can be released into the extracellular space and spread to other cells. Vascular abnormalities impair the supply of nutrients and removal of metabolic byproducts, cause microinfarcts, and promote the activation of glial cells. Therefore, a multiplex strategy to substantially reduce Tau formation, alpha-synuclein formation, or a combination thereof can be important in effectively treating neurodegenerative diseases.

The domain structure of Alpha-synuclein comprises an N-terminal A2 lipid-binding alpha-helix domain, a Non-amyloid β component (NAC) domain, and a C-terminal acidic domain. The lipid-binding domain consists of five KXKEGV imperfect repeats. The NAC domain consists of a GAV motif with a VGGAVVTGV consensus sequence and three GXXX sub-motifs—where X is any of Gly, Ala, Val, Ile, Leu, Phe, Tyr, Trp, Thr, Ser or Met. The C-terminal acidic domain contains a copper-binding motif with a DPDNEA consensus sequence. Molecularly, Alpha-synuclein is suggested to play a role in neuronal transmission and DNA repair.

In some cases, a region of Alpha-synuclein can be targeted utilizing compositions provided herein. In some cases, a region of the Alpha-synuclein mRNA can be targeted with the engineered polynucleotides disclosed herein for knockdown. In some cases, a region of the exon or intron of the Alpha-synuclein mRNA can be targeted. In some embodiments, a region of the non-coding sequence of the Alpha-synuclein mRNA, such as the 5′UTR and 3′UTR, can be targeted. In other cases, a region of the coding sequence of the Alpha-synuclein mRNA can be targeted. Suitable regions include but are not limited to a N-terminal A2 lipid-binding alpha-helix domain, a Non-amyloid β component (NAC) domain, or a C-terminal acidic domain.

In some aspects, an alpha-synuclein mRNA sequence is targeted. In some cases, any one of the 3,177 residues of the sequence may be targeted utilizing the compositions and methods provided herein. In some cases, a target residue may be located among residues 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, 2601-2700, 2701-2800, 2801-2900, 2901-3000, 3001-3100, and/or 3101-3177.

In some embodiments, the present disclosure provides compositions and methods of use thereof of circularized engineered polynucleotides (or precursor engineered polynucleotides configured to circularize in a cell) that are capable of facilitating RNA editing of SNCA. In some embodiments, a circularized engineered polynucleotide can knock down expression of SNCA, for example, by facilitating editing at a 3′ UTR of an SNCA gene. Said circularized engineered polynucleotides targeting a site in SNCA can be encoded for by an engineered polynucleotide construct of the present disclosure. Sequences of circularized engineered polynucleotides (or precursor engineered polynucleotides configured to circularize in a cell) that target the SNCA gene may comprise a targeting domain with at least about: 70%, 75%, 80%, 85%, 90%, 95%, or 100% sequence identity to any one of: SEQ ID NO: 40, SEQ ID NO: 41, SEQ ID NO: 42, SEQ ID NO: 43, SEQ ID NO: 44. Said circularized engineered polynucleotides (or precursor engineered polynucleotides configured to circularize in a cell) may be delivered via viral vector (e.g., encoded for and delivered via AAV) as disclosed herein and may be administered via any route of administration disclosed herein to a subject in need thereof. The subject may be human and may be at risk of developing or has developed Alzheimer's disease or Parkinson's disease. The subject may be human and may be at risk of developing or has developed a neurological disease in which overexpression of SNCA impacts disease pathology. Thus, the circularized engineered polynucleotides (or precursor engineered polynucleotides configured to circularize in a cell) may be used in a method of treatment of neurological diseases (e.g., Alzheimer's disease).

SERPINA1. In some embodiments, the present disclosure provides compositions and methods of use thereof of circularized engineered polynucleotides (or precursor engineered polynucleotides configured to circularize in a cell) that are capable of facilitating RNA editing of serpin family A member 1 (SERPINA1). For example, circularized engineered polynucleotides can facilitate correction of a G to A mutation at nucleotide position 9989 of a SERPINA1 gene. In some embodiments, a circularized engineered polynucleotide can target, for example, E342 of SERPINA1. Said circularized engineered polynucleotides targeting a site in SERPINA1 can be encoded for by an engineered polynucleotide construct of the present disclosure (e.g. an engineered precursor polynucleotide). Sequences of circularized engineered polynucleotides (or precursor engineered polynucleotides configured to circularize in a cell) configured to target SERPINA1 may comprise a targeting domain with at least about: 70%, 75%, 80%, 85%, 90%, 95%, or 100% sequence identity to SEQ ID NO: 64, or SEQ ID NO: 65. Said circularized engineered polynucleotides (or precursor engineered polynucleotides configured to circularize in a cell) may be delivered via viral vector (e.g., encoded for and delivered via AAV) as disclosed herein and may be administered via any route of administration disclosed herein to a subject in need thereof. The subject may be human and may be at risk of developing or has developed alpha-1 antitrypsin deficiency. Such alpha-1 antitrypsin deficiency may be at least partially caused by a mutation of SERPINA1, for which an engineered polynucleotide sequence described herein can facilitate editing in, thus correcting the mutation in SERPINA1 and reducing the incidence of alpha-1 antitrypsin deficiency in the subject. Thus, the circularized engineered polynucleotides (or precursor engineered polynucleotides configured to circularize in a cell) may be used in a method of treatment of alpha-1 antitrypsin deficiency.

LRRK2. Leucine-rich repeat kinase 2 (LRRK2) has been associated with familial and sporadic cases of Parkinson's Disease and immune-related disorders like Crohn's disease. Its aliases include LRRK2, AURA17, DARDARIN, PARK8, RIPK7, ROCO2, or leucine-rich repeat kinase 2. The LRRK2 gene is made up of 51 exons and encodes a 2527 amino-acid protein with a predicted molecular mass of about 286 kDa. The encoded product is a multi-domain protein with kinase and GTPase activities. LRRK2 can be found in various tissues and organs including but not limited to adrenal, appendix, bone marrow, brain, colon, duodenum, endometrium, esophagus, fat, gall bladder, heart, kidney, liver, lung, lymph node, ovary, pancreas, placenta, prostate, salivary gland, skin, small intestine, spleen, stomach, testis, thyroid, and urinary bladder. LRRK2 can be ubiquitously expressed but is generally more abundant in the brain, kidney, and lung tissue. Cellularly, LRRK2 has been found in astrocytes, endothelial cells, microglia, neurons, and peripheral immune cells.

Over 100 mutations have been identified in LRRK2; six of them—G2019S, R1441C/G/H, Y1699C, and I2020T—have been shown to cause Parkinson's Disease through segregation analysis. G2019S and R1441C are the most common disease-causing mutations in inherited cases. In sporadic cases, these mutations have shown age-dependent penetrance: The percentage of individuals carrying the G2019S mutation that develops the disease jumps from 17% to 85% when the age increases from 50 to 70 years old. In some cases, mutation-carrying individuals never develop the disease.

At its catalytic core, LRRK2 contains the Ras of complex proteins (Roc), C-terminal of ROC (COR), and kinase domains. Multiple protein-protein interaction domains flank this core: an armadillo repeats (ARM) region, an ankyrin repeat (ANK) region, a leucine-rich repeat (LRR) domain are found in the N-terminus joined by a C-terminal WD40 domain. The G2019S mutation is located within the kinase domain. It has been shown to increase the kinase activity; for R1441C/G/H and Y1699C, these mutations can decrease the GTPase activity of the Roc domain. Genome-wide association study has found that common variations in LRRK2 increase the risk of developing sporadic Parkinson's Disease. While some of these variations are nonconservative mutations that affect the protein's binding or catalytic activities, others modulate its expression. These results suggest that specific alleles or haplotypes can regulate LRRK2 expression.

Pro-inflammatory signals upregulate LRRK2 expression in various immune cell types, suggesting that LRRK2 is a critical regulator in the immune response. Studies have found that both systemic and central nervous system (CNS) inflammation are involved in Parkinson's Disease's symptoms. Moreover, LRRK2 mutations associated with Parkinson's Disease modulate its expression levels in response to inflammatory stimuli. Many mutations in LRRK2 are associated with immune-related disorders such as inflammatory bowel disease such as Crohn's Disease. For example, both G2019S and N2081D increase LRRK2′s kinase activity and are over-represented in Crohn's Disease patients in specific populations. Because of its critical role in these disorders, LRRK2 is an important therapeutic target for Parkinson's Disease and Crohn's Disease. In particular, many mutations, such as point mutations including G2019S, play roles in developing these diseases, making LRRK2 an attractive for therapeutic strategy such as RNA editing.

In some embodiments, the present disclosure provides compositions and methods of use thereof of circularized engineered polynucleotides (or precursor engineered polynucleotides configured to circularize in a cell) that are capable of facilitating RNA editing of LRRK2. In some embodiments, circularized engineered polynucleotides can target the following mutations in LRRK2: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T23561, G2385R, V2390M, E2395K, M2397T, L2466H, or Q2490NfsX3. Said circularized engineered polynucleotides targeting a site in LRRK2 can be encoded for by an engineered polynucleotide construct of the present disclosure (e.g. a precursor engineered polynucleotide). Sequences of circularized engineered polynucleotides (or precursor engineered polynucleotides configured to circularize in a cell) configured to target LRRK2 may comprise a targeting domain with at least about: 70%, 75%, 80%, 85%, 90%, 95%, or 100% sequence identity to SEQ ID NO: 39, SEQ ID NO: 53 or SEQ ID NO: 54. Said circularized engineered polynucleotides (or precursor engineered polynucleotides configured to circularize in a cell) may be delivered via viral vector (e.g., encoded for and delivered via AAV) as disclosed herein and may be administered via any route of administration disclosed herein to a subject in need thereof. The subject may be human and may be at risk of developing or has developed a disease or condition associated with mutations in LRRK2 (e.g. diseases of the central nervous system (CNS) or gastrointestinal (GI) tract). For example, such diseases of conditions can include Crohn's disease or Parkinson's disease. Such CNS or GI tract diseases (e.g. Crohn's disease or Parkinson's disease) may be at least partially caused by a mutation of LRRK2, for which an engineered polynucleotide sequence described herein can facilitate editing in, thus correcting the mutation in LRRK2 and reducing the incidence of the CNS or GI tract disease in the subject. Thus, the circularized engineered polynucleotides (or precursor engineered polynucleotides configured to circularize in a cell) may be used in a method of treatment of diseases such as Crohn's disease or Parkinson's disease.

DMD. In some embodiments, the present disclosure provides compositions and methods of use thereof of circularized engineered polynucleotides (or precursor engineered polynucleotides configured to circularize in a cell) that are capable of facilitating RNA editing of a Duchenne muscular dystrophy (DMD) gene. In some embodiments, circularized engineered polynucleotides can target an exon of a DMD gene, such as exon 51, 45, 53, 44, 46, 52, 50, 43, 6, 7, 8, 55, 2, 11, 17, 19, 21, 57, 59, 62, 63, 65, 66, 69, 74 and/or 75 in the DMD gene pre-mRNA that at least in part encodes a dystrophin protein. Said circularized engineered polynucleotides targeting a site in a DMD gene can be encoded for by an engineered polynucleotide construct of the present disclosure (e.g. a precursor engineered polynucleotide. Sequences of circularized engineered polynucleotides (or precursor engineered polynucleotides configured to circularize in a cell) configured to target DMD may comprise a targeting domain with at least about: 70%, 75%, 80%, 85%, 90%, 95%, or 100% sequence identity to SEQ ID NO: 56, or SEQ ID NO: 57. Said circularized engineered polynucleotides (or precursor engineered polynucleotides configured to circularize in a cell) may be delivered via viral vector (e.g., encoded for and delivered via AAV) as disclosed herein and may be administered via any route of administration disclosed herein to a subject in need thereof. The subject may be human and may be at risk of developing or has developed a disease or condition associated with mutations in a DMD gene such as DMD. DMD may be at least partially caused by a mutation of a DMD gene, for which an engineered polynucleotide sequence described herein can facilitate editing in, thus correcting the mutation in DMD gene and reducing the incidence of the DMD in the subject. Thus, the circularized engineered polynucleotides (or precursor engineered polynucleotides configured to circularize in a cell) may be used in a method of treatment of diseases such as DMD.

TUBB4A. In some embodiments, the present disclosure provides compositions and methods of use thereof of circularized engineered polynucleotides (or precursor engineered polynucleotides configured to circularize in a cell) that are capable of facilitating RNA editing of the TUBB4A gene. In some embodiments, circularized engineered polynucleotides can target the D249N mutation in TUBB4A, which can be caused by a 745G>A nucleotide mutation. Said circularized engineered polynucleotides targeting a site in a TUBB4A gene can be encoded for by an engineered polynucleotide construct of the present disclosure (e.g. a precursor engineered polynucleotide. Sequences of circularized engineered polynucleotides (or precursor engineered polynucleotides configured to circularize in a cell) configured to target TUBB4A may comprise a targeting domain with at least about: 70%, 75%, 80%, 85%, 90%, 95%, or 100% sequence identity to SEQ ID NO: 32, SEQ ID NO: 33, or SEQ ID NO: 34. Said circularized engineered polynucleotides (or precursor engineered polynucleotides configured to circularize in a cell) may be delivered via viral vector (e.g., encoded for and delivered via AAV) as disclosed herein and may be administered via any route of administration disclosed herein to a subject in need thereof. The subject may be human and may be at risk of developing or has developed a disease or condition associated with mutations in a TUBB4A gene such as Hypomyelination with Atrophy of Basal Ganglia and cerebellum (H-ABC). H-ABC may be at least partially caused by a mutation of a TUBB4A gene, for which an engineered polynucleotide sequence described herein can facilitate editing in, thus correcting the mutation in TUBB4A gene and reducing the incidence of the H-ABC in the subject. Thus, the circularized engineered polynucleotides (or precursor engineered polynucleotides configured to circularize in a cell) may be used in a method of treatment of diseases such as H-ABC.

### Numbered Embodiments

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. An engineered polynucleotide comprising:
a) a targeting domain that is at least partially complementary to a target RNA; and
b) a spacer domain,
wherein the engineered polynucleotide comprising the spacer domain has a lower Gibbs free energy (ΔG) of binding of the engineered polynucleotide to the target RNA, relative to a ΔG of binding of a corresponding engineered polynucleotide that lacks the spacer domain, as determined by Kelvin Probe Force Microscopy (KPFM), wherein;
   (i) the engineered polynucleotide does not comprise a 5′ reducing hydroxyl, a 3′ reducing hydroxyl, or both, capable of being exposed to a solvent,
   (ii) the engineered polynucleotide comprises a backbone comprising a plurality of sugar and phosphate moieties covalently linked together,
   (iii) the engineered polynucleotide is less susceptible to hydrolytic degradation than a comparable linear polynucleotide comprising a 5′ reducing hydroxyl, a 3′ reducing hydroxyl, or both,
   (iv) the engineered polynucleotide comprises a longer half-life in a human cell than to an otherwise comparable linear polynucleotide comprising a 5′ reducing hydroxyl, a 3′ reducing hydroxyl, or both
   (v) the engineered polynucleotide is circular, or
   (vi) any combination of i-v.

Embodiment 2. The engineered polynucleotide of embodiment 1, wherein the spacer domain is configured to not bind to the target RNA when the targeting domain binds to the target RNA.

Embodiment 3. The engineered polynucleotide of embodiments 1 or 2, wherein when the targeting domain binds to the target RNA, the spacer domain is separated from the targeting domain by at least 1 nucleotide, and if the spacer domain binds to the target RNA, the binding of the spacer domain does not produce an edit of the target RNA at the portion of the target RNA that binds to the spacer domain.

Embodiment 4. The engineered polynucleotide of any one of embodiments 1-3, wherein when the spacer domain is adjacent to a 5′ end or a 3′ end of the targeting domain, the spacer domain is not complementary to the target RNA.

Embodiment 5. An engineered polynucleotide comprising: a targeting domain that is at least partially complementary to a target RNA, an RNA editing entity recruiting domain, and a spacer domain, wherein the spacer comprises a polynucleotide and the spacer polynucleotide is not located in the targeting domain and is not located in the RNA editing entity recruiting domain and wherein the engineered polynucleotide comprising the spacer domain has a lower Gibbs free energy (ΔG) of binding of the engineered polynucleotide to the target RNA, relative to a ΔG of binding of a corresponding engineered polynucleotide that lacks the spacer domain, as determined by Kelvin Probe Force Microscopy (KPFM) and wherein;
(i) the engineered polynucleotide does not comprise a 5′ reducing hydroxyl, a 3′ reducing hydroxyl, or both, capable of being exposed to a solvent;
(ii) the engineered polynucleotide comprises a backbone comprising a plurality of sugar and phosphate moieties covalently linked together,
(iii) the engineered polynucleotide is less susceptible to hydrolytic degradation than a comparable linear polynucleotide comprising a 5′ reducing hydroxyl, a 3′ reducing hydroxyl, or both,
(iv) the engineered polynucleotide comprises a longer half-life in a human cell than to an otherwise comparable linear polynucleotide comprising a 5′ reducing hydroxyl, a 3′ reducing hydroxyl, or both
(v) the engineered polynucleotide is circular, or
(vi) any combination of i-v.

Embodiment 6. The engineered polynucleotide of any one of embodiments 1-5, wherein the spacer domain is configured to facilitate the engineered RNA adopting a conformation that facilitates at least partial binding to the target RNA.

Embodiment 7. The engineered polynucleotide of embodiment 6, wherein the spacer domain is configured to facilitate an increase in the binding specificity of the engineered polynucleotide to the target RNA, among a plurality of other RNA, relative to a binding specificity of the corresponding polynucleotide that lacks the spacer domain, as determined by sequencing of the target RNA and plurality of other RNA after contacting with the engineered polynucleotide or the corresponding polynucleotide that lacks the spacer domain.

Embodiment 8. The engineered polynucleotide of any one of embodiments 1-7, wherein the spacer domain is configured to facilitate a lower entropy (ΔS) of binding of the engineered polynucleotide to the target RNA, relative to the corresponding engineered polynucleotide that lacks the spacer domain, as determined by Kelvin Probe Force Microscopy (KPFM).

Embodiment 9. The engineered polynucleotide of any one of embodiments 1-8, wherein the spacer domain is configured to at least maintain an editing efficiency of the engineered polynucleotide to the target RNA, relative an editing efficiency of the corresponding engineered polynucleotide that lacks the spacer domain, as determined by sequencing of the target RNA after contacting with the engineered polynucleotide or the corresponding engineered polynucleotide that lacks the spacer domain.

Embodiment 10. The engineered polynucleotide of embodiment 9, wherein the at least maintain comprises an increase.

Embodiment 11. The engineered guide of embodiment 1, wherein the engineered polynucleotide has an increased editing efficiency to the target RNA by at least about 90%, relative to an otherwise comparable polynucleotide that comprises a 5′ reducing hydroxyl, a 3′ reducing hydroxyl, or both, wherein the editing efficiency is determined by (i) transfecting the target RNA into cell line 293T, (ii) transfecting the engineered polynucleotide and the otherwise comparable engineered polynucleotide that comprises the 5′ reducing hydroxyl, the 3′ reducing hydroxyl or both, into cell line 293T, and (iii) sequencing the target RNA.

Embodiment 12. The engineered polynucleotide of any one of embodiments 1-11, wherein the backbone comprises a polynucleotide, wherein each nucleotide of the polynucleotide is directly connected to two adjacent nucleotides by two different phosphorus-oxygen bonds in two different phosphorus containing groups covalently attached to a 3′ and a 5′ carbon of a sugar of the nucleotides.

Embodiment 13. The engineered polynucleotide of embodiment 12, wherein at least some sugars of the nucleotides comprised in a subsection contain 2′ hydroxyl groups.

Embodiment 14. The engineered polynucleotide of any one of embodiments 1-13, wherein the spacer domain has a sequence length of from about 1 nucleotide to about 1,000 nucleotides in length.

Embodiment 15. The engineered polynucleotide of any one of embodiments 1-14, wherein at least about 80% of the nucleotides of the spacer domain are non-complementary to the target RNA.

Embodiment 16. The engineered polynucleotide of any one of embodiments 1-15, wherein the targeting domain has a sequence length of from about 20 nucleotides to about 1,000 nucleotides in length.

Embodiment 17. The engineered polynucleotide of any one of embodiments 1-16, wherein the target RNA is an RNA selected from the group consisting of a pre-messenger RNA, a messenger RNA, a ribosomal RNA, a transfer RNA, a long non-coding RNA and a small RNA.

Embodiment 18. The engineered polynucleotide of any one of embodiments 1-17, wherein the engineered polynucleotide comprises a polynucleotide sequence with at least about: 70%, 75%, 80%, 85%, 90%, 95%, or 100% sequence homology to

(SEQ ID NO: 23)
5′ GATAAAAGGCGTACATAATTCTTGTGTCTACTGTACAGAATACTG
CCGCCAGCTGGATTTCCCAATTCTGAGTAACACTCTGCAATCCAAACA
GGGTTCA 3′.

Embodiment 19. The engineered polynucleotide of embodiment 5, wherein the targeting domain is within about 5 nucleotides of the RNA editing entity recruiting domain.

Embodiment 20. The engineered polynucleotide of embodiment 5, wherein the RNA editing entity recruiting domain recruits an RNA editing entity that, when associated with the engineered polynucleotide and the target RNA, performs a chemical transformation on a base of a nucleotide in the target RNA.

Embodiment 21. The engineered polynucleotide of embodiment 20, wherein the chemical transformation results in an increased level of a protein or fragment thereof after translation of the target RNA with the chemical transformation, relative to an otherwise comparable target RNA lacking the chemical transformation.

Embodiment 22. The engineered polynucleotide of embodiment 21, wherein the increased level is from about 5% to about 100%.

Embodiment 23. The engineered polynucleotide of embodiment 20, wherein the chemical transformation results in a decreased level of a protein or fragment thereof after translation of the target RNA with the chemical transformation, relative to an otherwise comparable target RNA lacking the chemical transformation.

Embodiment 24. The engineered polynucleotide of embodiment 23, wherein the reduced level is from about 5% to about 100%.

Embodiment 25. The engineered polynucleotide of any one of embodiments 20-24, wherein the chemical transformation converts a sense codon into a stop codon.

Embodiment 26. The engineered polynucleotide of any one of embodiments 20-24, wherein the chemical transformation converts a stop codon into a sense codon.

Embodiment 27. The engineered polynucleotide of any one of embodiments 20-24, wherein the chemical transformation converts a first sense codon into a second sense codon.

Embodiment 28. The engineered polynucleotide of any one of embodiments 20-24, wherein the chemical transformation converts a first stop codon into a second stop codon.

Embodiment 29. The engineered polynucleotide of any one of embodiments 20-28, wherein the engineered polynucleotide forms a secondary structure comprising: a stem-loop, a cruciform, a toe hold, a mismatch bulge, or any combination thereof

Embodiment 30. The engineered polynucleotide of embodiment 20, wherein the RNA editing entity recruiting domain comprises at least about: 70%, 75%, 80%, 85%, 90%, 95%, or 100% sequence homology to at least about 20 nucleotides of: an Alu domain, an APOBEC recruiting domain, a GluR2 domain, or a Cas13 recruiting domain.

Embodiment 31. The engineered polynucleotide of embodiment 30, wherein the RNA editing entity recruiting domain comprises at least about: 70%, 75%, 80%, 85%, 90%, 95%, or 100% sequence homology to at least about 20 nucleotides of the Alu domain.

Embodiment 32. The engineered polynucleotide of embodiment 31, wherein the RNA editing entity recruiting domain comprises at least about: 70%, 75%, 80%, 85%, 90%, 95%, or 100% sequence homology to the Alu domain.

Embodiment 33. The engineered polynucleotide of embodiment 30, wherein the RNA editing entity recruiting domain comprises at least about: 70%, 75%, 80%, 85%, 90%, 95%, or 100% sequence homology to at least about 20 nucleic acids of the APOBEC recruiting domain.

Embodiment 34. The engineered polynucleotide of embodiment 33, wherein the RNA editing entity recruiting domain comprises at least about: 70%, 75%, 80%, 85%, 90%, 95%, or 100% sequence homology to the APOBEC recruiting domain.

Embodiment 35. The engineered polynucleotide of embodiment 30, wherein the RNA editing entity recruiting domain comprises at least about: 70%, 75%, 80%, 85%, 90%, 95%, or 100% sequence homology to at least about 20 nucleic acids of the Cas13 recruiting domain, wherein the Cas13 recruiting domain comprises a Cas13a recruiting domain, a Cas13b recruiting domain, a Cas13c recruiting domain, or a Cas13d recruiting domain.

Embodiment 36. The engineered polynucleotide of embodiment 35, wherein the RNA editing entity recruiting domain comprises at least about: 70%, 75%, 80%, 85%, 90%, 95%, or 100% sequence homology to at least about 20 nucleic acids of the Cas13b recruiting domain.

Embodiment 37. The engineered polynucleotide of embodiment 36, wherein the sequence comprises at least about: 70%, 75%, 80%, 85%, 90%, 95%, or 100% sequence homology to the Cas13b recruiting domain.

Embodiment 38. The engineered polynucleotide of any one of embodiments 5-37, wherein the RNA editing entity recruiting domain is configured to at least transiently associate with an RNA editing entity.

Embodiment 39. The engineered polynucleotide of embodiment 38, wherein the RNA editing entity comprises an ADAR protein, an APOBEC protein, or both.

Embodiment 40. The engineered polynucleotide of embodiment 38, comprising the ADAR protein, which comprises ADAR1 or ADAR2.

Embodiment 41. The engineered polynucleotide of any one of embodiments 38-40, wherein when the RNA editing entity recruiting domain is at least transiently associated with the RNA editing entity, and when the targeting domain is at least transiently associated with the target RNA, the engineered polynucleotide is configured to facilitate an edit of a base of the target RNA.

Embodiment 42. The engineered polynucleotide of embodiments 38 or 40, wherein the RNA editing entity is an endogenous enzyme.

Embodiment 43. The engineered polynucleotide of embodiments 38 or 40, wherein the RNA editing entity is a recombinant enzyme.

Embodiment 44. The engineered polynucleotide of any one of embodiments 1-43, wherein the engineered polynucleotide further comprises a modification.

Embodiment 45. The engineered polynucleotide of embodiment 44, wherein the modification comprises a sugar modification on a nucleotide.

Embodiment 46. The engineered polynucleotide of embodiment 45, wherein a nucleotide of the engineered polynucleotide comprises a methyl group, a fluoro group, a methoxyethyl group, an ethyl group, a phosphate group, an amide group, an ester group, or any combination thereof

Embodiment 47. The engineered polynucleotide of any one of embodiments 1-46, wherein the engineered polynucleotide is at least partially genetically encodable.

Embodiment 48. The engineered polynucleotide of any one of embodiments 2-47, wherein the RNA editing entity is linked to the engineered polynucleotide by a linkage.

Embodiment 49. The engineered polynucleotide of embodiment 48, wherein the linkage is a direct or an indirect covalent linkage.

Embodiment 50. The engineered polynucleotide of any one of embodiments 2-49, wherein the RNA editing entity comprises a fusion polypeptide.

Embodiment 51. The engineered polynucleotide of any one of embodiments 1-50, wherein the engineered polynucleotide retains a half-life, in an aqueous solution at a physiological pH, that is at least about 4 times longer than an mRNA naturally present in a human cell.

Embodiment 52. The engineered polynucleotide of any one of embodiments 1-51, wherein the spacer domain comprises a polynucleotide sequence of: 5′AUAUA 3′.

Embodiment 53. The engineered polynucleotide of any one of embodiments 1-51, wherein the spacer domain comprises a polynucleotide sequence of: 5′AUAAU 3′.

Embodiment 54. The engineered polynucleotide of any one of embodiment 1-53, wherein the engineered polynucleotide further comprises a sequence with at least about 80% homology to a U7 or a U1 promoter sequence.

Embodiment 55. An engineered polynucleotide comprising: a targeting domain that is at least partially complementary to a target RNA, an RNA editing entity recruiting domain, and a spacer domain, wherein when the engineered polynucleotide is represented as a polynucleotide sequence in a circular 2-dimensional format with one nucleotide after the other, the spacer domain enlarges the engineered polynucleotide by the addition of one or more nucleotides, wherein the engineered polynucleotide does not comprise a 5′ reducing hydroxyl, a 3′ reducing hydroxyl, or both, capable of being exposed to a solvent.

Embodiment 56. A precursor engineered polynucleotide which, prior to processing, comprises, in order of 5′ to 3′: a first ribozyme domain; a first ligation domain; a first spacer domain; a targeting domain that is at least partially complementary to a target RNA, a second spacer domain, a second ligation domain, and a second ribozyme domain, and wherein the spacer domain is configured to not bind to the target RNA when the targeting domain binds to the target RNA.

Embodiment 57. The precursor engineered polynucleotide of embodiment 56, further comprising an RNA editing entity recruiting domain.

Embodiment 58. The precursor engineered polynucleotide of embodiment 56 or 57, wherein the first spacer domain, the second spacer domain or both comprises a polynucleotide sequence of: 5′AUAUA 3′.

Embodiment 59. The precursor engineered polynucleotide of embodiment 56 or 57, wherein the first spacer domain, the second spacer domain or both comprises a polynucleotide sequence of: 5′AUAAU 3′.

Embodiment 60. The precursor engineered polynucleotide of any one of embodiment 56-59 wherein the precursor engineered polynucleotide further comprises a sequence with at least about 80% homology to a U7 or a U1 promoter sequence.

Embodiment 61. The precursor engineered polynucleotide of any one of embodiments 56-60, wherein the first spacer domain, the second spacer domain, or both has a sequence length of from about 1 nucleotide to about 1,000 nucleotides in length.

Embodiment 62. The precursor engineered polynucleotide of any one of embodiments 56-61, wherein at least about 80% of the nucleotides of the first spacer domain, the second spacer domain, or both are non-complementary to the target RNA.

Embodiment 63. The precursor engineered polynucleotide of any one of embodiments 56-62, wherein the targeting domain has a sequence length of from about 20 nucleotides to about 1,000 nucleotides in length.

Embodiment 64. The precursor engineered polynucleotide of any one of embodiments 57-63, wherein the targeting domain or the RNA editing recruiting domain do not comprise the first spacer domain or the second spacer domain.

Embodiment 65. The precursor engineered polynucleotide of any one of embodiments 57-64, wherein the targeting domain is within 5 nucleotides of the RNA editing entity recruiting domain.

Embodiment 66. The precursor engineered polynucleotide of any one of embodiments 57-65, wherein the RNA editing entity recruiting domain comprises at least about: 70%, 75%, 80%, 85%, 90%, 95%, or 100% sequence homology to at least about 20 nucleotides of: an Alu domain, an APOBEC recruiting domain, a GluR2 domain, or a Cas13 recruiting domain.

Embodiment 67. The precursor engineered polynucleotide of embodiment 66, wherein the RNA editing entity recruiting domain comprises at least about: 70%, 75%, 80%, 85%, 90%, 95%, or 100% sequence homology to at least about 20 nucleotides of the Alu domain.

Embodiment 68. The precursor engineered polynucleotide of embodiment 67, wherein the RNA editing entity recruiting domain comprises at least about: 70%, 75%, 80%, 85%, 90%, 95%, or 100% sequence homology to the Alu domain.

Embodiment 69. The precursor engineered polynucleotide of embodiment 66, wherein the RNA editing entity recruiting domain comprises at least about: 70%, 75%, 80%, 85%, 90%, 95%, or 100% sequence homology to at least about 20 nucleic acids of the APOBEC recruiting domain.

Embodiment 70. The precursor engineered polynucleotide of embodiment 68, wherein the RNA editing entity recruiting domain comprises at least about: 70%, 75%, 80%, 85%, 90%, 95%, or 100% sequence homology to the APOBEC recruiting domain.

Embodiment 71. The precursor engineered polynucleotide of embodiment 66, wherein the RNA editing entity recruiting domain comprises at least about: 70%, 75%, 80%, 85%, 90%, 95%, or 100% sequence homology to at least about 20 nucleic acids of the Cas13 recruiting domain, wherein the Cas13 recruiting domain comprises a Cas13a recruiting domain, a Cas13b recruiting domain, a Cas13c recruiting domain, or a Cas13d recruiting domain.

Embodiment 72. The precursor engineered polynucleotide of embodiment 71, wherein the RNA editing entity recruiting domain comprises at least about: 70%, 75%, 80%, 85%, 90%, 95%, or 100% sequence homology to at least about 20 nucleic acids of the Cas13b recruiting domain.

Embodiment 73. The precursor engineered polynucleotide of embodiment 72, wherein the sequence comprises at least about: 70%, 75%, 80%, 85%, 90%, 95%, or 100% sequence homology to the Cas13b recruiting domain.

Embodiment 74. The precursor engineered polynucleotide of any one of embodiments 57-73, wherein the RNA editing entity recruiting domain is configured to at least transiently associate with an RNA editing entity.

Embodiment 75. The precursor engineered polynucleotide of embodiment 74, wherein the RNA editing entity comprises an ADAR protein, an APOBEC protein, or both.

Embodiment 76. The precursor engineered polynucleotide of embodiment 75, comprising the ADAR protein, which comprises ADAR1 or ADAR2.

Embodiment 77. The precursor engineered polynucleotide of any one of embodiments 74-76, wherein when the RNA editing entity recruiting domain is at least transiently associated with the RNA editing entity, and when the targeting domain is at least transiently associated with the target RNA, the engineered polynucleotide is configured to facilitate an edit of a base of the target RNA.

Embodiment 78. The precursor engineered polynucleotide of any one of embodiments 57-77, wherein the RNA editing entity is an isolated enzyme that naturally occurs in a human cell.

Embodiment 79. The precursor engineered polynucleotide of any one of embodiments 57-77, wherein the RNA editing entity is a recombinant enzyme.

Embodiment 80. The precursor engineered polynucleotide of any one of embodiments 56-79, further comprising a modification.

Embodiment 81. The precursor engineered polynucleotide of embodiment 80, wherein the modification comprises a sugar modification.

Embodiment 82. The precursor engineered polynucleotide of embodiment 80, wherein a nucleotide of the precursor engineered polynucleotide comprises a methyl group, a fluoro group, a methoxyethyl group, an ethyl group, a phosphate group, an amide group, an ester group, or any combination thereof.

Embodiment 83. The precursor engineered polynucleotide of any one of embodiments 56-82, wherein the engineered polynucleotide is at least partially genetically encodable.

Embodiment 84. The precursor engineered polynucleotide of any one of embodiments 57-83, wherein the RNA editing entity is linked to the precursor engineered polynucleotide by a linkage.

Embodiment 85. The precursor engineered polynucleotide of embodiment 84, wherein the linkage is a direct or an indirect covalent linkage.

Embodiment 86. A vector comprising the engineered polynucleotide of any one of embodiments 1-55 or the precursor engineered polynucleotide of any one of embodiments 56-85.

Embodiment 87. The vector of embodiment 86, wherein the vector comprises a polypeptide coat, wherein at least a portion of the engineered polynucleotide is present inside of the polypeptide coat.

Embodiment 88. The vector of embodiment 86 or 87, wherein the vector comprises a liposome, a viral vector, a nanoparticle, or any combination thereof.

Embodiment 89. A nucleic acid at least partially encoding the engineered polynucleotide of any one of embodiments 1-55 or the precursor engineered polynucleotide of any one of embodiments 56-85.

Embodiment 90. The nucleic acid of embodiment 89, wherein the nucleic acid is double stranded.

Embodiment 91. The nucleic acid of embodiment 89, wherein the nucleic acid comprises a targeting tag.

Embodiment 92. The nucleic acid of embodiment 91, comprising the targeting tag which comprises N-Acetylgalactosamine (GlaNAc).

Embodiment 93. A pharmaceutical composition in unit dose form comprising the engineered polynucleotide of any one of embodiments 1-55, the precursor engineered polynucleotide of any one of embodiments 56-85, the vector of any one of embodiments 86-88, or the nucleic acid of any one of embodiments 89-92; and a pharmaceutically acceptable excipient, diluent, or carrier.

Embodiment 94. A kit comprising the engineered polynucleotide of any one of embodiments 1-55, the precursor engineered polynucleotide of any one of embodiments 56-85, the vector of any one of embodiments 86-88, the nucleic acid of any one of embodiments 89-92, or the pharmaceutical composition of embodiment 93, and a container.

Embodiment 95. A method of treating a disease or condition in a subject in need thereof comprising: administering the engineered polynucleotide of any one of embodiments 1-55, the precursor engineered polynucleotide of any one of embodiments 56-85, the vector of any one of embodiments 86-88, the nucleic acid of any one of embodiments 89-92, or the pharmaceutical composition of embodiment 93.

Embodiment 96. The method of embodiment 95, wherein the method comprises administration of a second therapy.

Embodiment 97. The method of embodiment 95, wherein the administering is performed at least about: 1 time per day, 2 times per day, 3 times per day, 4 times per day, 5 times per day, or 6 times per day.

Embodiment 98. The method of embodiment 95, wherein the administering is performed for about: 1 day to about 8 days, 1 week to about 5 weeks, 1 month to about 12 months, 1 year to about 3 years, 3 years to about 10 years, 10 years to about 50 years, 25 years to about 100 years, or about 50 years to about 130 years.

Embodiment 99. The method of embodiment 95, wherein the administering is to a cell.

Embodiment 100. The method of embodiment 99, wherein the cell is a T-cell.

Embodiment 101. The method of embodiment 95, further comprising diagnosing the subject as having a disease.

Embodiment 102. The method of embodiment 95, wherein the subject is a human.

Embodiment 103. A method of preventing a disease or condition in a subject in need thereof comprising: prophylactically administering the engineered polynucleotide of any one of embodiments 1-55, the precursor engineered polynucleotide of any one of embodiments 56-85, the vector of any one of embodiments 86-88, the nucleic acid of any one of embodiments 89-92, or the pharmaceutical composition of embodiment 93.

Embodiment 104. The method of embodiment 103, wherein the method comprises administration of a second therapy.

Embodiment 105. The method of embodiment 103, wherein the administering is performed at least about: 1 time per day, 2 times per day, 3 times per day, 4 times per day, 5 times per day, or 6 times per day.

Embodiment 106. The method of embodiment 103, wherein the administering is performed for about: 1 day to about 8 days, 1 week to about 5 weeks, 1 month to about 12 months, 1 year to about 3 years, 3 years to about 10 years, 10 years to about 50 years, 25 years to about 100 years, or about 50 years to about 130 years.

Embodiment 107. The method of embodiment 103, wherein the administering is to a cell.

Embodiment 108. The method of embodiment 107, wherein the cell is a T-cell.

Embodiment 109. The method of embodiment 103, wherein the subject is a human.

Embodiment 110. A method of forming an engineered polynucleotide, the method comprising:
directly or indirectly ligating a nucleotide on a 3′ end of a precursor engineered polynucleotide to a nucleotide on a 5′ end of the precursor engineered polynucleotide with a ligating entity, thereby forming the engineered polynucleotide, wherein the engineered polynucleotide comprises:
   (a) a targeting domain that is at least partially complementary to a target RNA and,
   (b) a spacer domain, wherein the engineered polynucleotide does not comprise a 5′ reducing hydroxyl capable of being exposed to a solvent, wherein the engineered polynucleotide comprising the spacer domain has a lower Gibbs free energy (ΔG) of binding of the engineered polynucleotide to the target RNA, relative to a ΔG of binding of a corresponding engineered polynucleotide that lacks the spacer domain, as determined by Kelvin Probe Force Microscopy (KPFM), and wherein at least one of the following applies:
      (i) the spacer domain is configured to not bind to the target RNA when the targeting domain binds to the target RNA;
      (ii) when the targeting domain binds to the target RNA, the spacer domain is separated from the targeting domain by at least 1 nucleotide, and if the spacer domain binds to the target RNA, the binding of the spacer domain does not produce an edit of the target RNA at the portion of the target RNA that binds to the spacer domain; or
      (iii) when the spacer domain is adjacent to a 5′ end or a 3′ end of the targeting domain, the spacer domain is not complementary to the target RNA.

Embodiment 111. The method of embodiment 110, wherein the engineered polynucleotide is configured to facilitate an increase in the binding specificity to the target RNA, among a plurality of other RNA, as compared to the corresponding engineered polynucleotide that lacks the spacer domain, as determined by sequencing of the target RNA and plurality of other RNA after contacting with the engineered polynucleotide or the corresponding engineered polynucleotide.

Embodiment 112. The method of embodiment 110 or 111, wherein the spacer domain is configured to facilitate a lower entropy (ΔS) of binding of the engineered polynucleotide to the target RNA, relative to the corresponding engineered polynucleotide that lacks the spacer domain, as determined by Kelvin Probe Force Microscopy (KPFM).

Embodiment 113. The method of any one of embodiments 110-112, wherein the spacer domain is configured to facilitate the engineered RNA adopting a conformation that facilitates at least partial binding to the target RNA.

Embodiment 114. The method of embodiment 113, wherein the spacer domain is configured to at least maintain the editing efficiency of the engineered polynucleotide to the target RNA, relative an editing efficiency of the corresponding engineered polynucleotide that lacks the spacer domain, as determined by sequencing of the target RNA after contacting with the engineered polynucleotide or the corresponding engineered polynucleotide that lacks the spacer domain.

Embodiment 115. The method of embodiment 114, wherein the at least maintain comprises an increase.

Embodiment 116. The method of any one of embodiments 110-115, wherein the precursor engineered polynucleotide comprises, in order of 5′ to 3′: a first ribozyme domain; a first ligation domain; a first spacer domain; the targeting domain, a second spacer domain, a second ligation domain, and a second ribozyme domain.

Embodiment 117. The method of any one of embodiments 110-116, wherein the spacer domain comprises a polynucleotide sequence of: 5′AUAUA 3′.

Embodiment 118. The method of any one of embodiments 110-116, wherein the spacer domain comprises a polynucleotide sequence of: 5′AUAAU 3′.

Embodiment 119. The method of any one of embodiments 110-118, wherein the spacer domain has a sequence length of from about 1 nucleotide to about 1,000 nucleotides in length.

Embodiment 120. The method of any one of embodiments 110-119, wherein at least about 80% of the nucleotides of the spacer domain are non-complementary to the target RNA.

Embodiment 121. The method of any one of embodiments 110-120, wherein the targeting domain has a sequence length of from about 20 nucleotides to about 1,000 nucleotides in length.

Embodiment 122. The method of any one of embodiments 110-121, wherein the engineered polynucleotide further comprises an RNA editing entity recruiting domain.

Embodiment 123. The method of embodiment 122, wherein the targeting domain or the RNA editing recruiting domain do not comprise the spacer domain.

Embodiment 124. The method of embodiment 122, wherein the targeting domain is within 5 nucleotides of the RNA editing entity recruiting domain.

Embodiment 125. The method of embodiment 122, wherein the RNA editing entity recruiting domain recruits an RNA editing entity that, when associated with the engineered polynucleotide, performs a chemical transformation on a base of a nucleotide in the target RNA.

Embodiment 126. The method of embodiment 125, wherein the RNA editing entity comprises an ADAR protein, an APOBEC protein, or both.

Embodiment 127. The method of embodiment 126, comprising the ADAR protein, which comprises ADAR1 or ADAR2.

Embodiment 128. The method of embodiment 125, wherein the chemical transformation results in an increased level of a protein or fragment thereof after translation of the target RNA with the chemical transformation, relative to an otherwise comparable target RNA lacking the chemical transformation.

Embodiment 129. The method of embodiment 128, wherein the increased level is from about 5% to 100%.

Embodiment 130. The method of embodiment 125, wherein the chemical transformation results in a decreased level of a protein or fragment thereof after translation of the target RNA with the chemical transformation, relative to an otherwise comparable target RNA lacking the chemical transformation.

Embodiment 131. The method of embodiment 130, wherein the reduced level is from about 5% to 100%.

Embodiment 132. The method of any one of embodiments 125-131, wherein the chemical transformation converts a sense codon into a stop codon.

Embodiment 133. The method of any one of embodiments 125-131, wherein the chemical transformation converts a stop codon into a sense codon.

Embodiment 134. The method of any one of embodiments 125-131, wherein the chemical transformation converts a first sense codon into a second sense codon.

Embodiment 135. The method of any one of embodiments 125-131, wherein the chemical transformation converts a first stop codon into a second stop codon.

Embodiment 136. The method of any one of embodiments 125-135, wherein the engineered polynucleotide forms a secondary structure comprising: a stem-loop, a cruciform, a toe hold, a mismatch bulge, or any combination thereof.

Embodiment 137. The method of embodiment 125, wherein the RNA editing entity recruiting domain comprises at least about: 70%, 75%, 80%, 85%, 90%, 95%, or 100% sequence homology to at least about 20 nucleic acids of: an Alu domain, an APOBEC recruiting domain, a GluR2 domain, or a Cas13 recruiting domain.

Embodiment 138. The method of embodiment 137, wherein the RNA editing entity recruiting domain comprises at least about: 70%, 75%, 80%, 85%, 90%, 95%, or 100% sequence homology to at least about 20 nucleic acids of the Alu domain.

Embodiment 139. The method of embodiment 138, wherein the RNA editing entity recruiting domain comprises at least about: 70%, 75%, 80%, 85%, 90%, 95%, or 100% sequence homology to the Alu domain.

Embodiment 140. The method of embodiment 137, wherein the RNA editing entity recruiting domain comprises at least about: 70%, 75%, 80%, 85%, 90%, 95%, or 100% sequence homology to at least about 20 nucleic acids of the APOBEC recruiting domain.

Embodiment 141. The method of embodiment 140, wherein the RNA editing entity recruiting domain comprises at least about: 70%, 75%, 80%, 85%, 90%, 95%, or 100% sequence homology to the APOBEC recruiting domain.

Embodiment 142. The method of embodiment 137, wherein the RNA editing entity recruiting domain comprises at least about: 70%, 75%, 80%, 85%, 90%, 95%, or 100% sequence homology to at least about 20 nucleic acids of the Cas13 recruiting domain, wherein the Cas13 recruiting domain comprises a Cas13a recruiting domain, a Cas13b recruiting domain, a Cas13c recruiting domain, or a Cas13d recruiting domain.

Embodiment 143. The method of embodiment 142, wherein the RNA editing entity recruiting domain comprises at least about: 70%, 75%, 80%, 85%, 90%, 95%, or 100% sequence homology to at least about 20 nucleic acids of the Cas13b recruiting domain.

Embodiment 144. The method of embodiment 143, wherein the sequence comprises at least about: 70%, 75%, 80%, 85%, 90%, 95%, or 100% sequence homology to the Cas13b recruiting domain.

Embodiment 145. The method of any one of embodiments 122-144, wherein the RNA editing entity is an isolated enzyme naturally present in a mammalian cell.

Embodiment 146. The method of any one of embodiments 122-144, wherein the RNA editing entity is a recombinant enzyme.

Embodiment 147. The method of any one of embodiments 122-146, wherein the engineered polynucleotide comprises a modification.

Embodiment 148. The method of embodiment 147, wherein the modification comprises a sugar modification on a base of a nucleotide of the engineered polynucleotide.

Embodiment 149. The method of embodiment 148, wherein a nucleotide of the engineered polynucleotide comprises a methyl group, a fluoro group, a methoxyethyl group, an ethyl group, a phosphate group, an amide group, an ester group, or any combination thereof

Embodiment 150. The method of any one of embodiments 110-149, wherein the engineered polynucleotide is genetically encodable.

Embodiment 151. The method of any one of embodiments 122-150, wherein the RNA editing entity is linked to the engineered polynucleotide by a linkage.

Embodiment 152. The method of embodiment 151, wherein a linkage between the engineered polynucleotide and the RNA editing entity is a direct or an indirect covalent linkage.

Embodiment 153. The method of any one of embodiments 110-152, wherein the ligating entity comprises an RNA ligase.

Embodiment 154. The method of any one of embodiments 110-153, wherein the engineered polynucleotide retains a half-life, in an aqueous solution at a physiological pH, that is at least about 4 times longer than the comparable polynucleotide.

Embodiment 155. The method of any one of embodiments 110-154, wherein a therapeutically effective amount of the engineered polynucleotide dosed to a subject in need thereof is at least about 4 times less than the comparable engineered polynucleotide.

Embodiment 158. An engineered polynucleotide comprising: a targeting domain that is at least partially complementary to a target RNA; an RNA editing entity recruiting domain, wherein the RNA editing entity recruiting domain is configured to at least transiently associate with an RNA editing entity; and a spacer domain, wherein the targeting domain or the RNA editing recruiting domain do not comprise the spacer domain; wherein the engineered polynucleotide comprises a backbone comprising a plurality of sugar and phosphate moieties covalently linked together, and wherein the backbone does not comprise a 5′ reducing hydroxyl, a 3′ reducing hydroxyl, or both, capable of being exposed to a solvent.

Embodiment 159. An engineered polynucleotide comprising: a targeting domain that is at least partially complementary to a target RNA; and a spacer domain, wherein the engineered polynucleotide comprising the spacer domain has a lower Gibbs free energy (ΔG) of binding of the engineered polynucleotide to the target RNA, relative to a ΔG of binding of a corresponding engineered polynucleotide that lacks the spacer domain, as determined by Kelvin Probe Force Microscopy (KPFM); wherein the engineered polynucleotide comprises a backbone comprising a plurality of sugar and phosphate moieties covalently linked together, and wherein the backbone does not comprise a 5′ reducing hydroxyl, a 3′ reducing hydroxyl, or both, capable of being exposed to a solvent.

Embodiment 160. A precursor engineered linear polynucleotide which comprises, in order of 5′ to 3′: a first spacer domain; a targeting domain that is substantially complementary to a target RNA implicated in a disease or condition; and a second spacer domain, wherein the first or second spacer domain is not complementary to the target RNA, wherein a transcript of the precursor engineered linear polynucleotide self-circularizes upon insertion of the precursor engineered linear polynucleotide in a mammalian cell, thereby forming a circularized engineered polynucleotide; and wherein hybridization of the targeting domain with the target RNA facilitates an edit of a base of a nucleotide of the target RNA by an RNA editing enzyme.

Embodiment 161. The precursor engineered linear polynucleotide of embodiment 160, wherein the precursor engineered linear polynucleotide comprises a ribozyme domain 5′ to the first spacer domain or 3′ to the second spacer domain.

Embodiment 162. The precursor engineered linear polynucleotide of embodiment 161, wherein the precursor engineered linear polynucleotide comprises a ligation domain between the ribozyme domain and the first spacer domain or between the ribozyme domain and the second spacer domain.

Embodiment 163. The precursor engineered linear polynucleotide of any one of embodiments 160-162, wherein after self-circularization, the first spacer domain and the second spacer domain comprise a filler sequence that is from about 40% to about 70% of a total length of the circularized engineered polynucleotide.

Embodiment 164. The precursor engineered linear polynucleotide of embodiment 163, wherein after self-circularization, the first spacer domain and the second spacer domain comprise a filler sequence that is from about 50% to about 67% of the total sequence of the circularized engineered polynucleotide.

Embodiment 165. The precursor engineered linear polynucleotide of embodiment 163 or 164, wherein the filler sequence increases hybridization of the targeting domain to the target RNA, relative to an otherwise comparable circularized polynucleotide that lacks the filler sequence.

Embodiment 166. The precursor engineered linear polynucleotide of any one of embodiments 160-165, wherein the targeting domain comprises at least about 80%, at least about 85%, at least about 90%, at least about 92%, at least about 95%, at least about 97%, at least about 99%, or 100% complementarity to the target RNA.

Embodiment 167. The precursor engineered linear polynucleotide of any one of embodiments 160-166, wherein the target RNA is an RNA selected from the group consisting of a pre-messenger RNA, a messenger RNA, a ribosomal RNA, a transfer RNA, a long non-coding RNA, and a small RNA.

Embodiment 168. The precursor engineered linear polynucleotide of any one of embodiments 160-167, wherein the targeting domain is substantially complementary to a 3′ or 5′ untranslated region (UTR) of the target RNA.

Embodiment 169. The precursor engineered linear polynucleotide of any one of embodiments 160-168, wherein the targeting domain is substantially complementary to an intronic region of the target RNA.

Embodiment 170. The precursor engineered linear polynucleotide of any one of embodiments 160-169, wherein the targeting domain is substantially complementary to an upstream open reading frame (uORF) of the target RNA.

Embodiment 171. The precursor engineered linear polynucleotide of any one of embodiments 160-169, wherein the targeting domain is not is at least partially complementary to a portion of an upstream open reading frame (uORF) of the target RNA.

Embodiment 172. The precursor engineered linear polynucleotide of any one of embodiments 160-171, wherein the targeting domain comprises at least a single nucleotide that is mismatched to the target RNA.

Embodiment 173. The precursor engineered linear polynucleotide of embodiment 172, wherein the mismatched nucleotide is adjacent to two nucleotides, one on each side of the mismatched nucleotide, that are complementary to the target RNA.

Embodiment 174. The precursor engineered linear polynucleotide of any one of embodiments 160-173, wherein the edit of the base increases a level of a protein or fragment thereof, increases a length of a protein or fragment thereof, increases a functionality of a protein or fragment thereof, increases a stability of a protein or fragment thereof, or any combination thereof, after translation of the target RNA with the edit of the base, relative to a translated protein of an otherwise comparable target RNA lacking the edit.

Embodiment 175. The precursor engineered linear polynucleotide of embodiment 174, wherein the increased level is from about 5% to about 100%.

Embodiment 176. The precursor engineered linear polynucleotide of embodiment 174, wherein the increased length is from about 5% to about 100% of the protein or fragment thereof.

Embodiment 177. The precursor engineered linear polynucleotide of embodiment 174, wherein the increased stability is an increased half-life of the protein or fragment thereof.

Embodiment 178. The precursor engineered linear polynucleotide of any one of embodiments 160-177, wherein the edit of a base converts a sense codon into a stop codon.

Embodiment 179. The precursor engineered linear polynucleotide of embodiment 178, wherein the sense codon is implicated in a disease pathogenic pathway and wherein converting the sense codon to the stop codon reduces the disease pathogenic pathway.

Embodiment 180. The precursor engineered linear polynucleotide of any one of embodiments 160-177, wherein the edit of a base converts a stop codon into a sense codon.

Embodiment 181. The precursor engineered linear polynucleotide of embodiment 180, wherein the stop codon is implicated in a disease pathogenic pathway and wherein converting the stop codon to the sense codon reduces the disease pathogenic pathway.

Embodiment 182. The precursor engineered linear polynucleotide of any one of embodiments 160-177, wherein the edit of a base converts a first sense codon into a second sense codon.

Embodiment 183. The precursor engineered linear polynucleotide of embodiment 182, wherein the first sense codon is implicated in a disease pathogenic pathway and wherein converting the first sense codon to the second sense codon reduces the disease pathogenic pathway.

Embodiment 184. The precursor engineered linear polynucleotide of any one of embodiments 160-183, wherein the precursor engineered linear polynucleotide is from about 80 nucleotides to about 600 nucleotides; from about 80 nucleotides to about 300 nucleotides; from about 400 nucleotides to about 600 nucleotides; or from about 200 nucleotides to about 400 nucleotides.

Embodiment 185. The precursor engineered linear polynucleotide of embodiment 184, wherein the targeting domain is from about 20 nucleotides to about 150 nucleotides; or from about 100 nucleotides to about 200 nucleotides.

Embodiment 186. The precursor engineered linear polynucleotide of any one of embodiments embodiment 1-185, wherein the RNA editing enzyme comprises an ADAR protein or an APOBEC protein.

Embodiment 187. The precursor engineered linear polynucleotide embodiment 186, wherein the RNA editing enzyme comprises ADAR and wherein the ADAR is ADAR1.

Embodiment 188. The precursor engineered linear polynucleotide embodiment 187, wherein the ADAR1 is hADAR1.

Embodiment 189. The precursor engineered linear polynucleotide of embodiment 186, wherein the RNA editing enzyme comprises ADAR and wherein the ADAR is ADAR2.

Embodiment 190. The precursor engineered linear polynucleotide embodiment 189, wherein the ADAR2 is hADAR2.

Embodiment 191. The precursor engineered linear polynucleotide of embodiment 186, wherein the RNA editing entity comprises ADAR and wherein the ADAR is ADAR3.

Embodiment 192. The precursor engineered linear polynucleotide embodiment 191, wherein the ADAR3 is hADAR3.

Embodiment 193. The precursor engineered linear polynucleotide of embodiment 160-192, wherein the targeting domain does not comprise an aptamer.

Embodiment 194. The precursor engineered linear polynucleotide of any one of embodiments 160-193, wherein the precursor engineered linear polynucleotide does not comprise or encode a sequence encoding a sequence configured for RNA interference (RNAi).

Embodiment 195. The precursor engineered linear polynucleotide of any one of embodiments 160-194, wherein the precursor engineered linear polynucleotide does not comprise or encode a sequence encoding a short interfering RNA (siRNA), microRNA (miRNA), short hairpin RNA (shRNA), or dicer substrate.

Embodiment 196. The precursor engineered linear polynucleotide of embodiment 160, wherein the disease or condition comprises Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, or cystic fibrosis.

Embodiment 197. A precursor engineered polynucleotide which comprises, in order of 5′ to 3′: a first ribozyme domain; a first ligation domain; a first spacer domain; a targeting domain that is substantially complementary to a target RNA implicated in a disease or condition, a second ligation domain, and a second ribozyme domain, wherein the first spacer domain is not complementary to the target RNA, wherein a transcript of the precursor engineered linear polynucleotide self-circularizes upon insertion of the precursor engineered linear polynucleotide in a mammalian cell, thereby forming a circularized engineered polynucleotide; and wherein hybridization of the targeting domain with the target RNA facilitates an edit of a base of a nucleotide of the target RNA by an RNA editing enzyme.

Embodiment 198. The precursor engineered polynucleotide of embodiment 197, wherein the precursor engineered polynucleotide is linear.

Embodiment 199. The precursor engineered polynucleotide of embodiment 197 or 198, wherein the first spacer domain is a filler sequence that comprises a length of from about 50 nucleotides to about 400 nucleotides; or from about 200 nucleotides to about 100 nucleotides.

Embodiment 200. The precursor engineered polynucleotide of embodiment 199, wherein the filler sequence increases hybridization of the targeting domain to the target RNA, relative to an otherwise comparable circularized polynucleotide that lacks the filler sequence.

Embodiment 201. The precursor engineered polynucleotide of any one of embodiments 197-200, wherein the targeting domain comprises at least about 80%, at least about 85%, at least about 90%, at least about 92%, at least about 95%, at least about 97%, at least about 99%, or 100% complementarity to the target RNA.

Embodiment 202. The precursor engineered polynucleotide of any one of embodiments 197-201, further comprising an RNA editing enzyme recruiting domain.

Embodiment 203. The precursor engineered polynucleotide of any one of embodiments 197-202, wherein the first spacer domain, the second spacer domain or both comprises a polynucleotide sequence of: 5′AUAUA 3′.

Embodiment 204. The precursor engineered polynucleotide of any one of embodiments 197-202, wherein the first spacer domain, the second spacer domain or both comprises a polynucleotide sequence of: 5′AUAAU 3′.

Embodiment 205. The precursor engineered linear polynucleotide of any one of embodiments 197-204, wherein the circularized engineered polynucleotide has a lower Gibbs free energy (ΔG) of binding to the target RNA, relative to a ΔG of binding of a circularized engineered polynucleotide that lacks the first spacer domain, to the target RNA, as determined by Kelvin Probe Force Microscopy (KPFM).

Embodiment 206. The precursor engineered polynucleotide of any one of embodiments 197-205, wherein the precursor engineered polynucleotide further comprises a sequence with at least about 80%, at least about 85%, at least about 90%, at least about 92%, at least about 95%, at least about 97%, at least about 99%, or 100% sequence identity to a U7 or a U1 promoter sequence.

Embodiment 207. The precursor engineered polynucleotide of any one of embodiments 197-206, wherein the precursor engineered polynucleotide further comprises an RNA sequence motif.

Embodiment 208. The precursor engineered polynucleotide of embodiment 207, wherein the RNA sequence motif comprises a SmOPT sequence, a hnRNPA1 sequence, a MALAT1 sequence, a SIRLOIN sequence, or a combination thereof.

Embodiment 209. The precursor engineered polynucleotide of any one of embodiments 197-208, wherein the targeting domain comprises at least a single nucleotide that is mismatched to the target RNA.

Embodiment 210. The precursor engineered polynucleotide of embodiment 209, wherein the mismatched nucleotide is adjacent to two nucleotides, one on each side of the mismatched nucleotide, that are complementary to the target RNA.

Embodiment 211. The precursor engineered polynucleotide of any one of embodiments 197-210, wherein the precursor polynucleotide is about 80 nucleotides to about 600 nucleotides; about 80 nucleotides to about 300 nucleotides; about 400 nucleotides to about 600 nucleotides; about 200 nucleotides to about 400 nucleotides or about 20 nucleotides to about 150 nucleotides.

Embodiment 212. The precursor engineered polynucleotide of embodiment 197, wherein the RNA editing entity comprises an ADAR protein or an APOBEC protein.

Embodiment 213. The precursor engineered polynucleotide embodiment 212, wherein the RNA editing entity comprises ADAR and wherein the ADAR comprises ADAR1, ADAR2 or ADAR3.

Embodiment 214. The precursor engineered polynucleotide of embodiment 197, wherein the disease or condition comprises Rett syndrome, Huntington's disease, Parkinson's Disease, Alzheimer's disease, a muscular dystrophy, or Tay-Sachs Disease, alpha-1 antitrypsin deficiency (AATD), a dementia, a tauopathy, a synucleinopathy, Stargardt disease, or cystic fibrosis.

Embodiment 215. The precursor engineered polynucleotide of any one of embodiments 197-214, wherein the target RNA is an RNA selected from the group consisting of a pre-messenger RNA, a messenger RNA, a ribosomal RNA, a transfer RNA, a long non-coding RNA, and a small RNA.

Embodiment 216. The precursor engineered polynucleotide of any one of embodiments 197-215, wherein the precursor engineered polynucleotide does not comprise or encode a sequence encoding a sequence configured for RNA interference (RNAi).

Embodiment 217. The precursor engineered polynucleotide of any one of embodiments 197-216, wherein precursor engineered polynucleotide does not comprise or encode a sequence encoding a short interfering RNA (siRNA), microRNA (miRNA), short hairpin RNA (shRNA), or dicer substrate.

Embodiment 218. The precursor engineered polynucleotide of any one of embodiments 197-217, wherein the targeting domain is substantially complementary to at least a portion of a 3′ or 5′ untranslated region (UTR) of the target RNA.

Embodiment 219. The precursor engineered polynucleotide of any one of embodiments 197-218, wherein the targeting domain is substantially complementary to at least a portion of an intronic region of the target RNA.

Embodiment 220. The precursor engineered polynucleotide of any one of embodiments 197-219, wherein the targeting domain is substantially complementary to at least a portion of an upstream open reading frame (uORF) of the target RNA.

Embodiment 221. The precursor engineered polynucleotide of any one of embodiments 197-214, wherein the targeting domain is substantially complementary to at least a portion of an upstream open reading frame (uORF) of the target RNA.

Embodiment 222. An engineered polynucleotide encoding or comprising: a) a targeting domain that is substantially complementary to a target RNA implicated in a disease or

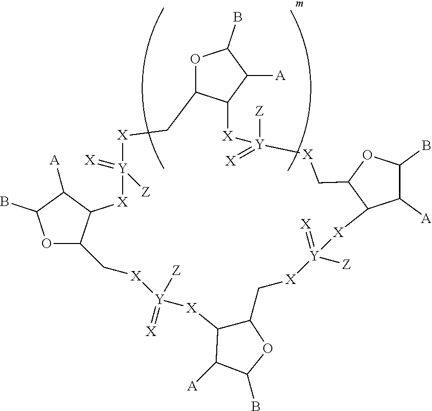

condition; and b) a spacer domain, wherein the engineered polynucleotide comprising the spacer domain has a lower Gibbs free energy (ΔG) of binding of the targeting domain to the target RNA, relative to a ΔG of binding of a targeting domain of a corresponding engineered polynucleotide that lacks the spacer domain, to the target RNA, as determined by Kelvin Probe Force Microscopy (KPFM), and wherein the engineered polynucleotide comprises a structure of Formula (I):
(I)
   wherein each X is O; each Y is P; each Z is O or S; each A is independently H, D, halogen, OM, SM, NRM, or NRR′; each B is independently uracil, thymine, adenine, cytosine, guanine, a salt of any of these, or a derivative of any of these; m is independently an integer from 0-1,000; and wherein: each M is independently an inorganic or organic cation, H, or D; and each R and R′ is independently H, D, halogen, or C₁-C₆ alkyl, wherein association of the targeting domain with a sequence of the target RNA facilitates an edit of a base of a nucleotide of the target RNA by an RNA editing entity.

Embodiment 223. The engineered polynucleotide of embodiment 222, wherein each unit m is independently in the (D)- or (L)-configuration.

Embodiment 224. The engineered polynucleotide of embodiment 222, wherein the targeting domain comprises at least about at least about 80%, at least about 85%, at least about 90%, at least about 92%, at least about 95%, at least about 97%, at least about 99%, or 100% complementarity to the target RNA.

Embodiment 225. The engineered polynucleotide of any one of embodiments 222-224, wherein Formula (I) is according to Formula (II)

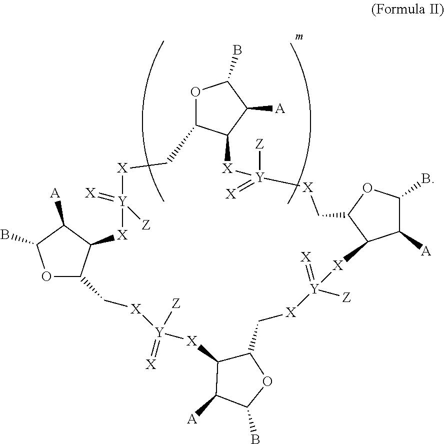

Embodiment 226. The engineered polynucleotide of any one of embodiments 222-225, wherein each Z is O and each R is H.

Embodiment 227. The engineered polynucleotide of any one of embodiments 222-226, wherein each m is an independent integer from about 30-600.

Embodiment 228. The engineered polynucleotide of any one of embodiments 222-227, wherein the edit of the base of the nucleotide of the target RNA by the RNA editing enzyme is determined in an in vitro assay comprising: (i) directly or indirectly introducing the target RNA into a primary cell line, (ii) directly or indirectly introducing the engineered polynucleotide into the primary cell line, and (iii) sequencing the target RNA.

Embodiment 229. The engineered polynucleotide of any one of embodiments 222-228, wherein the edit of the base results in an increased level of a protein or fragment thereof, an increased length of a protein or fragment thereof, an increased functionality of a protein or fragment thereof, an increased stability of a protein or fragment thereof, or any combination thereof after translation of the target RNA with the edit of the base, relative to a translated protein of an otherwise comparable target RNA lacking the edit.

Embodiment 230. The engineered polynucleotide of embodiment 229, wherein the increased level is from about 5% to about 100%.

Embodiment 231. The engineered polynucleotide of embodiment 229, wherein the increased length is from about 5% to about 100% of the protein or fragment thereof

Embodiment 232. The engineered polynucleotide of embodiment 229, wherein the increased stability is an increased half-life of the protein or fragment thereof.

Embodiment 233. The engineered polynucleotide of any one of embodiments 222-232, wherein the RNA editing entity comprises an ADAR protein, an APOBEC protein, or both.

Embodiment 234. The engineered polynucleotide of any one of embodiments 222-233, wherein the RNA editing entity comprises ADAR and wherein ADAR comprises ADAR1 or ADAR2.

Embodiment 235. The engineered polynucleotide of any one of embodiments 222-234, wherein the edit of a base converts a sense codon into a stop codon.

Embodiment 236. The engineered polynucleotide of embodiment 235, wherein the sense codon is implicated in a disease pathogenic pathway and wherein converting the sense codon to the stop codon reduces the disease pathogenic pathway

Embodiment 237. The engineered polynucleotide of any one of embodiments 222-234, wherein the edit of a base converts a stop codon into a sense codon.

Embodiment 238. The engineered polynucleotide of any one of embodiments 222-234, wherein the edit of a base converts a first sense codon into a second sense codon.

Embodiment 239. The engineered polynucleotide of embodiment 238, wherein the first sense codon is implicated in a disease pathogenic pathway and wherein converting the first sense codon to the second sense codon reduces the disease pathogenic pathway.

Embodiment 240. The engineered polynucleotide of any one of embodiments 222-238, wherein the edit of a base, converts a sense codon specifying a first amino acid into a second sense codon specifying a second amino acid.

Embodiment 241. The engineered polynucleotide of embodiment 240, wherein the first amino acid is a protease cleavage site.

Embodiment 242. The engineered polynucleotide of any one of embodiments 222-241, wherein the disease or condition is Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, or cystic fibrosis.

Embodiment 243. An engineered polynucleotide encoding or comprising: a) a targeting domain that is capable of hybridizing to a sequence of a target RNA; and b) a spacer domain, wherein the engineered polynucleotide comprising the spacer domain has a lower Gibbs free energy (ΔG) of binding of the engineered polynucleotide to the target RNA, relative to a ΔG of binding of a corresponding engineered polynucleotide that lacks the spacer domain, to the target RNA, as determined by Kelvin Probe Force Microscopy (KPFM), and wherein the engineered polynucleotide comprises a structure of Formula (I):
(I)
   wherein each X is O; each Y is P; each Z is O or S; each A is independently H, D, halogen, OM, SM, NRM, or NRR′; each B is independently uracil, thymine, adenine, cytosine, guanine,

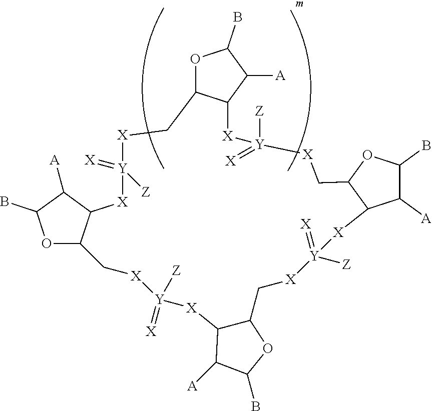

a salt of any of these, or a derivative of any of these; m is independently an integer from 0-1,000; and wherein: each M is independently an inorganic or organic cation, H, or D; and each R and R′ is independently H, D, halogen, or C₁-C₆ alkyl, wherein the targeting domain is configured to at least partially associate with an untranslated region of the target RNA, wherein the association of the targeting domain with the untranslated region of the target RNA facilitates a reduction in an expression level of a polypeptide encoded for by the target RNA.

Embodiment 244. An engineered polynucleotide encoding or comprising: a) a targeting domain that is capable of hybridizing to a sequence of a target RNA; and b) a spacer domain, wherein the engineered polynucleotide comprising the spacer domain has a lower Gibbs free energy (ΔG) of binding of the engineered polynucleotide to the target RNA, relative to a ΔG of binding of a corresponding engineered polynucleotide that lacks the spacer domain, to the target RNA, as determined by Kelvin Probe Force Microscopy (KPFM), and wherein the

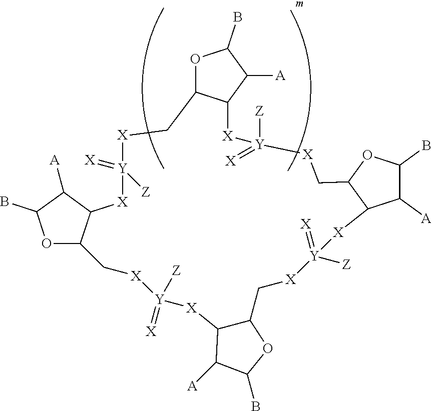

engineered polynucleotide comprises a structure of Formula (I):
(I)
   wherein each X is O; each Y is P; each Z is O or S; each A is independently H, D, halogen, OM, SM, NRM, or NRR′; each B is independently uracil, thymine, adenine, cytosine, guanine, a salt of any of these, or a derivative of any of these; m is independently an integer from 0-1,000; and wherein: each M is independently an inorganic or organic cation, H, or D; and each R and R′ is independently H, D, halogen, or C₁-C₆ alkyl, wherein the targeting domain is configured to at least partially associate with an untranslated region of the target RNA, wherein the association of the targeting domain with the untranslated region of the target RNA facilitates a reduction in an expression level of a polypeptide encoded for by the target RNA and wherein association of the targeting domain with a sequence of the target RNA facilitates an edit of a base of a nucleotide of the target RNA by an RNA editing entity.

Embodiment 245. The engineered polynucleotide of embodiment 243 or 244, wherein the association of the targeting domain with the untranslated region of the target RNA facilitates the reduction in the expression level of a polypeptide encoded for by the target RNA, relative to a level of the polypeptide encoded for by the target RNA in the absence of the engineered polynucleotide as determined by an in vitro assay comprising: (i) directly or indirectly introducing the target RNA into a first cell and a second cell of a primary cell line, (ii) directly or indirectly introducing the engineered polynucleotide into the first cell of the primary cell line, and (iii) comparing an amount of the polypeptide expressed from the target RNA in the first cell and the second cell.

Embodiment 246. The engineered polynucleotide of any one of embodiments 243-245, wherein the targeting domain is at least partially complementary to the target RNA.

Embodiment 247. The engineered polynucleotide of embodiment 246, wherein the targeting domain is at least partially complementary to the target RNA comprises a polynucleotide sequence with at least about 80%, at least about 85%, at least about 90%, at least about 92%, at least about 95%, at least about 97%, at least about 99%, or 100% sequence homology to a reverse complement to the target RNA.

Embodiment 248. The engineered polynucleotide of any one of embodiments 243-247, wherein each unit m is independently in the (D)- or (L)-configuration.

Embodiment 249. The engineered polynucleotide of any one of embodiments 243-248, wherein Formula (I) is according to Formula (II)

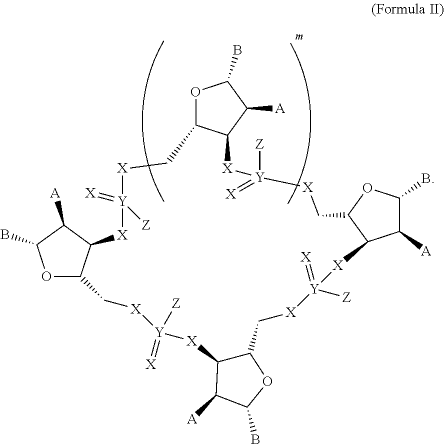

Embodiment 250. The engineered polynucleotide of any one of embodiments 243-249, wherein the reduced level is from about 5% to about 100%.

Embodiment 251. The engineered polynucleotide of any one of embodiments 243-250, wherein the primary cell line comprises a neuron cell, a photoreceptor cell, a retinal pigment epithelium cell, a glia cell, a myoblast cell, a myotube cell, a hepatocyte, a lung epithelial cell, or a fibroblast cell.

Embodiment 252. The engineered polynucleotide of any one of embodiments 243-251, wherein the target RNA is an RNA selected from the group consisting of a pre-messenger RNA, a messenger RNA, a ribosomal RNA, a transfer RNA, a long non-coding RNA and a small RNA.

Embodiment 253. The engineered polynucleotide of any one of embodiments 243-252, wherein the engineered polynucleotide does not comprise or encode a sequence encoding an sequence configured for RNA interference (RNAi).

Embodiment 254. The engineered polynucleotide of any one of embodiments 243-253, wherein the engineered polynucleotide does not comprise or encode a sequence encoding a short interfering RNA (siRNA), microRNA (miRNA), short hairpin RNA (shRNA), or dicer substrate.

Embodiment 255. The engineered polynucleotide of any one of embodiments 243-254, wherein the reduction in the expression level of the polypeptide encoded by the target RNA is at least partially independent of an RNA-directed RNA endonuclease.

Embodiment 256. The engineered polynucleotide of any one of embodiments 243-255, wherein the reduction in the expression level of the polypeptide encoded by the target RNA is at least partially independent of RISC- or Dicer.

Embodiment 257. The engineered polynucleotide of any one of embodiments 243-256, wherein the reduction in the expression level of the polypeptide encoded by the target RNA is at least partially independent of Argonaute.

Embodiment 258. The engineered polynucleotide of any one of embodiments 243-257, wherein the targeting domain is is at least partially complementary to at least a portion of a 3′ or 5′ untranslated region (UTR) of the target RNA.

Embodiment 259. The engineered polynucleotide of any one of embodiments 243-258, wherein the targeting domain is at least partially complementary to at least a portion of an intronic region of the target RNA.

Embodiment 260. The engineered polynucleotide of any one of embodiments 243-259, wherein the targeting domain is at least partially complementary to at least a portion of an upstream open reading frame (uORF) of the target RNA.

Embodiment 261. The engineered polynucleotide of any one of embodiments 243-259, wherein the targeting domain is not at least partially complementary to at least a portion of an upstream open reading frame (uORF) of the target RNA.

Embodiment 262. The engineered polynucleotide of any one of embodiments 243-261, wherein the target RNA is associated with Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, or cystic fibrosis.

Embodiment 263. The engineered polynucleotide of any one of embodiments 244-262, wherein the RNA editing entity comprises an ADAR protein, an APOBEC protein, or both.

Embodiment 264. The engineered polynucleotide of any one of embodiments 244-262, wherein the RNA editing entity comprises an ADAR protein.

Embodiment 265. An engineered polynucleotide encoding or comprising: a) a targeting domain that is at least partially complementary to a target RNA; and b) a spacer domain, wherein the targeting domain does not physically comprise the spacer domain; wherein the engineered polynucleotide comprises a structure of Formula (IV):

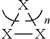

(IV) wherein in the engineered polynucleotide, each X is a nucleotide comprising a base that is independently uracil, thymine, adenine, cytosine, guanine, a salt of any of these, or a derivative of any of these, n is independently an integer from 0-1,000; and wherein each nucleotide is connected to two adjacent nucleotides by, independently for each connection, a phosphoester, phosphothioester, phosphothioate, or phosphoramidite linkage; and wherein: (i) the targeting domain is configured to at least partially associate with a coding region of the target RNA, wherein the association of the targeting domain with the coding region of the target RNA facilitates an edit of a base of a nucleotide of the target RNA by an RNA editing entity, (ii) the targeting domain is configured to at least partially associate with an untranslated region of the target RNA, wherein the association of the targeting domain with the untranslated region of the target RNA facilitates a reduction in a level of a polypeptide produced by the target RNA, or (iii) any combination thereof.

Embodiment 266. The engineered polynucleotide of embodiment 265, wherein the edit of the base of the nucleotide of the target RNA by the RNA editing entity is determined in an in vitro assay comprising:(i) directly or indirectly introducing the target RNA into a primary cell line, (ii) directly or indirectly introducing the engineered polynucleotide into the primary cell line, and (iii) sequencing the target RNA.

Embodiment 267. The engineered polynucleotide of embodiment 265 or 266, wherein the association of the targeting domain with the untranslated region of the target RNA facilitates the reduction in the expression level of a polypeptide encoded for by the target RNA, relative to a level of the polypeptide encoded for by the target RNA in the absence of the engineered polynucleotide as determined by an in vitro assay comprising: (i) directly or indirectly introducing the target RNA into a first cell and a second cell of a primary cell line, (ii) directly or indirectly introducing the engineered polynucleotide into the first cell of the primary cell line, and (iii) comparing an amount of the polypeptide expressed from the target RNA in the first cell and the second cell.

Embodiment 268. The engineered polynucleotide of any one of embodiments 265-267, wherein the engineered polynucleotide does not comprise a 5′ reducing hydroxyl, a 3′ reducing hydroxyl, or both, capable of being exposed to a solvent.

Embodiment 269. The engineered polynucleotide of any one of embodiments 265-268, wherein each 5′ hydroxyl, and each 3′ hydroxyl is independently bonded to a phosphorous by a covalent oxygen phosphorous bond.

Embodiment 270. The engineered polynucleotide of embodiment 269, wherein the phosphorous is contained in a phosphodiester group.

Embodiment 271. The engineered polynucleotide of any one of embodiments 265-270, wherein the engineered polynucleotide further comprises an RNA editing entity recruiting domain.

Embodiment 272. The engineered polynucleotide of any one of embodiments 265-271, wherein the engineered polynucleotide does not comprise or encode a sequence encoding an sequence configured for RNA interference (RNAi).

Embodiment 273. The engineered polynucleotide of any one of embodiments 265-272, wherein the engineered polynucleotide does not comprise or encode a sequence encoding a short interfering RNA (siRNA), microRNA (miRNA), short hairpin RNA (shRNA), or dicer substrate.

Embodiment 274. The engineered polynucleotide of any one of embodiments 265-273, wherein the reduction in the expression level of the polypeptide encoded by the target RNA is at least partially independent of an RNA-directed RNA endonuclease.

Embodiment 275. The engineered polynucleotide of any one of embodiments 265-274, wherein the reduction in the expression level of the polypeptide encoded by the target RNA is at least partially independent of Dicer or RISC.

Embodiment 276. The engineered polynucleotide of any one of embodiments 265-275, wherein the reduction in the expression level of the polypeptide encoded by the target RNA is at least partially independent of Argonaute.

Embodiment 277. The engineered polynucleotide of any one of embodiments 265-276, wherein the targeting domain is at least partially complementary to least a portion of a 3′ or 5′ untranslated region (UTR) of the target RNA.

Embodiment 278. The engineered polynucleotide of any one of embodiments 265-277, wherein the targeting domain is at least partially complementary to at least a portion of an intronic region of the target RNA.

Embodiment 279. The engineered polynucleotide of any one of embodiments 265-278, wherein the targeting domain is at least partially complementary to at least a portion of an upstream open reading frame (uORF) of the target RNA.

Embodiment 280. The engineered polynucleotide of any one of embodiments 265-278, wherein the targeting domain is not at least partially complementary to at least a portion of an upstream open reading frame (uORF) of the target RNA.

Embodiment 281. The engineered polynucleotide of any one of embodiments 265-280, wherein the target RNA is associated with Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, or cystic fibrosis.

Embodiment 282. An engineered circular polynucleotide encoding or comprising: a targeting domain that is substantially complementary to a target RNA implicated in a disease or condition, and a spacer domain that is not complementary to the target RNA, wherein the spacer domain enlarges the engineered circular polynucleotide by the addition of one or more nucleotides,

wherein hybridization of the targeting domain with the target RNA facilitates an edit of a base of a nucleotide of the target RNA by an RNA editing enzyme.

Embodiment 283. The engineered circular polynucleotide of embodiment 282, wherein the edit of the base of a nucleotide of the target RNA by the RNA editing entity is determined in an in vitro assay comprising:(i) directly or indirectly introducing the target RNA into a primary cell line, (ii) directly or indirectly introducing the engineered polynucleotide into a primary cell line, and (iii) sequencing the target RNA.

Embodiment 284. The engineered circular polynucleotide of embodiment 282 or 283, wherein the engineered polynucleotide does not comprise a 5′ reducing hydroxyl, a 3′ reducing hydroxyl, or both, capable of being exposed to a solvent.

Embodiment 285. The engineered circular polynucleotide of embodiment 284, wherein each 5′ hydroxyl, and each 3′ hydroxyl is independently bonded to a phosphorous by a covalent oxygen phosphorous bond.

Embodiment 286. The engineered circular polynucleotide of embodiment 285, wherein the phosphorous is contained in a phosphodiester group.

Embodiment 287. The engineered circular polynucleotide of any one of embodiments 282-286, further comprising an RNA editing enzyme recruiting domain, wherein the RNA editing enzyme recruiting domain recruits an RNA editing enzyme that, when associated with the engineered polynucleotide, performs a chemical transformation on a base of a nucleotide in the target RNA.

Embodiment 288. The engineered circular polynucleotide of any one of embodiments 282-287, wherein the engineered polynucleotide is about 40 nucleotides to about 200 nucleotides.

Embodiment 289. The engineered circular polynucleotide of embodiment 288, wherein the engineered polynucleotide is about 40 nucleotides to about 100 nucleotides.

Embodiment 290. The engineered circular polynucleotide of embodiment 288, wherein the engineered polynucleotide is about 140 nucleotides to about 200 nucleotides.

Embodiment 291. The engineered circular polynucleotide of embodiment 288, wherein the engineered polynucleotide is about 80 nucleotides to about 150 nucleotides.

Embodiment 292. The engineered circular polynucleotide of any one of embodiments 282-291, wherein the targeting domain is about 20 nucleotides to about 150 nucleotides.

Embodiment 293. The engineered circular polynucleotide of any one of embodiments 282-292, wherein the target RNA comprises a nonsense mutation.

Embodiment 294. The engineered circular polynucleotide of any one of embodiments 282-293, wherein the targeting domain comprises at least a single nucleotide that is mismatched to the target RNA.

Embodiment 295. The engineered circular polynucleotide of embodiment 294, wherein the mismatched nucleotide is adjacent to two nucleotides, one on each side of the mismatched nucleotide, that are complementary to the target RNA.

Embodiment 296. The engineered circular polynucleotide of any one of embodiments 282-295 wherein the engineered polynucleotide further comprises a sequence with at least about 80%, at least about 85%, at least about 90%, at least about 92%, at least about 95%, at least about 97%, at least about 99%, or 100% sequence identity to a U7 or a U1 promoter sequence.

Embodiment 297. The engineered circular polynucleotide of any one of embodiments 282-296, wherein the engineered polynucleotide further comprises an RNA sequence motif.

Embodiment 298. The engineered circular polynucleotide of embodiment 297, which comprises a SmOPT sequence, a hnRNPA1 sequence, a MALAT1 sequence, SIRLOIN sequence, or a combination thereof.

Embodiment 299. The engineered circular polynucleotide of embodiment 282, wherein the disease or condition comprises Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, or cystic fibrosis.

Embodiment 300. A vector comprising, the precursor engineered linear polynucleotide of any one of embodiments 160-196, the precursor engineered polynucleotide of any one of embodiments 197-221, or the engineered circular polynucleotide of any one of embodiments 222-299.

Embodiment 301. The vector of embodiment 300, wherein the vector comprises a polypeptide coat, wherein at least a portion of the engineered polynucleotide is present inside of the polypeptide coat.

Embodiment 302. The vector of embodiment 300 or 301, wherein the vector comprises a liposome, a viral vector, a nanoparticle, or any combination thereof.

Embodiment 303. The vector of embodiment 302, wherein the vector comprises an adeno-associated virus (AAV) vector.

Embodiment 304. The vector of embodiment 303, wherein the AAV vector is an AAV1 vector, AAV2 vector, AAV3 vector, AAV4 vector, AAV5 vector, AAV6 vector, AAV7 vector, AAV8 vector, an AAV9 vector, a chimera of any of these, or a variant of any of these.

Embodiment 305. The vector of embodiment 303 or 304, wherein the viral vector is a self-complementary adeno-associated viral (scAAV) vector.

Embodiment 306. The vector of embodiment 303 or 304, wherein the viral vector is a single-stranded AAV vector.

Embodiment 307. A nucleic acid at least partially encoding the precursor engineered linear polynucleotide of any one of embodiments 160-196, the precursor engineered polynucleotide of any one of embodiments 197-221, or the engineered circular polynucleotide of any one of embodiments 222-299.

Embodiment 308. The nucleic acid of embodiment 307, wherein the nucleic acid is double stranded.

Embodiment 309. The nucleic acid of embodiment 307, wherein the nucleic acid comprises a targeting tag.

Embodiment 310. The nucleic acid of embodiment 307, comprising the targeting tag which comprises N-Acetylgalactosamine (GlaNAc).

Embodiment 311. A pharmaceutical composition in unit dose form comprising the precursor engineered linear polynucleotide of any one of embodiments 160-196, the engineered circular polynucleotide of any one of embodiments 222-299, or the vector of any one of embodiments 300-306; and a pharmaceutically acceptable excipient, diluent, or carrier.

Embodiment 312. A kit comprising the precursor engineered linear polynucleotide of any one of embodiments 160-196, the precursor engineered polynucleotide of any one of embodiments 197-221, the engineered polynucleotide of any one of embodiments 222-299, the vector of any one of embodiments 300-306, the nucleic acid of any one of embodiments 307-310, or the pharmaceutical composition of embodiment 311, and a container.

Embodiment 313. A method of treating or preventing a disease or condition in a subject in need thereof comprising: administering a therapeutically effective amount of: the precursor engineered linear polynucleotide of any one of embodiments 160-196, the engineered circular polynucleotide of any one of embodiments 222-299, the vector of any one of embodiments 300-306, or the pharmaceutical composition of embodiment 311.

Embodiment 314. The method of embodiment 313, wherein the method comprises administration of a second therapy.

Embodiment 315. The method of embodiment 313, wherein the administering is to a cell.

Embodiment 316. The method of embodiment 315, wherein the cell is a T-cell.

Embodiment 317. The method of embodiment 313, further comprising diagnosing the subject as having a disease.

Embodiment 318. The method of embodiment 313, wherein the subject is a human.

Embodiment 319. A method of forming an engineered circular polynucleotide, the method comprising: directly or indirectly ligating a nucleotide on a 3′ end of a precursor engineered polynucleotide to a nucleotide on a 5′ end of the precursor engineered polynucleotide with a ligating entity, thereby forming the engineered circular polynucleotide, wherein the engineered circular polynucleotide comprises: a) a targeting domain that is substantially complementary to a target RNA implicated in a disease or condition, and, b) a spacer domain, wherein the targeting domain of the engineered circular polynucleotide comprising the spacer domain has a lower Gibbs free energy (ΔG) of binding of the targeting domain to the target RNA, relative to a ΔG of binding of a targeting domain of a corresponding circular polynucleotide that lacks the spacer domain, as determined by Kelvin Probe Force Microscopy (KPFM), and wherein at least one of the following applies: (i) the spacer domain is not complementary to the target RNA; (ii) when the targeting domain hybridizes to the target RNA, the spacer domain is separated from the targeting domain by at least 1 nucleotide, and if the spacer domain hybridizes to the target RNA, the hybridization of the spacer domain does not produce an edit of the target RNA at the portion of the target RNA that binds to the spacer domain; or (iii) when the spacer domain is adjacent to a 5′ end or a 3′ end of the targeting domain, the spacer domain is not complementary to the target RNA, wherein the targeting domain hybridizes with a coding region of the target RNA, wherein the hybridization of the targeting domain with the coding region of the target RNA facilitates an edit of a base of a nucleotide of the target RNA by an RNA editing enzyme.

Embodiment 320. The method of embodiment 319, wherein the edit of the base of a nucleotide of the target RNA by an RNA editing enzyme is determined in an in vitro assay comprising: (i) directly or indirectly introducing the target RNA into a primary cell line, (ii) directly or indirectly introducing the engineered circular polynucleotide into a primary cell line, and (iii) sequencing the target RNA.

Embodiment 321. The method of embodiment 319 or 320, wherein the engineered circular polynucleotide is facilitates an increase in the binding specificity to the target RNA, among a plurality of other RNA, as compared to the corresponding polynucleotide that lacks the spacer domain, as determined by sequencing of the target RNA and plurality of other RNA after contacting with the engineered polynucleotide or the corresponding polynucleotide.

Embodiment 322. The method of any one of embodiments 319-321, wherein the spacer domain is facilitates a lower entropy (ΔS) of binding of the engineered circular polynucleotide to the target RNA, relative to the corresponding circular polynucleotide that lacks the spacer domain, as determined by Kelvin Probe Force Microscopy (KPFM).

Embodiment 323. The method of any one of embodiments 319-322, wherein the spacer domain increases hybridization of the targeting domain to the target RNA, relative to an otherwise comparable circularized polynucleotide that lacks the spacer domain.

Embodiment 324. The method of embodiment 323, wherein the spacer domain increases the editing efficiency of the engineered circular polynucleotide to the target RNA, relative an editing efficiency of the corresponding polynucleotide that lacks the spacer domain, as determined by sequencing of the target RNA after contacting with the engineered polynucleotide or the corresponding polynucleotide that lacks the spacer domain.

Embodiment 325. The method of any one of embodiments 319-324, wherein the engineered polynucleotide further comprises an RNA editing enzyme recruiting domain.

Embodiment 326. The method of embodiment 325, wherein the RNA editing enzyme comprises an ADAR protein, an APOBEC protein, or both.

Embodiment 327. The method of embodiment 326, comprising the ADAR protein, which comprises ADAR1, ADAR2, or ADAR3.

Embodiment 328. The method of any one of embodiments 319-327, wherein the engineered polynucleotide does not comprise a 5′ reducing hydroxyl, a 3′ reducing hydroxyl, or both, capable of being exposed to a solvent.

Embodiment 329. The method of embodiment 328, wherein each 5′ hydroxyl, and each 3′ hydroxyl is independently bonded to a phosphorous by a covalent oxygen phosphorous bond.

Embodiment 330. The method of embodiment 329, wherein the phosphorous is contained in a phosphodiester group.

Embodiment 331. The method of any one of embodiments 319-330, wherein the targeting domain comprises at least a single nucleotide that is mismatched to the target RNA.

Embodiment 332. The method of embodiment 331, wherein the mismatched nucleotide is adjacent to two nucleotides, one on each side of the mismatched nucleotide, that are complementary to the target RNA.

Embodiment 333. The method of any one of embodiments 319-332, wherein the targeting domain is about 20 nucleotides to about 150 nucleotides.

Embodiment 334. The method of any one of embodiments 319-332, wherein the engineered polynucleotide is about 40 nucleotides to about 200 nucleotides.

Embodiment 335. The method of embodiment 334, wherein the engineered polynucleotide is about 40 nucleotides to about 100 nucleotides.

Embodiment 336. The method of embodiment 334, wherein the engineered polynucleotide is about 140 nucleotides to about 200 nucleotides.

Embodiment 337. The method of embodiment 334, wherein the engineered polynucleotide is about 80 nucleotides to about 150 nucleotides.

Embodiment 338. The method of embodiment 319, wherein the disease or condition comprises Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, or cystic fibrosis.

Embodiment 339. An engineered polynucleotide comprising: a targeting domain that is at least partially complementary to a target RNA; an RNA editing entity recruiting domain, wherein the RNA editing entity recruiting domain is configured to at least transiently associate with an RNA editing entity; and a spacer domain, wherein the targeting domain or the RNA editing recruiting domain do not comprise the spacer domain; wherein the engineered polynucleotide comprises a backbone comprising a plurality of sugar and phosphate moieties covalently linked together, and wherein the backbone does not comprise a 5′ reducing hydroxyl, a 3′ reducing hydroxyl, or both, capable of being exposed to a solvent, wherein the targeting domain is configured to at least partially associate with a coding region of the target RNA, wherein the association of the targeting domain with the coding region of the target RNA facilitates an edit of a base of a nucleotide of the target RNA by an RNA editing entity.

Embodiment 340. An engineered polynucleotide comprising: a targeting domain that is at least partially complementary to a target RNA; and a spacer domain, wherein the engineered polynucleotide comprising the spacer domain has a lower Gibbs free energy (ΔG) of binding of the engineered polynucleotide to the target RNA, relative to a ΔG of binding of a corresponding polynucleotide that lacks the spacer domain, as determined by Kelvin Probe Force Microscopy (KPFM); wherein the engineered polynucleotide comprises a backbone comprising a plurality of sugar and phosphate moieties covalently linked together, and wherein the backbone does not comprise a 5′ reducing hydroxyl, a 3′ reducing hydroxyl, or both, capable of being exposed to a solvent, wherein the targeting domain is configured to at least partially associate with a coding region of the target RNA, wherein the association of the targeting domain with the coding region of the target RNA facilitates an edit of a base of a nucleotide of the target RNA by an RNA editing entity.

Embodiment 341. The engineered polynucleotide of embodiment 340, wherein the edit of the base of a nucleotide of the target RNA by an RNA editing entity is determined in an in vitro assay comprising:(i) directly or indirectly introducing the target RNA into a primary cell line, (ii) directly or indirectly introducing the engineered polynucleotide into a primary cell line, and (iii) sequencing the target RNA.

### EXAMPLES

### Example 1

### Circular Guide RNAs can Increase RNA Editing 2×-4× in Multiple Cell Lines

In this example, plasmids encoding the circular and linear guide RNAs were transfected into different cell lines to determine the percent RNA editing of the RAB7A 3′UTR region. The RNA editing was determined by Sanger sequencing the target RNA after introducing the linear and circular guide RNAs. Target RNA was converted to cDNA by reverse transcriptase prior to sequencing. Sanger traces were analyzed to assess the editing efficiency. FIG. 1A and FIG. 1B show RAB7A 3′UTR editing efficiency of Design 1 (linear guide), Design 2 (linear guide), and Design 3 (circular guide) in different cell line backgrounds. The cell line background included 293T (human embryonic kidney), RD undifferentiated (human embryo rhabdomyosarcoma), LHCN (human skeletal myoblast) undifferentiated, and LHCN differentiated. Controls are shown as “no transfection”. FIG. 1A shows the percent RNA editing of the RAB7A 3′ UTR after 48 h post transfection and FIG. 1B shows the percent RNA editing of the RAB7A 3′ UTR after 96 h post transfection. In FIG. 1A-B, the circular guide Design 3 showed about a 2×-4× percent editing increase compared to the linear design 1 in all tested cell lines.

### Example 2

### An Engineered Guide RNA can Comprise a Spacer Domain, a Ligation Sequence Domain, a Ribozyme Sequence Domain, and a Targeting Domain

Referring to FIG. 2, an engineered guide RNA is shown comprising 2 ribozyme domains, 2 ligation sequence domains, 2 spacer domains, and a targeting domain (100.50). In this example, the engineered guide RNA will circularize in the cell by autocatalytic reactions of the encoded ribozymes. The engineered guide RNA will undergo intracellular RNA ligation of the 5′ and the 3′ end of the ligation sequences by an endogenous ligase to circularize the guide RNA. The circular guide RNA can perform the functions described herein (e.g. perform an edit in a target RNA).

### Example 3

### Circular Engineered Guide RNAs can be Formed by Circularization in the Cell

In this example, plasmids encoding circular and linear guide RNAs were transfected into a cell to determine if circularization occurs in a cell. RNA was extracted from the cell and the RNA was reverse transcribed into cDNA. PCR was performed with primers that annealed to the 100.50 region of the guide RNAs. The primers were designed to amplify to the 3′ end and the 5′ end, thus generating a product if circularization occurred. FIG. 3B showed the reverse transcriptase (RT)-PCR confirmation of the circularization of the engineered guide RNAs Design 3 and Design 5. The linear guide RNAs Design 1 and Design 4 did not produce a circular guide RNA because they were not amplified using the primers designed to detect circularization of the guide RNAs. The linear guide RNAs lacked ligation sequences which allow for circularization in a cell. The circular engineered guide RNAs can be designed for RNA editing or producing RNA knockdowns.

### Example 4

### RNA Editing can Occur at Multiple Positions of a Target RNA

Plasmids encoding circular guide RNAs were transfected to cells to determine the percent RNA editing of circular guide RNAs at different locations on the Rab7a 3′UTR region. The transfection was with 1 μg of plasmid per 20,000 WT HEK293 cells. The percent RNA editing was determined from Sanger sequencing the target RNA after reverse transcription of the target RNA to cDNA. Sanger traces were analyzed to assess the editing efficiency. FIG. 4 showed the percent RNA editing of different adenosine nucleotides in the RAB7A 3′UTR from Design 3 (circular guide) and a control GFP plasmid. The edited nucleotide positions are indicated in the Rab7a 3′UTR sequence. The Design 3 guide RNA was shown to edit nucleotides in the '13, −2, −1 and +1 positions in the Rab7a 3′ UTR sequence. The highest percent RNA editing occurred at the −3 position in the Rab7a 3′UTR sequence.

### Example 5

### Circular Guide RNAs have Increased RNA Editing Overtime Compared to Linear Guide RNAs in Cells

Plasmids encoding circular and linear guide RNAs that target the Rab7a UTR were introduced into HEK 293 cells. 1 microgram of plasmid was transfected into about 20,000 WT HEK293 cells to determine percent RNA editing overtime (48 hours and 96 hours). The percent RNA editing of the target RNA was determined by Sanger sequencing after the target RNA was converted to cDNA by reverse transcriptase. Sanger traces were analyzed to assess the editing efficiency. FIG. 5 shows the percent RNA editing of nucleotides in the RAB7A 3′UTR from linear guide RNAs Design 1 and Design 4, compared to the circular guide RNAs Design 5 and Design 3. A control GFP plasmid was included in the experiment. Design 5 and Design 3 guide RNAs were shown to have higher levels of RNA editing at 48 hour and 96 hours compared to the linear guide RNAs. The increase in editing may be from the increased stability of the circular guide RNAs compared to the linear guide RNAs.

### Example 6

### Circular and Linear Guide RNAs can Edit and Knockdown Alpha Synuclein (SNCA) RNA by Targeting the 3′ UTR

Plasmids encoding linear and circular guide RNAs were transfected into cells. The guide RNAs were designed to target the 3′ UTR SNCA mRNA. The target mRNA was converted to cDNA by reverse transcriptase and sequenced with Sanger sequencing after 40 and 72 hours to determine the percent of edited SNCA RNA and the knockdown of SNCA mRNA was determined at 40 and 72 hours by quantitative reverse transcriptase PCR. Sanger traces were analyzed to assess the editing efficiency. FIG. 6A shows the percent RNA editing and FIG. 6B shows the percent knockdown of 3′ UTR SNCA mRNA after 40 and 72 hours from linear guide RNAs (guides 1-7) and circular guide RNAs (guide 8, guide 9 and Rab7). The Rab7 circular RNA targeted the coding region of SNCA and was included as a control to show RNA editing occurred at a high percentage by circular guides if targeting a coding region. The bottom of the graphs indicates the presence or absence of exogenously added ADAR 1 or ADAR 2. The circular guide RNA showed lower editing levels compared to the linear guides 1-4 for editing the SNCA 3′UTR. However, the circular guide RNA showed increased knockdown of the SNCA mRNA compared to the linear guide RNAs. The increased knockdown efficiency of circular guides may be independent of ADAR.

### Example 7

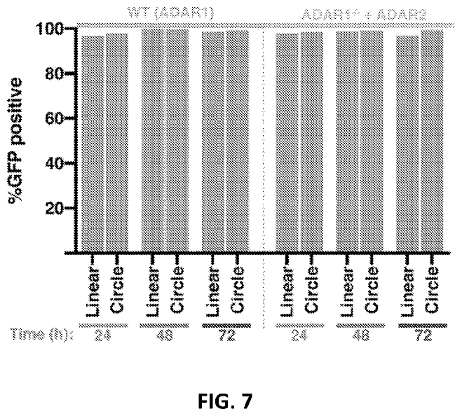

High levels of transduction were observed for circular and linear guides using scAAV2. The vector (scAAV GOI plasmid) was constructed with an eGFP-reporter and hU6-driven circular guide or a linear guide targeting the 3′UTR of Rab7a. The scAAV2 virus was produced via triple transfection of the GOI plasmid, pRC2 (Rep2/Cap2), and an adenovirus helper plasmid. Virus was purified and quantified. FIG. 7 shows cells which were infected at 1×10\hat{ }5 vgs/cell (viral genomes per cell) and harvested at 24 hours, 48 hours, and 72 hours post-infection. The cell lines used for the experiment were HEK293T (WT (ADAR1)) cells and HEK293T (ADAR1 knockout+ADAR2) cells which is indicated on the top of the graph. GFP positive cells indicate successful transduction of vectors encoding linear and circular guide RNAs. After transduction, GFP positivity was measured by flow cytometry and over 97% GFP positive cells were detected in all cell lines at all time points.

### Example 8

### Circular Guide RNAs can Edit a Target in Cells for an Extended Period of Time

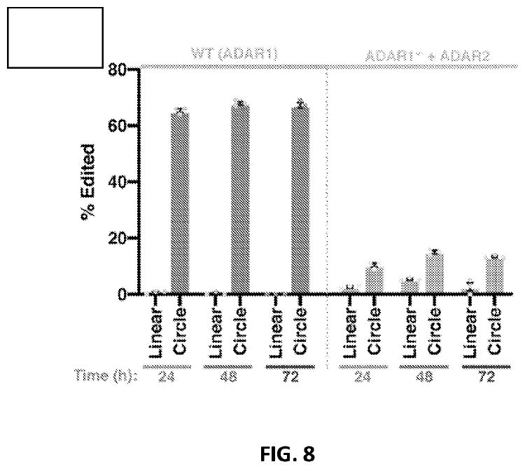

Editing was observed for over 72 hours with a circular guide. FIG. 8 shows the percent RNA editing of the Rab7a 3′ UTR in cells transduced with vectors encoding linear and circular engineered guide RNAs. The vector (scAAV GOI plasmid) was constructed with an eGFP-reporter and hU6-driven circular guide or a linear guide targeting the 3′UTR of Rab7a. The scAAV2 virus was produced via triple transfection of the GOI plasmid, pRC2 (Rep2/Cap2), and an adenovirus helper plasmid. Virus was purified and quantified. Cells were infected at 1×10\hat{ }5 vgs/cell and harvested at 24 hours, 48 hours, and 72 hours post-infection. The cell lines used for the experiment were HEK293T (WT (ADAR1)) cells and HEK293 (ADAR1 knockout+ADAR2) cells which are indicated on the top of the graph. The HEK293 (ADAR1 knockout+ADAR2) cells have been engineered to express ADAR2 but not ADAR1. The percent editing of the Rab7a 3′ UTR mRNA was measured at 24 hours, 48 hours and 72 hours for cells transduced with vectors encoding linear and circular guide RNAs. To determine editing, RNA was harvested from cells and Rab7a 3′UTR cDNA was produced by reverse transcription which included the following steps: RNA was incubated at 95° C. for 3 min with oligo dT primer, synthesis occurred at 48° C. for 1 hour followed by denaturing at 80° C. for 5 min. cDNA was amplified by PCR and the PCR amplicon of the 3′UTR of Rab7 was Sanger sequenced and analyzed by trace analysis to measure editing. 65-69% of Rab7a transcripts were A-G edited in WT HEK293T cells with circle guides and little editing was observed with the linear guides. Furthermore, ADAR2-only expressing cells had lower editing with circles than WT ADAR1 expressing cells.

### Example 9

### Circularization of Circular Guide RNAs Occurs for an Extended Period of Time

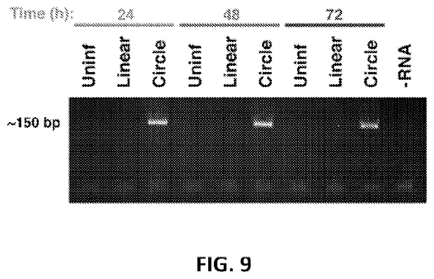

Circularization of circular RNA guides was observed for 72 hours. FIG. 9 shows an agarose gel with a PCR product indicating the presence or absence of the circularization of linear guide RNA, circular guide RNA, and control (uninfected) cell lines. The vector (scAAV GOI plasmid) was constructed with an eGFP-reporter and hU6-driven circular guide or a linear guide targeting the 3′UTR of Rab7a. The scAAV2 virus was produced via triple transfection of the GOI plasmid, pRC2 (Rep2/Cap2), and an adenovirus helper plasmid. Virus was purified and quantified. Cells were infected at 1×10\hat{ }5 vgs/cell and harvested at 24 hours, 48 hours, and 72 hours post-infection. The cell line used for the experiment was HEK293T. After transduction with scAAV2 virus carrying the GOI plasmid RNA was harvested from WT HEK293T cells and reverse transcribed to cDNA. Circular guide formation was assessed by PCR that was performed using primers that bind to the ends of the guide RNA and are directed outward such that a product is formed when guides are circularized. As indicated in the gel, circular guide RNA was detected in circular guide-infected cells at all time points.

### Example 10

### Circular Guides can be Made by Back-Splicing

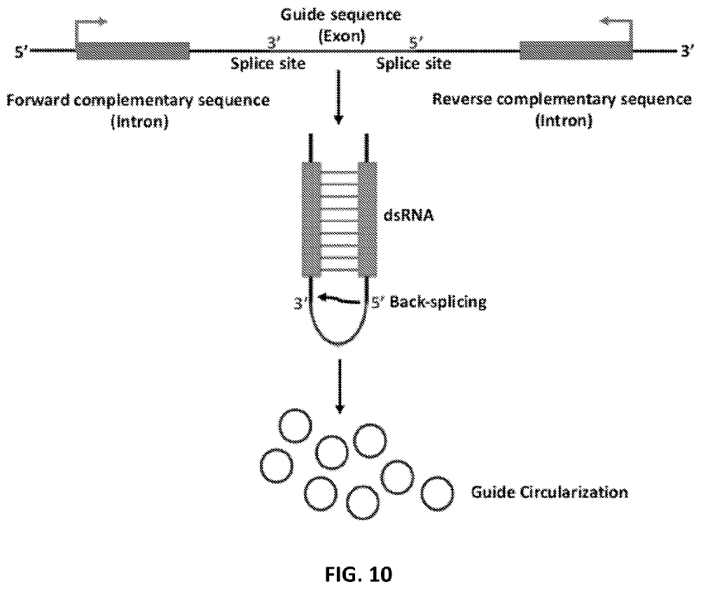

FIG. 10 shows an illustration of the method of making a circular guide RNA. The RNA is engineered from 5′ to 3′ to comprise a forward complementary sequence intron, an exon (which comprises the guide sequence), followed by a reverse complementary sequence intron. Once transcribed, the complementary sequence introns will hybridize and form dsRNA. The exon containing the guide sequence will be removed by splicing and ligated by endogenous ligases to form a circular guide. In some cases, the guide is circularized by back-splicing reactions.

### Example 11

### Pharmaceutical Compositions of Circular Guide RNAs and Treatment of a Subject

A subject will be diagnosed with a disease. The subject will be prescribed a dosing regimen of a pharmaceutical composition. A pharmaceutical composition will comprise a circular engineered guide RNA. The circular engineered guide RNA will comprise a targeting domain, a spacer domain and an RNA editing entity recruiting domain capable of recruiting an RNA editing entity that performs an editing of a nucleotide. The pharmaceutical composition will be co-administered to the subject with a recombinant RNA editing enzyme by injection. The pharmaceutical composition will comprise a nanoparticle which comprises the circular engineered guide RNA and the recombinant RNA editing enzyme. The dosing regimen will comprise an effective amount to treat the disease.

### Example 12

### Circular Guide RNAs with Aptamers

A circular engineered guide RNA will be formed by obtaining an engineered guide RNA. The engineered guide RNA will comprise a targeting domain, a spacer domain, and an RNA editing entity recruiting domain that is capable of recruiting an RNA editing entity that performs an editing of a nucleotide. An aptamer will be added to each end of the engineered guide RNA. A ligase will be contacted with the aptamers at each end of the engineered guide RNA to form a covalent linkage between the aptamers thereby forming the circular engineered guide RNA. The circular engineered guide RNA will substantially retain a secondary structure as compared to a secondary structure of a comparable engineered guide RNA that is not circular.

### Example 13

A subject will be diagnosed with a disease. The subject will be prescribed a dosing regimen of a pharmaceutical composition. The pharmaceutical composition will comprise a circular engineered guide RNA. The circular engineered guide RNA will comprise a targeting domain, a spacer domain and an RNA editing entity recruiting domain capable of recruiting an RNA editing entity that performs an editing of a nucleotide. The pharmaceutical composition will be administered to the subject by injection. A dosing regimen of the circular engineered guide RNA will be 2× less frequent than a subject receiving by injection a comparable engineered guide RNA that is not circular. The dosing regimen will comprise an effective amount to treat the disease.

### Example 14

### AAV-Delivered Circular Guide RNAs for Treatment of a Subject

A subject will be diagnosed with a disease. The subject will be prescribed a dosing regimen of a pharmaceutical composition. The pharmaceutical composition will comprise viral vector that comprises a plasmid encoding a circular engineered guide RNA. The circular engineered guide RNA will comprise a ribozyme domain, a ligation sequence domain, a spacer domain, a targeting domain and an RNA editing entity recruiting domain capable of recruiting an RNA editing entity that performs an editing of a nucleotide. The pharmaceutical composition will be administered to the subject by injection. A dosing regimen of the viral vector comprising the plasmid that encodes for the circular engineered guide RNA will be 2× less frequent than a subject receiving by injection a comparable engineered guide RNA that is not circular. The dosing regimen will comprise an effective amount to treat the disease.

### Example 15

### RAB7A Editing with Circular Guide RNAs

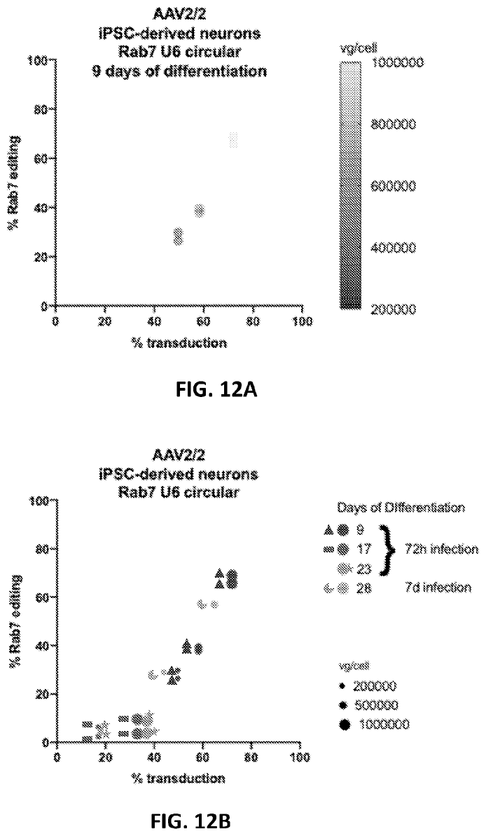

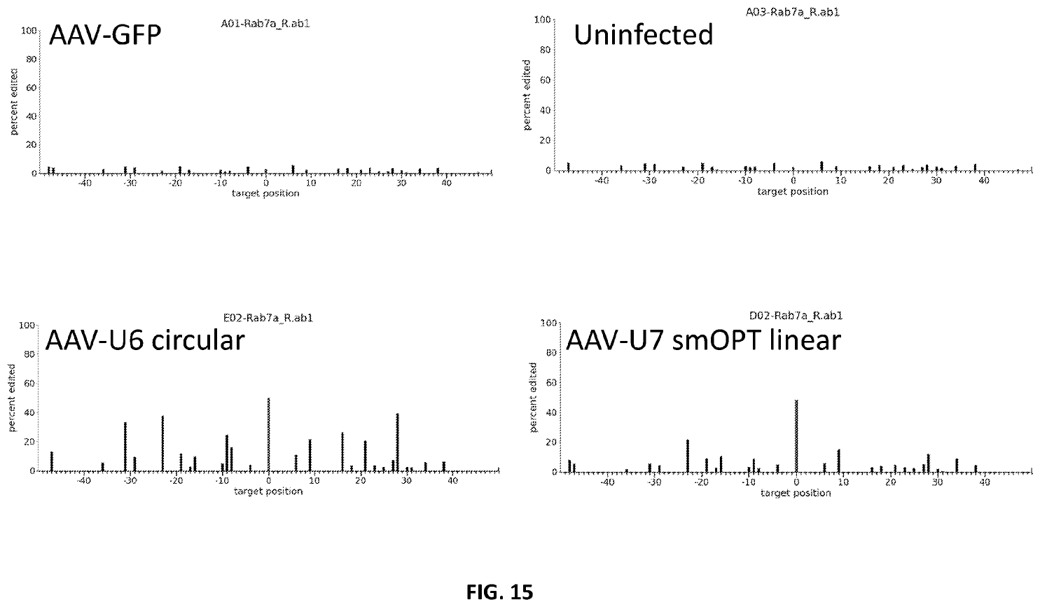

This example describes RAB7A editing with circular guide RNAs of the present disclosure in iPSC-derived neural cells, where the circular guide RNAs were delivered via AAV. iPSCs were induced to a neural lineage using dual SMAD inhibition for at least 6 days before infection with AAV2/2 expressing GFP (control) or Rab7 guides (Rab7U6 linear, Rab7 U7 smOPT and Rab7 U6 circular). The sequence of the guide RNAs in each of these constructs is GATAAAAGGCGTACATAATTCTTGTGTCTACTGTACAGAATACTGCCGCCAGCTGGA TTTCCCAATTCTGAGTAACACTCTGCAATCCAAACAGGGTTCA (SEQ ID NO: 23). Cells were generally plated at 1*10\hat{ }5 cells per well in a 24 well plate and infected at the specified vg/cell. We isolated mRNA 48 hours, 72 hours, or 7 days post infection and assessed Rab7 editing by ddPCR and Sanger sequencing. For FIGS. 12 and 15, transduction efficiency was assessed by quantifying images using ImageJ. Briefly, images were taken of each well that captured the GFP+ signal as well as a brightfield image of the same field of view. The GFP+ signal was thresholded and the area of positive signal was measured. Similarly, the area of the brightfield image that contained cells was thresholded and the area of positive signal was measured. The GFP+ area was divided by the cellular area to get percent transduction.

iPSCs were transduced with varying vg/cell for 48 or 72 h and were harvested at 8 or 9 days of differentiation. Rab7 editing was measured by ddPCR. Gels of PCR product from circular guide RNAs demonstrated that circular guide RNA PCR product was detectable in iPSC-derived neurons for up to at least 7 days post-transduction.

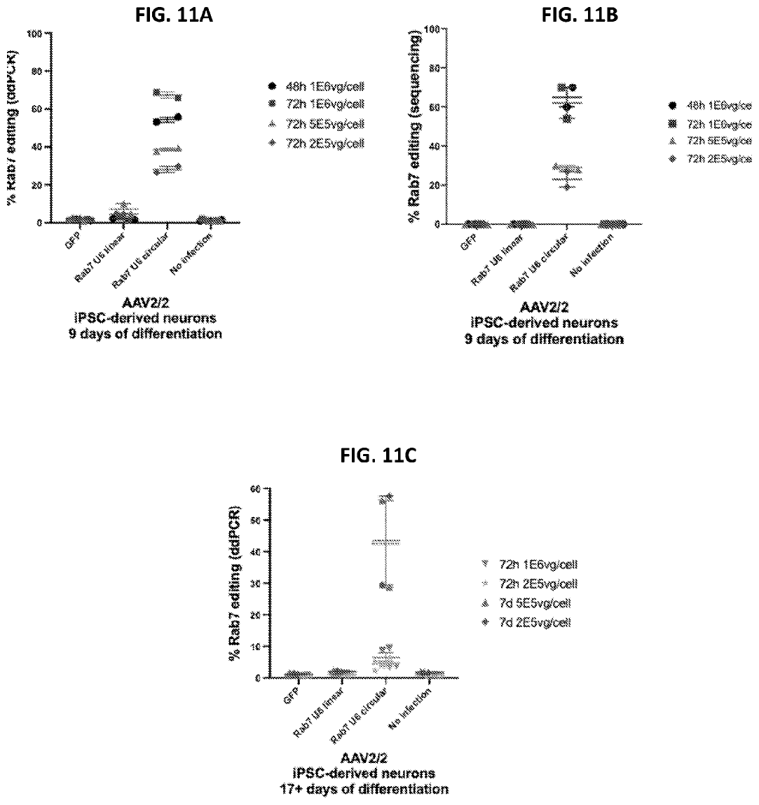

As shown in FIG. 11A, there was a dose dependent increase in Rab7 editing as the vg/cell increases. Data represent biological replicates, mean+/−SEM. Rab7 editing was also measured by Sanger sequencing. As shown in FIG. 11B, there is a dose dependent increase in Rab7 editing as the vg/cell increases. The ddPCR and Sanger sequencing approaches to quantify editing produce very similar results. Data represent biological replicates, mean+/−SEM. iPSCs were transduced with varying vg/cell for 72 h or 7 days and were harvested at 17 or more days of differentiation. Rab7 editing was measured by ddPCR. As shown in FIG. 11C, editing was much lower in fully mature neurons than in the more immature cultures at 72 h, but editing reached the same level as more immature cultures 7 days post infection. This data may imply that more mature neurons take longer to make circular guide RNA. Data represent biological replicates, mean+/−SEM.

FIG. 12A depicts the % transduction plotted against the Rab7 editing efficiency in cells harvested after 9 days of differentiation, 72 hours post infection. The grayscale of the dots represent the titer of virus that the cells were treated with. The viral titer is directly linked to transduction efficiency. FIG. 12B depicts the % transduction plotted against the Rab7 editing efficiency in cells harvested after varying days of differentiation and time points of infection. The dots and shapes represent the days of differentiation and the size of the dot represents the titer of virus that the cells were treated with. Editing and transduction may take longer in fully mature neurons than in the more immature cultures.

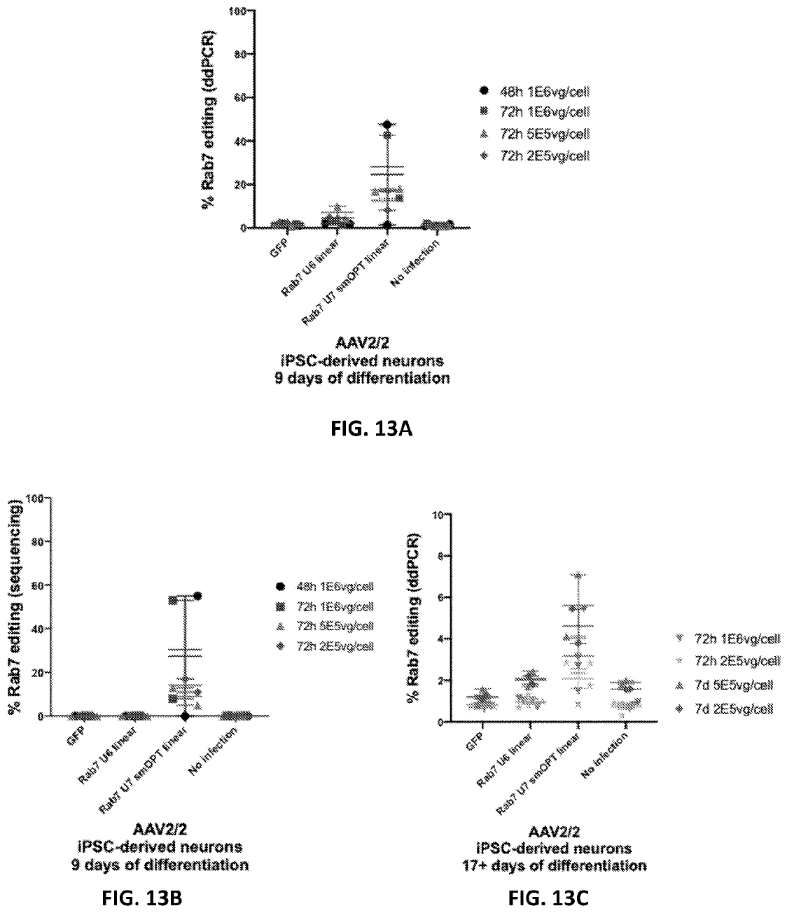

iPSCs were transduced with varying vg/cell for 48 or 72 h and were harvested at 8 or 9 days of differentiation. Rab7 editing was measured by ddPCR. As shown in FIG. 13A, there is a dose dependent increase in Rab7 editing as the vg/cell increases. Data represent biological replicates, mean+/−SEM. Rab7 editing was also measured by Sanger sequencing. As shown in FIG. 13B, there is a dose dependent increase in Rab7 editing as the vg/cell increases. The ddPCR and Sanger sequencing approaches to quantify editing produce very similar results. Data represent biological replicates, mean+/−SEM. For FIG. 13C, iPSCs were transduced with varying vg/cell for 72 h or 7 days and were harvested at 17 or more days of differentiation. Rab7 editing was measured by ddPCR. Data represent biological replicates, mean+/−SEM.

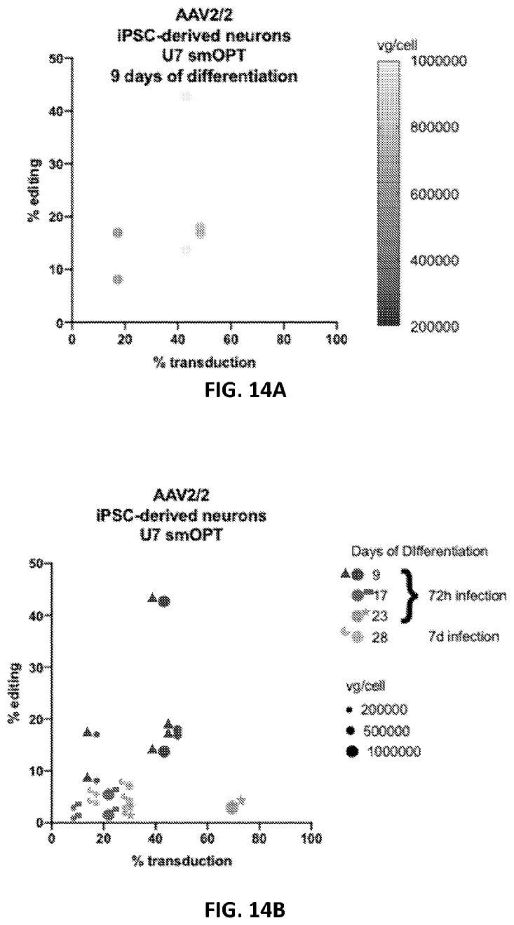

FIG. 14A depicts the % transduction plotted against the Rab7 editing efficiency in cells harvested after 9 days of differentiation, 72 h post infection. The grayscale of the dots represent the titer of virus that the cells were treated with. The viral titer is directly linked to transduction efficiency. FIG. 14B depicts the % transduction plotted against the Rab7 editing efficiency in cells harvested after varying days of differentiation. The dots and shapes represent the days of differentiation and the size of the dot represents the titer of virus that the cells were treated with.

FIG. 15 shows off-target editing profiles. The Rab7 U6 circular guide generates more off-target edits than the U7 smOPT linear guide.

### Example 16

### TUBB4A D249N Editing

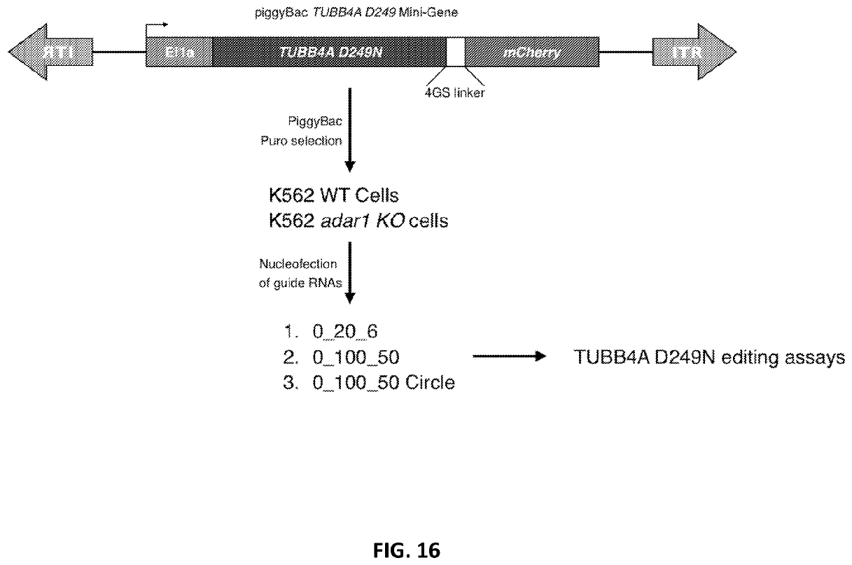

This example describes TUBB4A D249N editing using linear and circular guide RNAs. A TUBB4A D249N minigene construct was designed using a Piggybac backbone vector. FIG. 16 shows a diagram of the piggyBac TUBB4A D249N minigene and summarizes the experimental design. K562 cells that were either WT or adarl Knock Out (adarl KO) were transformed with the Piggybac backbone vector. A population of clones was selected via Puromycin drug selection. Linear and circular guide RNAs against the TUBB4A D249N minigene were designed and are described below in TABLE 3.

TABLE 3

| SEQ ID NO | Sequence |
|---|---|
| 0_20_6 (linear, | CAGGTCGGCGTTCAGCTGGC |
| SEQ ID NO: 32) | |
| 0_100_50 (linear, | GAAGTGCAGGCGAGGAAAGGGAACCATGTT |
| SEQ ID NO: 33) | GACGGCCAGCTTGCGCAGGTCGGCGTTCAG |
| | CTGGCCCGGGAAGCGCAGGCAGGTGGTGAC |
| | CCCGCTCATG |
| 0_100_50 | GCCATCAGTCGCCGGTCCCAAGCCCGGATA |
| (circular, | AAATGGGAGGGGGCGGGAAACCGCCTAACC |
| SEQ ID NO: 34) | ATGCCGACTGATGGCAGATAATGAAGTGCA |
| | GGCGAGGAAAGGGAACCATGTTGACGGCCA |
| | GCTTGCGCAGGTCGGCGTTCAGCTGGCCCG |
| | GGAAGCGCAGGCAGGTGGTGACCCCGCTCA |
| | TGATATACTGCCATCAGTCGGCGTGGACTG |
| | TAGAACACTGCCAATGCCGGTCCCAAGCCC |
| | GGATAAAAGTGGAGGGTACAGTCCACGC |

About 500K cells (WT and adarl KO) were nucleofected with 1 ug of guide RNAs. 18 hours post nucleofection, total RNA was purified from nucleofected cells, cDNA was made, and a PCR reaction was performed to amplify the TUBB4A D249N region. Editing efficiencies in WT, adarl KO and mock transfections at theTUBB4A D249N target adenosine were quantified using Sanger sequencing. A PCR reaction was performed to verify the presence of circular RNAs.

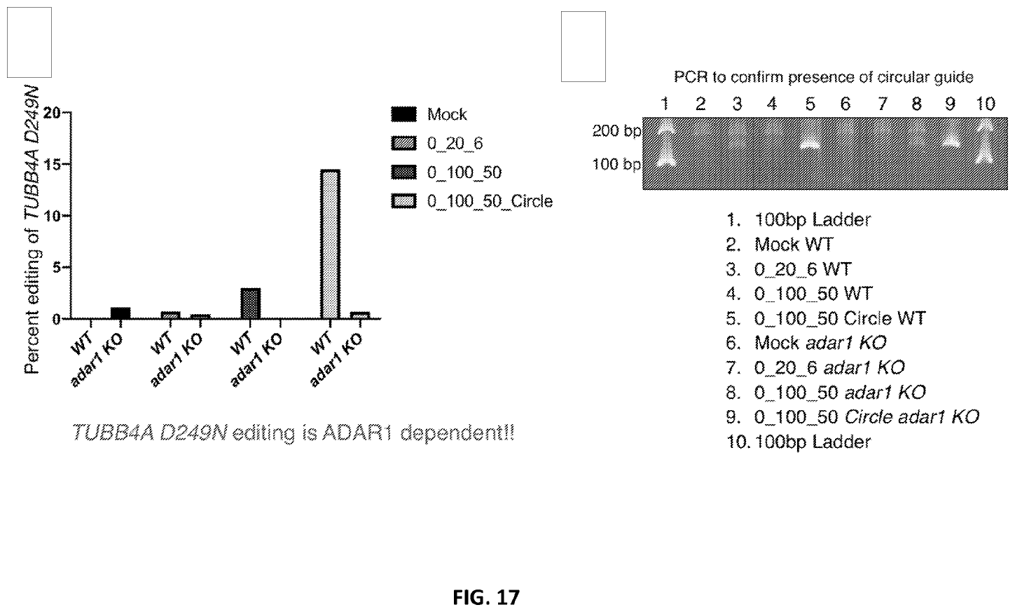

As shown in FIG. 17 at left, percent editing in TUBB4A D249N minigene is significant in K562 WT cells expressing only the circular guide. FIG. 17 at right shows PCR products with primers that are specific for circular guide RNAs. Notice only samples 5 and 9 show the right size PCR fragment.

### Example 16

### Rab7A Editing with Circular Guide RNAs Having Different Lengths of Spacers

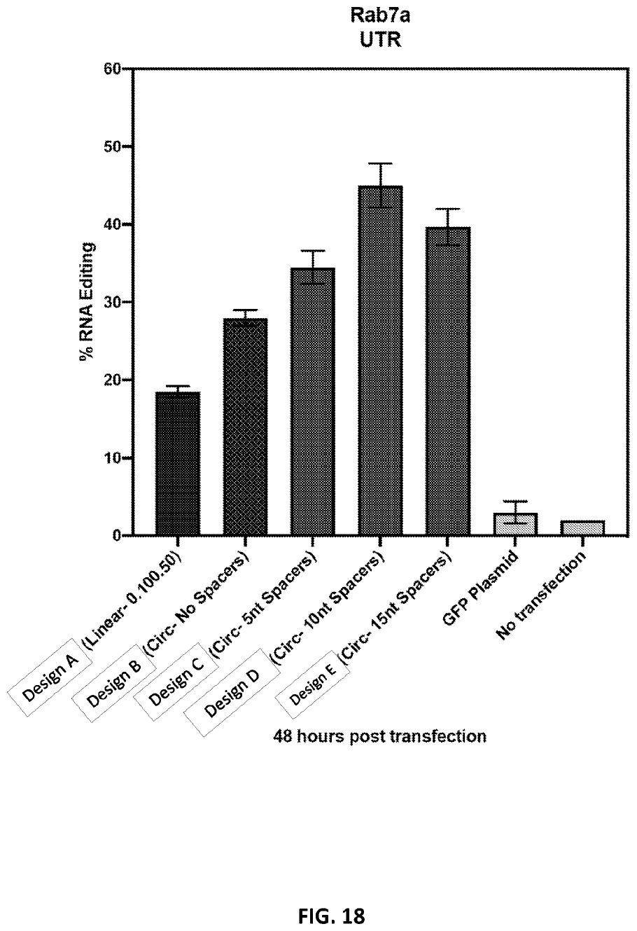

This example describes Rab7A editing with circular guide RNAs of the present disclosure having varying lengths of spacers. WT HEK293 cells (30,000 cells) were transfected with 100 ng of plasmid encoding for guide RNAs or controls. RNA was isolated 48 hours post-transfection and converted into a cDNA library. PCR and Sanger sequencing was carried out. Percent editing was determined using the EditR software. Sequences of guide RNA and spacers tested are shown below in TABLE 4. As seen in FIG. 18, circular guides with longer spacers facilitated higher levels of RNA editing.

TABLE 4

| Description | Sequence |
|---|---|
| Rab7A Guide | GATAAAAGGCGTACATAATT |
| Sequence | CTTGTGTCTACTGTACAGAA |
| | TACTGCCGCCAGCTGGATTT |
| | CCCAATTCTGAGTAACACTC |
| | TGCAATCCAAACAGGGTTCA |
| | (SEQ ID NO: 23) |
| Design C | 5′ spacer sequence: |
| (5 nt spacer): | ATAAT (SEQ ID NO: 7) |
| | 3′ spacer sequence: |
| | ATATA |
| | (SEQ ID NO: 6) |
| Design D | 5′ spacer sequence: |
| (10 nt | ATAATATAAT |
| spacer): | (SEQ ID NO: 35) |
| | 3′ spacer sequence: |
| | ATATAAATAT |
| | (SEQ ID NO: 36) |
| Design E | 5′ spacer sequence: |
| (15 nt | AATAAATAATATAAT |
| spacer): | (SEQ ID NO: 37) |
| | 3′ spacer sequence: |
| | ATATAAATATTAAAA |
| | (SEQ ID NO: 38) |

### Example 16

### LRRK2 Editing with Circular Guide RNAs

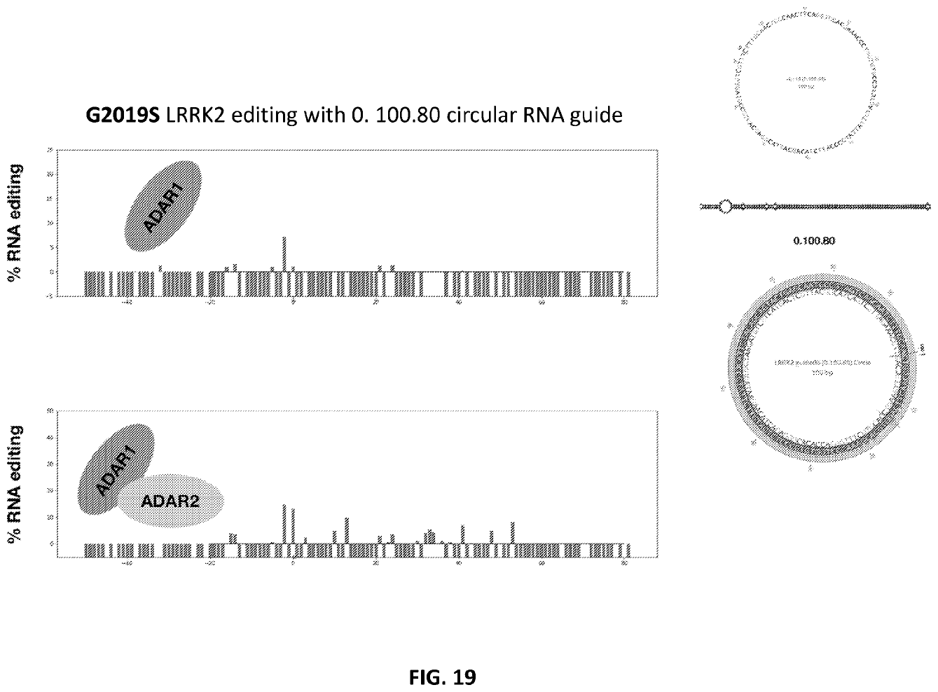

This example describes LRRK2 editing with circular guide RNAs of the present disclosure. 20,000 cells were transfected with 500 ng of plasmid encoding for guides. Cells tested include HEK293 cells, which express endogenous ADAR1, transfected with a piggyBac vector carrying the LRRK2 minigene carrying the G2019S mutation (corresponding data in FIG. 19, at top) and HEK293 cells, which express endogenous ADAR1, transfected with a piggyBac vector carrying ADAR2 and the LRRK2 minigene carrying the G2019S mutation.

RNA was isolated 48 hours post transfection and converted into a cDNA library. PCR and Sanger sequencing analyses were carried out. Percent editing was determined using the EditR program. The guide RNA was 100 bases long with the A/C mismatch engineered 80 bases from the 5′ end of the guide. The guide RNA has a sequence of

(SEQ ID NO: 39)
5′-CTGGCAACTTCAGGTGCACGAAACCCTGGTGTGCCCTCTGATGTT
CTTATCCCCATTCTACAGCAGTACTGAGCAATGCCGTAGTCAGCAATC
TTTGCAA-3′.

FIG. 19 shows graphs of on-target and off-target ADAR1 editing and ADAR1+ADAR2 editing of the G2019S mutation.

### Example 17

### SNCA Editing with Circular Guide RNAs

This example describes constructs of the present disclosure encoding for circular guide RNAs under the control of a U6 promoter, where the guide RNA is designed to target a start site, or translation initiation site (TIS) (also referred to as translation start site (TSS)) in the SNCA gene. Guide constructs were designed to target SNCA TIS adenosine at nucleotide position 26 of Exon 2 (corresponding to nucleotide position 264 of most SNCA variants, including Exon 1 and Exon 2). HEK293 cells were transfected with a plasmid encoding for a guide RNA of interest and RNA editing was assessed 48 hours post-transfection (unless otherwise specified). RNA editing was assessed for ADAR1 only, which is naturally expressed by HEK293 cells, and ADAR1 and ADAR2. In the latter experiment, HEK293 cells were co-transfected with a piggybac vector encoding for ADAR2. Levels of RNA editing were quantified by sequencing and analyzed using a software program.

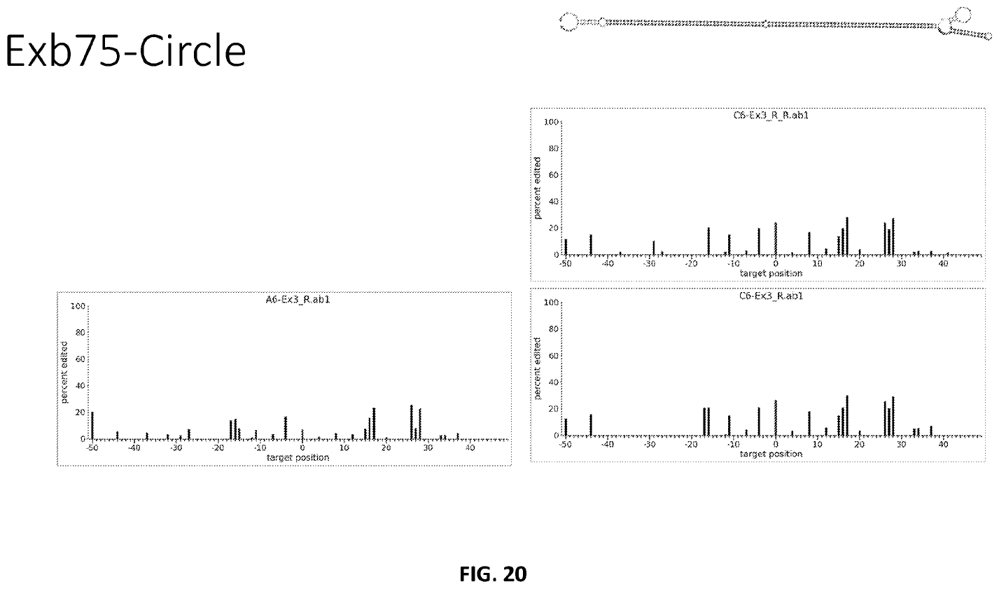

FIG. 20-FIG. 24 show plots of RNA editing at the target A to be edited (“0” on the x-axis) and at RNA editing at off-target positions (represented as black bars at positions that are not “0”). Biological replicates are shown in each column.

Sequences of guides shown in FIG. 20-FIG. 24 are described below in TABLE 5. TABLE 5 below also describes the percent on-target RNA editing observed.

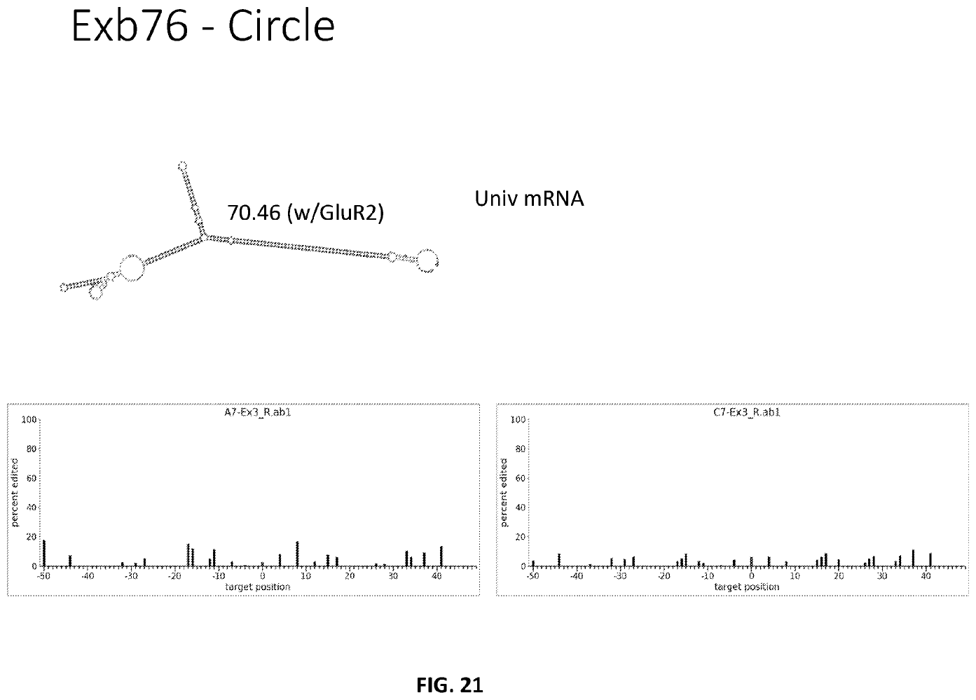

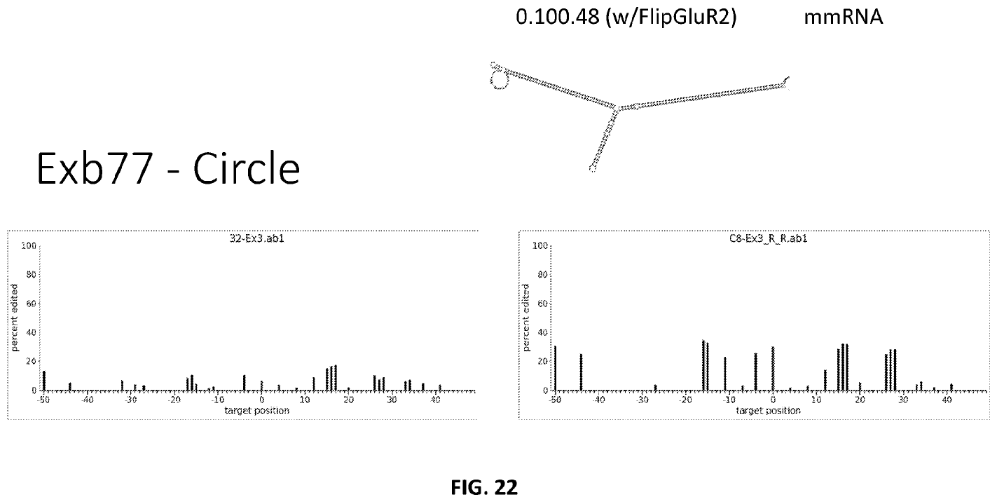

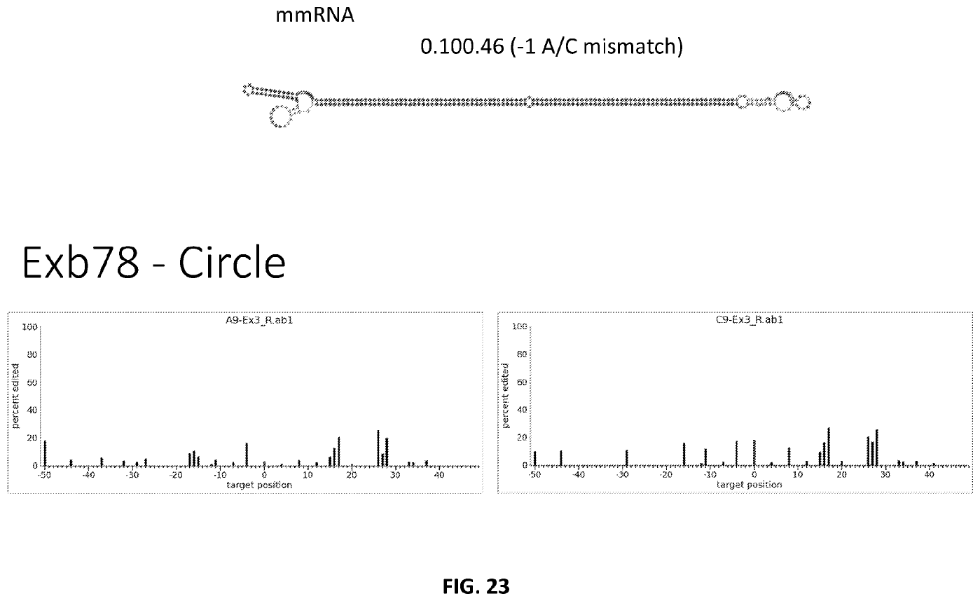

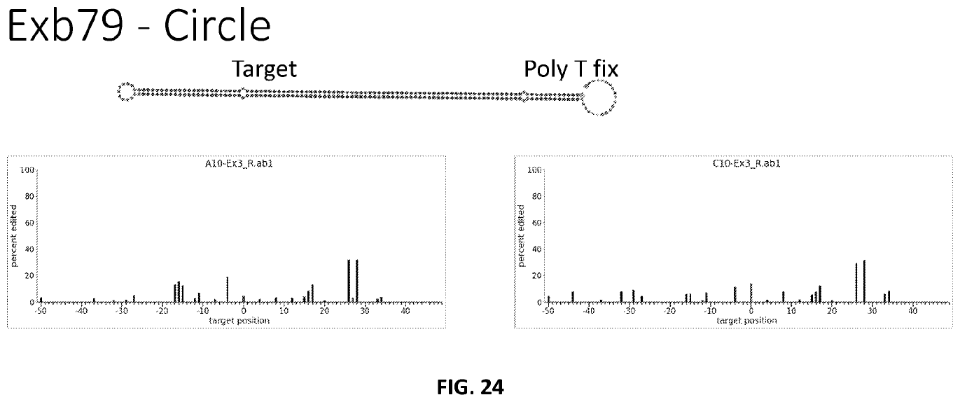

TABLE 5
Guide RNA Sequences

| | | Percent On-Target |
|---|---|---|
| | | Editing [ADAR1, |
| SEQ ID NO | Sequence | AD ARI + ADAR2] |
| FIG. 20 (SEQ | TAGGGATAGGGATAGGGACA | [3, 22] |
| ID NO: 40) | ACTCCCTCCTTGGCCTTTGA | |
| | AAGTCCTTTCATGAATACAT | |
| | CCACGGCTAATGAATTCCTT | |
| | TACACCACACTGTCGTCGAA | |
| | TGGCCACTCCCAGTTCTC | |
| FIG. 21 (SEQ | TAGGGATAGGGATAGGGACA | [2, 5] |
| ID NO: 41) | ACTCCCTCCTTGGCCTTTGA | |
| | AAGTCCTTTCATGAATACAT | |
| | CCACGGCTAAT [symbol] | |
| | [symbol] [symbol] | |
| | [symbol] | |
| | GAATTCCTTTACACCACA | |
| FIG. 22 | CAACTCCCTCCTTGGCCTTT | [6, 30] |
| (SEQ ID NO: | GAAAGTCCTTTCATGAATA [symbol] | |
| 42) | [symbol] [symbol] | |
| | [symbol] [symbol] | |
| | [symbol] CATCCACGGCTAATGAATT | |
| | CCTTTACACCACACTGTCGT | |
| | CGAATGGCCACTCCCAGTT | |
| | CTCA | |
| FIG. 23 | TAGGGATAGGGATAGGGACA | [2,18] |
| (SEQ ID NO: | ACTCCCTCCTTGGCCTTTGA | |
| 43) | AAGTCCTTTCATGAATACAT | |
| | CCATAGCTAATGAATTCCTT | |
| | TACACCACACTGTCGTCGAA | |
| | TGGCCACTCCCAGTTCTCA | |
| FIG. 24 | TAGGGATAGGGATAGGGACC | [4,13] |
| (SEQ ID NO: | CTGTTTGGTTCTCTCAGCAG | |
| 44) | CAGCCACAACTCCCTCCTTG | |
| | GCCTTTGAAAGTCCTTTCAT | |
| | GAATACATCCATAGCTAATG | |
| | AATTCCTTTACACCACA | |

### Example 18

### Circularized Guide RNA Containing a 3′ SmOPT Sequence and U7 Hairpin

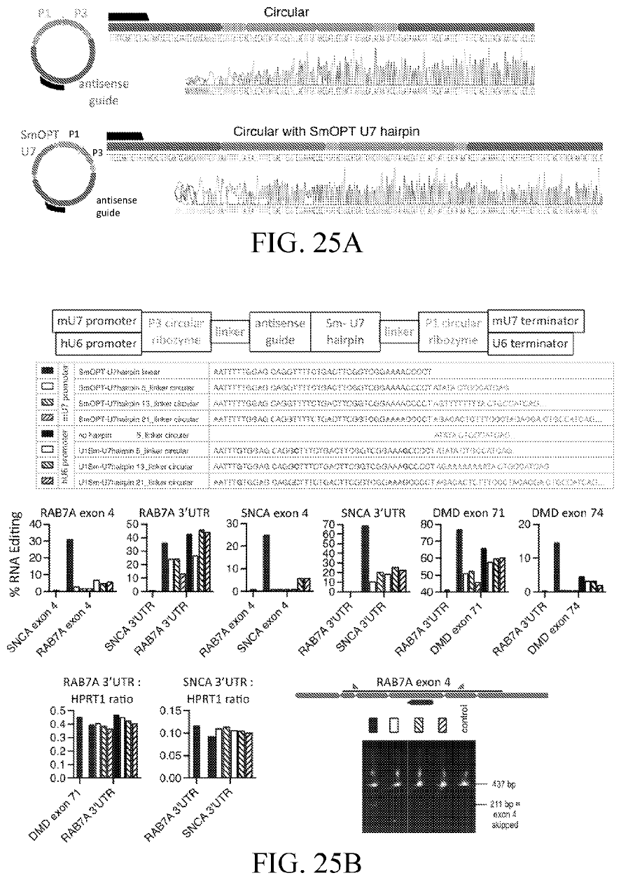

100 nt antisense (targeting) guide RNAs with a 3′ SmOPT sequence and U7 hairpin were inserted between P3 and P1 RtcB circular ribozyme sites and expressed using the mU7 or hU6 promoters. 293T cells were transfected with 1 ug of plasmid expressing the guide RNA construct (endogenous ADAR levels); RNA was measured 41 hr later. FIG. 25A illustrates the circular RNA forms; Sanger sequencing with a guide-specific primer shows that the ribozyme sites have been precisely ligated together, with the antisense guide and 3′ SmOPT U7 hairpin inside the circular RNA.

FIG. 25B lists several sequence variations of the Sm-binding domain, U7 stem-loop hairpin, and RNA linker sequences that were cloned between P3 and P1 RtcB circular ribozyme sites (top panel). Included within the circular RNA, before the Sm-binding domain, were 100 nt antisense guide RNAs targeting human RAB7A exon 4, RAB7A 3′UTR, SNCA exon 4, SNCA 3′UTR, DMD exon 71 Splice Acceptor, or DMD exon 74 Splice Acceptor. Guide RNAs were expressed using either the mU7 or hU6 promoter, each with its corresponding terminator sequence. In addition to a linear SmOPT U7 hairpin guide RNA, circularized guide RNAs containing an Sm-binding domain and U7 stem-loop hairpin could also edit the target transcript, as measured by Sanger sequencing (middle panel). As a negative control, linear SmOPT U7 hairpin guide RNAs that targeted a different gene were used. Furthermore, neither the linear or circular forms of the SmOPT U7 hairpin guide RNAs altered the gene expression level of the RAB7A 3′UTR or SNCA 3′UTR target transcripts, as compared to the HPRT1 housekeeping gene and measured by ddPCR (bottom left panel). The linear SmOPT U7 hairpin guide RNA caused only minimal inadvertent skipping of RAB7A exon 4, and the circular SmOPT U7 hairpin guide RNAs showed no detectable exon skipping, as measured by PCR and gel electrophoresis (bottom right panel).

### Example 19

### AAV Delivery of Circular Guides

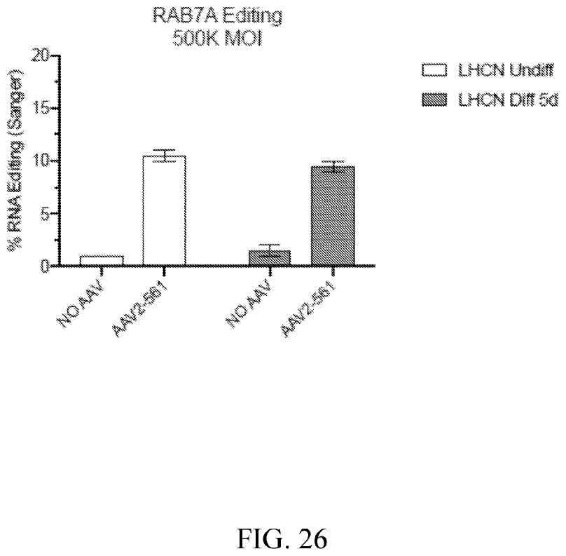

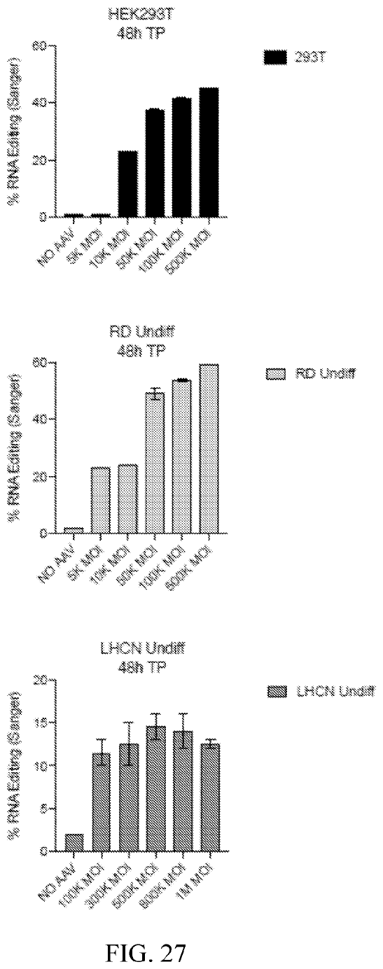

Rab7a targeting circular guides (0.100.50) were delivered via AAV2 into muscle and 293T cells. FIG. 26 shows a graph of percent RNA editing as determined by Sanger sequencing of cells transduced with an AAV2 vector carrying a Rab7a targeting circular guide (AAV2-561) and cells that were contacted with the Rab7a targeting circular without an AAV2 vector (NO AAV) in LHCN undifferentiated cells and LHCN differentiated cells (5 day differentiation). The guides in the AAV2 vector had increased editing as compared to the guides not in the AAV2 vector. FIG. 27 shows graphs of percent RNA editing as determined by Sanger sequencing in HEK293T cells, RD undifferentiated cells and LHCN undifferentiated cells by Rab7 targeting circular guides delivered by AAV2 vectors at different multiplicity of infections (MOI) and delivered absent from an AAV2 vector. Percent RNA editing is shown 48 hours post transfection. Transduction with a higher MOI lead to increased RNA editing.

### Example 20

### AAV Delivery of Circular Guides

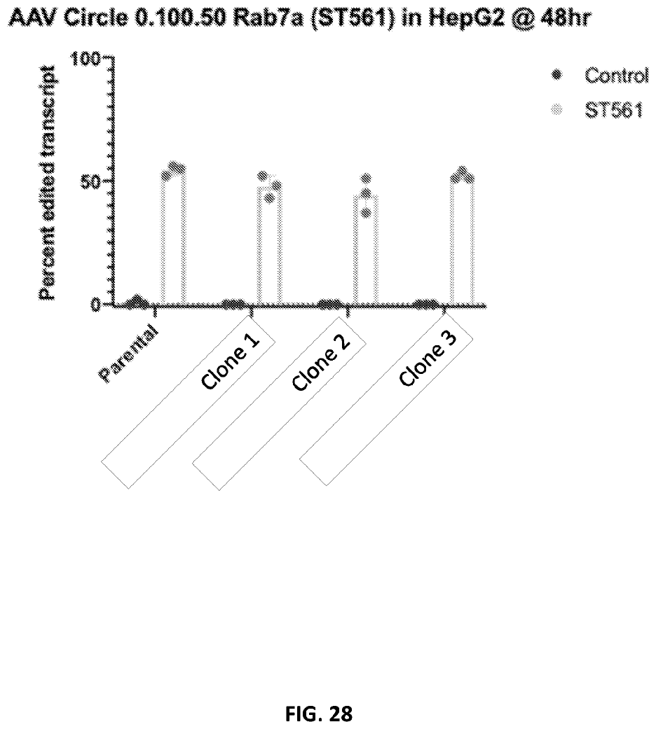

This example describes AAV delivery of circular guide RNAs targeting RAB7A in HepG2 cells engineered to express the SERPINA1 E342K mutation. RAB7A targeting circular guide RNAs having a sequence of GATAAAAGGCGTACATAATTCTTGTGTCTACTGTACAGAATACTGCCGCCAGCTGGA TTTCCCAATTCTGAGTAACACTCTGCAATCCAAACAGGGTTCA (SEQ ID NO: 23) were delivered via AAV2 into 50,000 cells with an MOI of 100,000, including parental HepG2 cells and three HepG2 clones (Clone 1, Clone 2, and Clone 3) carrying the SERPINA1 E342K mutation. RAB7A editing was quantified via a ddPCR dropoff assay 48 hours post-infection. As shown in FIG. 28 guide RNAs showed successful editing of RAB7A in the parental cell line and in all 3 clones.

### Example 21

### Circular Chassis with Filler Sequence

This example describes RAB7A editing with circular guide RNAs of the present disclosure having varying lengths of filler sequence. WT HEK293 cells (20,000 cells) were transfected with 500 ng of plasmid encoding for guide RNAs or controls. RNA was isolated 48 hours post-transfection and converted into a cDNA library. PCR and Sanger sequencing was carried out. Percent editing was determined using the EditR software. RNA editing was assessed for ADAR1 only, which is naturally expressed by HEK293 cells, and ADAR1 and ADAR2. In the latter experiment, HEK293 cells were co-transfected with a piggybac vector encoding for ADAR2 to overexpress ADAR2.

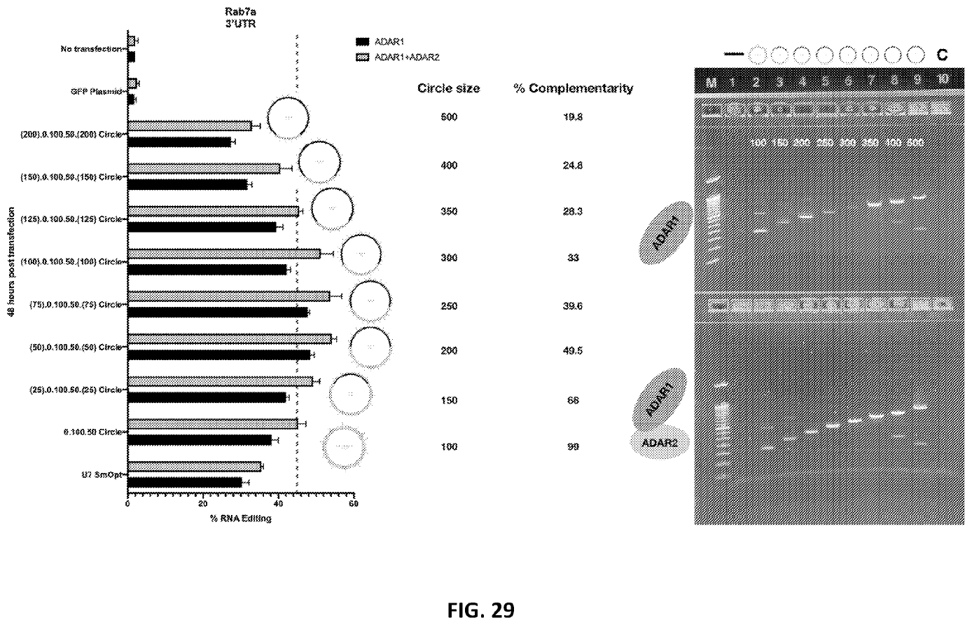

FIG. 29 shows, at left, a bar graph of percent editing of the RAB7A 3′UTR for various circular guide RNAs of different sizes and having different percentages of the entire circular chassis that was complementary to the target (e.g., the portion of the circular chassis that is the targeting domain was modulated by varying the amount of filler sequence). Surprisingly, the level of RNA editing could be modulated by varying the ratio of the guide RNA to the entire circular chassis. That is, neither a low or high ratio of guide RNA to the entire circular chassis exhibited the highest editing. Circular chassis wherein the guide RNA was ˜28% to ˜66% of the entire circular chassis provided the highest levels of RNA editing. These results were consistent for ADAR1 alone and ADAR1+ADAR2 overexpression. The gel at the right of the figure shows confirmation of circularization of the tested circular guide RNAs.

Sequences of guides are provided below in TABLE 6. The guide RNA is bolded and the filler sequence is underlined.

TABLE 6
Guide Sequences

| SEQ ID NO | Sequence |
|---|---|
| SEQ ID NO: 45 | GGAGTGCTTAGCGCGTGATTACTGCTGGAGGATTGGAA |
| (200).0.100.50.(200) Circle | TTGGCGATTCTTACGCGGAACCACGATAACGAGATAAC |
| | GTTAAGTCGCTAGACTTAAGGCGGCACGCGATCGAGC |
| | AACTCCTCACTACGATAGGTGAGCGCAGTCCAGTGTAG |
| | CTCAGTCGGCCAATTACGGTTCTATTAGACAGTTGCAC |
| | GTTGACATCAC |
| | GATAAAAGGCGTACATAATTCTTGTGTCTACTGTAC |
| | AGAATACTGCCGCCAGCTGGATTTCCCAATTCTGAG |
| | TAACACTCTGCAATCCAAACAGGGTTCA |
| | ACTCGTTCCTATAGTGTATAGACTCAATTGTAGTTCAC |
| | CGCTGAGTATCACTCACAGCACGATCTACTCAGACTTA |
| | ATCGCACTACTGGACGGAATCGCGTCGTCGGCCAATAA |
| | GCCTGTGTCCAGAGCTGCAGTAGTAACTCCACTGTAGC |
| | TGTTAGACAATAAGACGAGTGATACTGGCTAGCGAGG |
| | AGCGCGAGGAC |
| SEQ ID NO: 46 | GGAGTGCTTAGCGCGTGATTACTGCTGGAGGATTGGAA |
| (150).0.100.50.(150) Circle | TTGGCGATTCTTACGCGGAACCACGATAACGAGATAAC |
| | GTTAAGTCGCTAGACTTAAGGCGGCACGCGATCGAGC |
| | AACTCCTCACTACGATAGGTGAGCGCAGTCCAGTGTA |
| | GATAAAAGGCGTACATAATTCTTGTGTCTACTGTAC |
| | AGAATACTGCCGCCAGCTGGATTTCCCAATTCTGAG |
| | TAACACTCTGCAATCCAAACAGGGTTCA |
| | GCTCAGTCGGCCAATTACGGTTCTATTAGACAGTTGCA |
| | CGTTGACATCACACTCGTTCCTATAGTGTATAGACTCA |
| | ATTGTAGTTCACCGCTGAGTATCACTCACAGCACGATC |
| | TACTCAGACTTAATCGCACTACTGGACGGAATCGCG |
| SEQ ID NO: 47 | GGAGTGCTTAGCGCGTGATTACTGCTGGAGGATTGGAA |
| (125).0. 100.50.(125) Circle | TTGGCGATTCTTACGCGGAACCACGATAACGAGATAAC |
| | GTTAAGTCGCTAGACTTAAGGCGGCACGCGATCGAGC |
| | AACTCCTCACTA |
| | GATAAAAGGCGTACATAATTCTTGTGTCTACTGTAC |
| | AGAATACTGCCGCCAGCTGGATTTCCCAATTCTGAG |
| | TAACACTCTGCAATCCAAACAGGGTTCA |
| | CGATAGGTGAGCGCAGTCCAGTGTAGCTCAGTCGGCC |
| | AATTACGGTTCTATTAGACAGTTGCACGTTGACATCAC |
| | ACTCGTTCCTATAGTGTATAGACTCAATTGTAGTTCAC |
| | CGCTGAGTATCA |
| SEQ ID NO: 48 | GGAGTGCTTAGCGCGTGATTACTGCTGGAGGATTGGAA |
| (100).0.100.50.(100) Circle | TTGGCGATTCTTACGCGGAACCACGATAACGAGATAAC |
| | GTTAAGTCGCTAGACTTAAGGCGG |
| | GATAAAAGGCGTACATAATTCTTGTGTCTACTGTAC |
| | AGAATACTGCCGCCAGCTGGATTTCCCAATTCTGAG |
| | TAACACTCTGCAATCCAAACAGGGTTCA |
| | CACGCGATCGAGCAACTCCTCACTACGATAGGTGAGC |
| | GCAGTCCAGTGTAGCTCAGTCGGCCAATTACGGTTCTA |
| | TTAGACAGTTGCACGTTGACATCAC |
| SEQ ID NO: 49 | GGAGTGCTTAGCGCGTGATTACTGCTGGAGGATTGGAA |
| (75).0.100.50.(75) Circle | TTGGCGATTCTTACGCGGAACCACGATAACGAGATAA |
| | GATAAAAGGCGTACATAATTCTTGTGTCTACTGTAC |
| | AGAATACTGCCGCCAGCTGGATTTCCCAATTCTGAG |
| | TAACACTCTGCAATCCAAACAGGGTTCA |
| | CGTTAAGTCGCTAGACTTAAGGCGGCACGCGATCGAG |
| | CAACTCCTCACTACGATAGGTGAGCGCAGTCCAGTGTA |
| SEQ ID NO: 50 | GGAGTGCTTAGCGCGTGATTACTGCTGGAGGATTGGAA |
| (50).0.100.50.(50) Circle | TTGGCGATTCTT |
| | GATAAAAGGCGTACATAATTCTTGTGTCTACTGTAC |
| | AGAATACTGCCGCCAGCTGGATTTCCCAATTCTGAG |
| | TAACACTCTGCAATCCAAACAGGGTTCA |
| | ACGCGGAACCACGATAACGAGATAACGTTAAGTCGCT |
| | AGACTTAAGGCGG |
| SEQ ID NO: 51 | GGAGTGCTTAGCGCGTGATTACTGC |
| (25).0.100.50.(25) Circle | GATAAAAGGCGTACATAATTCTTGTGTCTACTGTAC |
| | AGAATACTGCCGCCAGCTGGATTTCCCAATTCTGAG |
| | TAACACTCTGCAATCCAAACAGGGTTCA |
| | TGGAGGATTGGAATTGGCGATTCTT |
| SEQ ID NO: 52 | GATAAAAGGCGTACATAATTCTTGTGTCTACTGTAC |
| U7 SmOPT | AGAATACTGCCGCCAGCTGGATTTCCCAATTCTGAG |
| | TAACACTCTGCAATCCAAACAGGGTTCA |
| | AATTTTTGGAGCAGGTTTTCTGACTTCGGTCGGAAAAC |
| | CCCT |

### Example 22

### Circular Chassis with Filler Sequence

This example describes LRRK2 editing with circular guide RNAs of the present disclosure having a filler sequence. This example describes LRRK2 editing with circular guide RNAs of the present disclosure. The details of the experimental set up and the guide are provided above in EXAMPLE 16. Briefly, 20,000 cells were transfected with 500 ng of plasmid encoding for guides. Cells tested include HEK293 cells, which express endogenous ADAR1, transfected with a piggyBac vector carrying the LRRK2 minigene carrying the G2019S mutation and HEK293 cells, which express endogenous ADAR1, transfected with a piggyBac vector carrying ADAR2 and the LRRK2 minigene carrying the G2019S mutation. Sequences are provided below in TABLE 7. The guide RNA is bolded and the filler sequence is underlined.

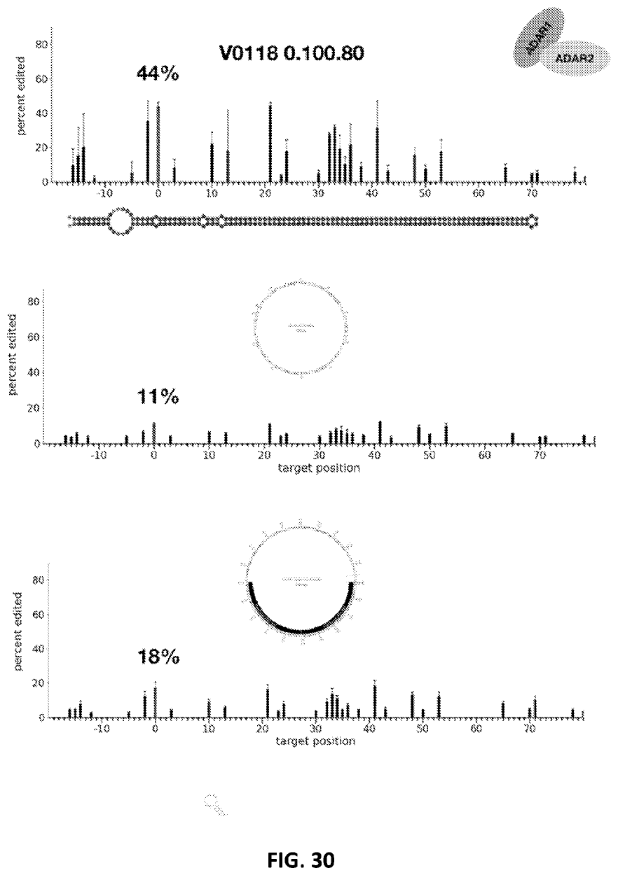

TABLE 7

| SEQ ID NO | Sequence |
|---|---|
| SEQ ID NO: 53 | 5′-CTGGCAACTTCAGGTGCA |
| Linear V0118 | CGAAACCCTGGTGTGCCCTCT |
| 0.100.80, | GATGTTTTTATCCCCATTCTA |
| top of FIG. 30 | CAGCATTACGGAGCAGTGCCG |
| | TAGTGTCGTTTCTTTGCAA-3′ |
| SEQ ID NO: 54 | CTGGCAACTTCAGGTGCACGA |
| Circular V0118 | AACCCTGGTGTGCCCTCTGAT |
| 0.100.80, | GTTTTTATCCCCATTCTACAG |
| middle of | CATTACGGAGCAGTGCCGTAG |
| FIG. 30 | TGTCGTTTCTTTGCAA |
| SEQ ID NO: 55 | GGAGTGCTTAGCGCGTGATTA |
| Circular with | CTGCTGGAGGATTGGAATTGG |
| Filler V0118 | CGATTCTTCTGGCAACTTCAG |
| 0.100.80, | GTGCACGAAACCCTGGTGTGC |
| bottom of | CCTCTGATGTTTTTATCCCCA |
| FIG. 30 | TTCTACAGCATTACGGAGCAG |
| | TGCCGTAGTGTCGTTTCTTTG |
| | CAAACGCGGAACCACGATAAC |
| | GAGATAACGTTAAGTCGCTAG |
| | ACTTAAGGCGG |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A precursor engineered linear polynucleotide which comprises:
a first spacer domain;
a targeting domain that is substantially complementary to a target RNA implicated in a disease or condition; and
a second spacer domain,
wherein the first or second spacer domain is not substantially complementary to the target RNA,
wherein a transcript of the precursor engineered linear polynucleotide circularizes upon insertion of the precursor engineered linear polynucleotide in a mammalian cell, thereby forming a circularized engineered polynucleotide; and
wherein hybridization of the targeting domain with the target RNA facilitates an edit of a base of a nucleotide of the target RNA by an RNA editing enzyme.

2. The precursor engineered linear polynucleotide of claim 1, wherein the precursor engineered linear polynucleotide comprises, in order of 5′ to 3′: the first spacer domain, the targeting domain, and the second spacer domain.

3. The precursor engineered linear polynucleotide of claim 1 or 2, wherein the precursor engineered linear polynucleotide comprises a ribozyme domain 5′ to the first spacer domain or 3′ to the second spacer domain.

4. The precursor engineered linear polynucleotide of claim 3, wherein the precursor engineered linear polynucleotide comprises a ligation domain between the ribozyme domain and the first spacer domain or between the ribozyme domain and the second spacer domain.

5. The precursor engineered linear polynucleotide of any one of claims 1-4, wherein after self-circularization, the first spacer domain and the second spacer domain are a filler sequences that are from about 40% to about 70% of a total length of the circularized engineered polynucleotide.

6. The precursor engineered linear polynucleotide of claim 5, wherein after self-circularization, the first spacer domain and the second spacer domain are filler sequences that are from about 50% to about 67% of the total sequence of the circularized engineered polynucleotide.

7. The precursor engineered linear polynucleotide of claim 5 or 6, wherein the filler sequence increases hybridization of the targeting domain to the target RNA, relative to an otherwise comparable circularized polynucleotide that lacks the filler sequence.

8. The precursor engineered linear polynucleotide of any one of claims 5-7, wherein after self-circularization the total length of the circularized engineered polynucleotide comprises about 150 nucleotides to about 400 nucleotides.

9. The precursor engineered linear polynucleotide of claim 8, wherein after self-circularization the total length of the circularized engineered polynucleotide comprises about 200 nucleotides to about 300 nucleotides.

10. The precursor engineered linear polynucleotide of any one of claims 1-9, wherein the targeting domain comprises at least about 80%, at least about 85%, at least about 90%, at least about 92%, at least about 95%, at least about 97%, at least about 99%, or 100% complementarity to the target RNA.

11. The precursor engineered linear polynucleotide of any one of claims 1-10, wherein the target RNA is an RNA selected from the group consisting of a pre-messenger RNA, a messenger RNA, a ribosomal RNA, a transfer RNA, a long non-coding RNA, and a small RNA.

12. The precursor engineered linear polynucleotide of any one of claims 1-11, wherein the targeting domain is substantially complementary to a 3′ or 5′ untranslated region (UTR) of the target RNA.

13. The precursor engineered linear polynucleotide of any one of claims 1-12, wherein the targeting domain is substantially complementary to an intronic region of the target RNA

14. The precursor engineered linear polynucleotide of any one of claims 1-13, wherein the targeting domain is substantially complementary to a translation initiation site (TIS).

15. The precursor engineered linear polynucleotide of any one of claims 1-14, wherein the targeting domain is substantially complementary to an upstream open reading frame (uORF) of the target RNA.

16. The precursor engineered linear polynucleotide of any one of claims 1-15, wherein the targeting domain comprises at least a single nucleotide that is mismatched to the target RNA.

17. The precursor engineered linear polynucleotide of any one of claims 1-16, wherein the edit of the base increases a level of a protein or fragment thereof, increases a length of a protein or fragment thereof, increases a functionality of a protein or fragment thereof, increases a stability of a protein or fragment thereof, or any combination thereof, after translation of the target RNA with the edit of the base, relative to a translated protein of an otherwise comparable target RNA lacking the edit.

18. The precursor engineered linear polynucleotide of any one of claims 1-17, wherein the edit of a base converts a sense codon into a stop codon.

19. The precursor engineered linear polynucleotide of claim 18, wherein the sense codon is implicated in a disease pathogenic pathway and wherein converting the sense codon to the stop codon reduces the disease pathogenic pathway.

20. The precursor engineered linear polynucleotide of any one of claims 1-17, wherein the edit of a base converts a stop codon into a sense codon.

21. The precursor engineered linear polynucleotide of claim 20, wherein the stop codon is implicated in a disease pathogenic pathway and wherein converting the stop codon to the sense codon reduces the disease pathogenic pathway.

22. The precursor engineered linear polynucleotide of any one of claims 1-17, wherein the edit of a base converts a first sense codon into a second sense codon.

23. The precursor engineered linear polynucleotide of claim 22, wherein the first sense codon is implicated in a disease pathogenic pathway and wherein converting the first sense codon to the second sense codon reduces the disease pathogenic pathway.

24. The precursor engineered linear polynucleotide of any one of claims 1-23, wherein the targeting domain is from about 20 nucleotides to about 150 nucleotides; or from about 100 nucleotides to about 200 nucleotides.

25. The precursor engineered linear polynucleotide of any one of claims claim 1-24, wherein the RNA editing enzyme comprises an ADAR protein or an APOBEC protein.

26. The precursor engineered linear polynucleotide claim 25, wherein the RNA editing enzyme comprises ADAR and wherein the ADAR is ADAR1.

27. The precursor engineered linear polynucleotide of claim 25, wherein the RNA editing enzyme comprises ADAR and wherein the ADAR is ADAR2.

28. The precursor engineered linear polynucleotide of claim 25, wherein the RNA editing enzyme comprises ADAR and wherein the ADAR is ADAR3.

29. The precursor engineered linear polynucleotide of any one of claims 1-28, wherein the disease or condition comprises Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, Hypomyelination with Atrophy of Basal Ganglia and Cerebellum (H-ABC) or cystic fibrosis.

30. The precursor engineered linear polynucleotide of any one of claims 1-29, wherein the target RNA comprises TUBB4A, and wherein TUBB4A comprises a D249N mutation.

31. The precursor engineered linear polynucleotide of claim 30, wherein the targeting domain comprises at least about 80%, at least about 85%, at least about 90%, at least about 92%, at least about 95%, at least about 97%, at least about 99%, or 100% sequence homology to SEQ ID NO: 32, SEQ ID NO: 33, or SEQ ID NO: 34.

32. The precursor engineered linear polynucleotide of any one of claims 1-29, wherein the target RNA comprises LRRK2, and wherein LRRK2 comprises a G2019S mutation.

33. The precursor engineered linear polynucleotide of claim 32, wherein the targeting domain comprises at least about 80%, at least about 85%, at least about 90%, at least about 92%, at least about 95%, at least about 97%, at least about 99%, or 100% sequence homology to SEQ ID NO: 39, SEQ ID NO: 53, or SEQ ID NO: 54.

34. The precursor engineered linear polynucleotide of any one of claims 1-29, wherein the target RNA comprises SERPINA1, and wherein SERPINA1 comprises a E342K mutation.

35. The precursor engineered linear polynucleotide of claim 34, wherein the targeting domain comprises at least about 80%, at least about 85%, at least about 90%, at least about 92%, at least about 95%, at least about 97%, at least about 99%, or 100% sequence homology to SEQ ID NO: 64, or SEQ ID NO: 65.

36. The precursor engineered linear polynucleotide of any one of claims 1-29, wherein the target RNA comprises SNCA.

37. The precursor engineered linear polynucleotide of claim 36, wherein the targeting domain comprises at least about 80%, at least about 85%, at least about 90%, at least about 92%, at least about 95%, at least about 97%, at least about 99%, or 100% sequence homology to SEQ ID NO: 40, SEQ ID NO: 41, SEQ ID NO: 42, SEQ ID NO: 43, SEQ ID NO: 44.

38. The precursor engineered linear polynucleotide of claim 37, wherein the targeting domain is designed to target the translation initiation site (TIS).

39. The precursor engineered linear polynucleotide of any one of claims 1-29, wherein the target RNA comprises APP.

40. The precursor engineered linear polynucleotide of claim 39, wherein the targeting domain comprises at least about 80%, at least about 85%, at least about 90%, at least about 92%, at least about 95%, at least about 97%, at least about 99%, or 100% sequence homology to SEQ ID NO: 61, SEQ ID NO: 62, or SEQ ID NO: 63.

41. The precursor engineered linear polynucleotide of any one of claims 1-29, wherein the target RNA comprises ABCA4.

42. The precursor engineered linear polynucleotide of claim 41, wherein the targeting domain comprises at least about 80%, at least about 85%, at least about 90%, at least about 92%, at least about 95%, at least about 97%, at least about 99%, or 100% sequence homology to SEQ ID NO: 58, SEQ ID NO: 59, or SEQ ID NO: 60.

43. The precursor engineered linear polynucleotide of any one of claims 1-29, wherein the target RNA comprises DMD.

44. The precursor engineered linear polynucleotide of claim 43, wherein the targeting domain comprises at least about 80%, at least about 85%, at least about 90%, at least about 92%, at least about 95%, at least about 97%, at least about 99%, or 100% sequence homology to SEQ ID NO: 56, or SEQ ID NO: 57.

45. A precursor engineered polynucleotide which comprises, in order of 5′ to 3′: a first ribozyme domain; a first ligation domain; a first spacer domain; a targeting domain that is substantially complementary to a target RNA implicated in a disease or condition, a second ligation domain, and a second ribozyme domain, wherein the first spacer domain is not substantially complementary to the target RNA, wherein a transcript of the precursor engineered linear polynucleotide circularizes upon insertion of the precursor engineered linear polynucleotide in a mammalian cell, thereby forming a circularized engineered polynucleotide; and wherein hybridization of the targeting domain with the target RNA facilitates an edit of a base of a nucleotide of the target RNA by an RNA editing enzyme.

46. An engineered circular polynucleotide comprising: a targeting domain that is substantially complementary to a target RNA implicated in a disease or condition, and a spacer domain that is not substantially complementary to the target RNA, wherein the spacer domain enlarges the engineered circular polynucleotide by the addition of one or more nucleotides, wherein hybridization of the targeting domain with the target RNA facilitates an edit of a base of a nucleotide of the target RNA by an RNA editing enzyme.

47. The engineered circular polynucleotide of claim 46, wherein the edit of the base of a nucleotide of the target RNA by the RNA editing entity is determined in an in vitro assay comprising:
(i) directly or indirectly introducing the target RNA into a primary cell line,
(ii) directly or indirectly introducing the engineered polynucleotide into a primary cell line, and
(iii) sequencing the target RNA.

48. The engineered circular polynucleotide of claim 46 or 47, wherein the engineered circular polynucleotide does not comprise a 5′ reducing hydroxyl, a 3′ reducing hydroxyl, or both, capable of being exposed to a solvent.

49. The engineered circular polynucleotide of any one of claims 46-48, further comprising an RNA editing enzyme recruiting domain, wherein the RNA editing enzyme recruiting domain recruits an RNA editing enzyme that, when associated with the engineered polynucleotide, performs a chemical transformation on a base of a nucleotide in the target RNA.

50. The engineered circular polynucleotide of any one of claims 46-49, wherein the targeting domain is about 20 nucleotides to about 150 nucleotides.

51. The engineered circular polynucleotide of any one of claims 46-50, wherein the target RNA comprises a nonsense mutation.

52. The engineered circular polynucleotide of any one of claims 46-51, wherein the targeting domain comprises at least a single nucleotide that is mismatched to the target RNA.

53. The engineered circular polynucleotide of claim 46, wherein the disease or condition comprises Rett syndrome, Huntington's disease, Parkinson's Disease, Alzheimer's disease, a muscular dystrophy, Tay-Sachs Disease, alpha-1 antitrypsin deficiency (AATD), a dementia, a tauopathy, a synucleinopathy, Stargardt disease, Hypomyelination with Atrophy of Basal Ganglia and Cerebellum (H-ABC), or cystic fibrosis.

54. A vector comprising, the precursor engineered linear polynucleotide of any one of claims 1-45, or the engineered circular polynucleotide of any one of claims 46-53.

55. The vector of claim 54, wherein the vector comprises an adeno-associated virus (AAV) vector.

56. The vector of claim 55, wherein the AAV vector is an AAV1 vector, AAV2 vector, AAV3 vector, AAV4 vector, AAVS vector, AAV6 vector, AAV7 vector, AAV8 vector, an AAV9 vector, a chimera of any of these, or a variant of any of these.

57. The vector of claim 55 or 56, wherein the viral vector is a self-complementary adeno-associated viral (scAAV) vector.

58. The vector of claim 55 or 56, wherein the viral vector is a single-stranded AAV vector.

59. A pharmaceutical composition in unit dose form comprising the precursor engineered linear polynucleotide of any one of claims 1-45, the engineered circular polynucleotide of any one of claims 45-53 or the vector of any one of claims 54-58; and a pharmaceutically acceptable: excipient, diluent, or carrier.

60. A method of treating or preventing a disease or condition in a subject in need thereof comprising: administering a therapeutically effective amount of: the precursor engineered linear polynucleotide of any one of claims 1-45, the engineered circular polynucleotide of any one of claims 46-53, the vector of any one of claims 54-58, or the pharmaceutical composition of claim 59.


SEQUENCE LISTING (85 sequences)

SEQ ID NO: 1 - DNA, 36 bp, Unknown
  source: /note=“Description of Unknown OTC pre-mRNA sequence”
ctcacagacaccgctcggtttgtaaaacttttcttc

SEQ ID NO: 2 - DNA, 36 bp, Artificial Sequence
  source: /note=“Description of Artificial Sequence Synthetic oligonucleotide”
ctcacagacaccgctcagtttgtaaaacttttcttc

SEQ ID NO: 3 - DNA, 36 bp, Artificial Sequence; identical to SEQ ID NO: 2
  source: /note=“Description of Artificial Sequence Synthetic oligonucleotide”

SEQ ID NO: 4 - DNA, 36 bp, Artificial Sequence
  source: /note=“Description of Artificial Sequence Synthetic oligonucleotide”
ctcacagacaccgctcatgtcttatctagcatgaca

SEQ ID NO: 5 - DNA, 36 bp, Unknown
  source: /note=“Description of Unknown OTC mRNA sequence”
ctcacagacaccgctcgtgtcttatctagcatgaca

SEQ ID NO: 6 - DNA, 5 bp, Artificial Sequence
  source: /note=“Description of Artificial Sequence Synthetic oligonucleotide”
atata

SEQ ID NO: 7 - DNA, 5 bp, Artificial Sequence
  source: /note=“Description of Artificial Sequence Synthetic oligonucleotide”
ataat

SEQ ID NO: 8 - RNA, 5 nt, Artificial Sequence
  source: /note=“Description of Artificial Sequence Synthetic oligonucleotide”
auaau

SEQ ID NO: 9 - RNA, 5 nt, Artificial Sequence
  source: /note=“Description of Artificial Sequence Synthetic oligonucleotide”
auaua

SEQ ID NO: 10 - DNA, 21 bp, Artificial Sequence
  source: /note=“Description of Artificial Sequence Synthetic oligonucleotide”
aaccatgccgactgatggcag

SEQ ID NO: 11 - DNA, 26 bp, Artificial Sequence
  source: /note=“Description of Artificial Sequence Synthetic oligonucleotide”
gatgtcaggtgcggctgactaccgtc

SEQ ID NO: 12 - RNA, 21 nt, Artificial Sequence
  source: /note=“Description of Artificial Sequence Synthetic oligonucleotide”
aaccaugccgacugauggcag

SEQ ID NO: 13 - RNA, 26 nt, Artificial Sequence
  source: /note=“Description of Artificial Sequence Synthetic oligonucleotide”
gaugucaggugcggcugacuaccguc

SEQ ID NO: 14 - DNA, 56 bp, Artificial Sequence
  source: /note=“Description of Artificial Sequence Synthetic oligonucleotide”
gccatcagtcgccggtcccaagcccggataaaatgggagggggcgggaaaccgcct

SEQ ID NO: 15 - RNA, 56 nt, Artificial Sequence
  source: /note=“Description of Artificial Sequence Synthetic oligonucleotide”
gccaucagucgccggucccaagcccggauaaaaugggagggggcgggaaaccgccu

SEQ ID NO: 16 - DNA, 55 bp, Artificial Sequence
  source: /note=“Description of Artificial Sequence Synthetic oligonucleotide”
aacactgccaatgccggtcccaagcccggataaaagtggagggtacagtccacgc

SEQ ID NO: 17 - RNA, 55 nt, Artificial Sequence
  source: /note=“Description of Artificial Sequence Synthetic oligonucleotide”
aacacugccaaugccggucccaagcccggauaaaaguggaggguacaguccacgc

SEQ ID NO: 18 - DNA, 97 bp, Artificial Sequence
  source: /note=“Description of Artificial Sequence Synthetic oligonucleotide”
tctgcgcgctcgctcgctcactgaggccgggcgaccaaaggtcgcccgacgcccgggctttgcccgggcggcctcagtgagcgagcgagcgcgcaga

SEQ ID NO: 19 - DNA, 73 bp, Artificial Sequence
  source: /note=“Description of Artificial Sequence Synthetic oligonucleotide”
tctacgccatattatccacagtccaacggccaggcggaggctagtaacaaggttatcctcggcatcctccgca

SEQ ID NO: 20 - DNA, 249 bp, Artificial Sequence
  source: /note=“Description of Artificial Sequence Synthetic polynucleotide”
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 21 - DNA, 57 bp, Artificial Sequence
  source: /note=“Description of Artificial Sequence Synthetic oligonucleotide”
ggccatcagtcgccggtcccaagcccggataaaatgggagggggcgggaaaccgcct

SEQ ID NO: 22 - DNA, 31 bp, Artificial Sequence
  source: /note=“Description of Artificial Sequence Synthetic oligonucleotide”
aaccatgccgactgatggcagataatataat

SEQ ID NO: 23 - DNA, 100 bp, Artificial Sequence
  source: /note=“Description of Artificial Sequence Synthetic polynucleotide”
gataaaaggcgtacataattcttgtgtctactgtacagaatactgccgccagctggatttcccaattctgagtaacactctgcaatccaaacagggttca

SEQ ID NO: 24 - DNA, 36 bp, Artificial Sequence
  source: /note=“Description of Artificial Sequence Synthetic oligonucleotide”
atataaatatctgccatcagtcggcgtggactgtag

SEQ ID NO: 25 - DNA, 55 bp, Artificial Sequence; identical to SEQ ID NO: 16
  source: /note=“Description of Artificial Sequence Synthetic oligonucleotide”

SEQ ID NO: 26 - DNA, 698 bp, Artificial Sequence
  source: /note=“Description of Artificial Sequence Synthetic polynucleotide”
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagacaccgggaccgatccagcctccggactctagaggatcgaacc

SEQ ID NO: 27 - DNA, 741 bp, Artificial Sequence
  source: /note=“Description of Artificial Sequence Synthetic polynucleotide”
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtga

SEQ ID NO: 28 - DNA, 195 bp, Artificial Sequence
  source: /note=“Description of Artificial Sequence Synthetic polynucleotide”
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 29 - DNA, 69 bp, Artificial Sequence
  source: /note=“Description of Artificial Sequence Synthetic oligonucleotide”
ctcacggaccagtgcaacatattcccaacatcccgttgcagcctatcattaaaccttggccggtcgcgg

SEQ ID NO: 30 - DNA, 145 bp, Artificial Sequence
  source: /note=“Description of Artificial Sequence Synthetic polynucleotide”
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 31 - DNA, 2599 bp, Artificial Sequence
  source: /note=“Description of Artificial Sequence Synthetic polynucleotide”
tctgcgcgctcgctcgctcactgaggccgggcgaccaaaggtcgcccgacgcccgggctttgcccgggcggcctcagtgagcgagcgagcgcgcagaggatcctctacgccatattatccacagtccaacggccaggcggaggctagtaacaaggttatcctcggcatcctccgcaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccatcagtcgccggtcccaagcccggataaaatgggagggggcgggaaaccgcctaaccatgccgactgatggcagataatataatgataaaaggcgtacataattcttgtgtctactgtacagaatactgccgccagctggatttcccaattctgagtaacactctgcaatccaaacagggttcaatataaatatctgccatcagtcggcgtggactgtagaacactgccaatgccggtcccaagcccggataaaagtggagggtacagtccacgctttttttacatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagacaccgggaccgatccagcctccggactctagaggatcgaacccttaagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtgaaccggtcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagagctcctcacggaccagtgcaacatattcccaacatcccgttgcagcctatcattaaaccttggccggtcgcggctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 32 - DNA, 20 bp, Artificial Sequence
  source: /note=“Description of Artificial Sequence Synthetic oligonucleotide”
caggtcggcgttcagctggc

SEQ ID NO: 33 - DNA, 100 bp, Artificial Sequence
  source: /note=“Description of Artificial Sequence Synthetic polynucleotide”
gaagtgcaggcgaggaaagggaaccatgttgacggccagcttgcgcaggtcggcgttcagctggcccgggaagcgcaggcaggtggtgaccccgctcatg

SEQ ID NO: 34 - DNA, 268 bp, Artificial Sequence
  source: /note=“Description of Artificial Sequence Synthetic polynucleotide”
gccatcagtcgccggtcccaagcccggataaaatgggagggggcgggaaaccgcctaaccatgccgactgatggcagataatgaagtgcaggcgaggaaagggaaccatgttgacggccagcttgcgcaggtcggcgttcagctggcccgggaagcgcaggcaggtggtgaccccgctcatgatatactgccatcagtcggcgtggactgtagaacactgccaatgccggtcccaagcccggataaaagtggagggtacagtccacgc

SEQ ID NO: 35 - DNA, 10 bp, Artificial Sequence
  source: /note=“Description of Artificial Sequence Synthetic oligonucleotide”
ataatataat

SEQ ID NO: 36 - DNA, 10 bp, Artificial Sequence
  source: /note=“Description of Artificial Sequence Synthetic oligonucleotide”
atataaatat

SEQ ID NO: 37 - DNA, 15 bp, Artificial Sequence
  source: /note=“Description of Artificial Sequence Synthetic oligonucleotide”
aataaataatataat

SEQ ID NO: 38 - DNA, 15 bp, Artificial Sequence
  source: /note=“Description of Artificial Sequence Synthetic oligonucleotide”
atataaatattaaaa

SEQ ID NO: 39 - DNA, 100 bp, Artificial Sequence
  source: /note=“Description of Artificial Sequence Synthetic polynucleotide”
ctggcaacttcaggtgcacgaaaccctggtgtgccctctgatgttcttatccccattctacagcagtactgagcaatgccgtagtcagcaatctttgcaa

SEQ ID NO: 40 - DNA, 118 bp, Artificial Sequence
  source: /note=“Description of Artificial Sequence Synthetic polynucleotide”
tagggatagggatagggacaactccctccttggcctttgaaagtcctttcatgaatacatccacggctaatgaattcctttacaccacactgtcgtcgaatggccactcccagttctc

SEQ ID NO: 41 - DNA, 134 bp, Artificial Sequence
  source: /note=“Description of Artificial Sequence Synthetic polynucleotide”
tagggatagggatagggacaactccctccttggcctttgaaagtcctttcatgaatacatccacggctaatgtggaatagtataacaatatgctaaatgttgttatagtatcccacgaattcctttacaccaca

SEQ ID NO: 42 - DNA, 146 bp, Artificial Sequence
  source: /note=“Description of Artificial Sequence Synthetic polynucleotide”
caactccctccttggcctttgaaagtcctttcatgaatacaccctatgatattgttgtaaatcgtataacaatatgataaggtgcatccacggctaatgaattcctttacaccacactgtcgtcgaatggccactcccagttctca

SEQ ID NO: 43 - DNA, 119 bp, Artificial Sequence
  source: /note=“Description of Artificial Sequence Synthetic polynucleotide”
tagggatagggatagggacaactccctccttggcctttgaaagtcctttcatgaatacatccatagctaatgaattcctttacaccacactgtcgtcgaatggccactcccagttctca

SEQ ID NO: 44 - DNA, 117 bp, Artificial Sequence
  source: /note=“Description of Artificial Sequence Synthetic polynucleotide”
tagggatagggatagggaccctgtttggttctctcagcagcagccacaactccctccttggcctttgaaagtcctttcatgaatacatccatagctaatgaattcctttacaccaca

SEQ ID NO: 45 - DNA, 500 bp, Artificial Sequence
  source: /note=“Description of Artificial Sequence Synthetic polynucleotide”
ggagtgcttagcgcgtgattactgctggaggattggaattggcgattcttacgcggaaccacgataacgagataacgttaagtcgctagacttaaggcggcacgcgatcgagcaactcctcactacgataggtgagcgcagtccagtgtagctcagtcggccaattacggttctattagacagttgcacgttgacatcacgataaaaggcgtacataattcttgtgtctactgtacagaatactgccgccagctggatttcccaattctgagtaacactctgcaatccaaacagggttcaactcgttcctatagtgtatagactcaattgtagttcaccgctgagtatcactcacagcacgatctactcagacttaatcgcactactggacggaatcgcgtcgtcggccaataagcctgtgtccagagctgcagtagtaactccactgtagctgttagacaataagacgagtgatactggctagcgaggagcgcgaggac

SEQ ID NO: 46 - DNA, 400 bp, Artificial Sequence
  source: /note=“Description of Artificial Sequence Synthetic polynucleotide”
ggagtgcttagcgcgtgattactgctggaggattggaattggcgattcttacgcggaaccacgataacgagataacgttaagtcgctagacttaaggcggcacgcgatcgagcaactcctcactacgataggtgagcgcagtccagtgtagataaaaggcgtacataattcttgtgtctactgtacagaatactgccgccagctggatttcccaattctgagtaacactctgcaatccaaacagggttcagctcagtcggccaattacggttctattagacagttgcacgttgacatcacactcgttcctatagtgtatagactcaattgtagttcaccgctgagtatcactcacagcacgatctactcagacttaatcgcactactggacggaatcgcg

SEQ ID NO: 47 - DNA, 350 bp, Artificial Sequence
  source: /note=“Description of Artificial Sequence Synthetic polynucleotide”
ggagtgcttagcgcgtgattactgctggaggattggaattggcgattcttacgcggaaccacgataacgagataacgttaagtcgctagacttaaggcggcacgcgatcgagcaactcctcactagataaaaggcgtacataattcttgtgtctactgtacagaatactgccgccagctggatttcccaattctgagtaacactctgcaatccaaacagggttcacgataggtgagcgcagtccagtgtagctcagtcggccaattacggttctattagacagttgcacgttgacatcacactcgttcctatagtgtatagactcaattgtagttcaccgctgagtatca

SEQ ID NO: 48 - DNA, 300 bp, Artificial Sequence
  source: /note=“Description of Artificial Sequence Synthetic polynucleotide”
ggagtgcttagcgcgtgattactgctggaggattggaattggcgattcttacgcggaaccacgataacgagataacgttaagtcgctagacttaaggcgggataaaaggcgtacataattcttgtgtctactgtacagaatactgccgccagctggatttcccaattctgagtaacactctgcaatccaaacagggttcacacgcgatcgagcaactcctcactacgataggtgagcgcagtccagtgtagctcagtcggccaattacggttctattagacagttgcacgttgacatcac

SEQ ID NO: 49 - DNA, 250 bp, Artificial Sequence
  source: /note=“Description of Artificial Sequence Synthetic polynucleotide”
ggagtgcttagcgcgtgattactgctggaggattggaattggcgattcttacgcggaaccacgataacgagataagataaaaggcgtacataattcttgtgtctactgtacagaatactgccgccagctggatttcccaattctgagtaacactctgcaatccaaacagggttcacgttaagtcgctagacttaaggcggcacgcgatcgagcaactcctcactacgataggtgagcgcagtccagtgta

SEQ ID NO: 50 - DNA, 200 bp, Artificial Sequence
  source: /note=“Description of Artificial Sequence Synthetic polynucleotide”
ggagtgcttagcgcgtgattactgctggaggattggaattggcgattcttgataaaaggcgtacataattcttgtgtctactgtacagaatactgccgccagctggatttcccaattctgagtaacactctgcaatccaaacagggttcaacgcggaaccacgataacgagataacgttaagtcgctagacttaaggcgg

SEQ ID NO: 51 - DNA, 150 bp, Artificial Sequence
  source: /note=“Description of Artificial Sequence Synthetic polynucleotide”
ggagtgcttagcgcgtgattactgcgataaaaggcgtacataattcttgtgtctactgtacagaatactgccgccagctggatttcccaattctgagtaacactctgcaatccaaacagggttcatggaggattggaattggcgattctt

SEQ ID NO: 52 - DNA, 142 bp, Artificial Sequence
  source: /note=“Description of Artificial Sequence Synthetic polynucleotide”
gataaaaggcgtacataattcttgtgtctactgtacagaatactgccgccagctggatttcccaattctgagtaacactctgcaatccaaacagggttcaaatttttggagcaggttttctgacttcggtcggaaaacccct

SEQ ID NO: 53 - DNA, 100 bp, Artificial Sequence
  source: /note=“Description of Artificial Sequence Synthetic polynucleotide”
ctggcaacttcaggtgcacgaaaccctggtgtgccctctgatgtttttatccccattctacagcattacggagcagtgccgtagtgtcgtttctttgcaa

SEQ ID NO: 54 - DNA, 100 bp, Artificial Sequence; identical to SEQ ID NO: 53
  source: /note=“Description of Artificial Sequence Synthetic polynucleotide”

SEQ ID NO: 55 - DNA, 200 bp, Artificial Sequence
  source: /note=“Description of Artificial Sequence Synthetic polynucleotide”
ggagtgcttagcgcgtgattactgctggaggattggaattggcgattcttctggcaacttcaggtgcacgaaaccctggtgtgccctctgatgtttttatccccattctacagcattacggagcagtgccgtagtgtcgtttctttgcaaacgcggaaccacgataacgagataacgttaagtcgctagacttaaggcgg

SEQ ID NO: 56 - DNA, 100 bp, Artificial Sequence
  source: /note=“Description of Artificial Sequence Synthetic polynucleotide”
aagtactcacgcagaatctactggccagaagttgatcagagtaacgggaccgcaaaacaaaaaatgaggtggtgaaggagacacacgcaaactcagccgc

SEQ ID NO: 57 - DNA, 100 bp, Artificial Sequence
  source: /note=“Description of Artificial Sequence Synthetic polynucleotide”
ctggttcaaactttggcagtaatgctggattaacaaatgttcatcatctccggaaaataaaatcaaaggttgtggtttgttcccccccttatgttgcttt

SEQ ID NO: 58 - DNA, 100 bp, Artificial Sequence
  source: /note=“Description of Artificial Sequence Synthetic polynucleotide”
gccggcaccattcactcccaggaggccaaagcactctccagtgagaactcggaccacagcctcccgctgctgggctggaggtgcctggataaatcttggt

SEQ ID NO: 59 - DNA, 100 bp, Artificial Sequence
  source: /note=“Description of Artificial Sequence Synthetic polynucleotide”
ccccagtgagcatcttgaatgtggttgtattgccggcaccattcactcccaggaggccaaagcactctccagtgagaactcggaccacagcctcccgctg

SEQ ID NO: 60 - DNA, 146 bp, Artificial Sequence
  source: /note=“Description of Artificial Sequence Synthetic polynucleotide”
ccccagtgagcatcttgaatgtggttgtattgccggcaccattcactcccaggaggccaaagcactctccagtgagaactcggaccacagcctcccgctggtggaatttttggagcaggttttctgacttcggtcggaaaacccct

SEQ ID NO: 61 - DNA, 20 bp, Artificial Sequence
  source: /note=“Description of Artificial Sequence Synthetic oligonucleotide”
tctgcacccatcttcacttc

SEQ ID NO: 62 - DNA, 60 bp, Artificial Sequence
  source: /note=“Description of Artificial Sequence Synthetic oligonucleotide”
tcatatcctgagtcatgtcggaattctgcacccatcttcacttcagagatctcctccgtc

SEQ ID NO: 63 - DNA, 60 bp, Artificial Sequence
  source: /note=“Description of Artificial Sequence Synthetic oligonucleotide”
tcatatcctgagtcatgccggaattctgcacccatcttcacttcagagacctcctccgtc

SEQ ID NO: 64 - RNA, 150 nt, Artificial Sequence
  source: /note=“Description of Artificial Sequence Synthetic polynucleotide”
accgauggguauggccucuaaaaacauggccccagcagcuucagucccuuucucgucgauggucagcacagccuuaugcacggccuggaggggagagaagcagaguggaauuuuuggagcagguuuucugacuucggucggaaaaccccu

SEQ ID NO: 65 - RNA, 150 nt, Artificial Sequence
  source: /note=“Description of Artificial Sequence Synthetic polynucleotide”
accgauggguauggccucuaaaaacauggccccagcagcuucagucccuuacucgucgauggucagcacagccuuaugcacggccuggaggggagagaagcagaguggaauuuuuggagcagguuuucugacuucggucggaaaaccccu

SEQ ID NO: 66 - RNA, 5 nt, Artificial Sequence
  source: /note=“Description of Artificial Sequence Synthetic oligonucleotide”
uaaua

SEQ ID NO: 67 - protein, 6 aa, Artificial Sequence
  source: /note=“Description of Artificial Sequence Synthetic peptide”
  MOD_RES (2)..(2): Any amino acid
KXKEGV

SEQ ID NO: 68 - protein, 9 aa, Artificial Sequence
  source: /note=“Description of Artificial Sequence Synthetic peptide”
VGGAVVTGV

SEQ ID NO: 69 - protein, 6 aa, Artificial Sequence
  source: /note=“Description of Artificial Sequence Synthetic peptide”
DPDNEA

SEQ ID NO: 70 - DNA, 52 bp, Artificial Sequence
  source: /note=“Description of Artificial Sequence Synthetic oligonucleotide”
agagtgttactcagaattgggaaatccagctagcggcagtattctgtacagt

SEQ ID NO: 71 - DNA, 100 bp, Artificial Sequence
  source: /note=“Description of Artificial Sequence Synthetic polynucleotide”
ctggcaacttcaggtgcacgaaaccctggtgtgccctctgatgttattatccccattctacagcattacggagcagtgccgtagtgtcgtttctttgcaa

SEQ ID NO: 72 - DNA, 100 bp, Artificial Sequence; identical to SEQ ID NO: 39
  source: /note=“Description of Artificial Sequence Synthetic polynucleotide”

SEQ ID NO: 73 - DNA, 157 bp, Artificial Sequence
  source: /note=“Description of Artificial Sequence Synthetic polynucleotide”
tttcccaattctgagtaacactctgcaatccaaacagggttcgtggatatactgccatcagtcggcgtggactgtagaaccatgccgactgatggcagataataccgtgataaaaggcgtacataattcttgtgtctactgtacagaatactgccgc

SEQ ID NO: 74 - DNA, 129 bp, Artificial Sequence
  source: /note=“Description of Artificial Sequence Synthetic polynucleotide”
  modified_base (1)..(9): a, c, t, g, unknown or other
nnnnnnnnnggttcgtggatatactgccatcagtcggcgtggactgtagaaccatgccgactgatggcagataataccgtgataaaaggcgtacataattcttgtgtctactgtacagaatactgccgc

SEQ ID NO: 75 - DNA, 198 bp, Artificial Sequence
  source: /note=“Description of Artificial Sequence Synthetic polynucleotide”
tttcccaattctgagtaacactctgcaatccaaacagggttcgtggaatttttggagcaggttttctgacttcggtcggaaaacccctatatactgccatcagtcggcgtggactgtagaaccatgccgactgatggcagataataccgtgataaaaggcgtacataattcttgtgtctactgtacagaatactgccg

SEQ ID NO: 76 - DNA, 170 bp, Artificial Sequence
  source: /note=“Description of Artificial Sequence Synthetic polynucleotide”
  modified_base (1)..(8): a, c, t, g, unknown or other
  variation (1)..(12): replace “ ”
  SITE (1)..(170): /note=“Variant nucleotides given in the sequence have no preference with respect to those in the annotations for variant positions”
nnnnnnnncgggttcgtggatttttggagcaggttttctgacttcggtcggaaaacccctatatactgccatcagtcggcgtggactgtagaaccatgccgactgatggcagataataccgtgataaaaggcgtacataattcttgtgtctactgtacagaatactgccg

SEQ ID NO: 77 - DNA, 42 bp, Artificial Sequence
  source: /note=“Description of Artificial Sequence Synthetic oligonucleotide”
aatttttggagcaggttttctgacttcggtcggaaaacccct

SEQ ID NO: 78 - DNA, 57 bp, Artificial Sequence
  source: /note=“Description of Artificial Sequence Synthetic oligonucleotide”
aatttttggagcaggttttctgacttcggtcggaaaacccctatatactgccatcag

SEQ ID NO: 79 - DNA, 63 bp, Artificial Sequence
  source: /note=“Description of Artificial Sequence Synthetic oligonucleotide”
aatttttggagcaggttttctgacttcggtcggaaaacccctagttttttttactgccatcag

SEQ ID NO: 80 - DNA, 73 bp, Artificial Sequence
  source: /note=“Description of Artificial Sequence Synthetic oligonucleotide”
aatttttggagcaggttttctgacttcggtcggaaaacccctagacactctttccctacacgactgccatcag

SEQ ID NO: 81 - DNA, 15 bp, Artificial Sequence
  source: /note=“Description of Artificial Sequence Synthetic oligonucleotide”
atatactgccatcag

SEQ ID NO: 82 - DNA, 57 bp, Artificial Sequence
  source: /note=“Description of Artificial Sequence Synthetic oligonucleotide”
aatttgtggagcaggctttctgacttcggtcggaaagcccctatatactgccatcag

SEQ ID NO: 83 - DNA, 64 bp, Artificial Sequence
  source: /note=“Description of Artificial Sequence Synthetic oligonucleotide”
aatttgtggagcaggctttctgacttcggtcggaaagcccctagaaaaaaaatactgccatcag

SEQ ID NO: 84 - DNA, 73 bp, Artificial Sequence
  source: /note=“Description of Artificial Sequence Synthetic oligonucleotide”
aatttgtggagcaggctttctgacttcggtcggaaagcccctagacactctttccctacacgactgccatcag

SEQ ID NO: 85 - protein, 5 aa, Artificial Sequence
  source: /note=“Description of Artificial Sequence Synthetic peptide”
GGGGS